(19) United States
(12) Patent Application Publication
(10) Pub. No.: US 2011/0178104 A1
(43) Pub. Date: Jul. 21, 2011

(54) Anti-Heparin Compounds

(75) Inventors: Richard W. Scott, Radnor, PA (US); Dahui Liu, Radnor, PA (US); Robert W. Kavash, Radnor, PA (US); Trevor Young, Radnor, PA (US); Michael J. Costanzo, Radnor, PA (US); Carol Ann Mulrooney, Auburn, MA (US)

(73) Assignee: POLYMEDIX INC., Radnor, PA (US)

(21) Appl. No.: 12/984,634

(22) Filed: Jan. 5, 2011

Related U.S. Application Data
(60) Provisional application No. 61/419,626, filed on Dec. 3, 2010.
(60) Provisional application No. 61/419,617, filed on Dec. 3, 2010.
(60) Provisional application No. 61/293,073, filed on Jan. 7, 2010.

(51) Int. Cl. A61K 31/505; A61K 31/4545; A61K 31/445; A61K 31/455; A61K 31/4045; A61K 31/195; A61K 31/167; C07D 239/28; C07D 401/12; C07D 211/60; C07D 213/56; C07D 209/18; C07C 229/00; C07C 233/81; A61P 7/02

(52) U.S. Cl. 514/256; 514/316; 514/330; 514/356; 514/419; 514/563; 514/616; 544/335; 546/189; 546/226; 546/316; 548/495; 562/442; 564/153

(57) ABSTRACT

The present invention provides compounds and methods for antagonizing the anticoagulant effect of an anticoagulant agent that is selected from UFH, LMWH, and a heparin/LMWH derivative in a patient comprising administering to the patient a compound of the invention or a salt thereof, or a composition comprising the same.

### CROSS-REFERENCE TO RELATED APPLICATIONS

This application claims priority to 1) U.S. provisional application Ser. No. 61/419,626 filed Dec. 3, 2010, 2) U.S. provisional application Ser. No. 61/419,617 filed Dec. 3, 2010, and 3) U.S. provisional application Ser. No. 61/293,073 filed Jan. 7, 2010, each of which is incorporated herein by reference in its entirety.

### REFERENCE TO GOVERNMENT GRANTS

The present invention was supported by funds from the U.S. Government (NIH/NHLBI SBIR Grant Nos. 1R43HL090113-01 and 2R44HL090113-02 and NHLBI SBIR Phase 2 Grant #5R44HL090113) and the U.S. Government may therefore have certain rights in the invention.

### FIELD OF THE INVENTION

The present invention is directed, in part, to compounds or pharmaceutically acceptable salts thereof, and compositions comprising the compounds and/or salts, and methods of antagonizing anticoagulant agents, such as unfractionated heparin, low molecular weight heparin, and/or a derivative of heparin or low molecular weight heparin, with one or more of the compounds, or pharmaceutically acceptable salts thereof, or compositions comprising the same.

### BACKGROUND OF THE INVENTION

Treatment and prevention of thrombosis are major clinical issues for medical and surgical patients. Heparin, a highly sulfated polysaccharide, is commonly used as prophylaxis against venous thromboembolism and to treat venous thrombosis, pulmonary embolism, unstable angina and myocardial infarction (see, for example, Walenga et al., “Factor Xa inhibition in mediating antithrombotic actions: application of a synthetic heparin pentasaccharide” In. Paris: Universite Pierre et Marie Curie, Paris VI; 1987; and Hirsh et. al., Chest, 2001, 119, 64-94). Heparin is also used as an anticoagulant during the extracorporeal blood circulation for kidney dialysis and coronary bypass surgery.

Although heparin is an efficacious anticoagulant, there are many limitations associated with its clinical use. For example, heparin's heterogeneity and polydispersity lead to nonspecific protein binding and poorly predictive pharmacokinetic properties upon subcutaneous (s.c.), and even intravenous, injection (see, for example, Bendetowicz et. al., Thromb. Hemostasis., 1994, 71, 305-313). As a result, infusions of unfractionated heparin (UFH) are performed in the hospital where its anticoagulant effect can be measured to minimize the risk of bleeding. In addition to hemorrhage, administration of UFH is associated with 1-2% incidence of heparin-induced thrombocytopenia (HIT) (see, for example, Morabia, Lancet, 1986, 1, 1278-1279; Mureebe et. al., Vasc. Endovasc. Surg., 2002, 36, 163-170; and Lubenow et. al., Chest, 2002, 122, 37-42).

To address some of the shortcomings of UFH, low molecular weight heparins (LMWHs) have been developed. LMWHs are fragments of UFH produced by chemical or enzymatic depolymerization (see, for example, Hirsh et. al., Blood, 1992, 79, 1-17). Due to their smaller size and lower polydispersity, LMWHs are more reproducibly bioavailable after s.c. administration and have more predictable pharmacokinetics leading to greater safety (see, for example, Ofosu et. al., “Mechanisms of action of low molecular weight heparins and heparinoids.” In: Hirsh J (ed). Antithrombotic Therapy, Bailliere's Clinical Haematology (Volume 3). London, UK: Bailliere Tindall, 1990, pp. 505-529). The smaller size of LMWHs is also associated with a lower ratio of anti-thrombin to anti-FXa activity (see, for example, Hirsh et. al., Chest, 2001, 119, 64-94). LMWHs are being used with greater frequency owing to their ease of administration, longer duration or action and reduced incidence of heparin-induced thrombocytopenia (see, for example, Hirsh et. al., Chest, 2004, 126 (Suppl 3), 188S-203S). LMWHs are commonly used to treat deep vein thrombosis, unstable angina, and acute pulmonary embolism, as well as thromboprophylactic agents in a wide range of clinical situations including orthopedic surgery, high risk pregnancy, and cancer therapy (see, for example, Hirsh et. al., Chest, 2004, 126 (Suppl 3), 188S-203S; Becker, J. Thrombosis and Thrombolysis, 1999, 7, 195; Antman et. al., Circulation, 1999, 100, 1593-601; Cohen et. al., New England J. Med., 1997, 337, 447; and Lee et. al., J Clin. Oncol., 2005, 23, 2123-9).

Fondaparinux is a heparin-derived pentasaccharide that represents the smallest fragment of heparin that is capable of accelerating antithrombin-mediated factor Xa inhibition (see, for example, Walenga et. al., Exp. Opin. Invest. Drugs, 2005, 14, 847-58). Fondaparinux is currently approved for the prophylaxis of deep vein thrombosis following hip repair and/or replacement, knee replacement and abdominal surgery and the treatment of DVT/PE when used in conjunction with warfarin. The most common complication of anticoagulation with LMWHs is hemorrhage. Many published clinical studies report 1% to 4% major (life-threatening) bleeding associated with LMWH therapy and there is a 5-fold increase in the overall death rate for acute coronary syndrome patients receiving anti-coagulant therapy that experience major bleeding (see, for example, Hirsh et. al., Chest, 2001, 119, 64-94; and Mehta et. al., J. Am. Coll. Cardiol., 2007, 50, 1742-1751).

Protamine, an arginine-rich heterogeneous peptide mixture isolated from fish sperm, is used routinely to neutralize the effects of heparin in patients who bleed while under treatment (see, for example, Ando et. al., in Kleinzeller, A. (ed): “Protamine: Molecular biology, biochemistry and biophysics” Vol 12. 1973. New York, Springer-Verlag, 1-109). Polycationic protamine binds to anionic heparin through electrostatic interactions, thereby neutralizing the anticoagulant effects of heparin. Although protamine is commonly used to neutralize UFH following coronary bypass surgery, it is unable to completely reverse the anticoagulant effects of LMWHs (see, for example, Hubbard et. al., Thromb. Haemost., 1985, 53, 86-89; Poon et. al., Thromb. Haemost., 1982, 47, 162-165; Massonnet-Castel et. al., Haemostasis, 1986, 16, 139-146; and Doutremepuich et. al., Semin. Thromb. Hemost., 1985, 11, 318-322) or fondaparinux (see, for example, Walenga, “Factor Xa inhibition in mediating antithrombotic actions: application of a synthetic heparin pentasaccharide” In. Paris: Universite Pierre et Marie Curie, Paris VI; 1987).

In addition, use of protamine for heparin reversal is associated with adverse reactions including systemic vasodilation and hypotension, bradycardia, pulmonary artery hypertension, pulmonary vasoconstriction, thrombocytopenia, and neutropenia (see, for example, Metz et. al., “Protamine and newer heparin antagonists” in Stoetling, R. K. (ed): Pharmacology and Physiology in Anesthetic Practice. Vol. 1. Philadelphia, Pa., JB Lippincott, 1-15, 1994; Weiler et. al., J. Allergy Clin. Immunol., 1985, 75, 297-303; Horrow, Anest. Analg., 1985, 64, 348-361; and Porsche et. al., Heart Lung J. Acute Crit. Care, 1999, 28, 418-428).

Therefore, there is a strong medical need for the development of a safe and effective antagonist for UFH and/or LMWH. The lack of an effective antagonist has limited the clinical use of the LMWHs and fondaparinux, especially in bypass procedures and instances where near term surgical procedures may be needed. There is also a strong medical need for an efficacious, nontoxic substitute for protamine. Further, efficacy against the anticoagulation properties of the LMWHs would substantially address an important and expanding medical market for which no effective antidote is available.

### SUMMARY OF THE INVENTION

The present invention provides, in part, compounds and methods for antagonizing an anticoagulant agent (including, for example, unfractionated heparin, low molecular weight heparin, and synthetically modified heparin or low molecular heparin derivatives) comprising administering to a mammal a compound of Formula I, Ia, Ia-1, Ia-2, Ia-3, II, IIa, III, IV, or V:

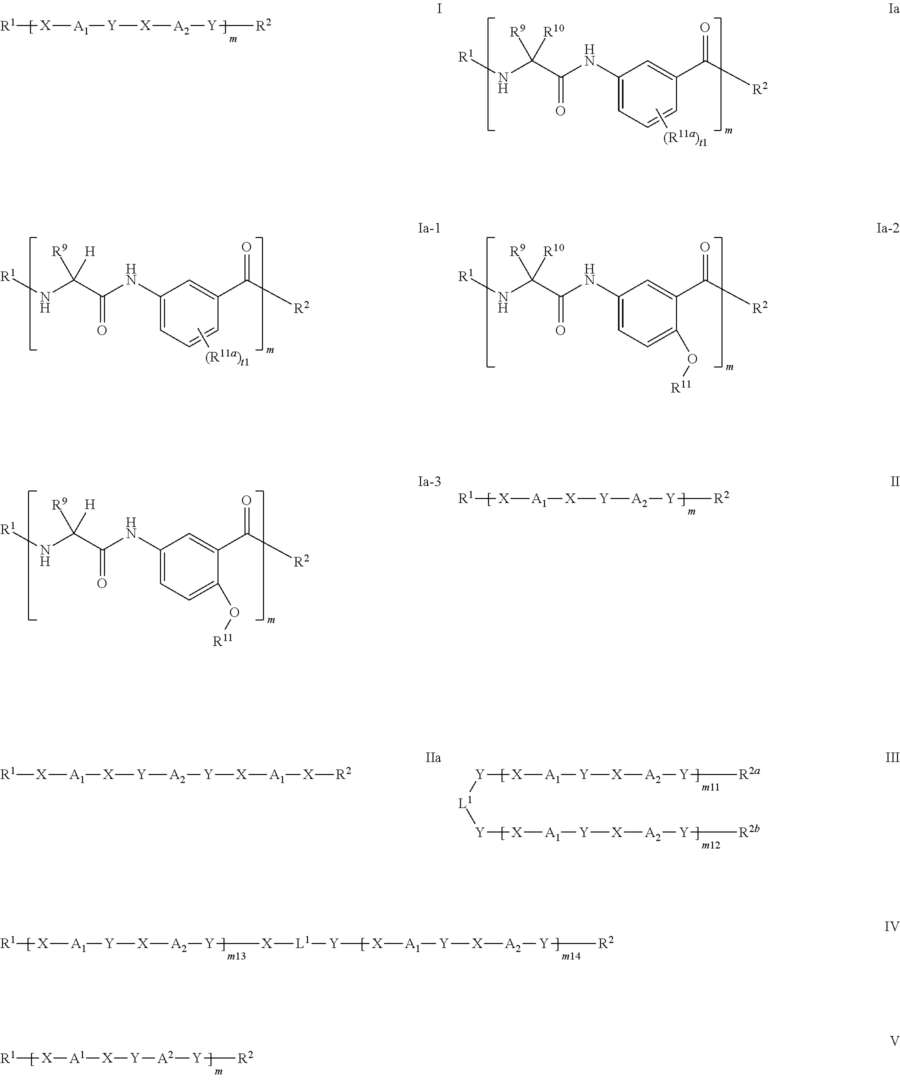

or pharmaceutically acceptable salt thereof, wherein the constituents are as defined below.

In some embodiments, the methods of the present invention can effectively antagonize unfractionated heparin. In some embodiments, the methods of the present invention can effectively antagonize low molecular weight heparin such as enoxaparin, reviparin, or tinzaparin. In some embodiments, the methods of the present invention can effectively antagonize a derivative of heparin or LMWH (for example, a synthetically modified heparin derivative, such as fondaparinux). In some embodiments, the methods of the present invention can effectively antagonize a synthetically modified heparin derivative, such as fondaparinux. In some embodiments, the methods of the present invention can rapidly antagonize the anticoagulant agent (including, for example, unfractionated heparin, low molecular weight heparin, and synthetically modified heparin or low molecular heparin derivatives). In some embodiments, the methods of the present invention can completely eliminate the anticoagulant effect of the anticoagulant agent (including, for example, unfractionated heparin, low molecular weight heparin, and synthetically modified heparin or low molecular heparin derivatives). In some embodiments, after the anticoagulant agent (including, for example, unfractionated heparin, low molecular weight heparin, and synthetically modified heparin or low molecular heparin derivatives) in a patient during anticoagulant therapy is antagonized (for example, completely eliminated) by any of the methods of the present invention, a new dose of an anticoagulant agent (including, for example, unfractionated heparin, low molecular weight heparin, and synthetically modified heparin or low molecular heparin derivatives) can effectively restore the anticoagulant therapy.

In some embodiments, the present invention provides methods for antagonizing an anticoagulant agent (including, for example, unfractionated heparin, low molecular weight heparin, and synthetically modified heparin or low molecular heparin derivatives) with low or no toxicity, hemodynamic and/or hematological adverse side effects. In some embodiments, the methods of the present invention have low or no side effects associated with use of protamine, such as systemic vasodilation and hypotension, bradycardia, pulmonary artery hypertension, pulmonary vasoconstriction, thrombocytopenia and neutropenia. In some embodiments, the methods of the present invention have low or no side effects associated with use of protamine, such as anaphylactic-type reactions involving both nonimmunogenic and immunogenic-mediated pathways. In some embodiments, the compounds and/or the salts used in the present invention have low or no antigenicity and/or immunogenicity compared to protamine compounds. In some embodiments, the present methods for antagonizing heparin (including, for example, unfractionated heparin, low molecular weight heparin, and synthetically modified heparin or low molecular heparin derivatives) can preserve hemodynamic stability, such as during and/or following infusion.

In some embodiments, the present methods for antagonizing an anticoagulant agent (including, for example, unfractionated heparin, low molecular weight heparin, and synthetically modified heparin or low molecular heparin derivatives) can be used in a patient who receives anticoagulant therapy, for example, uses fondaparinux for the prophylaxis of deep vein thrombosis following hip repair/replacement, knee replacement and abdominal surgery; or uses UFH or LMWH for coronary bypass surgery.

The present invention also provides novel compounds, such as compounds of Formula I, III, IV, or V, or pharmaceutically acceptable salts thereof, wherein the constituents are as defined below, and pharmaceutical compositions comprising one or more such compounds or salts thereof.

The present invention also provides novel compounds of Formula I, III, IV, or V (see Formulas above), or pharmaceutically acceptable salts thereof, wherein the constituents are as defined below, and compositions comprising one or more such compounds or salts thereof that can be administered for antagonizing an anticoagulant agent.

The present invention is also directed to use of the compounds and compositions of the invention in the preparation of medicaments for antagonizing an anticoagulant agent (including, for example, unfractionated heparin, low molecular weight heparin, and synthetically modified heparin or low molecular heparin derivatives).

### BRIEF DESCRIPTION OF THE DRAWINGS

FIG. 1 shows results for the neutralization of UFH activity in aPTT assays at a 1 mg/kg dose.

FIG. 2 shows results for neutralizing enoxaparin in both aPTT and factor Xa assays.

FIG. 3 shows results for neutralization of anti-FXa and extended bleeding times caused by enoxaparin.

FIG. 4 shows results from in vivo neutralization of fondaparinux in the rat.

FIG. 5 shows results from the mitigation of hemodynmic responses in the anesthetized rat.

### DESCRIPTION OF EMBODIMENTS

The present invention provides, in part, compounds and methods for antagonizing an anticoagulant agent (such as heparin including, for example, unfractionated heparin, low molecular weight heparin, and synthetically modified heparin or low molecular heparin derivatives) comprising administering to a mammal a compound of Formula I:

R¹—[—X-A₁-Y—X-A₂-Y—]ₘ—R² I

or pharmaceutically acceptable salt thereof, wherein:

each X is, independently, NR⁸, —N(R⁸)N(R⁸)—, O, or S;

each Y is, independently, C═O, C═S, O═S═O, —C(═O)C(═O)—, or —CR^{a}R^{b}—;

R^{a} and R^{b} are each, independently, hydrogen, a PL group, or an NPL group;

each R⁸ is, independently, hydrogen or alkyl;

A₁ and A₂ are each, independently, optionally substituted arylene or optionally substituted heteroarylene, wherein A₁ and A₂ are, independently, optionally substituted with one or more PL group(s), one or more NPL group(s), or a combination of one or more PL group(s) and one or more NPL group(s); or

each A₁ is, independently, optionally substituted arylene or optionally substituted heteroarylene, and each A₂ is a C₃ to C₈ cycloalkyl or —(CH₂)_{q}—, wherein q is 1 to 7, wherein A₁ and A₂ are, independently, optionally substituted with one or more PL group(s), one or more NPL group(s), or a combination of one or more PL group(s) and one or more NPL group(s); or

each A₂ is optionally substituted arylene or optionally substituted heteroarylene, and each A₁ is a C₃ to C₈ cycloalkyl or —(CH₂)_{q}—, wherein q is 1 to 7, wherein A₁ and A₂ are each, independently, optionally substituted with one or more PL group(s), one or more NPL group(s), or a combination of one or more PL group(s) and one or more NPL group(s);

R¹ is hydrogen, an amino acid connected by its carbonyl group, a PL group, or an NPL group, and R² is OH, OR⁶⁰⁰, NH₂, NHR⁶⁰⁰, N(R⁶⁰⁰)₂ (where each R⁶⁰⁰ is, independently, unsubstituted alkyl or aryl, or either alkyl or aryl substituted with OH, halo, cyano, nitro, amino, alkoxy, alkylthio, alkylamino, or dialkylamino), an amino acid connected by its amino group, an α amino acid amide connected by its α amino group (compare compound 311 to compound 310), or —X-A₁-Y—R¹¹ wherein R¹¹ is hydrogen, a PL group, or an NPL group; or

R¹ and R² are each, independently, hydrogen, a PL group, or an NPL group; or

R¹ and R² together are a single bond; or

R¹ is —Y-A₂-X—R¹², wherein R¹² is hydrogen, an amino acid connected by its carbonyl group, a PL group, or an NPL group, and R² is hydrogen, an amino acid connected by its amino group, an α amino acid amide connected by its α amino group, a PL group, or an NPL group; or

R¹ is hydrogen or an amino acid connected by its carbonyl group, and R² is OH, OR⁶⁰⁰, NH₂, NHR⁶⁰⁰, N(R⁶⁰⁰)₂ (where each R⁶⁰⁰ is, independently, unsubstituted alkyl or aryl, or either alkyl or aryl substituted with OH, halo, cyano, nitro, amino, alkoxy, alkylthio, alkylamino, or dialkylamino) an amino acid connected by its amino group, or an α amino acid amide connected by its α amino group;

each NPL group is, independently, —B(OR⁴)₂ or —(NR^{3′})_{q1NPL}—U^{NPL}-LK^{NPL}—(NR^{3″})_{q2NPL}—R^{4′}, wherein:

R³, R^{3′}, and R^{3″} are each, independently, hydrogen, alkyl, or alkoxy;

R⁴ and R^{4′} are each, independently, hydrogen, alkyl, alkenyl, alkynyl, cycloalkyl, aryl, or heteroaryl, wherein each of the alkyl, alkenyl, alkynyl, cycloalkyl, aryl, and heteroaryl is optionally substituted with one or more substitutents, wherein each substituent is, independently, alkyl, halo, or haloalkyl;

each U^{NPL} is, independently, absent or O, S, S(═O), S(═O)₂, NR³, —C(═O)—, —C(═O)—NR³—, —C(═O)—N═N—NR³—, —C(═O)—NR³—N═N—, —N═N—NR³—, —C(═N—N(R³)₂)—, —C(═NR³)—, —C(═O)O—, —C(═O)S—, —C(═S)—, —O—P(═O)₂O—, —S—C═N—, or —C(═O)—NR³—O—, wherein groups with two chemically nonequivalent termini can adopt both possible orientations;

each LK^{NPL} is, independently, —(CH₂)_{pNPL}— or C₂₋₈ alkenylenyl, wherein each of the —(CH₂)_{pNPL} and C₂₋₈ alkenylenyl is optionally substituted with one or more substituents, wherein each substituent is, independently, amino, hydroxyl, aminoalkyl, hydroxylalkyl, or alkyl;

each pNPL is, independently, an integer from 0 to 8;

q1NPL and q2NPL are each, independently, 0, 1, or 2;

each PL group is, independently, halo, hydroxyethoxymethyl, methoxyethoxymethyl, polyoxyethylene, or —(NR^{5′})_{q1PL}—U^{PL}-LK^{PL}—(NR^{5″})_{q2PL}—V, wherein:

R⁵, R^{5′}, and R^{5″} are each, independently, hydrogen, alkyl, or alkoxy;

each U^{PL} is, independently, absent or O, S, S(═O), S(═O)₂, NR⁵, —C(═O)—, —C(═O)—NR⁵—, —C(═O)—N═N—NR⁵—, —C(═O)—NR⁵—N═N—, —N═N—NR⁵—, —C(═N—N(R⁵)₂)—, —C(═NR⁵)—, —C(═O)O—, —C(═O)S—, —C(═S)—, —O—P(═O)₂O—, —S—C═N—, or —C(═O)—NR⁵—O—, wherein groups with two chemically nonequivalent termini can adopt either of the two possible orientations;

each V is, independently, nitro, cyano, amino, halo, hydroxy, alkoxy, alkylthio, alkylamino, dialkylamino, amido, alkylamido, dialkylamido, —NH(CH₂)ₚNH₂ wherein p is 1 to 5, —C(═O)NH(CH₂)ₚNH₂ wherein p is 1 to 5, —C(═O)NH(CH₂)ₚNHC(═NH)NH₂ wherein p is 1 to 5, —C(═O)NH(CH₂)ₚNHC(═O)NH₂ wherein p is 1 to 5, —NHC(═O)-alkyl, —N(CH₂CH₂NH₂)₂, diazamino, amidino, guanidino, ureido, carbamoyl, —C(═O)OH, —C(═O)OR^{c}, —C(═O)NH—OH, —O—NH—C(═NH)NH₂, —NH—S(═O)₂OH, S(═O)₂OH, NR^{d}R^{e}, semicarbazone, aryl, cycloalkyl, heterocycloalkyl, or heteroaryl, wherein each of the aryl and cycloalkyl is substituted with one or more substitutents, wherein each of the heterocycloalkyl and heteroaryl is optionally substituted with one or more substituents, and wherein each of the substituents for the aryl, cycloalkyl, heterocycloalkyl, and heteroaryl is, independently, nitro, cyano, amino, halo, hydroxy, alkoxy, alkylthio, alkylamino, dialkylamino, —NH(CH₂)ₚNH₂ wherein p is 1 to 5, —N(CH₂CH₂NH₂)₂, diazamino, amidino, guanidino, ureido, carbamoyl, —C(═O)OH, —C(═O)OR^{c}, —C(═O)NH—OH, —O—NH—C(═NH)NH₂, —NH—S(═O)₂OH, S(═O)₂OH, NR^{d}R^{e}, semicarbazone, aminosulfonyl, aminoalkoxy, aminoalkylhio, lower acylamino, or benzyloxycarbonyl;

each R^{c} is, independently, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl, or heterocycloalkylalkyl, each optionally substituted by one or more substitutents, wherein each substituent is, independently, OH, amino, halo, C₁₋₆ alkyl, C₁₋₆ haloalkyl, aryl, arylalkyl, heteroaryl, heteroarylalkyl, cycloalkyl, or heterocycloalkyl;

R^{d} and R^{e} are, independently, H, C₁₋₆ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl, or heterocycloalkylalkyl, wherein each of the C₁₋₆ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl and heterocycloalkylalkyl is optionally substituted by OH, amino, halo, C₁₋₆ alkyl, C₁₋₆haloalkyl, C₁₋₆haloalkyl, aryl, arylalkyl, heteroaryl, heteroarylalkyl, cycloalkyl, or heterocycloalkyl;

or R^{d} and R^{e} together with the N atom to which they are attached form a 4-, 5-, 6-, 7-, or 8-membered heterocycloalkyl;

each LK^{PL} is, independently, —(CH₂)_{pPL}— or C₂₋₈ alkenylenyl, wherein each of the —(CH₂)_{pNPL}— and C₂₋₈ alkenylenyl is optionally substituted with one or more substituents, wherein each substituent is, independently, amino, hydroxyl, aminoalkyl, hydroxylalkyl, or alkyl;

each pPL is, independently, an integer from 0-8;

q1PL and q2PL are each, independently, 0, 1, or 2; and

m is an integer from 1 to about 20.

In some embodiments of the compound of Formula I or pharmaceutically acceptable salt thereof, each X is, independently, NR⁸; each Y is C═O; and each A₂ is optionally substituted arylene or optionally substituted heteroarylene, and each A₁ is a C₃ to C₈ cycloalkyl or —(CH₂)_{q}—, wherein q is 1 to 7, wherein A₁ and A₂ are each, independently, optionally substituted with one or more PL group(s), one or more NPL group(s), or a combination of one or more PL group(s) and one or more NPL group(s). In some embodiments, each A₂ is optionally substituted phenyl, and each A₁ is a —(CH₂)—, wherein A₁ and A₂ are each, independently, optionally substituted with one or more PL group(s), one or more NPL group(s), or a combination of one or more PL group(s) and one or more NPL group(s).

In some embodiments of the compound of Formula I or pharmaceutically acceptable salt thereof, each NPL group is, independently, —(NR^{3′})_{q1NPL}—U^{NPL}-LK^{NPL}—(NR^{3″})_{q2NPL}—R^{4′}, wherein:

R³, R^{3′}, and R^{3″} are each, independently, hydrogen, alkyl, or alkoxy; and

R⁴ and R^{4′} are each, independently, hydrogen, alkyl, alkenyl, alkynyl, cycloalkyl, aryl, or heteroaryl, wherein each of the alkyl, alkenyl, alkynyl, cycloalkyl, aryl, and heteroaryl is optionally substituted with one or more substitutents, wherein each substituent is, independently, alkyl, halo, or haloalkyl.

In some embodiments of the compound of Formula I or pharmaceutically acceptable salt thereof:

each NPL group is, independently, —B(OR⁴)₂, R^{4′}, or OR^{4′}, and

R⁴ and R^{4′} are each, independently, alkyl, alkenyl, alkynyl, cycloalkyl, or aryl, each is optionally substituted with one or more substitutents, wherein each substituent is, independently, alkyl, halo, or haloalkyl.

In some embodiments of the compound of Formula I or pharmaceutically acceptable salt thereof, each NPL group is, independently, R^{4′} or OR^{4′}, and

each R^{4′} is, independently, alkyl, alkenyl, alkynyl, cycloalkyl, or aryl, each is optionally substituted with one or more substitutents, wherein each substituent is, independently, alkyl, halo, or haloalkyl.

In some embodiments of the compound of Formula I or pharmaceutically acceptable salt thereof, each NPL group is, independently, alkyl, alkenyl, alkynyl, cycloalkyl, aryl, or alkoxy, each is optionally substituted with one or more substitutents, wherein each substituent is, independently, alkyl, halo, or haloalkyl. In some embodiments, each NPL group is, independently, alkyl, haloalkyl, alkoxy, or haloalkoxy.

In some embodiments of the compound of Formula I or pharmaceutically acceptable salt thereof, each V is, independently, nitro, cyano, amino, hydroxy, alkoxy, alkylthio, alkylamino, dialkylamino, amido, alkylamido, dialkylamido, —NH(CH₂)ₚNH₂ wherein p is 1 to 5, —N(CH₂CH₂NH₂)₂, diazamino, amidino, guanidino, ureido, carbamoyl, —C(═O)OH, —C(═O)OR^{c}, —C(═O)NH—OH, —O—NH—C(═NH)NH₂, —NH—S(═O)₂OH, S(═O)₂OH, NR^{d}R^{e}, semicarbazone, aryl, heterocycloalkyl, or heteroaryl, wherein the aryl is substituted with one or more substitutents, wherein each of the heterocycloalkyl and heteroaryl is optionally substituted with one or more substituents, and wherein each of the substituents for the aryl, heterocycloalkyl, and heteroaryl is, independently, nitro, cyano, amino, hydroxy, alkoxy, alkylthio, alkylamino, dialkylamino, —NH(CH₂)ₚNH₂ wherein p is 1 to 5, —N(CH₂CH₂NH₂)₂, diazamino, amidino, guanidino, ureido, carbamoyl, —C(═O)OH, —C(═O)OR^{c}, —C(═O)NH—OH, —O—NH—C(═NH)NH₂, —NH—S(═O)₂OH, S(═O)₂OH, NR^{d}R^{e}, semicarbazone, aminosulfonyl, aminoalkoxy, aminoalkylhio, lower acylamino, or benzyloxycarbonyl.

In some embodiments of the compound of Formula I or pharmaceutically acceptable salt thereof, each V is, independently, hydroxy, amino, alkylamino, dialkylamino, amido, alkylamido, dialkylamido, —NH(CH₂)ₚNH₂ wherein p is 1 to 5, —N(CH₂CH₂NH₂)₂, guanidino, amidino, ureido, carbamoyl, —C(═O)OH, —C(═O)OR^{c}, —C(═O)NH—OH, —O—NH—C(═NH)NH₂, —NH—S(═O)₂OH, S(═O)₂OH, NR^{d}R^{e}, a substituted aryl group, heterocycloalkyl, or heteroaryl, wherein each of the heterocycloalkyl and heteroaryl is optionally substituted with one more substituents, wherein each substituent is, independently, alkyl, haloalkyl, alkoxy, haloalkoxy, amino, cyano, nitro, hydroxy, —NH(CH₂)ₚNH₂ wherein p is 1 to 5, —N(CH₂CH₂NH₂)₂, amidino, guanidino, aminosulfonyl, aminoalkoxy, aminoalkylhio, lower acylamino, or benzyloxycarbonyl; and wherein the substituted aryl group is substituted with one more substituents, wherein each substituent is, independently, amino, cyano, nitro, hydroxy, —NH(CH₂)ₚNH₂ wherein p is 1 to 5, —N(CH₂CH₂NH₂)₂, amidino, guanidino, aminosulfonyl, aminoalkoxy, aminoalkylhio, lower acylamino, or benzyloxycarbonyl.

In some embodiments of the compound of Formula I or pharmaceutically acceptable salt thereof, each V is, independently, hydroxy, amino, alkylamino, dialkylamino, amido, alkylamido, dialkylamido, —NH(CH₂)ₚNH₂ wherein p is 1 to 5, —N(CH₂CH₂NH₂)₂, guanidino, amidino, ureido, carbamoyl, —C(═O)OH, —C(═O)OR^{c}, —C(═O)NH—OH, —O—NH—C(═NH)NH₂, —NH—S(═O)₂OH, S(═O)₂OH, NR^{d}R^{e}, a substituted aryl group, heterocycloalkyl, or heteroaryl, wherein each of the heterocycloalkyl and heteroaryl is optionally substituted with one more substituents, wherein each substituent is, independently, amino, cyano, nitro, hydroxy, —NH(CH₂)ₚNH₂ wherein p is 1 to 5, —N(CH₂CH₂NH₂)₂, amidino, guanidino, aminosulfonyl, aminoalkoxy, aminoalkylhio, lower acylamino, or benzyloxycarbonyl; and wherein the substituted aryl group is substituted with one more substituents, wherein each substituent is, independently, amino, cyano, nitro, hydroxy, —NH(CH₂)ₚNH₂ wherein p is 1 to 5, —N(CH₂CH₂NH₂)₂, amidino, guanidino, aminosulfonyl, aminoalkoxy, aminoalkylhio, lower acylamino, or benzyloxycarbonyl.

In some embodiments of the compound of Formula I or pharmaceutically acceptable salt thereof, each V is, independently, hydroxy, amino, alkylamino, dialkylamino, amido, alkylamido, dialkylamido, —NH(CH₂)ₚNH₂ wherein p is 1 to 5, —N(CH₂CH₂NH₂)₂, guanidino, amidino, ureido, carbamoyl, —C(═O)NH—OH, —O—NH—C(═NH)NH₂, —NH—S(═O)₂OH, NR^{d}R^{e}, heterocycloalkyl, or heteroaryl, wherein each of the heterocycloalkyl and heteroaryl is optionally substituted with one more substituents, wherein each substituent is, independently, alkyl, haloalkyl, alkoxy, haloalkoxy, amino, cyano, nitro, hydroxy, —NH(CH₂)ₚNH₂ wherein p is 1 to 5, —N(CH₂CH₂NH₂)₂, amidino, guanidino, aminosulfonyl, aminoalkoxy, aminoalkylhio, lower acylamino, or benzyloxycarbonyl.

In some embodiments of the compound of Formula I or pharmaceutically acceptable salt thereof, each V is, independently, hydroxy, amino, alkylamino, dialkylamino, amido, alkylamido, dialkylamido, —NH(CH₂)ₚNH₂ wherein p is 1 to 5, —N(CH₂CH₂NH₂)₂, guanidino, amidino, ureido, carbamoyl, —C(═O)NH—OH, —O—NH—C(═NH)NH₂, —NH—S(═O)₂OH, NR^{d}R^{e}, heterocycloalkyl, or heteroaryl, wherein each of the heterocycloalkyl and heteroaryl is optionally substituted with one more substituents, wherein each substituent is, independently, amino, cyano, nitro, hydroxy, —NH(CH₂)ₚNH₂ wherein p is 1 to 5, —N(CH₂CH₂NH₂)₂, amidino, guanidino, aminosulfonyl, aminoalkoxy, aminoalkylhio, lower acylamino, or benzyloxycarbonyl.

In some embodiments of the compound of Formula I or pharmaceutically acceptable salt thereof, each V is, independently, hydroxy, amino, alkylamino, amido, alkylamido, arylamino, heteroarylamino, ureido, guanidino, carbamoyl, —C(═O)OH, —C(═O)OR^{c}, —C(═O)NH—OH, —O—NH—C(═NH)NH₂, —NH—S(═O)₂OH, S(═O)₂OH, a 3-8 membered heterocycloalkyl, a 5- to 10-membered heteroaryl, or a 6- to 10-membered substituted aryl, wherein the substituted aryl is substituted with one or more substituents, wherein each substituent is, independently, OH, amino, hydroxylalkyl, or aminoalkyl, and wherein each of the 3-8 membered heterocycloalkyl and the 5- to 10-membered heteroaryl is optionally substituted with one or more substituents, wherein each substituent is, independently, alkyl, haloalkyl, alkoxy, haloalkoxy, amino, cyano, nitro, hydroxy, —NH(CH₂)ₚNH₂ wherein p is 1 to 5, —N(CH₂CH₂NH₂)₂, amidino, guanidino, aminosulfonyl, aminoalkoxy, aminoalkylhio, lower acylamino, or benzyloxycarbonyl.

In some embodiments of the compound of Formula I or pharmaceutically acceptable salt thereof, each V is, independently, hydroxy, amino, alkylamino, amido, alkylamido, arylamino, heteroarylamino, ureido, guanidino, carbamoyl, —C(═O)OH, —C(═O)OR^{c}, —C(═O)NH—OH, —O—NH—C(═NH)NH₂, —NH—S(═O)₂OH, S(═O)₂OH, a 3-8 membered heterocycloalkyl, a 5- to 10-membered heteroaryl, or a 6- to 10-membered substituted aryl, wherein the substituted aryl is substituted with one or more substituents, wherein each substituent is, independently, OH, amino, hydroxylalkyl, or aminoalkyl.

In some embodiments of the compound of Formula I or pharmaceutically acceptable salt thereof, each V is, independently, amino, amido, heteroarylamino, ureido, guanidino, carbamoyl, C(═O)OH, —C(═O)OR^{c}, —C(═O)NH—OH, —O—NH—C(═NH)NH₂, —NH—S(═O)₂OH, S(═O)₂OH, aziridinyl, azetidinyl, pyrrolidinyl, piperidinyl, piperazinyl, morpholino, azepanyl, azocanyl, tetrazolyl, 1,2,4-oxadiazolyl, 1,3,4-oxadiazolyl, imidazolyl, pyridinyl, indolyl, or a substituted phenyl, wherein the substituted phenyl is substituted with one or more substituents, wherein each substituent is, independently, OH or amino.

In some embodiments of the compound of Formula I or pharmaceutically acceptable salt thereof, each V is, independently, amino, amido, alkylamino, dialkylamino, —NH(CH₂)ₚNH₂ wherein p is 1 to 5, —N(CH₂CH₂NH₂)₂, guanidino, amidino, ureido, heterocycloalkyl, or heteroaryl, wherein each of the heterocycloalkyl and heteroaryl is optionally substituted with one more substituents, wherein each substituent is, independently, amino, cyano, nitro, hydroxy, —NH(CH₂)ₚNH₂ wherein p is 1 to 5, —N(CH₂CH₂NH₂)₂, amidino, guanidino, aminosulfonyl, aminoalkoxy, aminoalkylhio, lower acylamino, or benzyloxycarbonyl.

In some embodiments of the compound of Formula I or pharmaceutically acceptable salt thereof, each V is, independently, amino, amido, alkylamino, dialkylamino, —NH(CH₂)ₚNH₂ wherein p is 1 to 5, —N(CH₂CH₂NH₂)₂, guanidino, amidino, ureido, pyrrodinyl, piperidinyl, piperazinyl, 4-methylpiperazinyl, pyridinyl, pyrimidinyl, pyrazinyl, or indolyl. In some embodiments, each V is, independently, amino, alkylamino, dialkylamino, amido, alkylamido, dialkylamido, —NH(CH₂)ₚNH₂ wherein p is 1 to 5, —N(CH₂CH₂NH₂)₂, guanidino, amidino, ureido, or indolyl. In some embodiments of the compound of Formula I or pharmaceutically acceptable salt thereof, each PL group is, independently, halo, hydroxyethoxymethyl, methoxyethoxymethyl, polyoxyethylene, or —(NR^{5′})_{q1PL}—U^{PL}—(CH₂)_{pPL}—(NR^{5″})_{q2PL}—V.

In some embodiments of the compound of Formula I or pharmaceutically acceptable salt thereof:

each PL group is, independently, halo, —(CH₂)_{pPL}—V, O—(CH₂)_{pPL}—V, and S—(CH₂)_{pPL}—V;

each pPL is an integer from 0 to 5; and

each V is, independently, hydroxy, amino, halo, alkylamino, dialkylamino, amido, alkylamido, dialkylamido, —NH(CH₂)ₚNH₂ wherein p is 1 to 5, —N(CH₂CH₂NH₂)₂, guanidino, amidino, ureido, carbamoyl, —C(═O)OH, —C(═O)OR^{c}, —C(═O)NH—OH, —O—NH—C(═NH)NH₂, —NH—S(═O)₂OH, S(═O)₂OH, NR^{d}R^{e}, a substituted aryl group, heterocycloalkyl, or heteroaryl, wherein each of the heterocycloalkyl and heteroaryl is optionally substituted with one more substituents, wherein each substituent is, independently, amino, halo, cyano, nitro, hydroxy, —NH(CH₂)ₚNH₂ wherein p is 1 to 5, —N(CH₂CH₂NH₂)₂, amidino, guanidino, aminosulfonyl, aminoalkoxy, aminoalkylhio, lower acylamino, or benzyloxycarbonyl; and wherein the substituted aryl group is substituted with one more substituents, wherein each substituent is, independently, amino, halo, cyano, nitro, hydroxy, —NH(CH₂)ₚNH₂ wherein p is 1 to 5, —N(CH₂CH₂NH₂)₂, amidino, guanidino, aminosulfonyl, aminoalkoxy, aminoalkylhio, lower acylamino, or benzyloxycarbonyl.

In some embodiments of the compound of Formula I or pharmaceutically acceptable salt thereof:

each PL group is, independently, halo, —(CH₂)_{pPL}—V, O—(CH₂)_{pPL}—V, and S—(CH₂)_{pPL}—V;

each pPL is an integer from 0 to 5; and

each V is, independently, hydroxy, amino, alkylamino, dialkylamino, amido, alkylamido, dialkylamido, —NH(CH₂)ₚNH₂ wherein p is 1 to 5, —N(CH₂CH₂NH₂)₂, guanidino, amidino, ureido, carbamoyl, —C(═O)NH—OH, —O—NH—C(═NH)NH₂, —NH—S(═O)₂OH, NR^{d}R^{e}, heterocycloalkyl, or heteroaryl, wherein each of the heterocycloalkyl and heteroaryl is optionally substituted with one more substituents, wherein each substituent is, independently, amino, halo, cyano, nitro, hydroxy, —NH(CH₂)ₚNH₂ wherein p is 1 to 5, —N(CH₂CH₂NH₂)₂, amidino, guanidino, aminosulfonyl, aminoalkoxy, aminoalkylhio, lower acylamino, or benzyloxycarbonyl.

In some embodiments of the compound of Formula I or pharmaceutically acceptable salt thereof:

each NPL group is, independently, —B(OR⁴)₂, R^{4′}, or OR^{4′},

R⁴ and R^{4′} are each, independently, alkyl, alkenyl, alkynyl, cycloalkyl, or aryl, each is optionally substituted with one or more substitutents, wherein each substituent is, independently, alkyl, halo, or haloalkyl;

each PL group is, independently, halo, —(CH₂)_{pPL}—V, O—(CH₂)_{pPL}—V, or S—(CH₂)_{pPL}—V;

each pPL is an integer from 0 to 5; and

each V is, independently, hydroxy, amino, halo, alkylamino, dialkylamino, amido, alkylamido, dialkylamido, —NH(CH₂)ₚNH₂ wherein p is 1 to 5, —N(CH₂CH₂NH₂)₂, guanidino, amidino, ureido, carbamoyl, —C(═O)OH, —C(═O)OR^{c}, —C(═O)NH—OH, —O—NH—C(═NH)NH₂, —NH—S(═O)₂OH, S(═O)₂OH, NR^{d}R^{e}, a substituted aryl group, heterocycloalkyl, or heteroaryl, wherein each of the heterocycloalkyl and heteroaryl is optionally substituted with one more substituents, wherein each substituent is, independently, amino, halo, cyano, nitro, hydroxy, —NH(CH₂)ₚNH₂ wherein p is 1 to 5, —N(CH₂CH₂NH₂)₂, amidino, guanidino, aminosulfonyl, aminoalkoxy, aminoalkylhio, lower acylamino, or benzyloxycarbonyl; and wherein the substituted aryl group is substituted with one more substituents, wherein each substituent is, independently, amino, halo, cyano, nitro, hydroxy, —NH(CH₂)ₚNH₂ wherein p is 1 to 5, —N(CH₂CH₂NH₂)₂, amidino, guanidino, aminosulfonyl, aminoalkoxy, aminoalkylhio, lower acylamino, or benzyloxycarbonyl.

In some embodiments of the compound of Formula I or pharmaceutically acceptable salt thereof:

each NPL group is, independently, R^{4′} or OR^{4′},

R⁴ and R^{4′} are each, independently, alkyl, alkenyl, alkynyl, cycloalkyl, or aryl, each is optionally substituted with one or more substitutents, wherein each substituent is, independently, alkyl, halo, or haloalkyl; each PL group is, independently, halo, —(CH₂)_{pPL}—V, O—(CH₂)_{pPL}—V, or S—(CH₂)_{pPL}—V;

each pPL is an integer from 0 to 5; and

each V is, independently, hydroxy, amino, alkylamino, dialkylamino, amido, alkylamido, dialkylamido, —NH(CH₂)ₚNH₂ wherein p is 1 to 5, —N(CH₂CH₂NH₂)₂, guanidino, amidino, ureido, carbamoyl, —C(═O)NH—OH, —O—NH—C(═NH)NH₂, —NH—S(═O)₂OH, NR^{d}R^{e}, heterocycloalkyl, or heteroaryl, wherein each of the heterocycloalkyl and heteroaryl is optionally substituted with one more substituents, wherein each substituent is, independently, amino, halo, cyano, nitro, hydroxy, —NH(CH₂)ₚNH₂ wherein p is 1 to 5, —N(CH₂CH₂NH₂)₂, amidino, guanidino, aminosulfonyl, aminoalkoxy, aminoalkylhio, lower acylamino, or benzyloxycarbonyl.

In some embodiments of the compound of Formula I or pharmaceutically acceptable salt thereof, each A₂ is phenyl optionally substituted with one or more substituents, wherein each substituent is, independently, OR^{4′}, halo, O—(CH₂)_{pPL}—V, or S—(CH₂)_{pPL}—V; and each A₁ is a —(CH₂)— group optionally substituted with one or more substituents, wherein each substituent is, independently, alkyl or —(CH₂)_{pPL}—V.

In some embodiments of the compound of Formula I or pharmaceutically acceptable salt thereof:

each A₂ is phenyl optionally substituted with one or more substituents, wherein each substituent is, independently, O-alkyl, halo, or O—(CH₂)_{pPL}—V, wherein pPL is an integer from 1 to 5;

each A₁ is a —(CH₂)— group optionally substituted with one or more substituents, wherein each substituent is, independently, CH₃ or —(CH₂)_{pPL}—V, wherein pPL is an integer from 1 to 5; and

each V is, independently, hydroxy, amino, alkylamino, dialkylamino, amido, alkylamido, dialkylamido, —NH(CH₂)ₚNH₂ wherein p is 1 to 5, —N(CH₂CH₂NH₂)₂, guanidino, amidino, ureido, carbamoyl, —C(═O)OH, —C(═O)OR^{c}, —C(═O)NH—OH, —O—NH—C(═NH)NH₂, —NH—S(═O)₂OH, S(═O)₂OH, NR^{d}R^{e}, a substituted aryl group, a substituted cycloalkyl group, heterocycloalkyl, or heteroaryl, wherein each of the heterocycloalkyl and heteroaryl is optionally substituted with one more substituents, wherein each substituent is, independently, amino, halo, cyano, nitro, hydroxy, —NH(CH₂)ₚNH₂ wherein p is 1 to 5, —N(CH₂CH₂NH₂)₂, amidino, guanidino, aminosulfonyl, aminoalkoxy, aminoalkylhio, lower acylamino, or benzyloxycarbonyl; and wherein each of the substituted aryl group and the substituted cycloalkyl group is substituted with one more substituents, wherein each substituent is, independently, amino, halo, cyano, nitro, hydroxy, —NH(CH₂)ₚNH₂ wherein p is 1 to 5, —N(CH₂CH₂NH₂)₂, amidino, guanidino, aminosulfonyl, aminoalkoxy, aminoalkylhio, lower acylamino, or benzyloxycarbonyl.

In some embodiments of the compound of Formula I or pharmaceutically acceptable salt thereof:

each A₂ is phenyl optionally substituted with one or more substituents, wherein each substituent is, independently, O-alkyl, halo, or O—(CH₂)_{pPL}—V, wherein pPL is an integer from 1 to 5;

each A₁ is a —(CH₂)— group optionally substituted with one or more substituents, wherein each substituent is, independently, CH₃ or —(CH₂)_{pPL}—V, wherein pPL is an integer from 1 to 5; and

each V is, independently, hydroxy, amino, alkylamino, dialkylamino, amido, alkylamido, dialkylamido, —NH(CH₂)ₚNH₂ wherein p is 1 to 5, —N(CH₂CH₂NH₂)₂, guanidino, amidino, ureido, carbamoyl, —C(═O)OH, —C(═O)OR^{c}, —C(═O)NH—OH, —O—NH—C(═NH)NH₂, —NH—S(═O)₂OH, S(═O)₂OH, NR^{d}R^{e}, a substituted aryl group, a substituted cycloalkyl group, heterocycloalkyl, or heteroaryl, wherein each of the heterocycloalkyl and heteroaryl is optionally substituted with one more substituents, wherein each substituent is, independently, amino, halo, cyano, nitro, hydroxy, —NH(CH₂)ₚNH₂ wherein p is 1 to 5, —N(CH₂CH₂NH₂)₂, amidino, guanidino, aminosulfonyl, aminoalkoxy, aminoalkylhio, lower acylamino, or benzyloxycarbonyl; and wherein each of the substituted aryl group and the substituted cycloalkyl group is substituted with one more substituents, wherein each substituent is, independently, amino, cyano, nitro, hydroxy, —NH(CH₂)ₚNH₂ wherein p is 1 to 5, —N(CH₂CH₂NH₂)₂, amidino, guanidino, aminosulfonyl, aminoalkoxy, aminoalkylhio, lower acylamino, or benzyloxycarbonyl.

In some embodiments of the compound of Formula I or pharmaceutically acceptable salt thereof:

each A₂ is phenyl optionally substituted with one or more substituents, wherein each substituent is, independently, O-alkyl, halo, or O—(CH₂)_{pPL}—V, wherein pPL is an integer from 1 to 5;

each A₁ is a —(CH₂)— group optionally substituted with one or more substituents, wherein each substituent is, independently, CH₃ or —(CH₂)_{pPL}—V, wherein pPL is an integer from 1 to 5; and

each V is, independently, hydroxy, amino, alkylamino, dialkylamino, amido, alkylamido, dialkylamido, —NH(CH₂)ₚNH₂ wherein p is 1 to 5, —N(CH₂CH₂NH₂)₂, guanidino, amidino, ureido, carbamoyl, —C(═O)OH, —C(═O)OR^{c}, —C(═O)NH—OH, —O—NH—C(═NH)NH₂, —NH—S(═O)₂OH, S(═O)₂OH, NR^{d}R^{e}, a substituted aryl group, heterocycloalkyl, or heteroaryl, wherein each of the heterocycloalkyl and heteroaryl is optionally substituted with one more substituents, wherein each substituent is, independently, amino, halo, cyano, nitro, hydroxy, —NH(CH₂)ₚNH₂ wherein p is 1 to 5, —N(CH₂CH₂NH₂)₂, amidino, guanidino, aminosulfonyl, aminoalkoxy, aminoalkylhio, lower acylamino, or benzyloxycarbonyl; and wherein the substituted aryl group is substituted with one more substituents, wherein each substituent is, independently, amino, cyano, nitro, hydroxy, —NH(CH₂)ₚNH₂ wherein p is 1 to 5, —N(CH₂CH₂NH₂)₂, amidino, guanidino, aminosulfonyl, aminoalkoxy, aminoalkylhio, lower acylamino, or benzyloxycarbonyl.

In some embodiments of the compound of Formula I or pharmaceutically acceptable salt thereof:

each A₂ is phenyl optionally substituted with one or more substituents, wherein each substituent is, independently, O—(CH₃); halo, or O—(CH₂)₂—V;

each A₁ is a —(CH₂)— group optionally substituted with one substituent, wherein each substituent is, independently, CH₃, (CH₂)—V, (CH₂)₂—V, (CH₂)₃—V, —(CH₂)₄—V, or —(CH₂)₅—V; and

each V is, independently, hydroxy, amino, alkylamino, arylamino, heteroarylamino, amido, alkylamido, dialkylamido, ureido, guanidino, carbamoyl, amido, alkylamido, —C(═O)OH, —C(═O)OR^{c}, —C(═O)NH—OH, —O—NH—C(═NH)NH₂, —NH—S(═O)₂OH, S(═O)₂OH, a 3-8 membered heterocycloalkyl, a 5- to 10-membered heteroaryl, or a 6- to 10-membered substituted aryl, wherein the substituted aryl is substituted with one or more substituents, wherein each substituent is, independently, OH, amino, hydroxylalkyl, or aminoalkyl.

In some embodiments of the compound of Formula I or pharmaceutically acceptable salt thereof:

each A₂ is phenyl optionally substituted with one or more substituents, wherein each substituent is, independently, O—(CH₃), halo, or O—(CH₂)₂—V;

each A₁ is a —(CH₂)— group optionally substituted with one substituent, wherein each substituent is, independently, CH₃, (CH₂)—V, (CH₂)₃—V, —(CH₂)₄—V, and —(CH₂)₅—V; and

each V is, independently, hydroxyl, amino, amido, heteroarylamino, ureido, guanidino, carbamoyl, C(═O)OH, —C(═O)OR^{c}, —C(═O)NH—OH, —O—NH—C(═NH)NH₂, —NH—S(═O)₂OH, S(═O)₂OH, aziridinyl, azetidinyl, pyrrolidinyl, piperidinyl, piperazinyl, morpholino, azepanyl, azocanyl, tetrazolyl, 1,2,4-oxadiazolyl, 1,3,4-oxadiazolyl, imidazolyl, pyridinyl, indolyl, or a substituted phenyl, wherein the substituted phenyl is substituted with one or more substituents, wherein each substituent is, independently, OH or amino.

In some embodiments of the compound of Formula I or pharmaceutically acceptable salt thereof:

each A₂ is phenyl optionally substituted with one or more substituents, wherein each substituent is, independently, O—(CH₃), halo, or O—(CH₂)₂—V;

each A₁ is a —(CH₂)— group optionally substituted with one substituent, wherein each substituent is, independently, (CH₂)—V, (CH₂)₃—V, —(CH₂)₄—V, and —(CH₂)₅—V; and

each V, is independently, hydroxyl, amino, amido, ureido, guanidino, carbamoyl, or indolyl.

In some embodiments of the compound of Formula I or pharmaceutically acceptable salt thereof:

each A₂ is phenyl optionally substituted with one or more substituents, wherein each substituent is, independently, O—(CH₃), halo, or O—(CH₂)₂—V;

each A₁ is a —(CH₂)— group optionally substituted with one substituent, wherein each substituent is, independently, (CH₂)—V, (CH₂)₃—V, —(CH₂)₄—V, and —(CH₂)₅—V; and

each V, is independently, amino, amido, ureido, guanidino, carbamoyl, or indolyl.

In some embodiments of the compound of Formula I or pharmaceutically acceptable salt thereof:

each A₂ is phenyl optionally substituted with one or more substituents, wherein each substituent is, independently, O—(CH₃), halo, or O—(CH₂)₂—V;

each A₁ is a —(CH₂)— group optionally substituted with one substituent, wherein each substituent is, independently, CH₃, —(CH₂)—V, —(CH₂)₂—V, —(CH₂)₃—V, —(CH₂)₄—V, or —(CH₂)₅—V;

each V is, independently, hydroxyl, amino, amido, heteroarylamino, ureido, guanidino, carbamoyl, C(═O)OH, —C(═O)OR^{c}, —C(═O)NH—OH, —O—NH—C(═NH)NH₂, —NH—S(═O)₂OH, S(═O)₂OH, aziridinyl, azetidinyl, pyrrolidinyl, piperidinyl, piperazinyl, morpholino, azepanyl, azocanyl, tetrazolyl, 1,2,4-oxadiazolyl, 1,3,4-oxadiazolyl, imidazolyl, pyridinyl, indolyl, or a substituted phenyl, wherein the substituted phenyl is substituted with one or more substituents, wherein each substituent is, independently, OH or amino; and

at least one of A₁ is a —(CH₂)— group substituted with one substituent, wherein each substituent is, independently, (CH₂)—V¹, (CH₂)₂—V¹, —(CH₂)₃—V¹, —(CH₂)₄—V¹, or —(CH₂)₅—V¹, wherein V¹ is indolyl.

In some embodiments of the compound of Formula I or pharmaceutically acceptable salt thereof:

R¹ is hydrogen, an amino acid connected by its carbonyl group, —C(═NR³)—NR^{3″}R^{4′}, —C(═O)—(CH₂)_{pNPL}—R^{4′}, —C(═O)—(CH₂)_{pPL}—V, —C(═O)-A₂-NH—C(═O)—(CH₂)_{pPL}—V; or —C(═O)-A₂-NH—C(═O)—(CH₂)_{pNPL}—R^{4′}; and

R² is OH, OR⁶⁰⁰, NH₂, NHR⁶⁰⁰, N(R⁶⁰⁰)₂ (where each R⁶⁰⁰ is, independently, unsubstituted alkyl or aryl, or either alkyl or aryl substituted with OH, halo, cyano, nitro, amino, alkoxy, alkylthio, alkylamino, or dialkylamino), an amino acid connected by its amino group, an α amino acid amide connected by its α amino group (compare compound 311 to compound 310), NH₂, —NH—(CH₂)_{pPL}—V, or —NH-A₁-C(═O)—NH₂.

In some embodiments of the compound of Formula I or pharmaceutically acceptable salt thereof:

R¹ is hydrogen, an amino acid connected by its carbonyl group, —C(═NR³)—NR^{3″}R^{4′}, —C(═O)—(CH₂)_{pNPL}—R^{4′}, —C(═O)—(CH₂)_{pPL}—V, —C(═O)-A₂-NH—C(═O)—(CH₂)_{pPL}—V, or —C(═O)-A₂-NH—C(═O)—(CH₂)_{pNPL}—R^{4′}, wherein each V is, independently, hydroxy, amino, alkylamino, dialkylamino, —NH(CH₂)ₚNH₂ wherein p is 1 to 5, —N(CH₂CH₂NH₂)₂, guanidino, amidino, ureido, carbamoyl, heterocycloalkyl, or heteroaryl, and where R³, R^{3″}, and R^{4′} are each, independently, H or alkyl; and

R² is OH, OR⁶⁰⁰, NH₂, NHR⁶⁰⁰, N(R⁶⁰⁰)₂ (where each R⁶⁰⁰ is, independently, unsubstituted alkyl or aryl, or either alkyl or aryl substituted with OH, halo, cyano, nitro, amino, alkoxy, alkylthio, alkylamino, or dialkylamino), an amino acid connected by its amino group, an α amino acid amide connected by its α amino group (compare compound 311 to compound 310), NH₂, —NH(CH₂)ₚNH₂ wherein p is 1 to 5, —NH—(CH₂)_{pPL}—V, or NH-A₁-C(═O)—NH₂, wherein V is hydroxy, amino, alkylamino, dialkylamino, —NH(CH₂)ₚNH₂ wherein p is 1 to 5, —N(CH₂CH₂NH₂)₂, guanidino, amidino, ureido, carbamoyl, heterocycloalkyl, or heteroaryl.

In some embodiments of the compound of Formula I or pharmaceutically acceptable salt thereof:

R¹ is hydrogen, an amino acid connected by its carbonyl group, —C(═NH)—NH₂, C(═O)—R^{4′}, —C(═O)—(CH₂)_{pPL}—V, —C(═O)-A₂-NH—C(═O)—(CH₂)_{pPL}—V, or —C(═O)-A₂-NH—C(═O)—R^{4′}, wherein each V is, independently, amino, alkylamino, dialkylamino, —NH(CH₂)ₚNH₂ wherein p is 1 to 5, —N(CH₂CH₂NH₂)₂, guanidino, amidino, ureido, carbamoyl, heterocycloalkyl, or heteroaryl, and where R^{4′} is alkyl; and

R² is OH, OR⁶⁰⁰, NH₂, NHR⁶⁰⁰, N(R⁶⁰⁰)₂ (where each R⁶⁰⁰ is, independently, unsubstituted alkyl or aryl, or either alkyl or aryl substituted with OH, halo, cyano, nitro, amino, alkoxy, alkylthio, alkylamino, or dialkylamino), an amino acid connected by its amino group, an α amino acid amide connected by its α amino group (compare compound 311 to compound 310), NH₂, —NH(CH₂)ₚNH₂ wherein p is 1 to 5, —NH—(CH₂)_{pPL}—V, or NH-A₁-C(═O)—NH₂, wherein V is amino, alkylamino, dialkylamino, —NH(CH₂)ₚNH₂ wherein p is 1 to 5, —N(CH₂CH₂NH₂)₂, guanidino, amidino, ureido, or carbamoyl.

In some embodiments of the compound of Formula I or pharmaceutically acceptable salt thereof, m is 3 or 4. In some embodiments, m is 3. In some embodiments, m is 4.

In some embodiments of the compound of Formula I or pharmaceutically acceptable salt thereof, at least one of A₂ group is different from other A₂ groups.

In some embodiments of the compound of Formula I or pharmaceutically acceptable salt thereof, at least one of A₂ group is different from other A₂ groups.

In some embodiments of the compound of Formula I or pharmaceutically acceptable salt thereof, all A₂ groups are the same.

In some embodiments of the compound of Formula I or pharmaceutically acceptable salt thereof, all A₂ groups are the same.

In some embodiments of the compound of Formula I or pharmaceutically acceptable salt thereof, at least one of A₁ group is different from other A₁ groups.

In some embodiments of the compound of Formula I or pharmaceutically acceptable salt thereof, all A₁ groups are the same.

In some embodiments of the compound of Formula I or pharmaceutically acceptable salt thereof, the compound is a compound of Formula Ia:

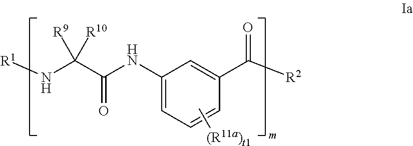

or pharmaceutically acceptable salt thereof, wherein:

each R⁹ is, independently, H, a PL group, or an NPL group;

each R¹⁰ is, independently, H, a PL group, or an NPL group;

or R⁹ and R¹⁰, taken together, constitute the side chain of a D or L α amino acid;

each R^{11a} is, independently, a PL group or an NPL group; and

each t1 is, independently, 0, 1, or 2.

In some embodiments of the compound of Formula Ia or pharmaceutically acceptable salt thereof, each R⁹ is, independently, a PL group or an NPL group. In some embodiments, each R⁹ is, independently, alkyl or (CH₂)_{pPL}—V wherein pPL is an integer from 1 to 5. In some embodiments, each R⁹ is, independently, (CH₂)_{pPL}—V wherein pPL is an integer from 1 to 5. In some embodiments, R⁹ and R¹⁰, taken together, constitute the side chain of a D or L α amino acid.

In some embodiments of the compound of Formula Ia or pharmaceutically acceptable salt thereof, each R¹⁰ is H.

In some embodiments of the compound of Formula Ia or pharmaceutically acceptable salt thereof, each R^{11a} is, independently, halo, alkyl, alkoxy, haloalkyl, haloalkoxy, —(CH₂)_{pPL}—V, —O(CH₂)_{pPL}—V, or —S(CH₂)_{pPL}—V, wherein pPL is an integer from 1 to 5. In some embodiments, each R^{11a} is, independently, halo, alkyl, alkoxy, haloalkyl, or haloalkoxy. In some embodiments, each R^{11a} is, independently, alkoxy. In some embodiments, each R^{11a} is methoxy.

In some embodiments of the compound of Formula Ia or pharmaceutically acceptable salt thereof, the compound is a compound of Formula Ia-1, Ia-2, or Ia-3:

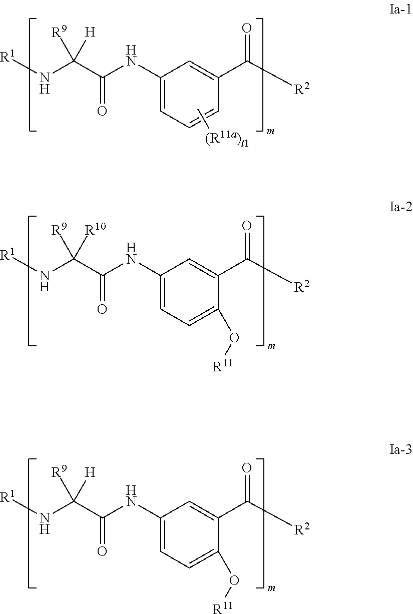

or pharmaceutically acceptable salt thereof, wherein each R¹¹ is, independently, H, alkyl, haloalkyl, or —(CH₂)_{pPL}—V, wherein pPL is an integer from 1 to 5.

In some embodiments of the compound of Formula Ia-2 or Ia-3, or pharmaceutically acceptable salt thereof, each R¹¹ is, independently, alkyl. In some embodiments, each R¹¹ is methyl.

The compounds of Formula I, Ia, Ia-1, Ia-2, or Ia-3 (such as the polymers and oligomers), or salts thereof, useful in the present invention can be made, for example, by methods described in U.S. Patent Application Publication No. 2006-0041023, U.S. Pat. No. 7,173,102, and International Application No. WO 2005/123660. In some embodiments, the compounds of Formula I, Ia, Ia-1, Ia-2, or Ia-3 (such as the polymers and oligomers), or salts thereof, useful in the present invention can be selected from those described in U.S. Patent Application Publication No. 2006-0041023, U.S. Pat. No. 7,173,102, and International Application No. WO 2005/123660. In some embodiments, the compound of Formula I, Ia, Ia-1, Ia-2, or Ia-3 (such as the polymers and oligomers), or salts thereof, useful in the present invention is a compound or salt thereof selected from those described in U.S. Patent Application Publication No. 2006-0041023, U.S. Pat. No. 7,173,102, and International Application No. WO 2005/123660.

In some embodiments, the compound of Formula I, Ia, Ia-1, Ia-2, or Ia-3 (such as the polymers and oligomers), or pharmaceutically acceptable salts thereof, useful in the present invention is a compound selected from Compounds 7-65, 67-72, 76-85, 89, and 90 in Table 1 herein below, or pharmaceutically acceptable salts thereof.

In some embodiments, the present invention provides compounds and methods for antagonizing an anticoagulant agent (including, for example, unfractionated heparin, low molecular weight heparin, and synthetically modified heparin or low molecular heparin derivatives) comprising administering to a mammal a compound of Formula II:

R¹—[—X-A₁-X-Y-A₂-Y—]ₘR² II

or pharmaceutically acceptable salt thereof, wherein:

each X is, independently, NR⁸, O, S, —N(R⁸)N(R⁸)—, —N(R⁸)—(N═N)—, —(N═N)—N(R⁸)—, —C(R⁷R^{7′})NR⁸—, —C(R⁷R^{7′})O—, or —C(R⁷R^{7′})S—;

each Y is, independently, C═O, C═S, O═S═O, —C(═O)C(═O)—, C(R⁶R^{6′})C═O, or C(R⁶R^{6′})C═S;

each R⁸ is, independently, hydrogen or alkyl;

each R⁷ and each R^{7′} are, independently, hydrogen or alkyl; or R⁷ and R^{7′} together form —(CH₂)ₚ—, wherein p is 4 to 8;

each R⁶ and each R^{6′} are, independently, hydrogen or alkyl; or R⁶ and R^{6′} together form —(CH₂)₂NR¹²(CH₂)₂—, wherein R¹² is hydrogen, —C(═N)CH₃, or —C(═NH)—NH₂;

A₁ and A₂ are each, independently, optionally substituted arylene or optionally substituted heteroarylene, wherein A₁ and A₂ are each, independently, optionally substituted with one or more PL group(s), one or more NPL group(s), or a combination of one or more PL group(s) and one or more NPL group(s);

or each A₂ is, independently, optionally substituted arylene or optionally substituted heteroarylene, and each A₁ is, independently, optionally substituted C₃ to C₈ cycloalkyl, wherein A₁ and A₂ are each, independently, optionally substituted with one or more PL group(s), one or more NPL group(s), or a combination of one or more PL group(s) and one or more NPL group(s);

R¹ is hydrogen, a PL group, or an NPL group, and R² is —X-A₁-X—R¹, wherein A₁ is as defined above and is optionally substituted with one or more PL group(s), one or more NPL group(s), or a combination of one or more PL group(s) and one or more NPL group(s); or

R¹ is hydrogen, a PL group, or an NPL group, and R² is —X-A′-X—R¹, wherein A′ is C₃ to C₈ cycloalkyl, aryl, or heteroaryl and is optionally substituted with one or more PL group(s), one or more NPL group(s), or a combination of one or more PL group(s) and one or more NPL group(s); or

R¹ is —Y-A₂-Y—R², and each R² is, independently, hydrogen, a PL group, or an NPL group; or

R¹ is —Y-A′ and R² is —X-A′, wherein each A′ is, independently, C₃ to C₈ cycloalkyl, aryl, or heteroaryl and is optionally substituted with one or more PL group(s), one or more NPL group(s), or a combination of one or more PL group(s) and one or more NPL group(s); or

R¹ and R² are, independently, a PL group or an NPL group; or

R¹ and R² together form a single bond;

each NPL is, independently, —B(OR⁴)₂ or —(NR^{3′})_{q1NPL}—U^{NPL}-LK^{NPL}—(NR^{3″})_{q2NPL}—R^{4′}, wherein:

R³, R^{3′}, and R^{3″} are each, independently, hydrogen, alkyl, or alkoxy;

R⁴ and R^{4′} are each, independently, hydrogen, alkyl, alkenyl, alkynyl, cycloalkyl, aryl, and heteroaryl, wherein each of the alkyl, alkenyl, alkynyl, cycloalkyl, aryl, and heteroaryl is optionally substituted with one or more alkyl or halo groups;

each U^{NPL} is, independently, absent or O, S, S(═O), S(═O)₂, NR³, —C(═O)—, —C(═O)—NR³—, —C(═O)—N═N—NR³—, —C(═O)—NR³—N═N—, —N═N—NR³—, —C(═N—N(R³)₂)—, —C(═NR³)—, —C(═O)O—, —C(═O)S—, —C(═S)—, —O—P(═O)₂O—, —S—C═N—, or —C(═O)—NR³—O—, wherein groups with two chemically nonequivalent termini can adopt both possible orientations;

each LK^{NPL} is, independently, —(CH₂)_{pNPL}— or C₂₋₈ alkenylenyl, wherein each of the —(CH₂)_{pNPL}— and C₂₋₈ alkenylenyl is optionally substituted with one or more substituents, wherein each substituent is, independently, amino, hydroxyl, aminoalkyl, hydroxylalkyl, or alkyl;

each pNPL is, independently, an integer from 0 to 8;

q1NPL and q2NPL are each, independently, 0, 1, or 2;

each PL is, independently, halo, hydroxyethoxymethyl, methoxyethoxymethyl, polyoxyethylene, or —(NR^{5′})_{q1PL}—U^{PL}-LK^{PL}—(NR^{5′})_{q2PL}—V, wherein:

R⁵, R^{5′}, and R^{5″} are each, independently, hydrogen, alkyl, and alkoxy;

each U^{PL} is, independently, absent or O, S, S(═O), S(═O)₂, NR⁵, —C(═O)—, —C(═O)—NR⁵—, —C(═O)—N═N—NR⁵—, —C(═O)—NR⁵—N═N—, —N═N—NR⁵—, —C(═N—N(R⁵)₂)—, —C(═NR⁵)—, —C(═O)O—, —C(═O)S—, —C(═S)—, —O—P(═O)₂O—, —S—C═N—, or —C(═O)—NR⁵—O—, wherein groups with two chemically nonequivalent termini can adopt either of the two possible orientations;

each V is, independently, nitro, cyano, amino, halo, hydroxy, alkoxy, alkylthio, alkylamino, dialkylamino, —NH(CH₂)ₚNH₂ wherein p is 1 to 5, —C(═O)NH(CH₂)ₚNH₂ wherein p is 1 to 5, —C(═O)NH(CH₂)ₚNHC(═NH)NH₂ wherein p is 1 to 5, —C(═O)NH(CH₂)ₚNHC(═O)NH₂ wherein p is 1 to 5, —NHC(═O)-alkyl, —N(CH₂CH₂NH₂)₂, diazamino, amidino, guanidino, ureido, carbamoyl, —C(═O)OH, —C(═O)OR^{c}, —C(═O)NH—OH, —O—NH—C(═NH)NH₂, —NH—S(═O)₂OH, S(═O)₂OH, NR^{d}R^{e}, semicarbazone, aryl, cycloalkyl, heterocycloalkyl, or heteroaryl, wherein each of the aryl and cycloalkyl is substituted with one or more substitutents, wherein each of the heterocycloalkyl, and heteroaryl is optionally substituted with one or more substituents, and wherein each of the substituents for the aryl, cycloalkyl, heterocycloalkyl, and heteroaryl is, independently, nitro, cyano, amino, halo, hydroxy, alkoxy, alkylthio, alkylamino, dialkylamino, —NH(CH₂)ₚNH₂ wherein p is 1 to 5, —N(CH₂CH₂NH₂)₂, diazamino, amidino, guanidino, ureido, carbamoyl, —C(═O)OH, —C(═O)OR^{c}, —C(═O)NH—OH, —O—NH—C(═NH)NH₂, —NH—S(═O)₂OH, S(═O)₂OH, NR^{d}R^{e}, semicarbazone, aminosulfonyl, aminoalkoxy, aminoalkylhio, lower acylamino, or benzyloxycarbonyl;

each LK^{PL} is, independently, —(CH₂)_{pPL}— or C₂₋₈ alkenylenyl, wherein each of the —(CH₂)_{pNPL}— and C₂₋₈ alkenylenyl is optionally substituted with one or more substituents, wherein each substituent is, independently, amino, hydroxyl, aminoalkyl, hydroxylalkyl, or alkyl;

each pPL is, independently, an integer from 0 to 8;

q1PL and q2PL are each, independently, 0, 1, or 2; and

m is an integer from 1 to about 20.

In some embodiments of the compound of Formula II or pharmaceutically acceptable salt thereof, each of the moiety of —Y-A₂-Y— is, independently, a moiety of Formula XI-1, XI-2, or XI-3:

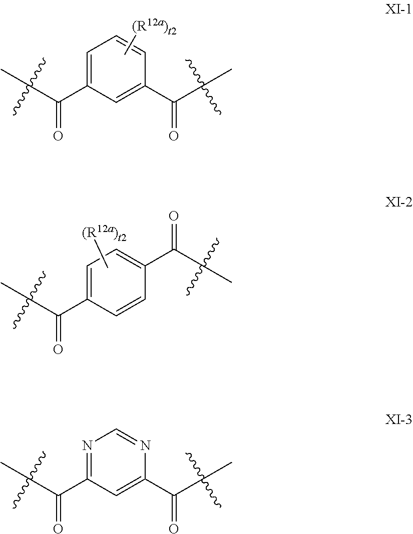

wherein each R^{12a} is, independently, a PL group or an NPL group; and t2 is 0, 1, or 2.

In some embodiments of the compound of Formula II or pharmaceutically acceptable salt thereof, each of the moiety of —Y-A₂-Y— is, independently, a moiety of Formula XI-1 or XI-2; and each R^{12a} is, independently, halo, alkyl, alkoxy, haloalkyl, haloalkoxy, —(CH₂)_{pPL}—V, —O(CH₂)_{pPL}—V, or —S(CH₂)_{pPL}—V, wherein pPL is an integer from 1 to 5. In some embodiments, each R^{12a} is, independently, halo, alkyl, alkoxy, haloalkyl, or haloalkoxy. In some embodiments, each R^{12a} is, independently, alkoxy. In some embodiments, each R^{12a} is methoxy.

In some embodiments of the compound of Formula II or pharmaceutically acceptable salt thereof, each of the moiety of —Y-A₂-Y— is, independently, a moiety of Formula XI-1 or XI-2; and t2 is 2. In some embodiments, each R^{12a} is, independently, alkoxy. In yet further embodiments, each R^{12a} is methoxy.

In some embodiments of the compound of Formula II or pharmaceutically acceptable salt thereof, each of the moiety of —Y-A₂-Y— is, independently, a moiety of Formula XI-1, and the moiety of Formula XI-1 is a moiety of Formula XI-1a:

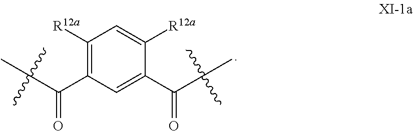

In some embodiments of the compound of Formula II or pharmaceutically acceptable salt thereof, each of the moiety of —X-A₁-X— is, independently, a moiety of Formula XII-1:

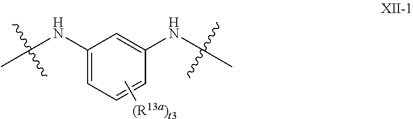

wherein each R^{13a} is, independently, a PL group or an NPL group; and t3 is 0, 1, or 2.

In some embodiments of the compound of Formula II or pharmaceutically acceptable salt thereof, each of the moiety of —X-A₁-X— is, independently, a moiety of Formula XII-2:

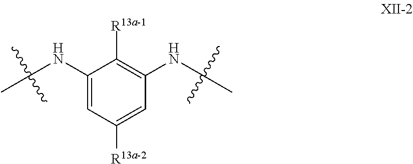

wherein each of R^{13a-1} and R^{13a-2} is, independently, H, a PL group, or an NPL group. In some embodiments, each of R^{13a-1} and R^{13a-2} is, independently, a PL group or an NPL group. In some embodiments, each of R^{13a-1} and R^{13a-2} is, independently, halo, alkyl, haloalkyl, —O(CH₂)_{pPL}—V, or —S(CH₂)_{pPL}—V, wherein pPL is an integer from 1 to 5. In some embodiments, each of R^{13a-1} and R^{13a-2} is, independently, haloalkyl (for example trifluoromethyl) or —S(CH₂)_{pPL}—V, wherein pPL is an integer from 1 to 5.

In some embodiments of the compound of Formula II or pharmaceutically acceptable salt thereof, each of the moiety of —X-A₁-X— is, independently, a moiety of Formula XII-3:

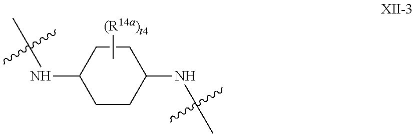

wherein each R^{14a} is, independently, a PL group or an NPL group; and t4 is 0, 1, or 2. In some embodiments, t4 is 0.

In some embodiments of the compound of Formula II or pharmaceutically acceptable salt thereof, each moiety of —Y-A₂-Y— is, independently, a moiety of Formula XI-1, XI-1a, XI-2, or XI-3; and each of the moiety of —X-A₁-X— is, independently, a moiety of Formula XII-1, XII-2, or XII-3.

In some embodiments of the compound of Formula II or pharmaceutically acceptable salt thereof, each moiety of —Y-A₂-Y— is, independently, a moiety of Formula XI-1 or XI-1a; and each of the moiety of —X-A₁-X— is, independently, a moiety of Formula XII-1 or XII-2. In some embodiments, each moiety of —Y-A₂-Y— is, independently, a moiety of Formula XI-1a; and each of the moiety of —X-A₁-X— is, independently, a moiety of Formula XII-2.

In some embodiments of the compound of Formula II or pharmaceutically acceptable salt thereof, each moiety of —Y-A₂-Y— is, independently, a moiety of Formula XI-1, XI-1a, XI-2, or XI-3; and each of the moiety of —X-A₁-X— is, independently, a moiety of Formula XII-3. In some embodiments, each moiety of —Y-A₂-Y— is, independently, a moiety of Formula XI-1a.

In some embodiments, the compound of Formula II or pharmaceutically acceptable salt thereof is a compound of Formula IIa:

R¹—X-A₁-X—Y-A₂-Y—X-A₁-X—R² IIa

or pharmaceutically acceptable salt thereof, wherein:

each X is, independently, NR⁸, O, S, or —N(R⁸)N(R⁸)—;

each Y is, independently, C═O, C═S, or O═S═O;

each R⁸ is, independently, hydrogen or alkyl;

A₁ and A₂ are each, independently, optionally substituted arylene or optionally substituted heteroarylene, wherein A₁ and A₂ are each, independently, optionally substituted with one or more PL group(s), one or more NPL group(s), or a combination of one or more PL group(s) and one or more NPL group(s);

R¹ is a PL group or an NPL group;

R² is R¹;

each NPL is, independently, —(NR^{3′})_{q1NPL}—U^{NPL}-LK^{NPL}—(NR^{3″})_{q2NPL}—R^{4′}, wherein:

R³, R^{3′}, and R^{3″} are each, independently, hydrogen, alkyl, or alkoxy;

R⁴ and R^{4′} are each, independently, hydrogen, alkyl, alkenyl, alkynyl, cycloalkyl, aryl, or heteroaryl, wherein each of the alkyl, alkenyl, alkynyl, cycloalkyl, aryl, and heteroaryl is optionally substituted with one or more alkyl or halo groups;

U^{NPL} is, independently, absent or O, S, S(═O), S(═O)₂, NR³, —C(═O)—, —C(═O)—N═N—NR³—, —C(═O)—NR³—N═N—, —N═N—NR³—, —C(═N—N(R³)₂)—, —C(═NR³)—, —C(═O)O—, —C(═O)S—, —C(═S)—, —O—P(═O)₂O—, —S—C═N—, or —C(═O)—NR³—O—, wherein groups with two chemically nonequivalent termini can adopt either of the two possible orientations;

each LK^{NPL} is, independently, —(CH₂)_{pNPL}— or C₂₋₈ alkenylenyl, wherein the —(CH₂)_{pNPL}— is optionally substituted with one or more substituents, wherein each substituent is, independently, amino, hydroxyl, or alkyl;

each pNPL is, independently, an integer from 0 to 8;

q1NPL and q2NPL are each, independently, 0, 1, or 2;

each PL is, independently, halo, hydroxyethoxymethyl, methoxyethoxymethyl, polyoxyethylene, or —(NR^{5′})_{q1PL}—U^{PL}-LK^{PL}—(NR^{5′})_{q2PL}—V, wherein:

R⁵, R^{5′}, and R^{5″} are each, independently, hydrogen, alkyl, or alkoxy;

each U^{PL} is, independently, absent or O, S, S(═O), S(═O)₂, NR⁵, —C(═O)—, —C(═O)—N═N—NR⁵—, —C(═O)—NR⁵—N═N—, —N═N—NR⁵—, —C(═N—N(R⁵)₂)—, —C(═NR⁵)—, —C(═O)O—, —C(═O)S—, —C(═S)—, —O—P(═O)₂O—, —R⁵⁰—, —R⁵S—, —S—C═N—, or —C(═O)—NR⁵—O—, wherein groups with two chemically nonequivalent termini can adopt both possible orientations;

each V is, independently, nitro, cyano, amino, halo, hydroxy, alkoxy, alkylthio, alkylamino, dialkylamino, —NH(CH₂)ₚNH₂ wherein p is 1 to 5, —N(CH₂CH₂NH₂)₂, diazamino, amidino, guanidino, ureido, carbamoyl, —C(═O)OH, —C(═O)OR^{c}, —C(═O)NH—OH, —O—NH—C(═NH)NH₂, —NH—S(═O)₂OH, S(═O)₂OH, NR^{d}R^{e}, semicarbazone, aryl, heterocycloalkyl, or heteroaryl, wherein the aryl is substituted with one or more substitutents, wherein each of the heterocycloalkyl and heteroaryl is optionally substituted with one or more substituents, and wherein each of each of the substituents for the aryl, heterocycloalkyl, and heteroaryl is, independently, nitro, cyano, amino, halo, hydroxy, alkoxy, alkylthio, alkylamino, dialkylamino, —NH(CH₂)ₚNH₂ wherein p is 1 to 5, —N(CH₂CH₂NH₂)₂, diazamino, amidino, guanidino, ureido, carbamoyl, —C(═O)OH, —C(═O)OR^{c}, —C(═O)NH—OH, O—NH—C(═NH)NH₂, —NH—S(═O)₂OH, S(═O)₂OH, NR^{d}R^{e}, semicarbazone, aminosulfonyl, aminoalkoxy, aminoalkylhio, lower acylamino, or benzyloxycarbonyl;

each LK^{PL} is, independently, —(CH₂)_{pPL}— or C₂₋₈ alkenylenyl, wherein the —(CH₂)_{pNPL}— is optionally substituted with one or more substituents, wherein each substituent is, independently, amino, hydroxyl, or alkyl;

each pPL is, independently, an integer from 0 to 8; and

q1PL and q2PL are each, independently, 0, 1, or 2.

In some embodiments of the compound of Formula II or IIa, or pharmaceutically acceptable salt thereof, each NPL group is, independently, —B(OR⁴)₂, R^{4′}, or OR^{4′}, and R⁴ and R^{4′} are each, independently, alkyl, alkenyl, alkynyl, cycloalkyl, or aryl, each is optionally substituted with one or more substitutents, wherein each substituent is, independently, alkyl, halo, or haloalkyl.

In some embodiments of the compound of Formula II or IIa, or pharmaceutically acceptable salt thereof, each NPL group is, independently, R^{4′} or OR^{4′}, and each R^{4′} is, independently, alkyl, alkenyl, alkynyl, cycloalkyl, or aryl, each is optionally substituted with one or more substitutents, wherein each substituent is, independently, alkyl, halo, or haloalkyl.

In some embodiments of the compound of Formula II or IIa, or pharmaceutically acceptable salt thereof, each NPL group is, independently, alkyl, alkenyl, alkynyl, cycloalkyl, aryl, or alkoxy, each is optionally substituted with one or more substitutents, wherein each substituent is, independently, alkyl, halo, or haloalkyl. In some embodiments, each NPL group is, independently, alkyl, haloalkyl, alkoxy, or haloalkoxy.

In some embodiments of the compound of Formula II or IIa, or pharmaceutically acceptable salt thereof, each V is, independently, nitro, cyano, amino, hydroxy, alkoxy, alkylthio, alkylamino, dialkylamino, —NH(CH₂)ₚNH₂ wherein p is 1 to 5, —N(CH₂CH₂NH₂)₂, diazamino, amidino, guanidino, ureido, carbamoyl, —C(═O)OH, —C(═O)OR^{c}, —C(═O)NH—OH, —O—NH—C(═NH)NH₂, —NH—S(═O)₂OH, S(═O)₂OH, NR^{d}R^{e}, semicarbazone, aryl, heterocycloalkyl, or heteroaryl, wherein the aryl is substituted with one or more substitutents, wherein each of the heterocycloalkyl and heteroaryl is optionally substituted with one or more substituents, and wherein each of each of the substituents for the aryl, heterocycloalkyl, and heteroaryl is, independently, nitro, cyano, amino, hydroxy, alkoxy, alkylthio, alkylamino, dialkylamino, —NH(CH₂)ₚNH₂ wherein p is 1 to 5, —N(CH₂CH₂NH₂)₂, diazamino, amidino, guanidino, ureido, carbamoyl, —C(═O)OH, —C(═O)OR^{c}, —C(═O)NH—OH, —O—NH—C(═NH)NH₂, —NH—S(═O)₂OH, S(═O)₂OH, NR^{d}R^{e}, semicarbazone, aminosulfonyl, aminoalkoxy, aminoalkylhio, lower acylamino, or benzyloxycarbonyl.

In some embodiments of the compound of Formula II or IIa, or pharmaceutically acceptable salt thereof, each V is, independently, hydroxy, amino, alkylamino, dialkylamino, —NH(CH₂)ₚNH₂ wherein p is 1 to 5, —N(CH₂CH₂NH₂)₂, guanidino, amidino, ureido, carbamoyl, —C(═O)OH, —C(═O)OR^{c}, —C(═O)NH—OH, —O—NH—C(═NH)NH₂, —NH—S(═O)₂OH, S(═O)₂OH, NR^{d}R^{e}, a substituted aryl group, heterocycloalkyl, or heteroaryl, wherein each of the heterocycloalkyl and heteroaryl is optionally substituted with one more substituents, wherein each substituent is, independently, alkyl, haloalkyl, alkoxy, haloalkoxy, amino, cyano, nitro, hydroxy, —NH(CH₂)ₚNH₂ wherein p is 1 to 5, —N(CH₂CH₂NH₂)₂, amidino, guanidino, aminosulfonyl, aminoalkoxy, aminoalkylhio, lower acylamino, or benzyloxycarbonyl; and wherein the substituted aryl group is substituted with one more substituents, wherein each substituent is, independently, amino, cyano, nitro, hydroxy, —NH(CH₂)ₚNH₂ wherein p is 1 to 5, —N(CH₂CH₂NH₂)₂, amidino, guanidino, aminosulfonyl, aminoalkoxy, aminoalkylhio, lower acylamino, or benzyloxycarbonyl.

In some embodiments of the compound of Formula II or IIa, or pharmaceutically acceptable salt thereof, each V is, independently, hydroxy, amino, alkylamino, dialkylamino, —NH(CH₂)ₚNH₂ wherein p is 1 to 5, —N(CH₂CH₂NH₂)₂, guanidino, amidino, ureido, carbamoyl, —C(═O)OH, —C(═O)OR^{c}, —C(═O)NH—OH, —O—NH—C(═NH)NH₂, —NH—S(═O)₂OH, S(═O)₂OH, NR^{d}R^{e}, a substituted aryl group, heterocycloalkyl, or heteroaryl, wherein each of the heterocycloalkyl and heteroaryl is optionally substituted with one more substituents, wherein each substituent is, independently, amino, cyano, nitro, hydroxy, —NH(CH₂)ₚNH₂ wherein p is 1 to 5, —N(CH₂CH₂NH₂)₂, amidino, guanidino, aminosulfonyl, aminoalkoxy, aminoalkylhio, lower acylamino, or benzyloxycarbonyl; and wherein the substituted aryl group is substituted with one more substituents, wherein each substituent is, independently, amino, cyano, nitro, hydroxy, —NH(CH₂)ₚNH₂ wherein p is 1 to 5, —N(CH₂CH₂NH₂)₂, amidino, guanidino, aminosulfonyl, aminoalkoxy, aminoalkylhio, lower acylamino, or benzyloxycarbonyl.

In some embodiments of the compound of Formula II or IIa, or pharmaceutically acceptable salt thereof, each V is, independently, hydroxy, amino, alkylamino, dialkylamino, —NH(CH₂)ₚNH₂ wherein p is 1 to 5, —N(CH₂CH₂NH₂)₂, guanidino, amidino, ureido, carbamoyl, —C(═O)NH—OH, —O—NH—C(═NH)NH₂, —NH—S(═O)₂OH, NR^{d}R^{e}, heterocycloalkyl, or heteroaryl, wherein each of the heterocycloalkyl and heteroaryl is optionally substituted with one more substituents, wherein each substituent is, independently, alkyl, haloalkyl, alkoxy, haloalkoxy, amino, cyano, nitro, hydroxy, —NH(CH₂)ₚNH₂ wherein p is 1 to 5, —N(CH₂CH₂NH₂)₂, amidino, guanidino, aminosulfonyl, aminoalkoxy, aminoalkylhio, lower acylamino, or benzyloxycarbonyl.

In some embodiments of the compound of Formula II or IIa, or pharmaceutically acceptable salt thereof, each V is, independently, hydroxy, amino, alkylamino, dialkylamino, —NH(CH₂)ₚNH₂ wherein p is 1 to 5, —N(CH₂CH₂NH₂)₂, guanidino, amidino, ureido, carbamoyl, —C(═O)NH—OH, —O—NH—C(═NH)NH₂, —NH—S(═O)₂OH, NR^{d}R^{e}, heterocycloalkyl, or heteroaryl, wherein each of the heterocycloalkyl and heteroaryl is optionally substituted with one more substituents, wherein each substituent is, independently, amino, cyano, nitro, hydroxy, —NH(CH₂)ₚNH₂ wherein p is 1 to 5, —N(CH₂CH₂NH₂)₂, amidino, guanidino, aminosulfonyl, aminoalkoxy, aminoalkylhio, lower acylamino, or benzyloxycarbonyl.

In some embodiments of the compound of Formula II or IIa, or pharmaceutically acceptable salt thereof, each V is, independently, hydroxy, amino, alkylamino, arylamino, heteroarylamino, ureido, carbamoyl, —C(═O)OH, —C(═O)OR^{c}, —C(═O)NH—OH, —O—NH—C(═NH)NH₂, —NH—S(═O)₂OH, S(═O)₂OH, a 3-8 membered heterocycloalkyl, a 5- to 10-membered heteroaryl, or a 6- to 10-membered substituted aryl, wherein the substituted aryl is substituted with one or more substituents, wherein each substituent is, independently, OH, amino, hydroxylalkyl, or aminoalkyl, and wherein each of the 3-8 membered heterocycloalkyl and the 5- to 10-membered heteroaryl is optionally substituted with one or more substituents, wherein each substituent is, independently, alkyl, haloalkyl, alkoxy, haloalkoxy, amino, cyano, nitro, hydroxy, —NH(CH₂)ₚNH₂ wherein p is 1 to 5, —N(CH₂CH₂NH₂)₂, amidino, guanidino, aminosulfonyl, aminoalkoxy, aminoalkylhio, lower acylamino, or benzyloxycarbonyl.

In some embodiments of the compound of Formula II or IIa, or pharmaceutically acceptable salt thereof, each V is, independently, hydroxy, amino, alkylamino, arylamino, heteroarylamino, ureido, carbamoyl, —C(═O)OH, —C(═O)OR^{c}, —C(═O)NH—OH, —O—NH—C(═NH)NH₂, —NH—S(═O)₂OH, S(═O)₂OH, a 3-8 membered heterocycloalkyl, a 5- to 10-membered heteroaryl, or a 6- to 10-membered substituted aryl, wherein the substituted aryl is substituted with one or more substituents, wherein each substituent is, independently, OH, amino, hydroxylalkyl, or aminoalkyl.

In some embodiments of the compound of Formula II or IIa, or pharmaceutically acceptable salt thereof, each V is, independently, amino, heteroarylamino, ureido, carbamoyl, C(═O)OH, —C(═O)OR^{c}, —C(═O)NH—OH, —O—NH—C(═NH)NH₂, —NH—S(═O)₂OH, S(═O)₂OH, aziridinyl, azetidinyl, pyrrolidinyl, piperidinyl, piperazinyl, morpholino, azepanyl, azocanyl, tetrazolyl, 1,2,4-oxadiazolyl, 1,3,4-oxadiazolyl, imidazolyl, pyridinyl, indolyl, or a substituted phenyl, wherein the substituted phenyl is substituted with one or more substituents, wherein each substituent is, independently, OH or amino.

In some embodiments of the compound of Formula II or IIa, or pharmaceutically acceptable salt thereof, each V is, independently, amino, alkylamino, dialkylamino, —NH(CH₂)ₚNH₂ wherein p is 1 to 5, —N(CH₂CH₂NH₂)₂, guanidino, amidino, ureido, heterocycloalkyl, or heteroaryl, wherein each of the heterocycloalkyl and heteroaryl is optionally substituted with one more substituents, wherein each substituent is, independently, amino, cyano, nitro, hydroxy, —NH(CH₂)ₚNH₂ wherein p is 1 to 5, —N(CH₂CH₂NH₂)₂, amidino, guanidino, aminosulfonyl, aminoalkoxy, aminoalkylhio, lower acylamino, or benzyloxycarbonyl.

In some embodiments of the compound of Formula II or IIa, or pharmaceutically acceptable salt thereof, each V is, independently, amino, alkylamino, dialkylamino, —NH(CH₂)ₚNH₂ wherein p is 1 to 5, —N(CH₂CH₂NH₂)₂, guanidino, amidino, ureido, pyrrodinyl, piperidinyl, piperazinyl, 4-methylpiperazinyl, pyridinyl, pyrimidinyl, pyrazinyl, or indolyl. In some embodiments, each V is, independently, amino, alkylamino, dialkylamino, —NH(CH₂)ₚNH₂ wherein p is 1 to 5, —N(CH₂CH₂NH₂)₂, guanidino, amidino, ureido, or indolyl.

In some embodiments of the compound of Formula II or IIa, or pharmaceutically acceptable salt thereof, each PL is, independently, halo, hydroxyethoxymethyl, methoxyethoxymethyl, polyoxyethylene, and —(NR^{5′})_{pPL}—U^{PL}—(CH₂)_{pPL}—(NR^{5′})_{q2PL}—V.

In some embodiments of the compound of Formula II or IIa, or pharmaceutically acceptable salt thereof:

each PL group is, independently, halo, —(CH₂)_{pPL}—V, O—(CH₂)_{pPL}—V, or S—(CH₂)_{pPL}—V;

each pPL is an integer from 0 to 5; and

each V is, independently, hydroxy, amino, halo, alkylamino, dialkylamino, NH(CH₂)ₚNH₂ wherein p is 1 to 5, —N(CH₂CH₂NH₂)₂, guanidino, amidino, ureido, carbamoyl, —C(═O)OH, —C(═O)OR^{c}, —C(═O)NH—OH, —O—NH—C(═NH)NH₂, —NH—S(═O)₂OH, S(═O)₂OH, NR^{d}R^{e}, a substituted aryl group, heterocycloalkyl, or heteroaryl, wherein each of the heterocycloalkyl and heteroaryl is optionally substituted with one more substituents, wherein each substituent is, independently, amino, halo, cyano, nitro, hydroxy, —NH(CH₂)ₚNH₂ wherein p is 1 to 5, —N(CH₂CH₂NH₂)₂, amidino, guanidino, aminosulfonyl, aminoalkoxy, aminoalkylhio, lower acylamino, or benzyloxycarbonyl; and wherein the substituted aryl group is substituted with one more substituents, wherein each substituent is, independently, amino, halo, cyano, nitro, hydroxy, —NH(CH₂)ₚNH₂ wherein p is 1 to 5, —N(CH₂CH₂NH₂)₂, amidino, guanidino, aminosulfonyl, aminoalkoxy, aminoalkylhio, lower acylamino, or benzyloxycarbonyl.

In some embodiments of the compound of Formula II or IIa, or pharmaceutically acceptable salt thereof:

each PL group is, independently, halo, —(CH₂)_{pPL}—V, O—(CH₂)_{pPL}—V, or S—(CH₂)_{pPL}—V; each pPL is an integer from 0 to 5; and

each V is, independently, hydroxy, amino, alkylamino, dialkylamino, —NH(CH₂)ₚNH₂ wherein p is 1 to 5, —N(CH₂CH₂NH₂)₂, guanidino, amidino, ureido, carbamoyl, —C(═O)NH—OH, —O—NH—C(═NH)NH₂, —NH—S(═O)₂OH, NR^{d}R^{e}, heterocycloalkyl, or heteroaryl, wherein each of the heterocycloalkyl and heteroaryl is optionally substituted with one more substituents, wherein each substituent is, independently, amino, halo, cyano, nitro, hydroxy, —NH(CH₂)ₚNH₂ wherein p is 1 to 5, —N(CH₂CH₂NH₂)₂, amidino, guanidino, aminosulfonyl, aminoalkoxy, aminoalkylhio, lower acylamino, or benzyloxycarbonyl.

In some embodiments of the compound of Formula II or IIa, or pharmaceutically acceptable salt thereof:

each NPL group is, independently, —B(OR⁴)₂, R^{4′}, or OR^{4′},

R⁴ and R^{4′} are each, independently, alkyl, alkenyl, alkynyl, cycloalkyl, or aryl, each is optionally substituted with one or more substitutents, wherein each substituent is, independently, alkyl, halo, or haloalkyl;

each PL group is, independently, halo, —(CH₂)_{pPL}—V, O—(CH₂)_{pPL}—V, or S—(CH₂)_{pPL}—V;

each pPL is an integer from 0 to 5; and

each V is, independently, hydroxy, amino, alkylamino, dialkylamino, —NH(CH₂)ₚNH₂ wherein p is 1 to 5, —N(CH₂CH₂NH₂)₂, guanidino, amidino, ureido, carbamoyl, —C(═O)OH, —C(═O)OR^{c}, —C(═O)NH—OH, —O—NH—C(═NH)NH₂, —NH—S(═O)₂OH, S(═O)₂OH, NR^{d}R^{e}, a substituted aryl group, heterocycloalkyl, or heteroaryl, wherein each of the heterocycloalkyl and heteroaryl is optionally substituted with one more substituents, wherein each substituent is, independently, amino, halo, cyano, nitro, hydroxy, —NH(CH₂)ₚNH₂ wherein p is 1 to 5, —N(CH₂CH₂NH₂)₂, amidino, guanidino, aminosulfonyl, aminoalkoxy, aminoalkylhio, lower acylamino, or benzyloxycarbonyl; and wherein the substituted aryl group is substituted with one more substituents, wherein each substituent is, independently, amino, halo, cyano, nitro, hydroxy, —NH(CH₂)ₚNH₂ wherein p is 1 to 5, —N(CH₂CH₂NH₂)₂, amidino, guanidino, aminosulfonyl, aminoalkoxy, aminoalkylhio, lower acylamino, or benzyloxycarbonyl.

In some embodiments of the compound of Formula II or IIa, or pharmaceutically acceptable salt thereof:

each X is, independently, NR⁸;

each Y is C═O;

A₁ and A₂ are each, independently, phenyl or a 6-membered heteroaryl, each optionally substituted with one or more substituents, wherein each substituent is, independently, alkyl, haloalkyl, halo, —O-alkyl, O—(CH₂)_{pPL}—V, or S—(CH₂)_{pPL}—V;

R¹ is —C(═O)—(CH₂)_{pPL}—V or —C(═O)—(CH₂)_{pNPL}—R^{4′};

R² is R¹;

R^{4′} is H or alkyl; and

each V is, independently, hydroxy, amino, alkylamino, dialkylamino, —NH(CH₂)ₚNH₂ wherein p is 1 to 4, —N(CH₂CH₂NH₂)₂, guanidino, amidino, ureido, carbamoyl, heterocycloalkyl, or heteroaryl.

In some embodiments of the compound of Formula II or IIa, or pharmaceutically acceptable salt thereof:

each X is NH;

each Y is C═O;

each A₁ is, independently, phenyl optionally substituted with one or two substituents, wherein each substituent is, independently, haloalkyl, halo, —O-alkyl, O—(CH₂)_{pPL}—V, or S—(CH₂)_{pPL}—V;

A₂ is phenyl or a 6-membered heteroaryl, each optionally substituted with one or two substituents, wherein each substituent is, independently, —O-alkyl;

R¹ is —C(═O)—(CH₂)_{pPL}—V;

R² is R¹; and

each V is, independently, hydroxy, amino, alkylamino, dialkylamino, —NH(CH₂)ₚNH₂ wherein p is 1 to 5, —N(CH₂CH₂NH₂)₂, guanidino, amidino, ureido, carbamoyl, heterocycloalkyl, or heteroaryl.

In some embodiments of the compound of Formula II or IIa, or pharmaceutically acceptable salt thereof:

each X is NH;

each Y is C═O;

each A₁ is, independently, phenyl optionally substituted with one or two substituents, wherein each substituent is, independently, haloalkyl, O—(CH₂)_{pPL}—V, or

S—(CH₂)_{pPL}—V;

A₂ is phenyl or pyrimidinyl, each optionally substituted with one or two substituents, wherein each substituent is, independently, —O-alkyl;

R¹ is —C(═O)—(CH₂)_{pPL}—V;

R² is R¹; and

each V is, independently, amino, alkylamino, dialkylamino, —NH(CH₂)ₚNH₂ wherein p is 1 to 4, —N(CH₂CH₂NH₂)₂, guanidino, amidino, ureido, carbamoyl, or indolyl.

In some embodiments of the compound of Formula IIa or pharmaceutically acceptable salt thereof, the moiety of —Y-A₂-Y— is a moiety of Formula XI-1, XI-2, or XI-3:

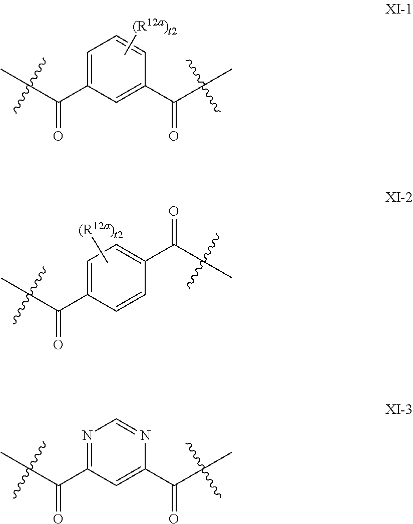

wherein each R^{12a} is, independently, a PL group or an NPL group; and t2 is 0, 1, or 2.

In some embodiments of the compound of Formula IIa or pharmaceutically acceptable salt thereof, the moiety of —Y-A₂-Y— is a moiety of Formula XI-1 or XI-2; and each R^{12a} is, independently, halo, alkyl, alkoxy, haloalkyl, haloalkoxy, —(CH₂)_{pPL}—V, —O(CH₂)_{pPL}—V, or —S(CH₂)_{pPL}—V, wherein pPL is an integer from 1 to 5. In some embodiments, each R^{12a} is, independently, halo, alkyl, alkoxy, haloalkyl, or haloalkoxy. In some embodiments, each R^{12a} is, independently, alkoxy. In some embodiments, each R^{12a} is methoxy.

In some embodiments of the compound of Formula IIa or pharmaceutically acceptable salt thereof, the moiety of —Y-A₂-Y— is a moiety of Formula XI-1 or XI-2; and t2 is 2. In some embodiments, each R^{12a} is, independently, alkoxy. In some embodiments, each R^{12a} is methoxy.

In some embodiments of the compound of Formula IIa or pharmaceutically acceptable salt thereof, the moiety of —Y-A₂-Y— is a moiety of Formula XI-1, and the moiety of Formula XI-1 is a moiety of Formula XI-1a:

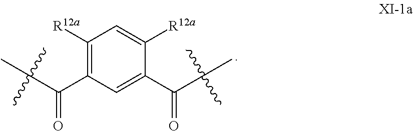

In some embodiments of the compound of Formula IIa or pharmaceutically acceptable salt thereof, each of the moiety of —X-A₁-X— is, independently, a moiety of Formula XII-1:

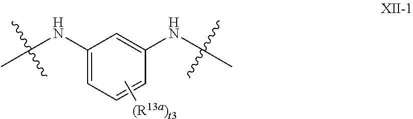

wherein each R^{13a} is, independently, a PL group or an NPL group; and t3 is 0, 1, or 2.

In some embodiments of the compound of Formula IIa or pharmaceutically acceptable salt thereof, each of the moiety of —X-A₁-X— is, independently, a moiety of Formula XII-2:

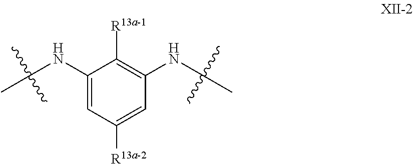

wherein each of R^{13a-1} and R^{13a-2} is, independently, H, a PL group, or an NPL group. In some embodiments, each of R^{13a-1} and R^{13a-2} are, independently, a PL group or an NPL group. In some embodiments, each of R^{13a-1} and R^{13a-2} are, independently, halo, alkyl, haloalkyl, —O(CH₂)_{pPL}—V, or —S(CH₂)_{pPL}—V, wherein pPL is an integer from 1 to 5. In some embodiments, each of R^{13a-1} and R^{13a-2} are, independently, haloalkyl (for example trifluoromethyl) or —S(CH₂)_{pPL}—V, wherein pPL is an integer from 1 to 5.

In some embodiments of the compound of Formula II or pharmaceutically acceptable salt thereof, each A₂ is, independently, optionally substituted arylene or optionally substituted heteroarylene, and each A₁ is, independently, optionally substituted C₃ to C₈ cycloalkyl, wherein A₁ and A₂ are each, independently, optionally substituted with one or more PL group(s), one or more NPL group(s), or a combination of one or more PL group(s) and one or more NPL group(s); R¹ is —Y-A₂-Y—R²; and each R² is, independently, hydrogen, a PL group, or an NPL group. In some embodiments, each X is NH; and each Y is C═O. In some embodiments, m is 1 or 2.

In some embodiments of the compound of Formula II or pharmaceutically acceptable salt thereof, each A₂ is, independently, optionally substituted phenyl, and each A₁ is, independently, optionally substituted C₃ to C₈ cycloalkyl, wherein A₁ and A₂ are each, independently, optionally substituted with one or more PL group(s), one or more NPL group(s), or a combination of one or more PL group(s) and one or more NPL group(s); R¹ is —Y-A₂-Y—R²; and each R² is, independently, hydrogen, a PL group, or an NPL group. In some embodiments, each X is NH; and each Y is C═O. In some embodiments, m is 1 or 2.

In some embodiments of the compound of Formula II or pharmaceutically acceptable salt thereof, each A₁ is, independently, C₅ to C₆ cycloalkyl; each A₂ is, independently, phenyl optionally substituted with one or more PL group(s), one or more NPL group(s), or a combination of one or more PL group(s) and one or more NPL group(s); R¹ is —Y-A₂-Y—R²; and each R² is, independently, hydrogen, a PL group, or an NPL group. In some embodiments, each X is NH; and each Y is C═O. In some embodiments, m is 1 or 2.

In some embodiments of the compound of Formula II or pharmaceutically acceptable salt thereof, each NPL group is, independently, —B(OR⁴)₂, R^{4′}, or OR^{4′}; R⁴ and R^{4′} are each, independently, alkyl, alkenyl, alkynyl, cycloalkyl, or aryl, each is optionally substituted with one or more substitutents, wherein each substituent is, independently, alkyl, halo, or haloalkyl; each PL group is, independently, halo, —(CH₂)_{pPL}—V, O—(CH₂)_{pPL}—V, or S—(CH₂)_{pPL}—V; each pPL is an integer from 0 to 5; and each V is, independently, hydroxy, amino, alkylamino, dialkylamino, —NH(CH₂)ₚNH₂ wherein p is 1 to 5, —N(CH₂CH₂NH₂)₂, guanidino, amidino, ureido, carbamoyl, —C(═O)OH, —C(═O)OR^{c}, —C(═O)NH—OH, —O—NH—C(═NH)NH₂, —NH—S(═O)₂OH, S(═O)₂OH, NR^{d}R^{e}, a substituted aryl group, heterocycloalkyl, and heteroaryl, wherein each of the heterocycloalkyl and heteroaryl is optionally substituted with one more substituents, wherein each substituent is, independently, amino, halo, cyano, nitro, hydroxy, —NH(CH₂)ₚNH₂ wherein p is 1 to 5, —N(CH₂CH₂NH₂)₂, amidino, guanidino, aminosulfonyl, aminoalkoxy, aminoalkylhio, lower acylamino, or benzyloxycarbonyl; and wherein the substituted aryl group is substituted with one more substituents, wherein each substituent is, independently, amino, halo, cyano, nitro, hydroxy, —NH(CH₂)ₚNH₂ wherein p is 1 to 5, —N(CH₂CH₂NH₂)₂, amidino, guanidino, aminosulfonyl, aminoalkoxy, aminoalkylhio, lower acylamino, or benzyloxycarbonyl. In some embodiments, each X is NH; and each Y is C═O. In some embodiments, m is 1 or 2.

In some embodiments of the compound of Formula II or pharmaceutically acceptable salt thereof, each A₁ is C₆ cycloalkyl; each A₂ is, independently, phenyl optionally substituted with one or more substituents, wherein each substituent is, independently, haloalkyl, halo, —O-alkyl, O—(CH₂)_{pPL}—V, or S—(CH₂)_{pPL}—V; R¹ is —Y-A₂-Y—R²; each R² is, independently, NH—(CH₂)_{pPL}—V; and each V is, independently, hydroxy, amino, alkylamino, dialkylamino, —NH(CH₂)ₚNH₂ wherein p is 1 to 5, —N(CH₂CH₂NH₂)₂, guanidino, amidino, ureido, carbamoyl, heterocycloalkyl, or heteroaryl. In some embodiments, each X is NH; and each Y is C═O. In some embodiments, m is 1 or 2.

In some embodiments of the compound of Formula II or pharmaceutically acceptable salt thereof, each A₁ is C₆ cycloalkyl; each A₂ is, independently, phenyl optionally substituted with one or more substituents, wherein each substituent is, independently, haloalkyl, —O-alkyl, O—(CH₂)_{pPL}—V, or S—(CH₂)_{pPL}—V; R¹ is —Y-A₂-Y—R²; each R² is, independently, NH—(CH₂)_{pPL}—V; and each V is, independently, amino, alkylamino, dialkylamino, —NH(CH₂)ₚNH₂ wherein p is 1 to 5, —N(CH₂CH₂NH₂)₂, guanidino, amidino, ureido, carbamoyl, or indolyl. In some embodiments, each X is NH; and each Y is C═O. In some embodiments, m is 1 or 2.

In some embodiments of the compound of Formula II or pharmaceutically acceptable salt thereof, each of the moiety of —Y-A₂-Y— is a moiety of Formula XI-1 or XI-1a:

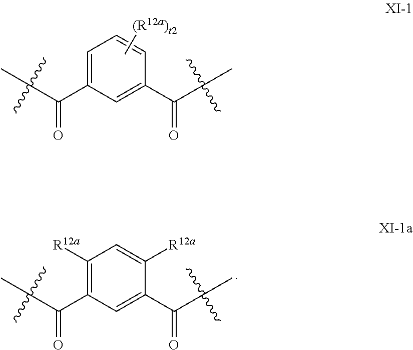

wherein each R^{12a} is, independently, a PL group or an NPL group; and t2 is 0, 1, or 2; and each of the moiety of —X-A₁-X— is, independently, a moiety of Formula XII-3:

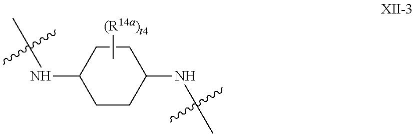

wherein each R^{14a} is, independently, a PL group or an NPL group.

In some embodiments of the compound of Formula II or pharmaceutically acceptable salt thereof, each of the moiety of —Y-A₂-Y— is a moiety of Formula XI-1a, and each of the moiety of —X-A₁-X— is a moiety of Formula XII-3 wherein t4 is 0. In some embodiments, each R^{12a} is, independently, halo, alkyl, alkoxy, haloalkyl, haloalkoxy, —(CH₂)_{pPL}—V, —O(CH₂)_{pPL}—V, or —S(CH₂)_{pPL}—V, wherein pPL is an integer from 1 to 5. In some embodiments, each R^{12a} is, independently, alkoxy or —O(CH₂)_{pPL}—V, wherein pPL is an integer from 1 to 5. In some embodiments, R¹ is —Y-A₂-Y—R²; and each R² is, independently, hydrogen, a PL group, or an NPL group. In some embodiments, m is 1, 2, or 3. In some embodiments, m is 1 or 2.

The compounds of Formula II or IIa (such as the polymers and oligomers) or pharmaceutically acceptable salts thereof useful in the present invention can be made, for example, by methods described in U.S. Patent Application Publication No. 2006-0041023, U.S. Pat. No. 7,173,102, International Publication No. WO 2004/082643, International Publication No. WO2006093813, and U.S. patent application Ser. No. 12/510,593 filed Jul. 28, 2009. In some embodiments, the compounds of Formula II or IIa (such as the polymers and oligomers) or pharmaceutically acceptable salts thereof useful in the present invention can be selected from those described in U.S. Patent Application Publication No. 2006-0041023, U.S. Pat. No. 7,173,102, International Publication No. WO 2004/082643, International Publication No. WO2006093813, and U.S. patent application Ser. No. 12/510,593 filed Jul. 28, 2009. In some embodiments, the compounds of Formula II or IIa (such as the polymers and oligomers) or pharmaceutically acceptable salts thereof useful in the present invention is a compound selected from Compounds 1-3, 5, 6, and 86-88 in Table 1 herein below, or pharmaceutically acceptable salt thereof.

In some embodiments, the compound(s) useful in the method of present invention can be chosen from one or more of the compounds (i.e., genuses, sub-genuses, and species) disclosed in U.S. Patent Application Publication No. 2006-0041023, U.S. Pat. No. 7,173,102, International Publication No. WO 2005/123660, International Publication No. WO 2004/082643, International Publication No. WO 2006/093813, and U.S. patent application Ser. No. 12/510,593 filed Jul. 28, 2009, each of which is hereby incorporated by reference in its entirety.

In some embodiments, the present invention provides compounds and methods for antagonizing an anticoagulant agent (including, for example, unfractionated heparin, low molecular weight heparin, and synthetically modified heparin or low molecular heparin derivatives) comprising administering to a mammal a compound of Formula III:

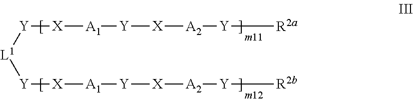

or pharmaceutically acceptable salt thereof, wherein:

each X is, independently, NR⁸;

each Y is C═O;

each R⁸ is, independently, hydrogen or alkyl;

each A₂ is optionally substituted arylene or optionally substituted heteroarylene, and each A₁ is —(CH₂)_{q}—, wherein q is 1 to 7, wherein A₁ and A₂ are each, independently, optionally substituted with one or more PL group(s), one or more NPL group(s), or a combination of one or more PL group(s) and one or more NPL group(s);

R^{2a} and R^{2b} are each, independently, hydrogen, a PL group, an NPL group or —X-A₁-Y—R¹¹, wherein R¹¹ is hydrogen, a PL group, or an NPL group; or

R^{2a} and R^{2b} are as described above for R¹ and R² under Formula I;

L¹ is C₁₋₁₀alkylene optionally substituted with one or more substitutents, wherein each substituent is, independently, alkyl, halo, haloalkyl, aminoalkyl, hydroxylalkyl, V, or —(CH₂)_{pPL}—V, wherein pPL is an integer from 1 to 5;

each NPL group is, independently, —B(OR⁴)₂ or —(NR^{3′})_{q1NPL}—U^{NPL}-LK^{NPL}—(NR^{3″})_{q2NPL}—R⁴, wherein:

R³, R^{3′}, and R^{3″} are each, independently, hydrogen, alkyl, or alkoxy;

R⁴ and R^{4′} are each, independently, hydrogen, alkyl, alkenyl, alkynyl, cycloalkyl, aryl, or heteroaryl, wherein each of the alkyl, alkenyl, alkynyl, cycloalkyl, aryl, and heteroaryl is optionally substituted with one or more substitutents, wherein each substituent is, independently, alkyl, halo, or haloalkyl;

each U^{NPL} is, independently, absent or O, S, S(═O), S(═O)₂, NR³, —C(═O)—, —C(═O)—NR³—, —C(═O)—N═N—NR³—, —C(═O)—NR³—N═N—, —N═N—NR³—, —C(═N—N(R³)₂)—, —C(═NR³)—, —C(═O)O—, —C(═O)S—, —C(═S)—, —O—P(═O)₂O—, —S—C═N—, or —C(═O)—NR³—O—, wherein groups with two chemically nonequivalent termini can adopt both possible orientations;

each LK^{NPL} is, independently, —(CH₂)_{pNPL}— and C₂₋₈ alkenylenyl, wherein each of the —(CH₂)ₚNPL and C₂₋₈ alkenylenyl is optionally substituted with one or more substituents, wherein each substituent is, independently, amino, hydroxyl, aminoalkyl, hydroxylalkyl, or alkyl; each pNPL is, independently, an integer from 0 to 8;

q1NPL and q2NPL are each, independently, 0, 1, or 2;

each PL group is, independently, halo, hydroxyethoxymethyl, methoxyethoxymethyl, polyoxyethylene, or —(NR^{5′})_{q1PL}—U^{PL}-LK^{PL}—(NR^{5″})_{q2PL}—V, wherein:

R⁵, R^{5′}, and R^{5″} are each, independently, hydrogen, alkyl, or alkoxy;

each U^{PL} is, independently, absent or O, S, S(═O), S(═O)₂, NR⁵, —C(═O)—, —C(═O)—NR⁵—, —C(═O)—N═N—NR⁵—, —C(═O)—NR⁵—N═N—, —N═N—NR⁵—, —C(═N—N(R⁵)₂)—, —C(═NR⁵)—, —C(═O)O—, —C(═O)S—, —C(═S)—, —O—P(═O)₂O—, —S—C═N—, or —C(═O)—NR⁵—O—, wherein groups with two chemically nonequivalent termini can adopt either of the two possible orientations;

each V is, independently, nitro, cyano, amino, halo, hydroxy, alkoxy, alkylthio, alkylamino, dialkylamino, —NH(CH₂)ₚNH₂ wherein p is 1 to 5, —C(═O)NH(CH₂)ₚNH₂ wherein p is 1 to 5, —C(═O)NH(CH₂)ₚNHC(═NH)NH₂ wherein p is 1 to 5, —C(═O)NH(CH₂)ₚNHC(═O)NH₂ wherein p is 1 to 5, —NHC(═O)-alkyl, —N(CH₂CH₂NH₂)₂, diazamino, amidino, guanidino, ureido, carbamoyl, —C(═O)OH, —C(═O)OR^{c}, —C(═O)NH—OH, —O—NH—C(═NH)NH₂, —NH—S(═O)₂OH, S(═O)₂OH, NR^{d}R^{e}, semicarbazone, aryl, cycloalkyl, heterocycloalkyl, or heteroaryl, wherein each of the aryl and cycloalkyl is substituted with one or more substitutents, wherein each of the heterocycloalkyl and heteroaryl is optionally substituted with one or more substituents, and wherein each of the substituents for the aryl, cycloalkyl, heterocycloalkyl, and heteroaryl is, independently, nitro, cyano, amino, halo, hydroxy, alkoxy, alkylthio, alkylamino, dialkylamino, —NH(CH₂)ₚNH₂ wherein p is 1 to 5, —N(CH₂CH₂NH₂)₂, diazamino, amidino, guanidino, ureido, carbamoyl, —C(═O)OH, —C(═O)OR^{c}, —C(═O)NH—OH, —O—NH—C(═NH)NH₂, —NH—S(═O)₂OH, S(═O)₂OH, NR^{d}R^{e}, semicarbazone, aminosulfonyl, aminoalkoxy, aminoalkylhio, lower acylamino, or benzyloxycarbonyl;

each R^{c} is, independently, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl, or heterocycloalkylalkyl, each optionally substituted by one or more substitutents, wherein each substituent is, independently, OH, amino, halo, C₁₋₆ alkyl, C₁₋₆ haloalkyl, aryl, arylalkyl, heteroaryl, heteroarylalkyl, cycloalkyl, or heterocycloalkyl;

R^{d} and R^{e} are, independently, H, C₁₋₆ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl, or heterocycloalkylalkyl, wherein each of the C₁₋₆ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl, or heterocycloalkylalkyl is optionally substituted by OH, amino, halo, C₁₋₆ alkyl, C₁₋₆ haloalkyl, C₁₋₆ haloalkyl, aryl, arylalkyl, heteroaryl, heteroarylalkyl, cycloalkyl, or heterocycloalkyl;

or R^{d} and R^{e} together with the N atom to which they are attached form a 4-, 5-, 6-, 7-, or 8-membered heterocycloalkyl;

each LK^{PL} is, independently, —(CH₂)_{pPL}— or C₂₋₈ alkenylenyl, wherein each of the —(CH₂)_{pNPL}— and C₂₋₈ alkenylenyl is optionally substituted with one or more substituents, wherein each substituent is, independently, amino, hydroxyl, aminoalkyl, hydroxylalkyl, or alkyl;

each pPL is, independently, an integer from 0 to 8;

q1PL and q2PL are each, independently, 0, 1, or 2;

m11 is an integer from 1 to about 20; and

m12 is an integer from 1 to about 20.

In some embodiments of the compound of Formula III or pharmaceutically acceptable salt thereof:

each moiety of X-A₁-Y—X-A₂-Y is, independently, a moiety of:

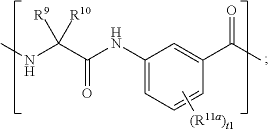

each R⁹ is, independently, H, a PL group, or an NPL group;

each R¹⁰ is, independently, H, a PL group, or an NPL group;

each R^{11a} is, independently, a PL group or an NPL group; and

each t1 is independently 0, 1, or 2.

In some embodiments of the compound of Formula III or pharmaceutically acceptable salt thereof, each R⁹ is, independently, a PL group or an NPL group; and each R¹⁰ is H; or each R⁹ and R¹⁰, taken together, constitute the side chain of a D or L α amino acid.

In some embodiments of the compound of Formula III or pharmaceutically acceptable salt thereof, each R⁹ is, independently, alkyl or (CH₂)_{pPL}—V where pPL is an integer from 1 to 5; each R¹⁰ is H; and each R^{11a} is, independently, halo, alkyl, alkoxy, haloalkyl, haloalkoxy, —(CH₂)_{pPL}—V, —O(CH₂)_{pPL}—V, or —S(CH₂)_{pPL}—V, wherein pPL is an integer from 1 to 5.

In some embodiments of the compound of Formula III or pharmaceutically acceptable salt thereof:

each R⁹ is, independently, alkyl, —(CH₂)—V, —(CH₂)₂—V, —(CH₂)₃—V, —(CH₂)₄—V, or —(CH₂)₅—V;

each R¹⁰ is H;
or each R⁹ and R¹⁰, taken together, constitute the side chain of a D or L α amino acid;

each V is, independently, hydroxyl, amino, heteroarylamino, ureido, guanidino, carbamoyl, C(═O)OH, —C(═O)OR^{c}, —C(═O)NH—OH, —O—NH—C(═NH)NH₂, —NH—S(═O)₂OH, S(═O)₂OH, aziridinyl, azetidinyl, pyrrolidinyl, piperidinyl, piperazinyl, morpholino, azepanyl, azocanyl, tetrazolyl, 1,2,4-oxadiazolyl, 1,3,4-oxadiazolyl, imidazolyl, pyridinyl, indolyl, or a substituted phenyl, wherein the substituted phenyl is substituted with one or more substituents, wherein each substituent is, independently, OH or amino; and

each R^{11a} is, independently, alkoxy.

In some embodiments of the compound of Formula III or pharmaceutically acceptable salt thereof:

each R⁹ is, independently, CH₃, —(CH₂)—V, —(CH₂)₂—V, —(CH₂)₃—V, —(CH₂)₄—V, and —(CH₂)₅—V;

each R¹⁰ is H;

or each R⁹ and R¹⁰, taken together, constitute the side chain of a D or L α amino acid;

each V is, independently, amino, alkylamino, dialkylamino, —NH(CH₂)ₚNH₂ wherein p is 1 to 5, —N(CH₂CH₂NH₂)₂, guanidino, amidino, ureido, or indolyl; and

each R^{11a} is, independently, alkoxy.

In some embodiments of the compound of Formula III or pharmaceutically acceptable salt thereof:

each R⁹ is, independently, CH₃, —(CH₂)—V, —(CH₂)₂—V, —(CH₂)₃—V, —(CH₂)₄—V, and —(CH₂)₅—V;

each R¹⁰ is H;

each V is, independently, amino, alkylamino, dialkylamino, —NH(CH₂)ₚNH₂ wherein p is 1 to 5, —N(CH₂CH₂NH₂)₂, guanidino, amidino, ureido, or indolyl; and

each R^{11a} is methoxy.

In some embodiments of the compound of Formula III or pharmaceutically acceptable salt thereof, each moiety of X-A₁-Y—X-A₂-Y is, independently, a moiety of:

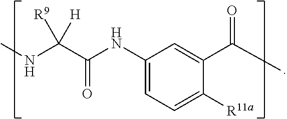

In some embodiments of the compound of Formula III or pharmaceutically acceptable salt thereof, R^{2a} and R^{2b} are each, independently, NH₂, amidino, alkylamino, dialkylamino, —NH(CH₂)ₚNH₂ wherein p is 1 to 5, or —NH—(CH₂)_{pPL}—V¹⁰, wherein V is amino, alkylamino, dialkylamino, —NH(CH₂)ₚNH₂ wherein p is 1 to 5, —N(CH₂CH₂NH₂)₂, guanidino, amidino, ureido, or carbamoyl; and L¹ is C₅₋₁₀alkylene optionally substituted with one or more substitutents, wherein each substituent is, independently, alkyl, halo, haloalkyl, aminoalkyl, or hydroxylalkyl. In some embodiments, R^{2a} and R^{2b} are as described above for R¹ and R² under Formula I.

In some embodiments of the compound of Formula III or pharmaceutically acceptable salt thereof, each of R^{2a} and R^{2b} is NH₂; and L¹ is C₅₋₁₀alkylene, such as, for example C₇₋₁₀alkylene or C₇₋₉alkylene.

In some embodiments of the compound of Formula III or pharmaceutically acceptable salt thereof, m11 is an integer from 1 to about 10; and m12 is an integer from 1 to about 10. In some embodiments, m11 is an integer from 3 to 7; and m12 is an integer from 3 to 7. In some embodiments, m11 is an integer from 3 to 5; and m12 is an integer from 3 to 5.

In some embodiments, the present invention provides compounds and methods for antagonizing an anticoagulant agent (including, for example, unfractionated heparin, low molecular weight heparin, and synthetically modified heparin or low molecular heparin derivatives) comprising administering to a mammal a compound of Formula IV:

R¹—[—X-A₁-Y—X-A₂-Y—]ₘ₁₃—X-L¹-Y—[—X-A₁-Y—X-A₂-Y—]ₘ₁₄—R² IV

or pharmaceutically acceptable salt thereof, wherein:

each X is, independently, NR⁸;

each Y is C═O;

each R⁸ is, independently, hydrogen or alkyl;

each A₂ is optionally substituted arylene or optionally substituted heteroarylene, and each A₁ is —(CH₂)_{q}—, wherein q is 1 to 7, wherein A₁ and A₂ are each, independently, optionally substituted with one or more PL group(s), one or more NPL group(s), or a combination of one or more PL group(s) and one or more NPL group(s);

R¹ is hydrogen, a PL group, or an NPL group, and R² is —X-A₁-Y—R¹¹, wherein R¹¹ is hydrogen, a PL group, or an NPL group; or

R¹ and R² are each, independently, hydrogen, a PL group, or an NPL group; or

R¹ and R² together are a single bond; or

R¹ is —Y-A₂-X—R¹², wherein R¹² is hydrogen, a PL group, or an NPL group, and R² is hydrogen, a PL group, or an NPL group; or

R¹ and R² are, alone or in combination, are the R¹ and R² substituents described above for Formula I;

L¹ is C₁₋₁₀alkylene optionally substituted with one or more substitutents, wherein each substituent is, independently, alkyl, halo, haloalkyl, aminoalkyl, hydroxylalkyl, V, or —(CH₂)_{pPL}—V wherein pPL is an integer from 1 to 5;

each V is, independently, hydroxy, amino, alkylamino, dialkylamino, amido, alkylamido, dialkylamido, —NH(CH₂)ₚNH₂ wherein p is 1 to 5, —C(═O)NH(CH₂)ₚNH₂ wherein p is 1 to 5, —C(═O)NH(CH₂)ₚNHC(═NH)NH₂ wherein p is 1 to 5, —C(═O)NH(CH₂)ₚNHC(═O)NH₂ wherein p is 1 to 5, —NHC(═O)-alkyl, —N(CH₂CH₂NH₂)₂, guanidino, amidino, ureido, carbamoyl, —C(═O)OH, —C(═O)OR^{c}, —C(═O)NH—OH, —O—NH—C(═NH)NH₂, —NH—S(═O)₂OH, S(═O)₂OH, NR^{d}R^{e}, a substituted aryl group, heterocycloalkyl, or heteroaryl, wherein each of the heterocycloalkyl and heteroaryl is optionally substituted with one more substituents, wherein each substituent is, independently, amino, halo, cyano, nitro, hydroxy, —NH(CH₂)ₚNH₂ wherein p is 1 to 5, —N(CH₂CH₂NH₂)₂, amidino, guanidino, aminosulfonyl, aminoalkoxy, aminoalkylhio, lower acylamino, or benzyloxycarbonyl; and wherein the substituted aryl group is substituted with one more substituents, wherein each substituent is, independently, amino, halo, cyano, nitro, hydroxy, —NH(CH₂)ₚNH₂ wherein p is 1 to 5, —N(CH₂CH₂NH₂)₂, amidino, guanidino, aminosulfonyl, aminoalkoxy, aminoalkylhio, lower acylamino, or benzyloxycarbonyl;

each NPL group is, independently, —B(OR⁴)₂ or —(NR^{3′})_{q1NPL}—U^{NPL}-LK^{NPL}—(NR^{3″})_{q2NPL}—R^{4′}, wherein:

R³, R^{3′}, and R^{3″} are each, independently, hydrogen, alkyl, or alkoxy;

R⁴ and R^{4′} are each, independently, hydrogen, alkyl, alkenyl, alkynyl, cycloalkyl, aryl, or heteroaryl, wherein each of the alkyl, alkenyl, alkynyl, cycloalkyl, aryl, and heteroaryl is optionally substituted with one or more substitutents, wherein each substituent is, independently, alkyl, halo, or haloalkyl;

each U^{NPL} is, independently, absent or O, S, S(═O), S(═O)₂, NR³, —C(═O)—, —C(═O)—NR³—, —C(═O)—N═N—NR³—, —C(═O)—NR³—N═N—, —N═N—NR³—, —C(═N—N(R³)₂)—, —C(═NR³)—, —C(═O)O—, —C(═O)S—, —C(═S)—, —O—P(═O)₂O—, —S—C═N—, or —C(═O)—NR³—O—, wherein groups with two chemically nonequivalent termini can adopt both possible orientations;

each LK^{NPL} is, independently, —(CH₂)_{pNPL}— or C₂₋₈ alkenylenyl, wherein each of the —(CH₂)ₚNPL and C₂₋₈ alkenylenyl is optionally substituted with one or more substituents, wherein each substituent is, independently, amino, hydroxyl, aminoalkyl, hydroxylalkyl, or alkyl;

each pNPL is, independently, an integer from 0 to 8;

q1NPL and q2NPL are each, independently, 0, 1, or 2;

each PL group is, independently, halo, hydroxyethoxymethyl, methoxyethoxymethyl, polyoxyethylene, or —(NR^{5′})_{q1PL}—U^{PL}-LK^{PL}—(NR^{5″})_{q2PL}—V, wherein:

R⁵, R^{5′}, and R^{5″} are each, independently, hydrogen, alkyl, or alkoxy;

each U^{PL} is, independently, absent or O, S, S(═O), S(═O)₂, NR⁵, —C(═O)—, —C(═O)—NR⁵—, —C(═O)—N═N—NR⁵—, —C(═O)—NR⁵—N═N—, —N═N—NR⁵—, —C(═N—N(R⁵)₂)—, —C(═NR⁵)—, —C(═O)O—, —C(═O)S—, —C(═S)—, —O—P(═O)₂O—, —S—C═N—, or —C(═O)—NR⁵—O—, wherein groups with two chemically nonequivalent termini can adopt either of the two possible orientations;

each R^{c} is, independently, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl, or heterocycloalkylalkyl, each optionally substituted by one or more substitutents, wherein each substituent is, independently, OH, amino, halo, C₁₋₆ alkyl, C₁₋₆ haloalkyl, aryl, arylalkyl, heteroaryl, heteroarylalkyl, cycloalkyl, or heterocycloalkyl;

R^{d} and R^{e} are, independently, H, C₁₋₆ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl, or heterocycloalkylalkyl, wherein each of the C₁₋₆ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl, and heterocycloalkylalkyl is optionally substituted by OH, amino, halo, C₁₋₆ alkyl, C₁₋₆haloalkyl, C₁₋₆haloalkyl, aryl, arylalkyl, heteroaryl, heteroarylalkyl, cycloalkyl or heterocycloalkyl;

or R^{d} and R^{e} together with the N atom to which they are attached form a 4-, 5-, 6-, 7-, or 8-membered heterocycloalkyl;

each LK^{PL} is, independently, —(CH₂)_{pPL}— or C₂₋₈ alkenylenyl, wherein each of the —(CH₂)_{pNPL}— and C₂₋₈ alkenylenyl is optionally substituted with one or more substituents, wherein each substituent is, independently, amino, hydroxyl, aminoalkyl, hydroxylalkyl, or alkyl;

each pPL is, independently, an integer from 0 to 8;

q1PL and q2PL are each, independently, 0, 1, or 2;

m13 is an integer from 1 to about 10; and

m14 is an integer from 1 to about 10.

In some embodiments of the compound of Formula IV or pharmaceutically acceptable salt thereof:

each moiety of X-A₁-Y—X-A₂-Y is, independently, a moiety of:

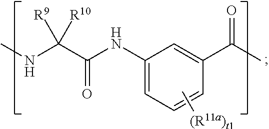

each R⁹ is, independently, H, a PL group, or an NPL group;

each R¹⁰ is, independently, H, a PL group, or an NPL group;

or or R⁹ and R¹⁰, taken together, constitute the side chain of a D or L α amino acid;

each R^{11a} is, independently, a PL group or an NPL group; and

each t1 is independently 0, 1, or 2.

In some embodiments of the compound of Formula IV or pharmaceutically acceptable salt thereof, each R⁹ is, independently, a PL group or an NPL group; and each R¹⁰ is H; or R⁹ and R¹⁰, taken together, constitute the side chain of a D or L α amino acid.

In some embodiments of the compound of Formula IV or pharmaceutically acceptable salt thereof, each R⁹ is, independently, alkyl or (CH₂)_{pPL}—V wherein pPL is an integer from 1 to 5; each R¹⁰ is H; or R⁹ and R¹⁰, taken together, constitute the side chain of a D or L α amino acid; and each R^{11a} is, independently, halo, alkyl, alkoxy, haloalkyl, haloalkoxy, —(CH₂)_{pPL}—V, —O(CH₂)_{pPL}—V, or —S(CH₂)_{pPL}—V, wherein pPL is an integer from 1 to 5.

In some embodiments of the compound of Formula IV or pharmaceutically acceptable salt thereof:

each R⁹ is, independently, alkyl, —(CH₂)—V, —(CH₂)₂—V, —(CH₂)₃—V, —(CH₂)₄—V, or —(CH₂)₅—V;

each R¹⁰ is H;

or R⁹ and R¹⁰, taken together, constitute the side chain of a D or L α amino acid;

each V is, independently, hydroxyl, amino, heteroarylamino, ureido, guanidino, carbamoyl, C(═O)OH, —C(═O)OR^{c}, —C(═O)NH—OH, —O—NH—C(═NH)NH₂, —NH—S(═O)₂OH, S(═O)₂OH, aziridinyl, azetidinyl, pyrrolidinyl, piperidinyl, piperazinyl, morpholino, azepanyl, azocanyl, tetrazolyl, 1,2,4-oxadiazolyl, 1,3,4-oxadiazolyl, imidazolyl, pyridinyl, indolyl, or a substituted phenyl, wherein the substituted phenyl is substituted with one or more substituents, wherein each substituent is, independently, OH or amino; and

each R^{11a} is, independently, alkoxy.

In some embodiments of the compound of Formula IV or pharmaceutically acceptable salt thereof, each R⁹ is, independently, CH₃, —(CH₂)—V, —(CH₂)₂—V, —(CH₂)₃—V, —(CH₂)₄—V, or —(CH₂)₅—V; each R¹⁰ is H; each V is, independently, amino, alkylamino, dialkylamino, —NH(CH₂)ₚNH₂ wherein p is 1 to 5, —N(CH₂CH₂NH₂)₂, guanidino, amidino, ureido, or indolyl; and each R^{11a} is, independently, alkoxy.

In some embodiments of the compound of Formula IV or pharmaceutically acceptable salt thereof, each R⁹ is, independently, CH₃, —(CH₂)—V, —(CH₂)₂—V, —(CH₂)₃—V, —(CH₂)₄—V, or —(CH₂)₅—V; each R¹⁰ is H; each V is, independently, amino, alkylamino, dialkylamino, —NH(CH₂)ₚNH₂ wherein p is 1 to 5, —N(CH₂CH₂NH₂)₂, guanidino, amidino, ureido, or indolyl; and each R^{11a} is methoxy.

In some embodiments of the compound of Formula IV or pharmaceutically acceptable salt thereof, each moiety of X-A₁-Y—X-A₂-Y is, independently, a moiety of:

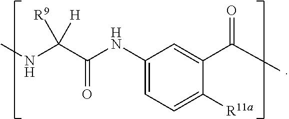

In some embodiments of the compound of Formula IV or pharmaceutically acceptable salt thereof:

the moiety of —X-L¹-Y— is a moiety of —NH-L¹-C(═O)—;

R¹ is H or alkyl;

R² is NH₂, amidino, alkylamino, dialkylamino, —NH(CH₂)ₚNH₂ wherein p is 1 to 5, or —NH—(CH₂)_{pPL}—V¹⁰, wherein V¹⁰ is amino, alkylamino, dialkylamino, —NH(CH₂)ₚNH₂ wherein p is 1 to 5, —N(CH₂CH₂NH₂)₂, guanidino, amidino, ureido, or carbamoyl; and

L¹ is C₁₋₃alkylene optionally substituted with one or more substitutents, wherein each substituent is, independently, alkyl, halo, haloalkyl, aminoalkyl, hydroxylalkyl, V¹¹, or —(CH₂)_{pPL}—V¹¹ wherein pPL is an integer from 1 to 5, wherein each V″ is, independently, amino, alkylamino, dialkylamino, —NH(CH₂)ₚNH₂ wherein p is 1 to 5, —N(CH₂CH₂NH₂)₂, guanidino, amidino, ureido, or carbamoyl.

In some embodiments of the compound of Formula IV or pharmaceutically acceptable salt thereof:

the moiety of —X-L¹-Y— is a moiety of —NH-L¹-C(═O)—;

R¹ is H;

R² is NH₂; and

L¹ is C₁alkylene optionally substituted with one or more substitutents, wherein each substituent is, independently, alkyl, halo, haloalkyl, aminoalkyl, hydroxylalkyl, V″, or —(CH₂)_{pPL}—V¹¹ wherein pPL is an integer from 1 to 5, wherein V¹¹ is amino, alkylamino, dialkylamino, —NH(CH₂)ₚNH₂ wherein p is 1 to 5, —N(CH₂CH₂NH₂)₂, guanidino, amidino, ureido, or carbamoyl.

In some embodiments of the compound of Formula IV or pharmaceutically acceptable salt thereof, m13 is an integer from 1 to about 5; and m14 is an integer from 1 to about 5. In some embodiments, m13 is an integer from 1 to 3; and m12 is an integer from 1 to 3. In some embodiments, the sum of m13 and m14 is an integer from 3 to 5. In some embodiments, the sum of m13 and m14 is 4.

Additional compounds or salts thereof that are useful in antagonizing an anticoagulant agent (including, for example, unfractionated heparin, low molecular weight heparin, and synthetically modified heparin or low molecular heparin derivatives) can be selected from, for example, Compounds 4, 66, 73, 74, and 75 of Table 1 herein below, or their pharmaceutically acceptable salts thereof.

In another aspect, the present invention provides novel compounds and pharmaceutically acceptable salts thereof. In some embodiments, the present invention provides a novel compound of Formula I or pharmaceutically acceptable salt thereof. In some embodiments, the present invention provides a novel compound of Formula II or IIa or pharmaceutically acceptable salt thereof. In some embodiments, the present invention provides a novel compound of Formula III or pharmaceutically acceptable salt thereof. In some embodiments, the present invention provides a novel compound of Formula IV or pharmaceutically acceptable salt thereof. In some embodiments, the present invention provides a compound selected from Compounds 3-5, 7, and 9-88, or pharmaceutically acceptable salt thereof. In some embodiments, the present invention provides a compound selected from Compounds 4, 66, 73, 74, and 75, or pharmaceutically acceptable salt thereof. In some embodiments, the present invention provides a compound selected from Compounds 66, 73, 74, and 75, or pharmaceutically acceptable salt thereof. In some embodiments, the present invention provides a compound selected from Compounds 71, 84, and 85, or pharmaceutically acceptable salt thereof. In some embodiments, the present invention further provides a pharmaceutical composition comprising a novel compound of the present invention or pharmaceutically salt thereof and a pharmaceutically acceptable carrier.

The present invention also provides, in part, compounds of Formula V:

R¹—[—X-A¹-X—Y-A²-Y—]ₘ—R² V

or a pharmaceutically acceptable salt thereof, wherein:

each of the moiety of —X-A¹-X— is, independently, a moiety of Formula XXI-1, XXI-2, XXI-3, XXI-4, XXI-5, XXI-6, XXI-7, or XXI-8:

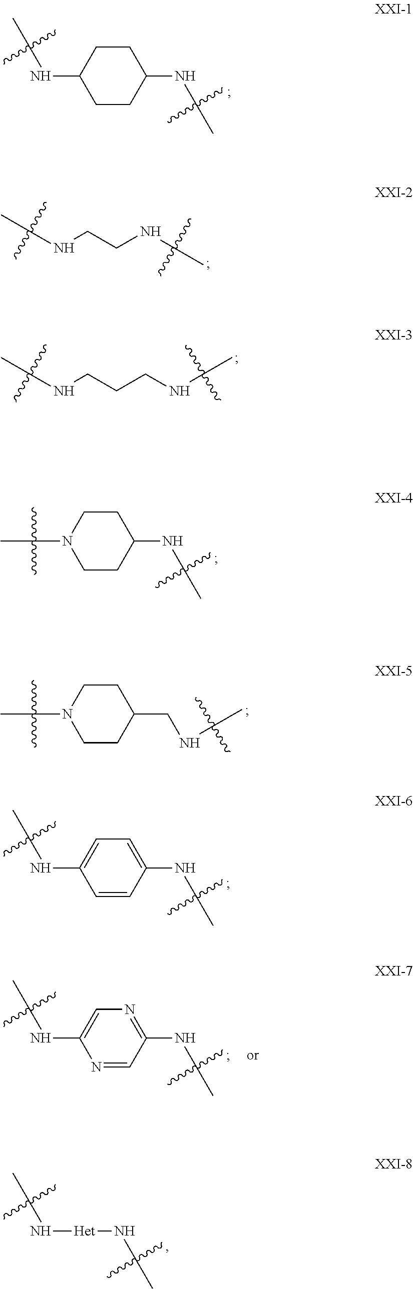

where Het is any 5 or 6-membered ring heterocycle;

each of the moiety of —Y-A²-Y— is, independently, a moiety of Formula XXII-1, XXII-2, XXII-3, XXII-4, or XXII-5:

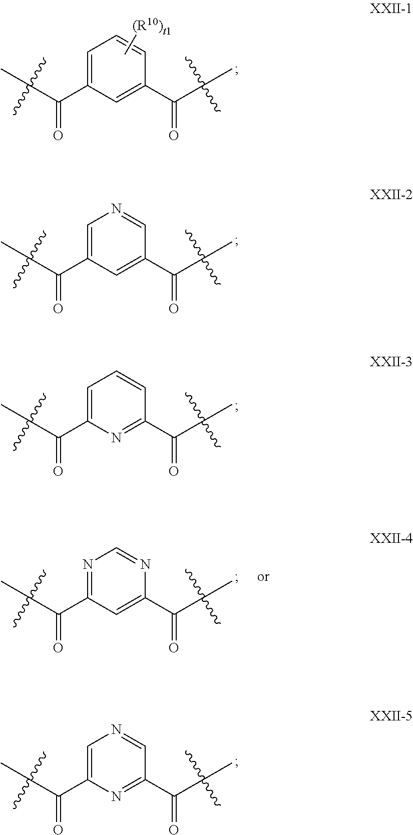

R¹ is hydrogen, —C(═O)R¹¹, or —Y-A²-Y—R¹²;

R² is —OH, —NH₂, —NH(C₁₋₄alkyl), —N(C₁₋₄alkyl)₂, —NH—C(═NH)NH₂, —NH(CH₂)ₚNH₂ wherein p is an integer from 1 to 5, —NH(CH₂)ₚNH(C₁₋₄alkyl) wherein p is an integer from 1 to 5, —NH(CH₂)ₚN(C₁₋₄alkyl)₂ wherein p is an integer from 1 to 5, —NH(CH₂)ₚNHC(═NH)NH₂ wherein p is an integer from 1 to 5, R^{12a}, or —X-A¹-X—R¹³;

each R¹⁰ is, independently, —C(═O)NH₂, —C(═O)NH(CH₂)ₚNH₂ wherein p is an integer from 1 to 5, —C(═O)NH(CH₂)ₚNH(C₁₋₄alkyl) wherein p is an integer from 1 to 5, —C(═O)NH(CH₂)ₚN(C₁₋₄alkyl)₂ wherein p is an integer from 1 to 5, —C(═O)NH(CH₂)ₚNHC(═NH)NH₂ wherein p is an integer from 1 to 5, —OCH₃, or —OR^{10a};

each R^{10a} is, independently, C₁₋₈alkyl substituted with R^{A};

each R^{A} is, independently, —NH₂, —NH(C₁₋₄alkyl), —N(C₁₋₄alkyl)₂, —NH—C(═NH)NH₂, —C(═O)NH₂, or —C(═O)OH;

each R¹¹ is, independently, C₁₋₈alkyl or aryl, each substituted with 0, 1, 2, or 3 substituents each independently selected from —OCH₃, —OR^{11a}, —C(═O)NH₂, —C(═O)NH(CH₂)ₚNH₂ wherein p is an integer from 1 to 5, —C(═O)NH(CH₂)ₚNH(C₁₋₄alkyl) wherein p is an integer from 1 to 5, —C(═O)NH(CH₂)ₚN(C₁₋₄alkyl)₂ wherein p is an integer from 1 to 5, —C(═O)NH(CH₂)ₚNHC(═NH)NH₂ wherein p is an integer from 1 to 5, —NH₂, —NH(C₁₋₄alkyl), —N(C₁₋₄alkyl)₂, or —NH—C(═NH)NH₂;

each R^{11a} is, independently, C₁₋₈alkyl substituted with R^{B};

each R^{B} is, independently, —NH₂, —NH(C₁₋₄alkyl), —N(C₁₋₄alkyl)₂, —NH—C(═NH)NH₂, or —C(═O)NH₂;

R¹² is —OH, —NH₂, —NH(C₁₋₄alkyl), —N(C₁₋₄alkyl)₂, —NH—C(═NH)NH₂, —NH(CH₂)ₚNH₂ wherein p is an integer from 1 to 5, —NH(CH₂)ₚNH(C₁₋₄alkyl) wherein p is an integer from 1 to 5, —NH(CH₂)ₚN(C₁₋₄alkyl)₂ wherein p is an integer from 1 to 5, —NH(CH₂)ₚNHC(═NH)NH₂ wherein p is an integer from 1 to 5, or R^{12a};

R^{12a} is a moiety of Formula XXXI:

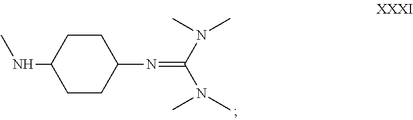

R¹³ is hydrogen or —C(═O)R¹¹;

t1 is 0, 1, or 2; and

m is 1, 2, 3, or 4, provided that:

(a) the compound of Formula V, or pharmaceutically acceptable salt thereof, comprises at least one moiety of Formula XXI-4;

(b) the compound of Formula V, or pharmaceutically acceptable salt thereof, comprises at least one moiety of Formula XXI-5;

(c) the compound of Formula V, or pharmaceutically acceptable salt thereof, comprises at least two different moieties of Formulas XXI-1, XXI-2, XXI-3, XXI-4, or XXI-5;

(d) the compound of Formula V, or pharmaceutically acceptable salt thereof, comprises at least one moiety of Formula XXI-4 and at least one moiety of Formula XXI-1, XXI-2, XXI-3, or XXI-5;

(e) the compound of Formula V, or pharmaceutically acceptable salt thereof, comprises at least one moiety of Formula XXI-5 and at least one moiety of Formula XXI-1, XXI-2, XXI-3, or XXI-4;

(f) the compound of Formula V, or pharmaceutically acceptable salt thereof, comprises at least one moiety of Formula XXII-2;

(g) the compound of Formula V, or pharmaceutically acceptable salt thereof, comprises at least one moiety of Formula XXII-3;

(h) the compound of Formula V, or pharmaceutically acceptable salt thereof, comprises at least one moiety of Formula XXII-2 and at least one moiety of Formula XXII-1, XXII-3, or XXII-4;

(i) the compound of Formula V, or pharmaceutically acceptable salt thereof, comprises at least one moiety of Formula XXII-3 and at least one moiety of Formula XXII-1, XXII-2, or XXII-4;

(j) the compound of Formula V, or pharmaceutically acceptable salt thereof, comprises at least two different moieties of Formulas XXII-1, XXII-2, XXII-3 and XXII-4;

(k) the compound of Formula V, or pharmaceutically acceptable salt thereof, comprises at least one moiety of Formula XXI-6, XXI-7, or XXI-8;

(l) the compound of Formula V, or pharmaceutically acceptable salt thereof, comprises at least two different moieties of Formula XXI-6, XXI-7, or XXI-8;

(m) the compound of Formula V, or pharmaceutically acceptable salt thereof, comprises at least one moiety of Formula XXI-6 and at least one moiety of Formula XXI-7 or XXI-8;

(n) the compound of Formula V, or pharmaceutically acceptable salt thereof, comprises at least one moiety of Formula XXI-7 and at least one moiety of Formula XXI-8;

(o) the compound of Formula V, or pharmaceutically acceptable salt thereof, comprises at least one moiety of Formula XXII-5;

(p) the compound of Formula V, or pharmaceutically acceptable salt thereof, comprises at least one moiety of Formula XXII-5 and at least one moiety of Formula XXII-1, XXII-2, XXII-3, or XXII-4;

(q) the compound of Formula V, or pharmaceutically acceptable salt thereof, comprises at least two different moieties of Formulas XXII-1, XXII-2, XXII-3, XXII-4, and XXII-5; or

(r) the compound of Formula V, or pharmaceutically acceptable salt thereof, comprises at least one moiety of Formula XXXI, or a compound selected from Compound 201-427, or a pharmaceutically acceptable salt thereof.

In some embodiments, the compound of Formula V, or pharmaceutically acceptable salt thereof, comprises at least one moiety of Formula XXI-4. In some embodiments, the compound of Formula V, or pharmaceutically acceptable salt thereof, comprises at least one moiety of Formula XXI-5. In some embodiments, the compound of Formula V, or pharmaceutically acceptable salt thereof, comprises at least two different moieties of Formulas XXI-1, XXI-2, XXI-3, XXI-4, or XXI-5. In some embodiments, the compound of Formula V, or pharmaceutically acceptable salt thereof, comprises at least one moiety of Formula XXI-4 and at least one moiety of Formula XXI-1, XXI-2, XXI-3, or XXI-5. In some embodiments, the compound of Formula V, or pharmaceutically acceptable salt thereof, comprises at least one moiety of Formula XXI-4 or XXI-5 and at least one moiety of Formula XXI-1.

In some embodiments, the compound of Formula V, or pharmaceutically acceptable salt thereof, comprises at least one moiety of Formula XXII-2. In some embodiments, the compound of Formula V, or pharmaceutically acceptable salt thereof, comprises at least one moiety of Formula XXII-3. In some embodiments, the compound of Formula V, or pharmaceutically acceptable salt thereof, comprises at least one moiety of Formula XXII-2 and at least one moiety of Formula XXII-1, XXII-3, or XXII-4. In some embodiments, the compound of Formula V, or pharmaceutically acceptable salt thereof, comprises at least one moiety of Formula XXII-3 and at least one moiety of Formula XXII-1, XXII-2, or XXII-4. In some embodiments, the compound of Formula V, or pharmaceutically acceptable salt thereof, comprises at least two different moieties of Formulas XXII-1, XXII-2, XXII-3 and XXII-4. In some embodiments, the compound of Formula V, or pharmaceutically acceptable salt thereof, comprises at least one moiety of Formula XXXI. In some embodiments, the compound of Formula V, or pharmaceutically acceptable salt thereof, which comprises a moiety of Formula XXII-1, which is a moiety of XXII-1-a or XXII-1-b:

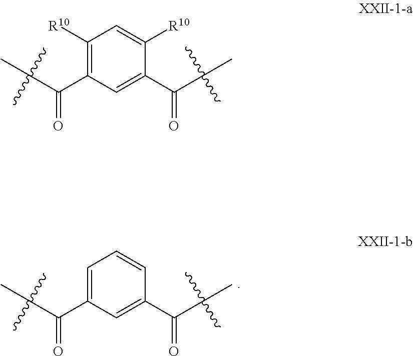

In some embodiments, the compound of Formula V, or pharmaceutically acceptable salt thereof, which comprises a moiety of Formula XXII-1, which is a moiety of XXII-1-a:

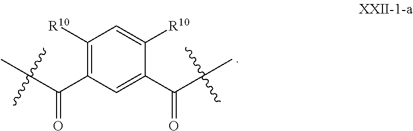

In some embodiments, the compound of Formula V, or pharmaceutically acceptable salt thereof, which comprises a moiety of Formula XXII-1, which is a moiety of XXII-1-b:

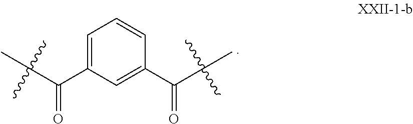

The present invention also provides, in part, a compound of Formula V:

R¹—[—X-A¹-X—Y-A²-Y—]ₘR² V

or a pharmaceutically acceptable salt thereof, wherein:

each of the moiety of —X-A¹-X— is, independently, a moiety of Formula XXI-1, XXI-2, XXI-3, XXI-4, or XXI-5:

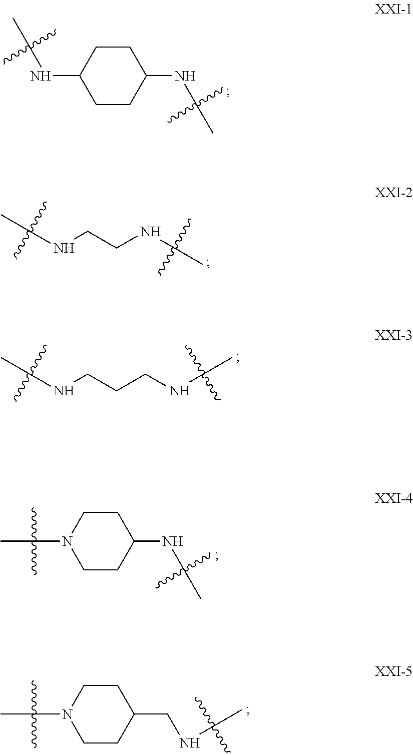

each of the moiety of —Y-A²-Y— is, independently, a moiety of Formula XXII-1, XXII-2, XXII-3, or XXII-4:

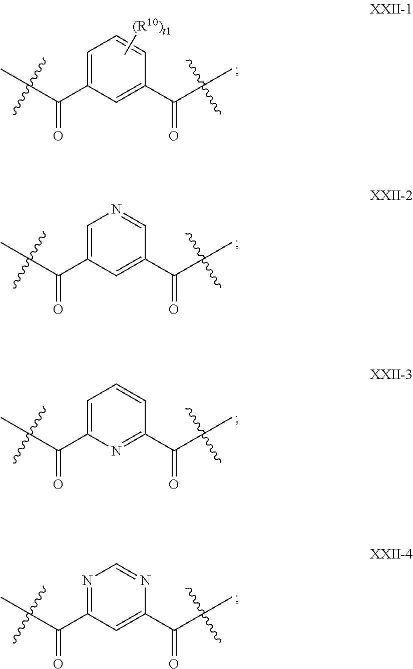

R¹ is hydrogen, —C(═O)R¹¹, or —Y-A²-Y—R¹²;

R² is —OH, —NH₂, —NH(C₁₋₄alkyl), —N(C₁₋₄alkyl)₂, —NH—C(═NH)NH₂, —NH(CH₂)ₚNH₂ wherein p is an integer from 1 to 5, —NH(CH₂)ₚNH(C₁₋₄alkyl) wherein p is an integer from 1 to 5, —NH(CH₂)ₚN(C₁₋₄alkyl)₂ wherein p is an integer from 1 to 5, —NH(CH₂)ₚNHC(═NH)NH₂ wherein p is an integer from 1 to 5, R^{12a}, or —X-A¹-X—R¹³;

each R¹⁰ is, independently, —C(═O)NH₂, C(═O)NH(CH₂)ₚNH₂ wherein p is an integer from 1 to 5, —C(═O)NH(CH₂)ₚNH(C₁₋₄alkyl) wherein p is an integer from 1 to 5, —C(═O)NH(CH₂)ₚN(C₁₋₄alkyl)₂ wherein p is an integer from 1 to 5, —C(═O)NH(CH₂)ₚNHC(═NH)NH₂ wherein p is an integer from 1 to 5, —OCH₃, or —OR^{10a};

each R^{10a} is, independently, C₁₋₈alkyl substituted with R^{A};

each R^{A} is, independently, —NH₂, —NH(C₁₋₄alkyl), —N(C₁₋₄alkyl)₂, —NH—C(═NH)NH₂, —C(═O)NH₂, or —C(═O)OH;

each R¹¹ is, independently, C₁₋₈alkyl or aryl, each substituted with 0, 1, 2, or 3 substituents each independently selected from —OCH₃, —OR^{11a}, —C(═O)NH₂, —C(═O)NH(CH₂)ₚNH₂ wherein p is an integer from 1 to 5, —C(═O)NH(CH₂)ₚNH(C₁₋₄alkyl) wherein p is an integer from 1 to 5, —C(═O)NH(CH₂)ₚN(C₁₋₄alkyl)₂ wherein p is an integer from 1 to 5, —C(═O)NH(CH₂)ₚNHC(═NH)NH₂ wherein p is an integer from 1 to 5, —NH₂, —NH(C₁₋₄alkyl), —N(C₁₋₄alkyl)₂, or —NH—C(═NH)NH₂;

each R^{11a} is, independently, C₁₋₈alkyl substituted with R^{B};

each R^{B} is, independently, —NH₂, —NH(C₁₋₄alkyl), —N(C₁₋₄alkyl)₂, —NH—C(═NH)NH₂, —C(═O)NH₂;

R¹² is —OH, —NH₂, —NH(C₁₋₄alkyl), —N(C₁₋₄alkyl)₂, —NH—C(═NH)NH₂, —NH(CH₂)ₚNH₂ wherein p is an integer from 1 to 5, —NH(CH₂)ₚNH(C₁₋₄alkyl) wherein p is an integer from 1 to 5, —NH(CH₂)ₚN(C₁₋₄alkyl)₂ wherein p is an integer from 1 to 5, —NH(CH₂)ₚNHC(═NH)NH₂ wherein p is an integer from 1 to 5, or R^{12a};

R^{12a} is a moiety of Formula XXXI:

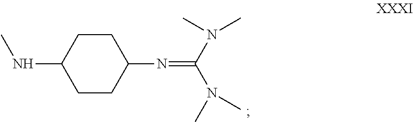

R¹³ is hydrogen or —C(═O)R¹¹;

t1 is 0, 1, or 2; and

m is 1, 2, 3, or 4,

provided that:

(a) the compound of Formula V, or pharmaceutically acceptable salt thereof, comprises at least one moiety of Formula XXI-4;

(b) the compound of Formula V, or pharmaceutically acceptable salt thereof, comprises at least one moiety of Formula XXI-5;

(c) the compound of Formula V, or pharmaceutically acceptable salt thereof, comprises at least two different moieties of Formulas XXI-1, XXI-2, XXI-3, XXI-4, or XXI-5;

(d) the compound of Formula V, or pharmaceutically acceptable salt thereof, comprises at least one moiety of Formula XXI-4 and at least one moiety of Formula XXI-1, XXI-2, XXI-3, or XXI-5;

(e) the compound of Formula V, or pharmaceutically acceptable salt thereof, comprises at least one moiety of Formula XXI-5 and at least one moiety of Formula XXI-1, XXI-2, XXI-3, or XXI-4;

(f) the compound of Formula V, or pharmaceutically acceptable salt thereof, comprises at least one moiety of Formula XXII-2;

(g) the compound of Formula V, or pharmaceutically acceptable salt thereof, comprises at least one moiety of Formula XXII-3;

(h) the compound of Formula V, or pharmaceutically acceptable salt thereof, comprises at least one moiety of Formula XXII-2 and at least one moiety of Formula XXII-1, XXII-3, or XXII-4;

(i) the compound of Formula V, or pharmaceutically acceptable salt thereof, comprises at least one moiety of Formula XXII-3 and at least one moiety of Formula XXII-1, XXII-2, or XXII-4;

(j) the compound of Formula V, or pharmaceutically acceptable salt thereof, comprises at least two different moieties of Formulas XXII-1, XXII-2, XXII-3 and XXII-4; or

(k) the compound of Formula V, or pharmaceutically acceptable salt thereof, comprises at least one moiety of Formula XXXI,

or administering to a mammal a compound selected from Compound 201-427, or a pharmaceutically acceptable salt thereof.

In some embodiments, the compound of Formula V or pharmaceutically acceptable salt thereof comprises at least one moiety of Formula XXI-4. In some embodiments, the compound of Formula V or pharmaceutically acceptable salt thereof comprises at least one moiety of Formula XXI-5. In some embodiments, the compound of Formula V or pharmaceutically acceptable salt thereof comprises at least two different moieties of Formulas XXI-1, XXI-2, XXI-3, XXI-4, or XXI-5 (for example, at least one moiety of Formula XXI-4 and at least one moiety of Formula XXI-5). In some embodiments, the compound of Formula V or pharmaceutically acceptable salt thereof comprises at least one moiety of Formula XXI-4 and at least one moiety of Formula XXI-1, XXI-2, XXI-3, or XXI-5. In some embodiments, the compound of Formula V or pharmaceutically acceptable salt thereof comprises at least one moiety of Formula XXI-4 and at least one moiety of Formula XXI-1. In some embodiments, the compound of Formula V or pharmaceutically acceptable salt thereof comprises at least one moiety of Formula XXI-5 and at least one moiety of Formula XXI-1, XXI-2, XXI-3, or XXI-4. In some embodiments, the compound of Formula V or pharmaceutically acceptable salt thereof comprises at least one moiety of Formula XXI-5 and at least one moiety of Formula XXI-1. In some embodiments, the compound of Formula V or pharmaceutically acceptable salt thereof comprises at least one moiety of Formula XXI-4 or XXI-5 and at least one moiety of Formula XXI-1. In some embodiments, the compound of Formula V or pharmaceutically acceptable salt thereof comprises at least one moiety of Formula XXII-2. In some embodiments, the compound of Formula V or pharmaceutically acceptable salt thereof comprises at least one moiety of Formula XXII-3. In some embodiments, the compound of Formula V or pharmaceutically acceptable salt thereof comprises at least one moiety of Formula XXII-2 and at least one moiety of Formula XXII-1, XXII-3, or XXII-4. In some embodiments, the compound of Formula V or pharmaceutically acceptable salt thereof comprises at least one moiety of Formula XXII-2 and at least one moiety of Formula XXII-1. In some embodiments, the compound of Formula V or pharmaceutically acceptable salt thereof comprises only one moiety of Formula XXII-2 and one or more moieties of Formula XXII-1. In some embodiments, the compound of Formula V or pharmaceutically acceptable salt thereof comprises at least one moiety of Formula XXII-3 and at least one moiety of Formula XXII-1, XXII-2, or XXII-4. In some embodiments, the compound of Formula V or pharmaceutically acceptable salt thereof comprises at least one moiety of Formula XXII-3 and at least one moiety of Formula XXII-1. In some embodiments, the compound of Formula V or pharmaceutically acceptable salt thereof comprises only one moiety of Formula XXII-3 and one or more moieties of Formula XXII-1. In some embodiments, the compound of Formula V or pharmaceutically acceptable salt thereof comprises at least two different moieties of Formulas XXII-1, XXII-2, XXII-3 and XXI-4. In some embodiments, the compound of Formula V or pharmaceutically acceptable salt thereof comprises at least one moiety of Formula XXXI.

In some embodiments of the compound of Formula V or pharmaceutically acceptable salt thereof, the moiety of Formula XXII-1 is a moiety of XXII-1-a or XIIX-1-b:

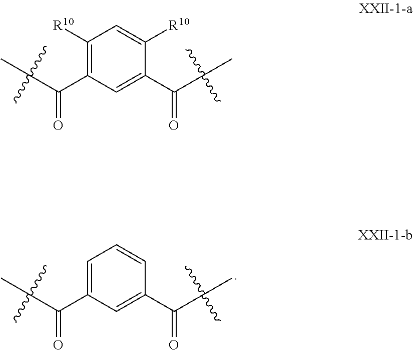

In some embodiments of the compound of Formula V or pharmaceutically acceptable salt thereof, the moiety of Formula XXII-1 is a moiety of XXII-1-a:

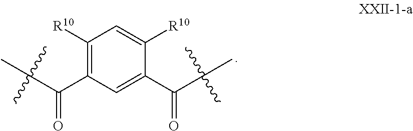

In some embodiments of the compound of Formula V or pharmaceutically acceptable salt thereof, the moiety of Formula XXII-1 is a moiety of XXII-1-b:

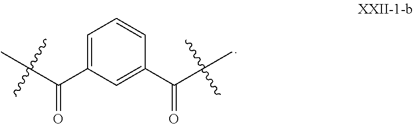

In some embodiments of the compound of Formula V or pharmaceutically acceptable salt thereof, R¹ is hydrogen.

In some embodiments of the compound of Formula V or pharmaceutically acceptable salt thereof: R¹ is —C(═O)R¹¹; R¹¹ is aryl substituted with 1, 2, or 3 substituents each independently selected from —OCH₃, —OR^{11a}, —C(═O)NH₂, —C(═O)NH(CH₂)ₚNH₂ wherein p is an integer from 1 to 5, —C(═O)NH(CH₂)ₚNH(C₁₋₄alkyl) wherein p is an integer from 1 to 5, —C(═O)NH(CH₂)ₚN(C₁₋₄alkyl)₂ wherein p is an integer from 1 to 5, or —C(═O)NH(CH₂)ₚNHC(═NH)NH₂ wherein p is an integer from 1 to 5; each R^{11a} is, independently, C₃₋₆ alkyl substituted with R^{B}; and each R^{B} is, independently, —NH₂ or —NH—C(═NH)NH₂.

In some embodiments of the compound of Formula V or pharmaceutically acceptable salt thereof, R¹ is —Y-A²-Y—R¹² and R¹² is R^{12a}, —NH₂, —NH(CH₂)ₚN(C₁₋₄alkyl)₂ wherein p is an integer from 1 to 5, or —NH(CH₂)ₚNHC(═NH)NH₂ wherein p is an integer from 1 to 5.

In some embodiments of the compound of Formula V or pharmaceutically acceptable salt thereof, R¹ is —Y-A²-Y—R¹² and R¹² is —NH₂, —NH(CH₂)ₚN(C₁₋₄alkyl)₂ wherein p is an integer from 1 to 5, or —NH(CH₂)ₚNHC(═NH)NH₂ wherein p is an integer from 1 to 5.

In some embodiments of the compound of Formula V or pharmaceutically acceptable salt thereof, R¹ is —Y-A²-Y—R¹² and R¹² is —NH₂ or —NH(CH₂)ₚN(CH₃)₂ wherein p is 2 or 3.

In some embodiments of the compound of Formula V or pharmaceutically acceptable salt thereof, R² is OH.

In some embodiments of the compound of Formula V or pharmaceutically acceptable salt thereof, R² is —NH₂.

In some embodiments of the compound of Formula V or pharmaceutically acceptable salt thereof, R² is —NH(CH₂)ₚNH₂ wherein p is an integer from 1 to 5 or —NH(CH₂)ₚN(C₁₋₄ alkyl)₂ wherein p is an integer from 1 to 5.

In some embodiments of the compound of Formula V or pharmaceutically acceptable salt thereof, R² is —NH(CH₂)ₚN(C₁₋₄alkyl)₂ wherein p is 2 or 3.

In some embodiments of the compound of Formula V or pharmaceutically acceptable salt thereof, R² is —NH(CH₂)ₚN(CH₃)₂ wherein p is 2 or 3.

In some embodiments of the compound of Formula V or pharmaceutically acceptable salt thereof, R² is R^{12a} (i.e., a moiety of Formula XXXI).

In some embodiments of the compound of Formula V or pharmaceutically acceptable salt thereof, R¹ is —Y-A²-Y—R¹² and R¹² is —NH(CH₂)ₚN(CH₃)₂ wherein p is 2 or 3; and R² is —NH(CH₂)ₚN(CH₃)₂ wherein p is 2 or 3.

In some embodiments of the compound of Formula V or pharmaceutically acceptable salt thereof: R² is —X-A¹-X—R¹³; R¹³ is —C(═O)R¹¹; R¹¹ is aryl substituted with 1, 2, or 3 substituents each independently selected from —OCH₃, —OR^{11a}, —C(═O)NH₂, —C(═O)NH(CH₂)ₚNH₂ wherein p is an integer from 1 to 5, —C(═O)NH(CH₂)ₚNH(C₁₋₄alkyl) wherein p is an integer from 1 to 5, —C(═O)NH(CH₂)ₚN(C₁₋₄alkyl)₂ wherein p is an integer from 1 to 5, or —C(═O)NH(CH₂)ₚNHC(═NH)NH₂ wherein p is an integer from 1 to 5; each R^{11a} is, independently, C₂₋₆ alkyl substituted with R^{B}; and each R^{B} is, independently, —NH₂ or —NH—C(═NH)NH₂.

In some embodiments of the compound of Formula V or pharmaceutically acceptable salt thereof: R² is —X-A¹-X—R¹³; R¹³ is —C(═O)R¹¹; R¹¹ aryl substituted with 1, 2, or 3 substituents each independently selected from —OCH₃, —OR^{11a}, —C(═O)NH₂, —C(═O)NH(CH₂)ₚNH₂ wherein p is an integer from 1 to 5, —C(═O)NH(CH₂)ₚNH(C₁₋₄alkyl) wherein p is an integer from 1 to 5, —C(═O)NH(CH₂)ₚN(C₁₋₄alkyl)₂ wherein p is an integer from 1 to 5, or —C(═O)NH(CH₂)ₚNHC(═NH)NH₂ wherein p is an integer from 1 to 5; each R^{11a} is, independently, C₃₋₆ alkyl (such as C₄₋₆ alkyl) substituted with R^{B}; and each R^{B} is, independently, —NH₂ or —NH—C(═NH)NH₂.

In some embodiments of the compound of Formula V or pharmaceutically acceptable salt thereof: each R¹⁰ is; independently, —OCH₃, or —OR^{10a}; each R^{10a} is, independently, C₁₋₈alkyl substituted with R^{A}; each R^{A} is, independently, —NH₂, —NH—C(═NH)NH₂, or —C(═O)NH₂. In some embodiments, R^{A} is —C(═O)OH.

In some embodiments of the compound of Formula V or pharmaceutically acceptable salt thereof: each R¹⁰ is; independently, —OCH₃, or —OR^{10a}; each R^{10a} is, independently, methyl or C₂₋₆alkyl (such as C₃₋₆alkyl or C₄₋₆alkyl), each substituted with R^{A}; each R^{A} is, independently, —NH₂, —NH—C(═NH)NH₂, or —C(═O)NH₂.

In some embodiments of the compound of Formula V or pharmaceutically acceptable salt thereof: each R¹⁰ is; independently, —OCH₃, or —OR^{10a}; each R^{10a} is, independently, methyl or C₂₋₆alkyl (such as C₃₋₆alkyl or C₄₋₆alkyl), each substituted with R^{A}; each R^{A} is, independently, —C(═O)OH.

In some embodiments of the compound of Formula V or pharmaceutically acceptable salt thereof: each R¹⁰ is; independently, —OCH₃, or —OR^{10a}; each R^{10a} is, independently, methyl or C₃₋₆alkyl (such as C₄₋₆alkyl), each substituted with R^{A}; each R^{A} is, independently, —NH₂, —NH—C(═NH)NH₂, or —C(═O)NH₂.

In some embodiments of the compound of Formula V or pharmaceutically acceptable salt thereof, m is 1. In some embodiments of the compound of Formula V or pharmaceutically acceptable salt thereof, m is 2. In some embodiments of the compound of Formula V or pharmaceutically acceptable salt thereof, m is 3. In some embodiments of the compound of Formula V or pharmaceutically acceptable salt thereof, m is 4.

The present invention also provides compositions comprising any one or more of the compounds of any of the preceding embodiments, or pharmaceutically acceptable salts thereof. In some embodiments, the composition is a pharmaceutical composition comprising any one or more of the compounds of any of the preceding embodiments, or pharmaceutically acceptable salts thereof, and a pharmaceutically acceptable carrier. In some embodiments, the composition comprises a compound of Formula V, or pharmaceutically acceptable salt thereof. In some embodiments, the composition comprises a compound selected from Compounds 201-427, or pharmaceutically acceptable salt thereof. In some embodiments, the compound, or composition comprising the same, can be selected from any combination of Compounds 201-427. In some embodiments, the compound, or composition comprising the same, can be selected from any combination of Compounds 201-427, excluding any one or more of Compounds 201-427.

The present invention also provides methods for antagonizing an anticoagulant agent (such as heparin including, for example, unfractionated heparin, low molecular weight heparin, and synthetically modified heparin or low molecular heparin derivatives) comprising administering to a mammal a compound of any of the preceding embodiments, or pharmaceutically acceptable salts thereof, such as those selected from Compound 201-427 or pharmaceutically salt thereof. In some embodiments, the compound, or composition comprising the same, that is administered can be selected from any combination of Compounds 201-427. In some embodiments, the compound, or composition comprising the same, that is administered can be selected from any combination of Compounds 201-427, excluding any one or more of Compounds 201-427.

The compounds of Formula V or Compounds 201-427 disclosed herein (such as the polymers and oligomers) or pharmaceutically acceptable salts thereof useful in the present invention can be made, for example, by methods described in U.S. Patent Application Publication No. 2006-0041023, U.S. Pat. No. 7,173,102, International Publication No. WO 2004/082643, International Publication No. WO2006093813, and U.S. patent application Ser. No. 12/510,593 filed Jul. 28, 2009. In some embodiments, the compounds of Formula V or Compounds 201-427 disclosed herein (such as the polymers and oligomers) or pharmaceutically acceptable salts thereof useful in the present invention can be selected from those described in U.S. Patent Application Publication No. 2006-0041023, U.S. Pat. No. 7,173,102, International Publication No. WO 2004/082643, International Publication No. WO2006093813, and U.S. patent application Ser. No. 12/510,593 filed Jul. 28, 2009.

In some embodiments, the compound(s) useful in the method of present invention can be chosen from one or more of the compounds (i.e., genuses, sub-genuses, and species) disclosed in U.S. Patent Application Publication No. 2006-0041023, U.S. Pat. No. 7,173,102, International Publication No. WO 2005/123660, International Publication No. WO 2004/082643, International Publication No. WO 2006/093813, and U.S. patent application Ser. No. 12/510,593 filed Jul. 28, 2009, each of which is hereby incorporated by reference in its entirety.

Additional compounds, or pharmaceutically acceptable salts thereof, that are useful in antagonizing an anticoagulant agent (including, for example, unfractionated heparin, low molecular weight heparin, and synthetically modified heparin or low molecular heparin derivatives) can be selected from, for example, Compounds 201-427 disclosed herein, or their pharmaceutically acceptable salts thereof. In some embodiments, the compound, or composition comprising the same, can be selected from any combination of Compounds 201-427. In some embodiments, the compound, or composition comprising the same, can be selected from any combination of Compounds 201-427, excluding any one or more of Compounds 201-427.

At various places in the present specification, substituents of compounds of the invention are disclosed in groups or in ranges. It is specifically intended that the invention include each and every individual subcombination of the members of such groups and ranges. For example, the term “C₁₋₆ alkyl” is specifically intended to individually disclose methyl, ethyl, C₃ alkyl, C₄ alkyl, C₅ alkyl, and C₆ alkyl.

For compounds of the invention in which a variable appears more than once, each variable can be a different moiety selected from the Markush group defining the variable. For example, each of NPL groups and PL groups can be a different moiety selected from the Markush group defining the variable. For example, where a structure is described having two R groups that are simultaneously present on the same compound, the two R groups can represent different moieties selected from the Markush groups defined for R. In another example, when an optionally multiple substituent is designated in the form:

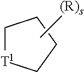

then it is understood that substituent R can occur s number of times on the ring, and R can be a different moiety at each occurrence. Further, in the above example, where the variable T¹ is defined to include hydrogens, such as when T¹ is CH₂, NH, etc., any floating substituent such as R in the above example, can replace a hydrogen of the T¹ variable as well as a hydrogen in any other non-variable component of the ring.

It is further appreciated that certain features of the invention, which are, for clarity, described in the context of separate embodiments, can also be provided in combination in a single embodiment. Conversely, various features of the invention which are, for brevity, described in the context of a single embodiment, can also be provided separately or in any suitable subcombination.

Unless defined otherwise, all technical and scientific terms have the same meaning as is commonly understood by one of ordinary skill in the art to which the embodiments disclosed belongs.

As used herein, the terms “comprising” (and any form of comprising, such as “comprise”, “comprises”, and “comprised”), “having” (and any form of having, such as “have” and “has”), “including” (and any form of including, such as “includes” and “include”), or “containing” (and any form of containing, such as “contains” and “contain”), are inclusive or open-ended and do not exclude additional, un-recited elements or method steps.

As used herein, the terms “a” or “an” means “at least one” or “one or more” unless the context clearly indicates otherwise.

As used herein, the term “about” means that the numerical value is approximate and small variations would not significantly affect the practice of the disclosed embodiments. Where a numerical limitation is used, unless indicated otherwise by the context, “about” means the numerical value can vary by ±10% and remain within the scope of the disclosed embodiments.

As used herein, the term “n-membered”, where n is an integer, typically describes the number of ring-forming atoms in a moiety, where the number of ring-forming atoms is n. For example, pyridine is an example of a 6-membered heteroaryl ring and thiophene is an example of a 5-membered heteroaryl ring.

As used herein, the term “alkyl” refers to a saturated hydrocarbon group which is straight-chained or branched. An alkyl group can contain from 1 to 20, from 2 to 20, from 1 to 10, from 1 to 8, from 1 to 6, from 1 to 4, or from 1 to 3 carbon atoms. Examples of alkyl groups include, but are not limited to, methyl (Me), ethyl (Et), propyl (e.g., n-propyl and isopropyl), butyl (e.g., n-butyl, isobutyl, t-butyl), pentyl (e.g., n-pentyl, isopentyl, neopentyl), and the like.

As used herein, the term “alkylene” or “alkylenyl” refers to a divalent alkyl linking group. An example of an alkylene (or alkylenyl) is methylene or methylenyl (i.e., —CH₂—).

As used herein, the term “alkenyl” refers to an alkyl group having one or more double carbon-carbon bonds. Examples of alkenyl groups include, but are not limited to, ethenyl, propenyl, cyclohexenyl, and the like.

As used herein, the term “alkenylenyl” refers to a divalent linking alkenyl group.

As used herein, the term “alkynyl” refers to an alkyl group having one or more triple carbon-carbon bonds. Examples of alkynyl groups include, but are not limited to, ethynyl, propynyl, and the like.

As used herein, the term “alkynylenyl” refers to a divalent linking alkynyl group.

As used herein, the term “haloalkyl” refers to an alkyl group having one or more halogen substituents. Examples of haloalkyl groups include, but are not limited to, CF₃, C₂F₅, CHF₂, CCl₃, CHCl₂, C₂Cl₅, CH₂CF₃, and the like.

As used herein, the term “aryl” refers to monocyclic or polycyclic (e.g., having 2, 3 or 4 fused rings) aromatic hydrocarbons. In some embodiments, aryl groups have from 6 to about 20 carbon atoms. In some embodiments, aryl groups have from 6 to 10 carbon atoms. Examples of aryl groups include, but are not limited to, phenyl, naphthyl, anthracenyl, phenanthrenyl, indanyl, indenyl, and the like.

As used herein, the term “cycloalkyl” refers to non-aromatic cyclic hydrocarbons including cyclized alkyl, alkenyl, and alkynyl groups that contain up to 20 ring-forming carbon atoms. Cycloalkyl groups can include mono- or polycyclic ring systems such as fused ring systems, bridged ring systems, and spiro ring systems. In some embodiments, polycyclic ring systems include 2, 3, or 4 fused rings. A cycloalkyl group can contain from 3 to about 15, from 3 to 10, from 3 to 8, from 3 to 6, from 4 to 6, from 3 to 5, or from 5 to 6 ring-forming carbon atoms. Ring-forming carbon atoms of a cycloalkyl group can be optionally substituted by oxo or sulfido. Examples of cycloalkyl groups include, but are not limited to, cyclopropyl, cyclobutyl, cyclopentyl, cyclohexyl, cycloheptyl, cyclopentenyl, cyclohexenyl, cyclohexadienyl, cycloheptatrienyl, norbornyl, norpinyl, norcarnyl, adamantyl, and the like. Also included in the definition of cycloalkyl are moieties that have one or more aromatic rings fused (i.e., having a bond in common with) to the cycloalkyl ring, for example, benzo or thienyl derivatives of pentane, pentene, hexane, and the like (e.g., 2,3-dihydro-1H-indene-1-yl, or 1H-inden-2(3H)-one-1-yl).

As used herein, the term “heteroaryl” refers to an aromatic heterocycle having up to 20 ring-forming atoms and having at least one heteroatom ring member (ring-forming atom) such as sulfur, oxygen, or nitrogen. In some embodiments, the heteroaryl group has at least one or more heteroatom ring-forming atoms, each of which are, independently, sulfur, oxygen, or nitrogen. In some embodiments, the heteroaryl group has from 1 to about 20 carbon atoms, from 1 to 5, from 1 to 4, from 1 to 3, or from 1 to 2, carbon atoms as ring-forming atoms. In some embodiments, the heteroaryl group contains 3 to 14, 3 to 7, or 5 to 6 ring-forming atoms. In some embodiments, the heteroaryl group has 1 to 4, 1 to 3, or 1 to 2 heteroatoms. Heteroaryl groups include monocyclic and polycyclic (e.g., having 2, 3 or 4 fused rings) systems. Examples of heteroaryl groups include, but are not limited to, pyridyl, pyrimidinyl, pyrazinyl, pyridazinyl, triazinyl, furyl, quinolyl, isoquinolyl, thienyl, imidazolyl, thiazolyl, indolyl (such as indol-3-yl), pyrryl, oxazolyl, benzofuryl, benzothienyl, benzthiazolyl, isoxazolyl, pyrazolyl, triazolyl, tetrazolyl, indazolyl, 1,2,4-thiadiazolyl, isothiazolyl, benzothienyl, purinyl, carbazolyl, benzimidazolyl, indolinyl, and the like.

As used herein, the term “heterocycloalkyl” refers to non-aromatic heterocycles having up to 20 ring-forming atoms including cyclized alkyl, alkenyl, and alkynyl groups, where one or more of the ring-forming carbon atoms is replaced by a heteroatom such as an O, N, or S atom. Heterocycloalkyl groups can be mono or polycyclic (e.g., fused, bridged, or spiro systems). In some embodiments, the heterocycloalkyl group has from 1 to about 20 carbon atoms, or 3 to about 20 carbon atoms. In some embodiments, the heterocycloalkyl group contains 3 to 14, 3 to 7, or 5 to 6 ring-forming atoms. In some embodiments, the heterocycloalkyl group has 1 to 4, 1 to 3, or 1 to 2 heteroatoms. In some embodiments, the heterocycloalkyl group contains 0 to 3 double bonds. In some embodiments, the heterocycloalkyl group contains 0 to 2 triple bonds. Example of heterocycloalkyl groups include, but are not limited to, morpholino, thiomorpholino, piperazinyl, tetrahydrofuranyl, tetrahydrothienyl, 2,3-dihydrobenzofuryl, 1,3-benzodioxole, benzo-1,4-dioxane, piperidinyl, pyrrolidinyl, isoxazolidinyl, isothiazolidinyl, pyrazolidinyl, oxazolidinyl, thiazolidinyl, imidazolidinyl, pyrrolidin-2-one-3-yl, and the like. In addition, ring-forming carbon atoms and heteroatoms of a heterocycloalkyl group can be optionally substituted by oxo or sulfido. For example, a ring-forming S atom can be substituted by 1 or 2 oxo (i.e., form a S(O) or S(O)₂). For another example, a ring-forming C atom can be substituted by oxo (i.e., form carbonyl). Also included in the definition of heterocycloalkyl are moieties that have one or more aromatic rings fused (i.e., having a bond in common with) to the nonaromatic heterocyclic ring including, but not limited to, pyridinyl, thiophenyl, phthalimidyl, naphthalimidyl, and benzo derivatives of heterocycles such as indolene, isoindolene, isoindolin-1-one-3-yl, 4,5,6,7-tetrahydrothieno[2,3-c]pyridine-5-yl, 5,6-dihydrothieno[2,3-c]pyridin-7(4H)-one-5-yl, and 3,4-dihydroisoquinolin-1(2H)-one-3yl groups. Ring-forming carbon atoms and heteroatoms of the heterocycloalkyl group can be optionally substituted by oxo or sulfido.

As used herein, the term “halo” refers to halogen groups including, but not limited to fluoro, chloro, bromo, and iodo.

As used herein, the term “alkoxy” refers to an —O-alkyl group. Examples of alkoxy groups include, but are not limited to, methoxy, ethoxy, propoxy (e.g., n-propoxy and isopropoxy), t-butoxy, and the like.

As used herein, the term “haloalkoxy” refers to an —O-haloalkyl group. An example of an haloalkoxy group is OCF₃.

As used herein, the term “alkylthio” refers to an —S-alkyl group. An example of an alkylthio group is —SCH₂CH₃.

As used herein, the term “arylalkyl” refers to a C₁₋₆ alkyl substituted by aryl and “cycloalkylalkyl” refers to C₁₋₆ alkyl substituted by cycloalkyl.

As used herein, the term “heteroarylalkyl” refers to a C₁₋₆ alkyl group substituted by a heteroaryl group, and “heterocycloalkylalkyl” refers to a C₁₋₆ alkyl substituted by heterocycloalkyl.

As used herein, the term “amino” refers to NH₂.

As used herein, the term “alkylamino” refers to an amino group substituted by an alkyl group. An example of an alkylamino is —NHCH₂CH₃.

As used herein, the term “arylamino” refers to an amino group substituted by an aryl group. An example of an alkylamino is —NH(phenyl).

As used herein, the term “aminoalkyl” refers to an alkyl group substituted by an amino group. An example of an aminoalkyl is —CH₂CH₂NH₂.

As used herein, the term “aminosulfonyl” refers to —S(═O)₂NH₂.

As used herein, the term “aminoalkoxy” refers to an alkoxy group substituted by an amino group. An example of an aminoalkoxy is —OCH₂CH₂NH₂.

As used herein, the term “aminoalkylthio” refers to an alkylthio group substituted by an amino group. An example of an aminoalkylthio is —SCH₂CH₂NH₂.

As used herein, the term “amidino” refers to —C(═NH)NH₂.

As used herein, the term “acylamino” refers to an amino group substituted by an acyl group (e.g., —O—C(═O)—H or —O—C(═O)-alkyl). An example of an acylamino is —NHC(═O)H or —NHC(═O)CH₃. The term “lower acylamino” refers to an amino group substituted by a loweracyl group (e.g., —O—C(═O)—H or —O—C(═O)—C₁₋₆alkyl). An example of a lower acylamino is —NHC(═O)H or —NHC(═O)CH₃.

As used herein, the term “carbamoyl” refers to —C(═O)—NH₂.

As used herein, the term “cyano” refers to —CN.

As used herein, the term “dialkylamino” refers to an amino group substituted by two alkyl groups.

As used herein, the term “diazamino” refers to —N(NH₂)₂.

As used herein, the term “guanidino” refers to —NH(═NH)NH₂.

As used herein, the term “heteroarylamino” refers to an amino group substituted by a heteroaryl group. An example of an alkylamino is —NH-(2-pyridyl).

As used herein, the term “hydroxyalkyl” or “hydroxylalkyl” refers to an alkyl group substituted by a hydroxyl group. Examples of a hydroxylalkyl include, but are not limited to, —CH₂OH and —CH₂CH₂OH.

As used herein, the term “nitro” refers to —NO₂.

As used herein, the term “semicarbazone” refers to ═NNHC(═O)NH₂.

As used herein, the term “ureido” refers to —NHC(═O)—NH₂.

As used used herein, the phrase “optionally substituted” means that substitution is optional and therefore includes both unsubstituted and substituted atoms and moieties. A “substituted” atom or moiety indicates that any hydrogen on the designated atom or moiety can be replaced with a selection from the indicated substituent group, provided that the normal valency of the designated atom or moiety is not exceeded, and that the substitution results in a stable compound. For example, if a methyl group is optionally substituted, then 3 hydrogen atoms on the carbon atom can be replaced with substituent groups.

As used herein, the term, “compound” refers to all stereoisomers, tautomers, and isotopes of the compounds described in the present invention.

As used herein, the phrase “substantially isolated” refers to a compound that is at least partially or substantially separated from the environment in which it is formed or detected.

As used herein, the phrase “pharmaceutically acceptable” refers to those compounds, materials, compositions, and/or dosage forms which are, within the scope of sound medical judgment, suitable for use in contact with tissues of humans and animals.

As used herein, the term “animal” includes, but is not limited to, humans and non-human vertebrates such as wild, domestic and farm animals.

As used herein, the term “contacting” refers to the bringing together of an indicated moiety in an in vitro system or an in vivo system. For example, “contacting” a heparin with a compound of the invention includes the administration of a compound of the present invention to an individual or patient, such as a human, having been administered a heparin, as well as, for example, introducing a compound of the invention into a sample containing a cellular or purified preparation containing the heparin, or before an individual has been administered a heparin.

As used herein, the term “individual” or “patient,” used interchangeably, refers to any animal, including mammals, such as mice, rats, other rodents, rabbits, dogs, cats, swine, cattle, sheep, horses, or primates, such as humans.

As used herein, the phrase “therapeutically effective amount” refers to the amount of active compound or pharmaceutical agent that elicits the biological or medicinal response that is being sought in a tissue, system, animal, individual or human by a researcher, veterinarian, medical doctor or other clinician.

The compounds described herein can be asymmetric (e.g., having one or more stereocenters). All stereoisomers, such as enantiomers and diastereomers, are intended to be included within the scope of the invention unless otherwise indicated. Compounds of the present invention that contain asymmetrically substituted carbon atoms can be isolated in optically active or racemic forms. Methods of preparation of optically active forms from optically active starting materials are known in the art, such as by resolution of racemic mixtures or by stereoselective synthesis. Many geometric isomers of olefins, C═N double bonds, and the like can also be present in the compounds described herein, and all such stable isomers are contemplated in the present invention. Cis and trans geometric isomers of the compounds of the present invention are also included within the scope of the invention and can be isolated as a mixture of isomers or as separated isomeric forms. Where a compound capable of stereoisomerism or geometric isomerism is designated in its structure or name without reference to specific R/S or cis/trans configurations, it is intended that all such isomers are contemplated.

Resolution of racemic mixtures of compounds can be carried out by any of numerous methods known in the art, including, for example, fractional recrystallizaion using a chiral resolving acid which is an optically active, salt-forming organic acid. Suitable resolving agents for fractional recrystallization methods include, but are not limited to, optically active acids, such as the D and L forms of tartaric acid, diacetyltartaric acid, dibenzoyltartaric acid, mandelic acid, malic acid, lactic acid, and the various optically active camphorsulfonic acids such as β-camphorsulfonic acid. Other resolving agents suitable for fractional crystallization methods include, but are not limited to, stereoisomerically pure forms of α-methylbenzylamine (e.g., S and R forms, or diastereomerically pure forms), 2-phenylglycinol, norephedrine, ephedrine, N-methylephedrine, cyclohexylethylamine, 1,2-diaminocyclohexane, and the like.

Resolution of racemic mixtures can also be carried out by elution on a column packed with an optically active resolving agent (e.g., dinitrobenzoylphenylglycine). Suitable elution solvent compositions can be determined by one skilled in the art.

Compounds of the invention also include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Examples of prototropic tautomers include, but are not limited to, ketone-enol pairs, amide-imidic acid pairs, lactam-lactim pairs, amide-imidic acid pairs, enamine-imine pairs, and annular forms where a proton can occupy two or more positions of a heterocyclic system including, but not limited to, 1H- and 3H-imidazole, 1H-, 2H- and 4H-1,2,4-triazole, 1H- and 2H-isoindole, and 1H- and 2H-pyrazole. Tautomeric forms can be in equilibrium or sterically locked into one form by appropriate substitution.

Compounds of the invention also include hydrates and solvates, as well as anhydrous and non-solvated forms.

All compounds and pharmaceutically acceptable salts thereof can be prepared or be present together with other substances such as water and solvents (e.g., hydrates and solvates) or can be isolated.

Compounds of the invention can also include all isotopes of atoms occurring in the intermediates or final compounds. Isotopes include those atoms having the same atomic number but different mass numbers. For example, isotopes of hydrogen include tritium and deuterium.

In some embodiments, the compounds of the invention, or salts thereof, are substantially isolated. Partial separation can include, for example, a composition enriched in the compound of the invention. Substantial separation can include compositions containing at least about 50%, at least about 60%, at least about 70%, at least about 80%, at least about 90%, at least about 95%, at least about 97%, or at least about 99% by weight of the compound of the invention, or salt thereof. Methods for isolating compounds and their salts are routine in the art.

Compounds of the invention are intended to include compounds with stable structures. As used herein, the phrases “stable compound” and “stable structure” refer to a compound that is sufficiently robust to survive isolation to a useful degree of purity from a reaction mixture, and formulation into an efficacious therapeutic agent.

The present invention also includes quaternary ammonium salts of the compounds described herein, where the compounds have one or more tertiary amine moiety. As used herein, the phrase “quaternary ammonium salts” refers to derivatives of the disclosed compounds with one or more tertiary amine moieties wherein at least one of the tertiary amine moieties in the parent compound is modified by converting the tertiary amine moiety to a quaternary ammonium cation via alkylation (and the cations are balanced by anions such as Cl⁻, CH₃COO⁻, and CF₃COO⁻), for example methylation or ethylation.

Some of the compounds of the present invention may be capable of adopting amphiphilic conformations that allow for the segregation of polar and nonpolar regions of the molecule into different spatial regions and provide the basis for a number of uses. For example, some compounds of the invention may adopt amphiphilic conformations that are capable of binding to heparin (including, for example, unfractionated heparin, low molecular weight heparin, and synthetically modified heparin or low molecular heparin derivatives). Although not wishing to be bound by any particular theory, it is believed that compounds of the invention can interact with heparin through electrostatic interactions.

Many of the compounds of Formula I, Ia, Ia-1, Ia-2, Ia-3, II, IIa, III, IV, and V are significantly smaller and easier to prepare than their naturally occurring counterparts. Moreover, the non-peptidic compounds of the present invention are significantly less toxic towards human erythrocytes, much less expensive to prepare, and are expected to be much more stable in vivo.

The compounds of the invention may be useful as anti-heparin agents (i.e., antagonizing the anticoagulant effect of an anticoagulant such as unfractionated heparin, low molecular heparin, and a derivative of heparin or low molecular heparin) in a number of applications. For example, compounds of the invention may be used therapeutically to antagonize the anticoagulant effect of an anticoagulant agent (for example unfractionated heparin, low molecular heparin, or a derivative of heparin or low molecular heparin), present in patients such as animals, including humans and non-human vertebrates such as wild, domestic and farm animals. The anticoagulant effect of the anticoagulant agent (for example unfractionated heparin, low molecular heparin, or a derivative of heparin or low molecular heparin) present in a patient may be antagonized by administering to the patient an effective amount of a compound of the invention or a salt thereof, or a pharmaceutical composition comprising a compound of the invention or a salt thereof. The compound or salt, or composition thereof, can be administered systemically or topically and can be administered to any body site or tissue.

As used herein, unless specified otherwise, “heparin” refers to naturally occurring unfractionated heparin and low molecular weight heparin, which can be used as an anticoagulant in diseases that feature thrombosis, as well as for prophylaxis in situations that lead to a high risk of thrombosis. Natural heparins have polysaccharide chains of varying lengths, or molecular weights (including salts). Natural heparin has polysaccharide chains of molecular weight from about 5000 to over 40,000 Daltons. Low-molecular-weight heparins (LMWHs), in contrast, are fragments of unfractionated heparins, and have short chains of polysaccharide (including salts). LMWHs have an average molecular weight of less than 8000 Da and at least 60% of all chains have a molecular weight less than 8000 Da. Examples of LMWH include, but are limited to, enoxaparin, reviparin, and tinzaparin. As used herein, the term “heparin” further includes anticoagulant agents that are derivatives of unfractionated heparin and/or LMWH, for example, by chemical modification or through enzymatic process. Examples of such heparin derivatives (for example, chemically modified unfractionated heparin and/or LMWH) include fondaparinux.

Although the disclosed compounds are suitable, other functional groups can be incorporated into the compound with an expectation of similar results. In particular, thioamides and thioesters are anticipated to have very similar properties. The distance between aromatic rings can impact the geometrical pattern of the compound and this distance can be altered by incorporating aliphatic chains of varying length, which can be optionally substituted or can comprise an amino acid, a dicarboxylic acid or a diamine. The distance between and the relative orientation of monomers within the compounds can also be altered by replacing the amide bond with a surrogate having additional atoms. Thus, replacing a carbonyl group with a dicarbonyl alters the distance between the monomers and the propensity of dicarbonyl unit to adopt an anti arrangement of the two carbonyl moiety and alter the periodicity of the compound. Pyromellitic anhydride represents still another alternative to simple amide linkages which can alter the conformation and physical properties of the compound. Modern methods of solid phase organic chemistry (E. Atherton and R. C. Sheppard, Solid Phase Peptide Synthesis A Practical Approach IRL Press Oxford 1989) now allow the synthesis of homodisperse compounds with molecular weights approaching 5,000 Daltons. Other substitution patterns are equally effective.

The compounds of the invention also include derivatives referred to as prodrugs. As used herein, the term “prodrug” refers to a derivative of a known direct acting drug, which derivative has enhanced delivery characteristics and therapeutic value as compared to the drug, and is transformed into the active drug by an enzymatic or chemical process.

It is understood that the present invention encompasses the use, where applicable, of stereoisomers, diastereomers and optical stereoisomers of the compounds of the invention, as well as mixtures thereof, for antagonizing the anticoagulant effect of heparin. Additionally, it is understood that stereoisomers, diastereomers, and optical stereoisomers of the compounds of the invention, and mixtures thereof, are within the scope of the invention. By way of non-limiting example, the mixture may be a racemate or the mixture may comprise unequal proportions of one particular stereoisomer over the other. Additionally, the compounds of the invention can be provided as a substantially pure stereoisomers, diastereomers and optical stereoisomers (such as epimers).

The compounds of the invention can be provided in the form of an acceptable salt (i.e., a pharmaceutically acceptable salt) for antagonizing the anticoagulant effect of heparin. Salts can be provided for pharmaceutical use, or as an intermediate in preparing the pharmaceutically desired form of the compounds of the invention. One example of a salt that can be considered to be acceptable is the hydrochloride acid addition salt. Hydrochloride acid addition salts are often acceptable salts when the pharmaceutically active agent has an amine group that can be protonated. Since the compounds of the invention may be polyionic, such as a polyamine, the acceptable salt can be provided in the form of a poly(amine hydrochloride).

In some embodiments, the methods of the present invention can effectively antagonize the anticoagulant effect of unfractionated heparin. In some embodiments, the methods of the present invention can effectively antagonize the anticoagulant effect of a low molecular weight heparin such as enoxaparin. In some embodiments, the methods of the present invention can effectively antagonize the anticoagulant effect of a synthetically modified heparin derivative such as fondaparinux.

As used herein, the term “antagonize” or “antagonizing” refers to reducing or completely eliminating the anticoagulant effect of heparin. In some embodiments, the method of the present invention can antagonize greater than about 50%, 60%, 70%, 80%, 85%, 88%, 90%, 92%, 95%, 98%, 99%, 99.2%, 99.5%, 99.8%, or 99.9% of the anticoagulant effect of heparin.

In some embodiments, the compound or salt thereof used in the present invention binds to heparin (including, for example, unfractionated heparin, low molecular weight heparin, and synthetically modified heparin or low molecular heparin derivatives) with an EC₅₀ of less than about 100, 90, 80, 70, 60, 50, 40, 30, 20, 15, 10, 5, 2, 1, 0.9, 0.8, 0.7, 0.6, 0.5, 0.4, 0.3, 0.2, 0.1, 0.09, 0.08, 0.07, 0.06, 0.05, 0.02, 0.01, 0.001, 0.0001, or 0.00001 μg/mL. In some embodiments, the compound or salt thereof used in the present invention binds to heparin (including, for example, unfractionated heparin, low molecular weight heparin, and synthetically modified heparin or low molecular heparin derivatives) with an EC₅₀ less than about 30, 20, 15, 10, 5, 2, 1, 0.9, 0.8, 0.7, 0.6, 0.5, 0.4, 0.3, 0.2, 0.1, 0.01, 0.001, 0.0001, or 0.00001 μg/mL.

In some embodiments, the compound or salt thereof used in the present invention binds to heparin (including, for example, unfractionated heparin, low molecular weight heparin, and synthetically modified heparin or low molecular heparin derivatives) with an EC₅₀ less than about 100, 90, 80, 70, 60, 50, 40, 30, 20, 15, 10, 5, 2, 1, 0.9, 0.8, 0.7, 0.6, 0.5, 0.4, 0.3, 0.2, 0.1, 0.09, 0.08, 0.07, 0.06, 0.05, 0.02, 0.01, 0.001, 0.0001, or 0.00001 μM. In some embodiments, the compound or salt thereof used in the present invention binds to heparin (including, for example, unfractionated heparin, low molecular weight heparin, and synthetically modified heparin or low molecular heparin derivatives) with an EC₅₀ less than about 30, 20, 15, 10, 5, 2, 1, 0.9, 0.8, 0.7, 0.6, 0.5, 0.4, 0.3, 0.2, 0.1, 0.01, 0.001, 0.0001, or 0.00001 μM. In some embodiments, the compound or salt thereof used in the present invention binds to heparin (including, for example, unfractionated heparin, low molecular weight heparin, and synthetically modified heparin or low molecular heparin derivatives) with an EC₅₀ less than about 500, 200, 100, 90, 80, 70, 60, 50, 40, 30, 20, 15, 10, 5, 2, 1, 0.1, 0.01, 0.001, 0.0001, or 0.00001 μM.

In some embodiments, the compound or salt thereof used in the present invention binds to heparin (including, for example, unfractionated heparin, low molecular weight heparin, and synthetically modified heparin or low molecular heparin derivatives) with an EC₅₀ of less than that of protamine (including protamine salt such as protamine sulfate).

In some embodiments, the compound or salt thereof used in the present invention can effectively antagonize the anticoagulant effect of an anticoagulant agent (including, for example, unfractionated heparin, low molecular weight heparin, and synthetically modified heparin or low molecular heparin derivatives) with a dosage of less than about 10, 9, 8, 7, 6, 5, 4, 3, 2, or 1 equivalent (by weight) to the heparin. In some embodiments, the compound or salt thereof used in the present invention can effectively antagonize the anticoagulant effect of heparin with a dosage of less than about 5, 4, 2, or 1 equivalent (by weight) to that of the heparin. In some embodiments, the compound or salt thereof used in the present invention can antagonize (or neutralize) greater than about 40%, 50%, 60%, 70%, 80, 90%, 95%, 98%, 99%, or 99.5% of the anticoagulant effect of an anticoagulant agent (including, for example, unfractionated heparin, low molecular weight heparin, and synthetically modified heparin or low molecular heparin derivatives) with a dosage of less than about 10, 9, 8, 7, 6, 5, 4, 3, 2, or 1 equivalent (by weight) to that of the heparin. In some embodiments, the compound or salt thereof used in the present invention can antagonize (or neutralize) 100% (i.e., completely) of the anticoagulant effect of an anticoagulant agent (including, for example, unfractionated heparin, low molecular weight heparin, and synthetically modified heparin or low molecular heparin derivatives) with a dosage of less than about 10, 9, 8, 7, 6, 5, 4, 3, 2, or 1 equivalent (by weight) to that of the heparin. In some embodiments, the compound or salt thereof used in the present invention antagonizes the anticoagulant effect of an anticoagulant agent (including, for example, unfractionated heparin, low molecular weight heparin, and synthetically modified heparin or low molecular heparin derivatives) more effectively than protamine.

In some embodiments, the compound or salt thereof used in the present invention can effectively antagonize the anticoagulant effect of an anticoagulant agent (including, for example, unfractionated heparin, low molecular weight heparin, and synthetically modified heparin or low molecular heparin derivatives) through antagonizing the AT activity of the heparin, the anti-factor Xa activity of the heparin, the anti-factor IIa activity of the heparin, or any combination thereof.

In some embodiments, the method of the present invention can rapidly antagonize the anticoagulant effect of an anticoagulant agent (including, for example, unfractionated heparin, low molecular weight heparin, and synthetically modified heparin or low molecular heparin derivatives), for example, antagonize (or neutralize) greater than about 40%, 50%, 60%, 70%, 80, 90%, 95%, 98%, 99%, or 99.5% of the anticoagulant effect of the heparin in less than about 30, 20, 15, 10, 8, 5, 2, 1, 0.9, 0.8, 0.7, 0.6, 0.5, 0.4, 0.3, 0.2, or 0.1 minute. In some embodiments, the method of the present invention can antagonize (or neutralize) greater than about 40%, 50%, 60%, 70%, 80, 90%, 95%, 98%, 99%, or 99.5% of the anticoagulant effect of heparin in less than about 10, 8, 5, 2, or 1 minute. In some embodiments, the method of the present invention can antagonize (or neutralize) greater than about 40%, 50%, 60%, 70%, 80, 90%, 95%, 98%, 99%, or 99.5% of the anticoagulant effect of heparin in less than about 60, 55, 50, 45, 40, 35, 30, 25, 20, 15, 10, 8, 5, 2, or 1 second.

In some embodiments, after the anticoagulant effect of heparin in a patient during anticoagulant therapy is antagonized (for example, by 80% or more) by methods of the present invention, a new dose of heparin can effectively restore the anticoagulant therapy, for example, greater than about 80% or 90% fo the anticoagulant effect of heparin of the new dose can be achieved in less than about 20, 15, 10, 8, 5, 2, or 1 minute.

In some embodiments, the present invention provides methods for antagonizing the anticoagulant effect of heparin with low or no toxicity, hemodynamic and/or hematological adverse side effects. In some embodiments, the methods of the present invention have low or no side effects associated with use of protamine such as one or more selected from systemic vasodilation and hypotension, bradycardia, pulmonary artery hypertension, pulmonary vasoconstriction, thrombocytopenia, and neutropenia. In some embodiments, the methods of the present invention have low or no side effects associated with use of protamine such as anaphylactic-type reactions involving both nonimmunogenic and immunogenic-mediated pathways. In some embodiments, the compounds and/or the salts used in the present invention have low or no antigenicity and/or immunogenicity comparing to those of protamine molecules. In some embodiments, the present methods for antagonizing the anticoagulant effect of heparin can preserve hemodynamic stability, such as during and/or following infusion.

In some embodiments, the present methods for antagonizing the anticoagulant effect of heparin can be used in a patient who receives anticoagulant therapy, for example, who uses fondaparinux for the prophylaxis of deep vein thrombosis following hip repair/replacement, knee replacement and abdominal surgery; or uses UFH or LMWH for coronary bypass surgery.

Polyamides and polyesters that are useful for the present invention can be prepared by typical condensation polymerization and addition polymerization processes (see, for example, G. Odian, Principles of Polymerization, John Wiley & Sons, Third Edition (1991), and M. Steven, Polymer Chemistry, Oxford University Press (1999)). Most commonly, the polyamides are prepared by a) thermal dehydration of amine salts of carboxylic acids, b) reaction of acid chlorides with amines, and c) aminolysis of esters. Methods a) and c) are of limited use in polymerizations of aniline derivatives which are generally prepared utilizing acid chlorides. The skilled chemist, however, will recognize that there are many alternative active acylating agents, for example phosphoryl anhydrides, active esters or azides, which may replace an acid chloride and which, depending of the particular polymer being prepared, may be superior to an acid chloride. The acid chloride route is probably the most versatile and has been used extensively for the synthesis of aromatic polyamides.

Homopolymers derived from substituted aminobenzoic acid derivatives can also prepared in a stepwise fashion. A stepwise process comprises coupling an N-protected amino acid to an amine (or hydroxy group) and subsequently removing the amine-protecting group and repeating the process. These techniques have been highly refined for synthesis of specific peptides, allow for the synthesis of specific sequences, and both solid-phase and solution techniques for peptide synthesis are directly applicable to the present invention. An alternative embodiment of the present invention is the corresponding polysulfonamides that can be prepared in analogous fashion by substituting sulfonyl chlorides for carboxylic acid chlorides.

The most common method for the preparation of polyureas is the reaction of diamines with diisocyanates (see, Yamaguchi et al., Polym. Bull., 2000, 44, 247). This exothermic reaction can be carried out by solution techniques or by interfacial techniques. One skilled in organic and polymer chemistry will appreciate that the diisocyanate can be replaced with a variety of other bis-acylating agents, such as phosgene or N,N′-(diimidazolyl)carbonyl, with similar results. Polyurethanes are prepared by comparable techniques using a diisocyanate and a dialcohol or by reaction of a diamine with a bis-chloroformate.

The syntheses of compounds of the invention can be carried out by routine and/or known methods such as those disclosed in, for example, U.S. Patent Application Publication Nos. 2005-0287108, 2006-0041023, U.S. Pat. No. 7,173,102, International Publication Nos. WO 2005/123660, WO 2004/082643, and WO 2006/093813, and U.S. application Ser. No. 12/510,593 filed Jul. 28, 2009, each of which is incorporated herein by reference in its entirety. Numerous pathways are available to incorporate polar and nonpolar side chains. Phenolic groups on the monomer can be alkylated. Alkylation of the commercially available phenol will be accomplished with standard Williamson ether synthesis for the non-polar side chain with ethyl bromide as the alkylating agent. Polar sidechains can be introduced with bifunctional alkylating agents such as BOC—NH(CH₂)₂Br. Alternately, the phenol group can be alkylated to install the desired polar side chain function by employing the Mitsonobu reaction with BOC—NH(CH₂)₂—OH, triphenyl phosphine, and diethyl acetylenedicarboxylate. Standard conditions for reduction of the nitro groups and hydrolysis of the ester afford the amino acid. With the aniline and benzoic acid in hand, coupling can be effected under a variety of conditions. Alternatively, the hydroxy group of the (di)nitrophenol can be converted to a leaving group and a functionality introduced under nucleophilic aromatic substitution conditions. Other potential scaffolds that can be prepared with similar sequences are methyl 2-nitro-4-hydroxybenzoate and methyl 2-hydroxy-4-nitrobenzoate.

The compounds of the invention can also be designed using computer-aided computational techniques, such as de novo design techniques, to embody the amphiphilic properties. In general, de novo design of compounds is performed by defining a three-dimensional framework of the backbone assembled from a repeating sequence of monomers using molecular dynamics and quantum force field calculations. Next, side groups are computationally grafted onto the backbone to maximize diversity and maintain drug-like properties. The best combinations of functional groups are then computationally selected to produce a cationic, amphiphilic structures. Representative compounds can be synthesized from this selected library to verify structures and test their biological activity. Novel molecular dynamic and coarse grain modeling programs have also been developed for this approach because existing force fields developed for biological molecules, such as peptides, were unreliable in these oligomer applications (see, Car et al., Phys. Rev. Lett., 1985, 55, 2471-2474; Siepmann et al., Mol. Phys., 1992, 75, 59-70; Martin et al., J. Phys. Chem., 1999, 103, 4508-4517; and Brooks et al., J. Comp. Chem., 1983, 4, 187-217). Several chemical structural series of compounds have been prepared. See, for example, International Publication No. WO 2002/100295, which is incorporated herein by reference in its entirety. The compounds of the invention can be prepared in a similar manner. Molecular dynamic and coarse grain modeling programs can be used for a design approach. See, for example, U.S. application Ser. No. 10/446,171, filed May 28, 2003, and U.S. application Ser. No. 10/459,698, filed Jun. 12, 2003, each of which is incorporated herein by reference in its entirety.

After verifying the suitability of the force field by comparing computed predictions of the structure and thermodynamic properties to molecules that have similar torsional patterns and for which experimental data are available, the fitted torsions can then be combined with bond stretching, bending, one-four, van der Waals, and electrostatic potentials borrowed from the CHARMM (see, Brooks et al., J. Comp. Chem., 1983, 4,187-217) and TraPPE (Martin et al., J. Phys. Chem., 1999, 103, 4508-4517; and Wick et al., J. Phys. Chem., 2000, 104, 3093-3104) molecular dynamics force fields. To identify conformations that can adopt periodic folding patterns with polar groups and apolar groups lined up on the opposite sides, initial structures can be obtained with the Gaussian package (see, Frisch et al., Gaussian 98 (revision A.7) Gaussian Inc., Pittsburgh, Pa. 1998). Then, the parallelized plane-wave Car-Parrinello CP-MD (see, Car et al., Phys. Rev. Lett., 1985, 55, 2471-2474) program, (see, Rothlisberger et al., J. Chem. Phys., 1996, 3692-3700) can be used to obtain energies at the minimum and constrained geometries. The conformations of the compounds without side-chains can be investigated in the gas phase. Both MD and MC methods can be used to sample the conformations. The former is useful for global motions of the compound. With biasing techniques (see, Siepmann et al., Mol. Phys., 1992, 75, 59-70; Martin et al., J. Phys. Chem., 1999, 103, 4508-4517; and Vlugt et al., Mol. Phys., 1998, 94, 727-733), the latter allows efficient sampling for compounds with multiple local minimum configurations that are separated by relatively large barriers.

The potential conformations are examined for positions to attach pendant groups that will impart amphiphilic character to the secondary structure. Compounds selected from the gas phase studies with suitable backbone conformations and with side-chains at the optimal positions to introduce amphiphilicity can be further evaluated in a model interfacial system. n-hexane/water can be chosen because it is simple and cheap for calculations while it mimics well the lipid/water bilayer environment. Compound secondary structures that require inter-compound interactions can be identified by repeating the above-mentioned calculations using a periodically repeated series of unit cells of various symmetries (so called variable cell molecular dynamics or Monte Carlo technique) with or without solvent. The results of these calculations can guide the selection of candidates for synthesis.

An example of the design, synthesis, and testing of arylamide polymers and oligomers, a related group of compounds of the invention, is presented in Tew et al., Proc. Natl. Acad. Sci. USA, 2002, 99, 5110-5114, which is incorporated herein by reference in its entirety.

Compounds of the invention can be synthesized by solid-phase synthetic procedures well know to those of skill in the art (see, Tew et al., Proc. Natl. Acad. Sci. USA, 2002, 99, 5110-5114; Barany et al., Int. J. Pept. Prot. Res., 1987, 30, 705-739; Solid-phase Synthesis: A Practical Guide, Kates, S. A., and Albericio, F., eds., Marcel Dekker, New York (2000); and Dörwald, F. Z., Organic Synthesis on Solid Phase: Supports, Linkers, Reactions, 2nd Ed., Wiley-VCH, Weinheim (2002)).

The compounds of the invention can be administered in any conventional manner by any route where they are active. Administration can be systemic, topical, or oral. For example, administration can be, but is not limited to, parenteral, subcutaneous, intravenous, intramuscular, intraperitoneal, transdermal, oral, buccal, or ocular routes, or intravaginally, by inhalation, by depot injections, or by implants. Thus, modes of administration for the compounds of the invention (either alone or in combination with other pharmaceuticals) can be, but are not limited to, sublingual, injectable (including short-acting, depot, implant and pellet forms injected subcutaneously or intramuscularly), or by use of vaginal creams, suppositories, pessaries, vaginal rings, rectal suppositories, intrauterine devices, and transdermal forms such as patches and creams. The selection of the specific route of administration and the dose regimen is to be adjusted or titrated by the clinician according to methods known to the clinician to obtain the desired clinical response. The amount of compounds of the invention to be administered is that amount which is therapeutically effective. The dosage to be administered will depend on the characteristics of the subject being treated, e.g., the particular animal treated, age, weight, health, types of concurrent treatment, if any, and frequency of treatments, and can be easily determined by one of skill in the art (e.g., by the clinician). The standard dosing for protamine can be used and adjusted (i.e., increased or decreased) depending upon the factors described above.

The pharmaceutical compositions and/or formulations containing the compounds of the invention and a suitable carrier can be solid dosage forms which include, but are not limited to, tablets, capsules, cachets, pellets, pills, powders and granules; topical dosage forms which include, but are not limited to, solutions, powders, fluid emulsions, fluid suspensions, semi-solids, ointments, pastes, creams, gels and jellies, and foams; and parenteral dosage forms which include, but are not limited to, solutions, suspensions, emulsions, and dry powder; comprising an effective amount of a compound of the invention. It is also known in the art that the active ingredients can be contained in such formulations with pharmaceutically acceptable diluents, fillers, disintegrants, binders, lubricants, surfactants, hydrophobic vehicles, water soluble vehicles, emulsifiers, buffers, humectants, moisturizers, solubilizers, preservatives and the like. The means and methods for administration are known in the art and an artisan can refer to various pharmacologic references for guidance (see, for example, Modern Pharmaceutics, Banker & Rhodes, Marcel Dekker, Inc. (1979); and Goodman & Gilman's The Pharmaceutical Basis of Therapeutics, 6th Edition, MacMillan Publishing Co., New York (1980)).

The compounds of the invention can be formulated for parenteral administration by injection, such as by bolus injection or continuous infusion. The compounds of the invention can be administered by continuous infusion subcutaneously over a period of about 15 minutes to about 24 hours. Formulations for injection can be presented in unit dosage form, such as in ampoules or in multi-dose containers, with an added preservative. The compositions can take such forms as suspensions, solutions or emulsions in oily or aqueous vehicles, and can contain formulatory agents such as suspending, stabilizing and/or dispersing agents.

For oral administration, the compounds of the invention can be formulated readily by combining these compounds with pharmaceutically acceptable carriers well known in the art. Such carriers enable the compounds of the invention to be formulated as tablets, pills, dragees, capsules, liquids, gels, syrups, slurries, suspensions and the like, for oral ingestion by a patient to be treated. Pharmaceutical preparations for oral use can be obtained by, for example, adding a solid excipient, optionally grinding the resulting mixture, and processing the mixture of granules, after adding suitable auxiliaries, if desired, to obtain tablets or dragee cores. Suitable excipients include, but are not limited to, fillers such as sugars, including, but not limited to, lactose, sucrose, mannitol, and sorbitol; cellulose preparations such as, but not limited to, maize starch, wheat starch, rice starch, potato starch, gelatin, gum tragacanth, methyl cellulose, hydroxypropylmethyl-cellulose, sodium carboxymethylcellulose, and polyvinylpyrrolidone (PVP). If desired, disintegrating agents can be added, such as, but not limited to, the cross-linked polyvinyl pyrrolidone, agar, or alginic acid or a salt thereof such as sodium alginate.

Dragee cores can be provided with suitable coatings. For this purpose, concentrated sugar solutions can be used, which can optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments can be added to the tablets or dragee coatings for identification or to characterize different combinations of active compound doses.

Pharmaceutical preparations which can be used orally include, but are not limited to, push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds can be dissolved or suspended in suitable liquids, such as fatty oils, liquid paraffin, or liquid polyethylene glycols. In addition, stabilizers can be added. All formulations for oral administration should be in dosages suitable for such administration.

For buccal administration, the compositions can take the form of, such as, tablets or lozenges formulated in a conventional manner.

For administration by inhalation, the compounds of the invention for use according to the present invention are conveniently delivered in the form of an aerosol spray presentation from pressurized packs or a nebulizer, with the use of a suitable propellant, such as dichlorodifluoromethane, trichlorofluoromethane, dichlorotetrafluoroethane, carbon dioxide or other suitable gas. In the case of a pressurized aerosol the dosage unit can be determined by providing a valve to deliver a metered amount. Capsules and cartridges of, such as gelatin for use in an inhaler or insufflator can be formulated containing a powder mix of the compound and a suitable powder base such as lactose or starch.

The compounds of the invention can also be formulated in rectal compositions such as suppositories or retention enemas, such as containing conventional suppository bases such as cocoa butter or other glycerides.

In addition to the formulations described previously, the compounds of the invention can also be formulated as a depot preparation. Such long acting formulations can be administered by implantation (for example subcutaneously or intramuscularly) or by intramuscular injection. Depot injections can be administered at about 1 to about 6 months or longer intervals. Thus, for example, the compounds can be formulated with suitable polymeric or hydrophobic materials (for example as an emulsion in an acceptable oil) or ion exchange resins, or as sparingly soluble derivatives, for example, as a sparingly soluble salt.

In transdermal administration, the compounds of the invention, for example, can be applied to a plaster, or can be applied by transdermal, therapeutic systems that are consequently supplied to the organism.

The pharmaceutical compositions of the compounds of the invention also can comprise suitable solid or gel phase carriers or excipients. Examples of such carriers or excipients include, but are not limited to, calcium carbonate, calcium phosphate, various sugars, starches, cellulose derivatives, gelatin, and polymers such as polyethylene glycols.

The compounds of the invention can also be administered in combination with other active ingredients such as, for example, anti-heparin agent, including, but not limited to, protamine molecules.

Thus, the present invention also provides methods for antagonizing the anticoagulant effect of heparin in an animal comprising administering to the animal in need thereof an effective amount of a compound of the invention. The present invention also provides methods for antagonizing the anticoagulant effect of heparin in an animal comprising administering to the animal in need thereof a composition of the invention. The present invention also provides methods for antagonizing the anticoagulant effect of heparin comprising contacting the heparin with an effective amount of a compound or salt of the invention. The present invention also provides methods for antagonizing the anticoagulant effect of heparin comprising contacting the heparin with a composition of the invention.

The present invention also provides compounds of the invention, or compositions comprising the same, for use in antagonizing the anticoagulant effect of heparin in a patient. The present invention also provides compounds of the invention, or compositions comprising the same, for use in antagonizing the anticoagulant effect of heparin. The present invention also provides compounds of the invention, or compositions comprising the same, for use in preparation of a medicament for antagonizing the anticoagulant effect of heparin in a patient.

In order that the invention disclosed herein may be more efficiently understood, examples are provided below. It should be understood that these examples are for illustrative purposes only and are not to be construed as limiting the invention in any manner.

### EXAMPLES

### Example 1

### Synthesis

### Synthesis of Compound 1

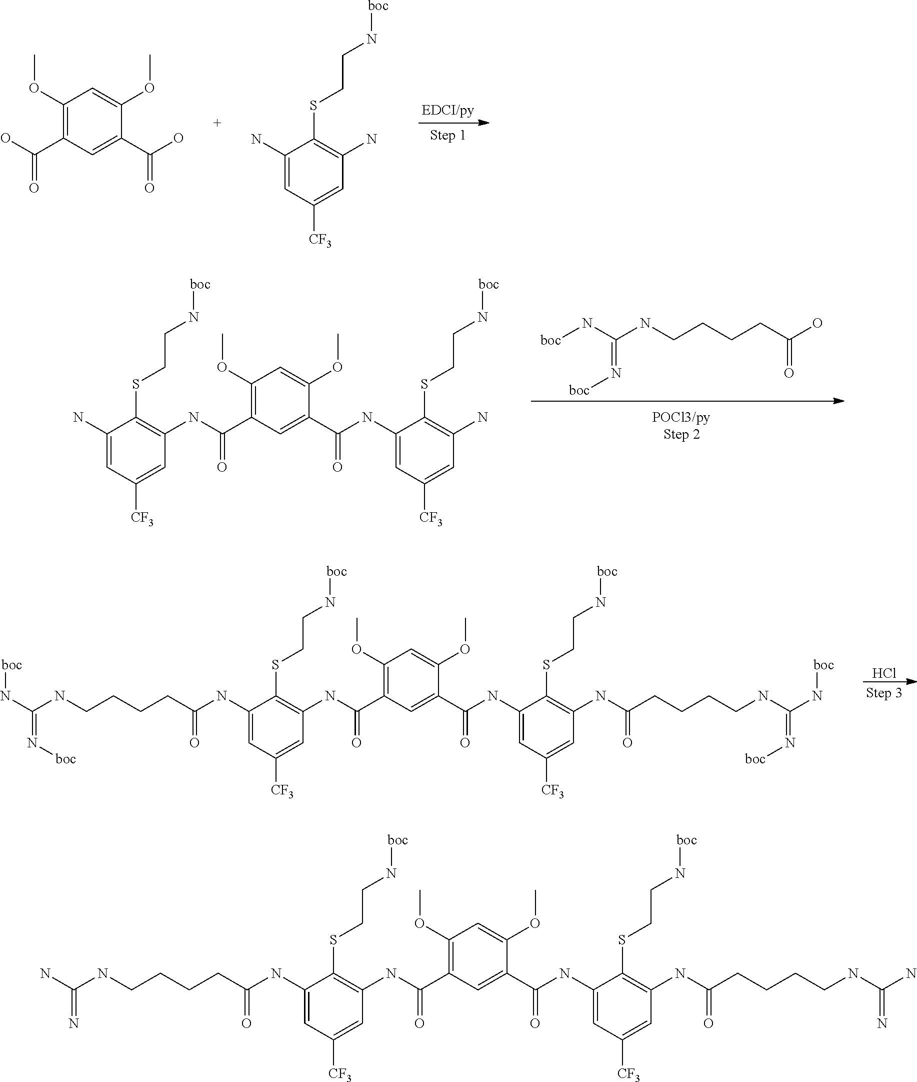

Step 1: The diacid and dianiline (2 equiv.) were mixed in pyridine, and EDCI was added. The reaction mixture was stirred at room temperature for 24 hours before the solvent was removed. The resulting solid was washed with water and recrystallized in DCM/Hexane.

Step 2: Product from step 1 and 5-bisBocguanidino pentoic acid were mixed and dissolved in pyridine. The solution was cooled to 0° C. before POCl₃ was added to the mixture. The reaction mixture was stirred at 0° C. for 2 hours before it is quenched with ice water. The product was purified by column chromatography.

Step 3: Product from step 2 was treated with HCl in ethyl acetate for 6 hours. The product was collected by filtration. The purification was done by reverse phase column chromatography.

Compound 6, 87 and 88 are made by similar procedure using different diacid in the first step.

| Compound | Diacids |
|---|---|
| 6 | 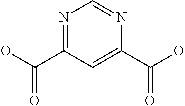 |
| 87 | 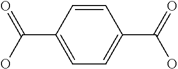 |
| 88 | 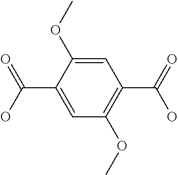 |

### Synthesis of Compound 4

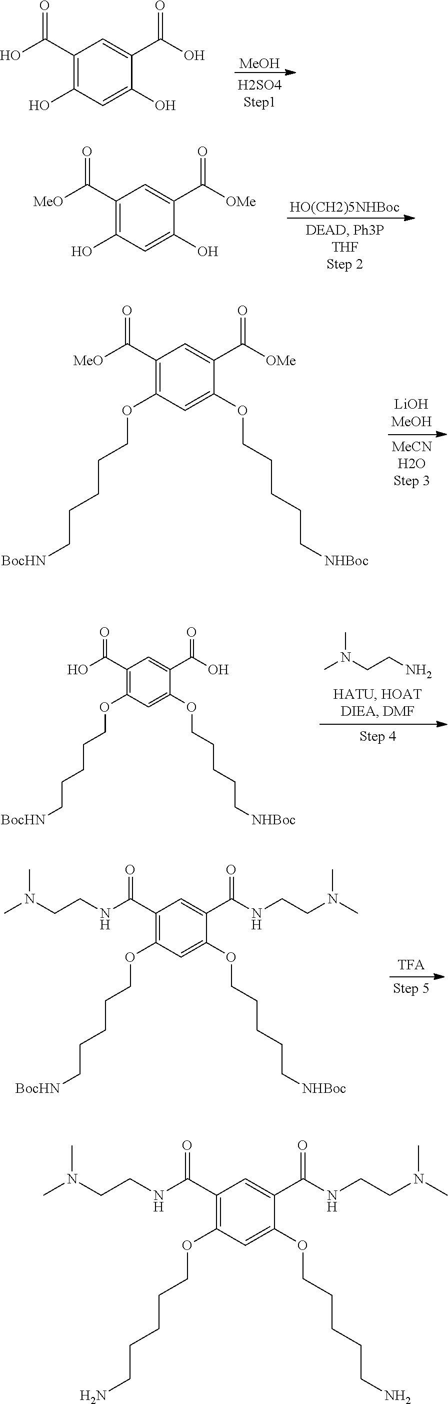

Step 1: A solution of acid (3.18 g) and concentrated H₂SO₄ (˜4 mL) in methanol (64 mL) was heated under reflux for 2 days. The product was obtained upon cooling and was filtered off and washed with a small amount of MeOH to give pure methyl ester.

Step 2: A flame dried 100 mL round bottom flask was charged with diol 2 (1.32 g, 5.84 mmol), 5-N-tert-butoxycarbonylamino-1-pentanol (2.37 g, 11.7 mmol), Ph₃P (3.06 g, 11.7 mmol), and THF (15 mL). The resulting solution was cooled to 0° C. under Argon, and DEAD (2.16 mL) was added to the solution dropwise to give a dard red solution. The mixture was then warmed to room temperature and stirred until no starting material remained (ca. 10 h). THF was removed and the residue was purified by column chromatography (DCM/hexane/ether=4:4:1) to give pure product.

Step 3: To the solution of diester (3.11 mmol) in methanol (10 mL), there was added 2 N LiOH (5.1 mL) slowly. The resulting solution was stirred at room temperature overnight, the solvent was then removed in vacuo. The residue was redissolved in water (150 mL), and the aqueous solution was acidified to pH=2 using 6 N HCl. Pure product was obtained by filtration.

Step 4: The diacid, N,N-dimethylethane-1,2-diamine (2 equiv.), HOAT (2 equiv.), HATU (2 equiv.) and DIEA (5 equiv.) were mixed in DMF and stirred at room temperature overnight. The solution was diluted with water, and the product was purified by reverse phase chromatography.

Step 5: Product from step 4 was treated with 50% TFA in DCM for 3 hours. The solution was concentrated to an oil and triturated with cold ether. The product was collected by filtration and dried under vacuum.

### Synthesis of Compound 2

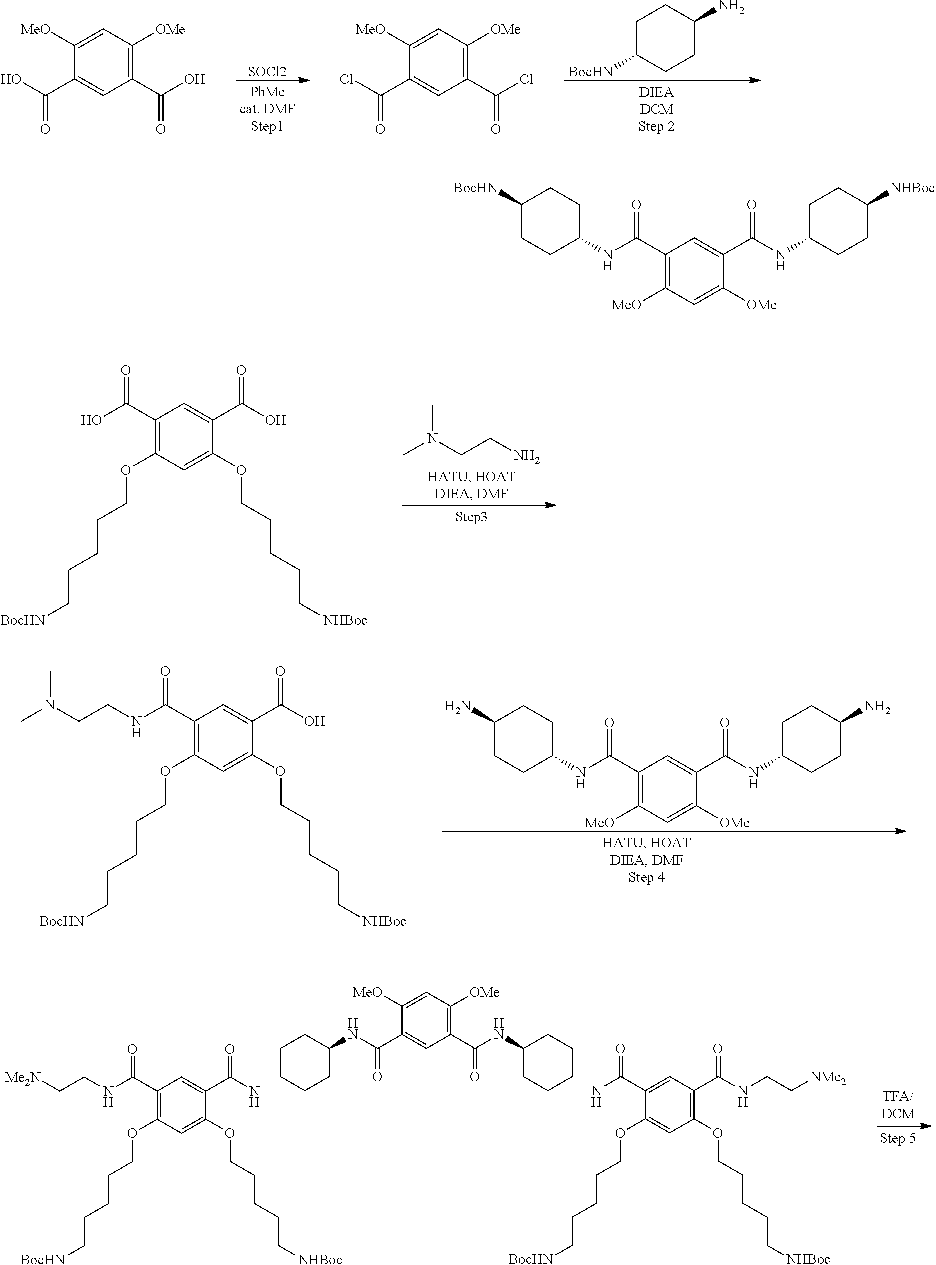

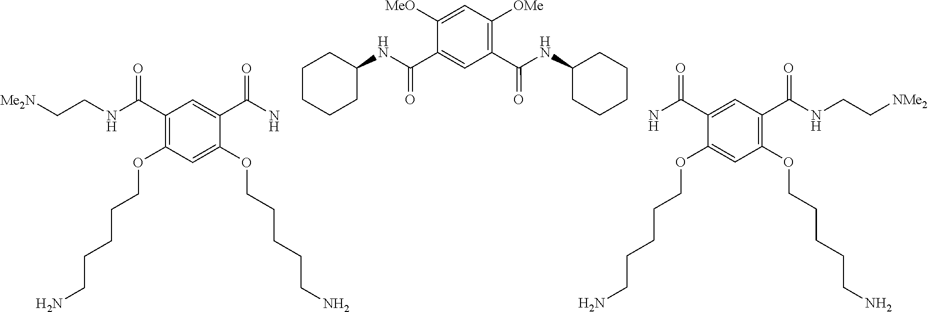

Step 1: One 1 L round bottom flask was fitted with a magnetic stirrer condenser, drying tube and a heating mantel. Diacid (20 g) was added and slurried in toluene (256 mL). DMF (1 mL) was added, followed by SOCl₂ (64 mL). The resulting slurry was heated at reflux and complete solution was obtained after 10 minutes. The reaction mixture was cooled to room temperature after 90 minutes of reflux and stirred overnight. The product crystallized out from the solution. The mixture was cooled at 5° C. for one hour. The solid was collected by filtration and washed with cold toluene. Yield: 19.71 g.

Step 2: The mono Boc protected amine was dissolved in DCM and DIEA was added. Acid chloride was added to the solution and the reaction mixture was stirred at room temperature for 2 hours and the product precipitated out. The product was collected by filtration.

Step 3: The diacid, N,N-dimethylethane-1,2-diamine (1 equiv.), HOAT (1 equiv.), HATU (1 equiv.) and DIEA (2 equiv.) were mixed in DMF and stirred at room temperature overnight. The solution was diluted with water, and the product was purified by reverse phase chromatography.

Step 4: Diamine, acid (2.2 equiv.), HOAT (2.2 equiv.), HATU (2.2 equiv.) and DIEA (5 equiv.) were dissolved in DMF and stirred at room temperature overnight. The mixture was added water and extracted with DCM. The organic layer was concentrated to generate the crude solid. The product was purified by reverse phase chromatography.

Step 5: Product from step 4 was treated with 50% TFA in DCM for 3 hours. The solution was concentrated to an oil and triturated with cold ether. The product was collected by filtration and dried under vacuum.

### Synthesis of Compound 3

Compound 3 was made by similar procedure as compound 2 except one extra step.

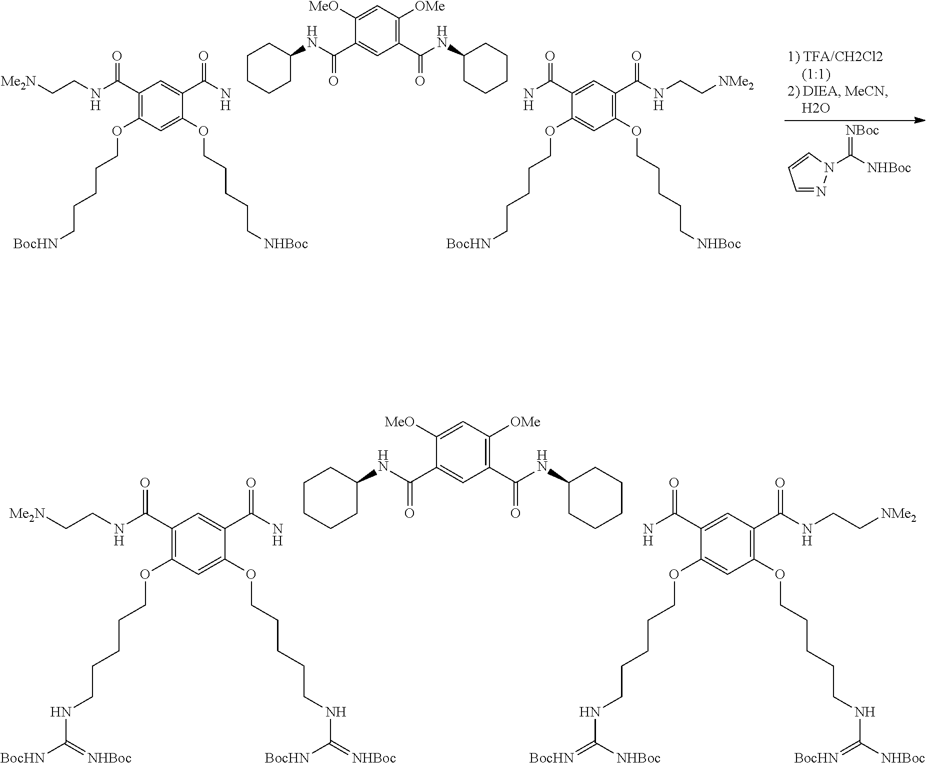

The Boc of the precursor was removed by treatment of 50% TFA/DCM. After the solid was washed and dried under vacuum, it was dissolved in acetonitrile and water, DIEA (15 equiv.) was added and followed by di-Boc pyrazole. The reaction mixture was stirred at room temperature overnight. The solvent was removed and the solid was redissolved in DCM. After trituration with hexane/diethyl ether, the product was collected by filtration and dried under vacuum.

Synthesis of compound 103, 104, 105 and 106 were synthesized using similar method as compound 3.

### Synthesis of Compound 5

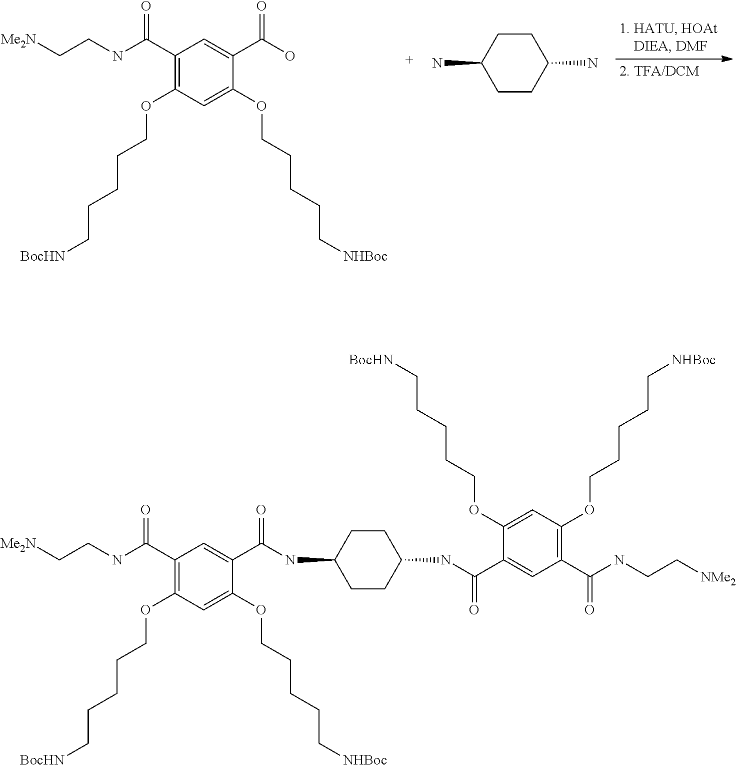

The damine, monoacid (2. equiv.), HATU (2. equiv.) and HOAT (2. equiv.) were mixed and dissolved in DMF. DIEA (4 equiv.) was added to the DMF solution and the reaction mixture was stirred at room temperature overnight. The solution was diluted with water and extracted with DCM. The organic layer was washed with water before the solvent was removed.

The solid was treated with 50% TFA in DCM for 3 hours before the solution was concentrated. The product was precipitated with diethyl ether and purified by reverse phase chromatography.

### Synthesis of Compound 86

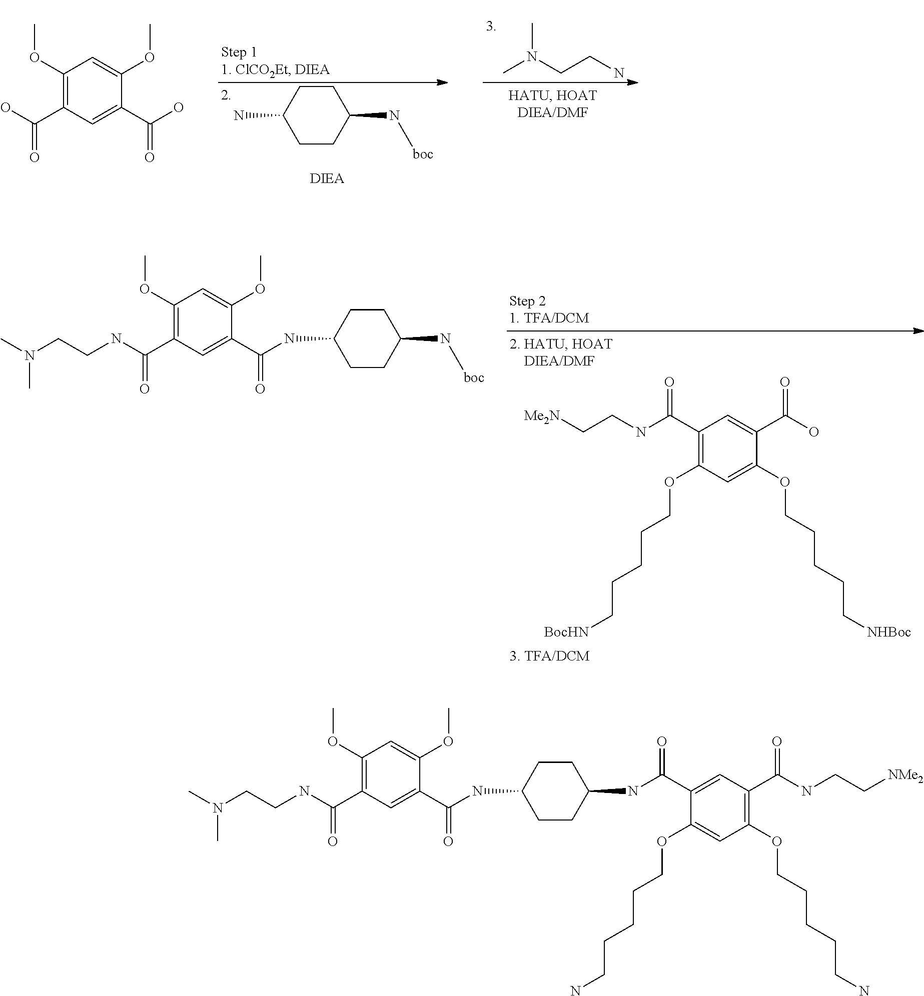

Step 1: The diacid was suspended in chloroform and ethyl chloroformate (2.2 equiv.) was added. DIEA (2.2 equiv.) was added to the mixture and stirred for 2 hours before monoBoc hexyldiamine (2.2 equiv.) was added. The reaction mixture was stirred for 4 hours before it was added N,N-dimethyl ethylenediamine (1.5 equiv.). The reaction mixture was stirred overnight. The solution was diluted with DCM and washed with water. After the solvent was removed, the product was purified by reverse phase column chromatography.

Step 2: Product from step 3 was treated with 50% TFA in DCM for 2 hours before the solvent was removed. The solid was dried under vacuum at 35° C. for 2 hours before it was dissolved in DMF. HATU, HOAT and monoacid was added to the solution. Then DIEA was added. The mixture was stirred overnight at room temperature. After diluted with water, the product was extracted with DCM. The organic layer was washed with water, concentrated to solid and dried under vacuum overnight. The solid was treated with 50% TFA/DCM for 2 hours. The final product was purified by reverse phase column chromatography.

### Synthesis of Compound 89

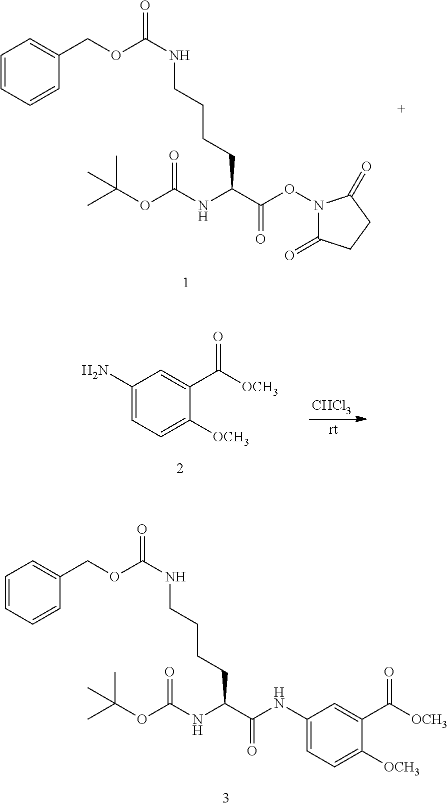

A mixture of 47.75 g (100.0 mmol) of 1 and 18.12 g (100.0 mmol) of 2 in 500 mL of anhydrous CHCl₃ was stirred at room temperature under Ar and, after 30 minutes, a clear orange solution was observed. The reaction was monitored by tlc and found to be complete after 60 hours. The reaction was concentrated in vacuo to a brown syrup that was dissolved between EtOAc and water. The layers were separated and the aqueous layer was extracted twice more with EtOAc. The EtOAc fractions were combined and washed four times with water (followed removal of byproduct HOSu by tlc). The EtOAc layer was then washed once with 10% citric acid (aqueous), twice with water, three times (carefully) with saturated NaHCO₃, and once with brine. The EtOAc layer was dried over Na₂SO₄, filtered, and concentrated to afford 53.48 g (98%) of 3.

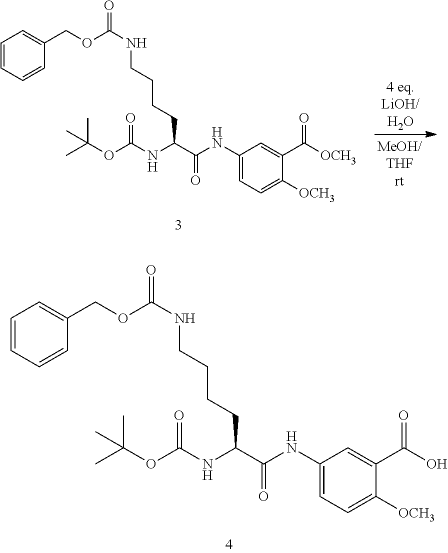

A solution of 26.74 g (49.19 mmol) of 3 in a mixture of 294 mL of THF and 196 mL of MeOH was treated with 98 mL of 2.0 M LiOH (aqueous) (196 mmol) and the resultant mixture was stirred at room temperature for 18 hours. The reaction mixture was cooled in an ice bath then treated with 196 mL of cold 1.0 M HCl (aqueous) to neutralize. The quenched reaction was partially concentrated in vacuo to an aqueous slurry that was extracted with EtOAc until tlc showed the extraction was complete. The EtOAc layer was dried over Na₂SO₄, filtered, and concentrated to afford 25.71 g (99%) of 4 as a beige solid.

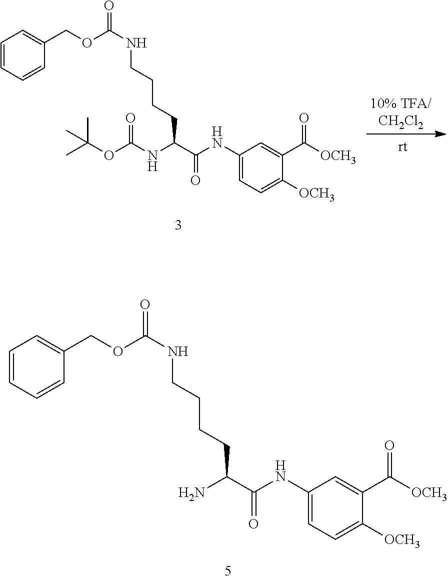

3 (26.74 g, 49.19 mmol) was introduced to a 1 L round bottom flask that was equipped with a ground glass stopper (secured by a Keck clamp) and treated with 385 mL of a cold 10% solution (v/v) of TFA in CH₂Cl₂ (500 mmol of TFA). The resultant brick red solution was allowed to warm to room temperature. The reaction was followed by tlc and all of 3 was consumed after 24 hours. The reaction was diluted with twice its volume of CH₃CN and concentrated in vacuo without heating to a brown syrup. This residue was dissolved in EtOAc and extracted (carefully) three times with saturated NaHCO₃. The aqueous fractions were combined, treated with solid NaHCO₃ to ensure pH of 8, and backwashed twice with EtOAc. The EtOAc fractions were combined, dried over Na₂SO₄, filtered, concentrated, and subjected to high vacuum to afford 24.83 g of 5.

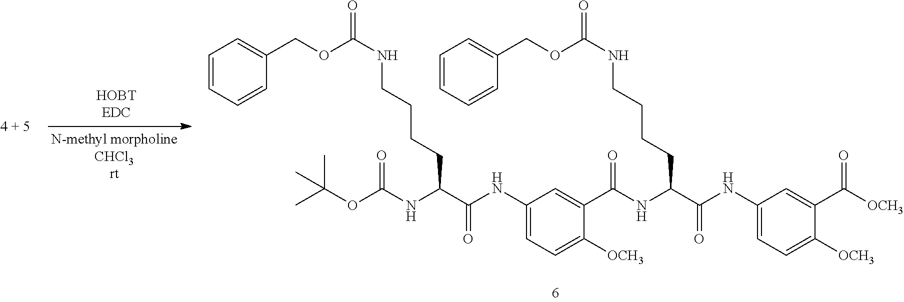

A mixture of 1.06 g (2.00 mmol) of 4 and 1.01 g (2.00 mmol) of 5 was dissolved in 60 mL of anhydrous CHCl₃. Added 0.54 g (4.0 mmol) of HOBT, 0.46 g (2.4 mmol) of EDC, and 0.33 ml, (3.0 mmol) of N-methyl morpholine and stirred the resultant suspension at room temperature under Ar. The reaction became an orange solution and, after 24 hours, tlc and MS/HPLC showed it to be complete. The reaction mixture was diluted with CH₂Cl₂ and extracted twice with water, twice with saturated NaHCO₃ and once with brine. The CH₂Cl₂ fraction was dried over Na₂SO₄, filtered, and concentrated in vacuo to afford 1.98 g of brown crusty foam that was subjected to flash silica gel chromatography (1:1 hexane/EtOAc to 1:3 hexane/EtOAc). Obtained 1.71 g (89%) of 6.

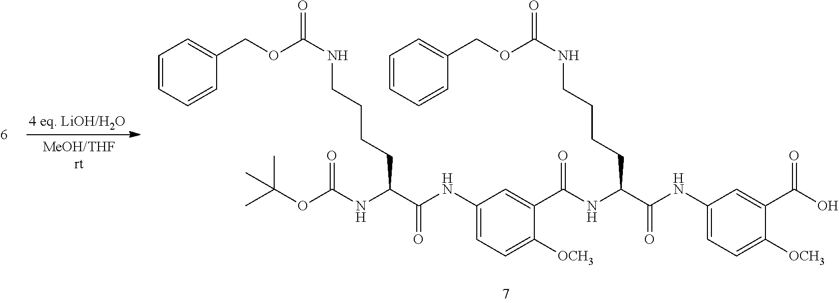

A solution of 0.33 g (0.346 mmol) of 6 in a mixture of 2.1 mL of THF and 1.4 mL of MeOH was treated with 0.70 mL of 2.0 M LiOH (aqueous) (1.4 mmol) and the resultant mixture was stirred at room temperature for 8 hours. The reaction mixture was cooled in an ice bath then treated with 1.4 mL of cold 1.0 M HCl (aqueous) to neutralize. The quenched reaction was partially concentrated in vacuo to an aqueous slurry that was extracted with EtOAc until tlc showed the extraction was complete. The EtOAc layer was dried over Na₂SO₄, filtered, and concentrated to afford 0.321 g (99%) of 7.

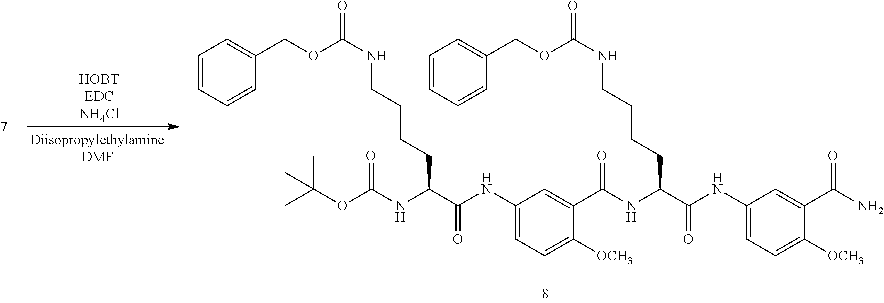

A mixture of 7 (0.798 g, 0.849 mmol), HOBT (0.224 g, 1.70 mmol), EDC (0.278 g, 1.70 mmol), and NH₄Cl (0.099 g, 1.7 mmol) was dissolved in 8 mL of DMF under an Ar atmosphere. DIEA (0.59 mL, 3.4 mmol) was added and the reaction mixture stirred at room temperature for 8 hours. The mixture was poured into a mixture of 5 mL 1 N HCl and extracted with EtOAc. The organic phase was washed with H₂O and brine, dried (Na₂SO₄) and the solvent evaporated to yield 0.729 g (91%) of 8 that was used without further purification in the subsequent reaction.

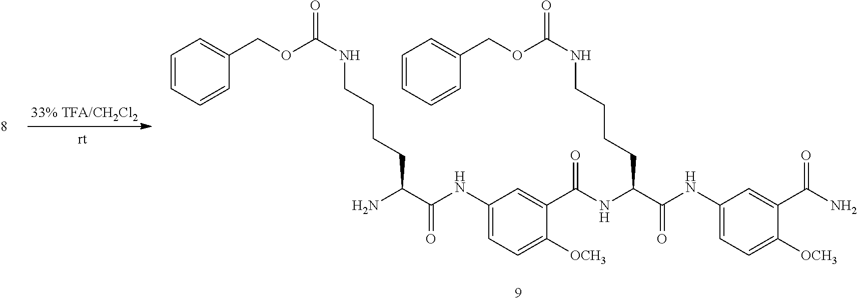

Compound 8 (0.900 g, 0.96 mmol) was stirred at room temperature in 4.5 mL of a 33% solution (v/v) of TFA/CH₂Cl₂ for 1.5 hours. Et₂O was added, and the solid filtered or the mixture centrifuged and the solvent decanted. The resultant solid was triturated with Et₂O and dried to yield 0.75 g (82%) of mono-TFA salt 9 as a white powder.

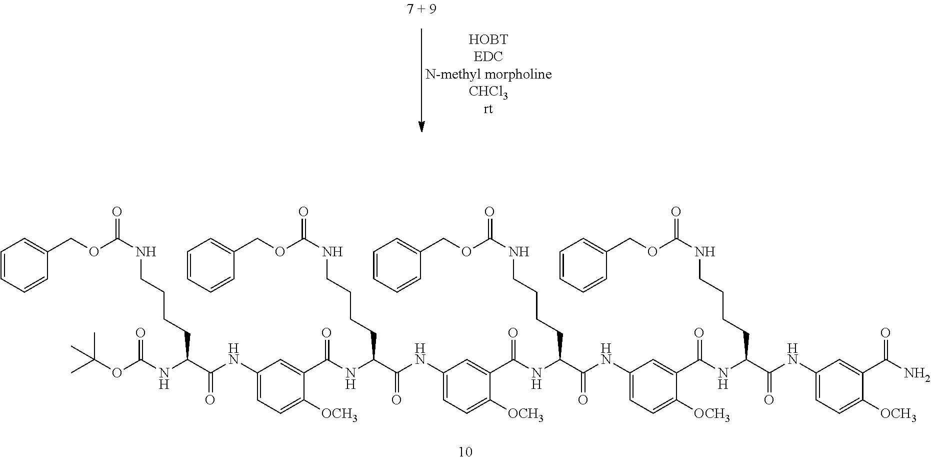

A mixture of 0.321 g (0.341 mmol) of 7 and 0.286 g (0.341 mmol) of 9 (free based from its TFA salt by extraction between saturated NaHCO₃ and EtOAc) was dissolved in 15 mL of anhydrous CHCl₃. Added 0.092 g (0.68 mmol) of HOBT, 0.079 g (0.41 mmol) of EDC, and 0.056 mL (0.51 mmol) of N-methyl morpholine and stirred the resultant suspension at room temperature under Ar. The reaction became a yellow solution and, after 40 hours, tlc and MS/HPLC showed it to be complete. The reaction mixture was diluted with CH₂Cl₂ and extracted twice with water, twice with saturated NaHCO₃, once with 10% citric acid (aqueous), and twice with brine. The CH₂Cl₂ fraction was dried over Na₂SO₄, filtered, and concentrated in vacuo to afford 0.607 g of beige wax that was subjected to flash silica gel chromatography (CH₂Cl₂ to 97:3 CH₂Cl₂/MeOH). Obtained 0.411 g (68%) of 10 as a beige solid.

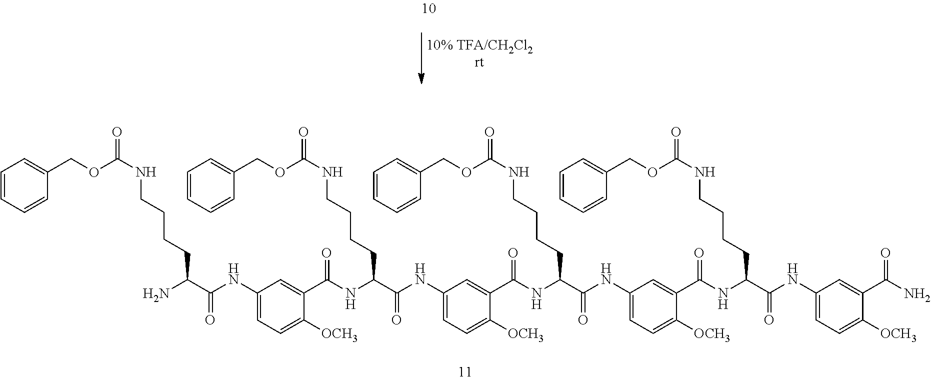

Compound 10 (0.411 g, 0.233 mmol) was introduced to a 100 mL round bottom flask that was equipped with a ground glass stopper (secured by a Keck clamp) and treated with 5 mL of a cold 10% solution (v/v) of TFA in CH₂Cl₂. The resultant brick red solution was allowed to warm to room temperature. The reaction was followed by tlc and all of 10 was consumed after 24 hours. The reaction was diluted with CH₃CN and concentrated in vacuo without heating to a brown syrup. This residue was dissolved in CH₂Cl₂ and extracted three times with saturated NaHCO₃. The aqueous fractions were combined and backwashed twice with CH₂Cl₂. The CH₂Cl₂ fractions were combined, dried over Na₂SO₄, filtered, and concentrated to afford 0.394 g (101% of theoretical) of a sample of crude 11 as a beige amorphous solid. This crude product was used without further purification in the subsequent reaction.

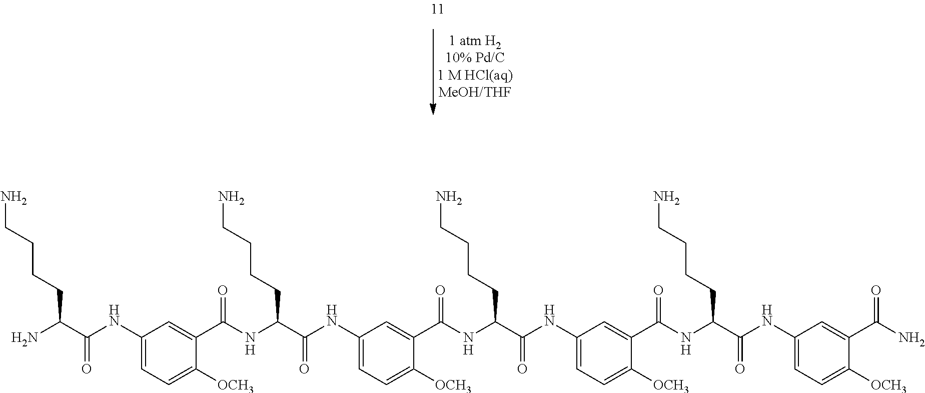

Introduced 0.197 g (assumed 0.118 mmol) of the crude sample of 11 to a 250 ml round bottom flask that was equipped with an adapter containing a three way stopcock to which a balloon was attached. Dissolved 11 in a mixture of 5 mL of THF and 5 mL of MeOH, added 0.59 ml of 1.0 M HCl (aqueous), and bubbled Ar through the reaction solution for 15 minutes. Carefully added a small scoop of 10% Pd/C and exposed the reaction to H₂ at 1 atm via the balloon. Stirred vigorously, followed the reaction by MS/HPLC, and recharged the balloon with H₂ as needed. After 60 hours, the completed reaction was suctioned filtered through Celite using MeOH to assist transfer and to wash the collected solids. The filtrate was concentrated to afford 0.150 g of beige waxy solid. The final product was purified by reverse phase column chromatography.

### Synthesis of Compound 12

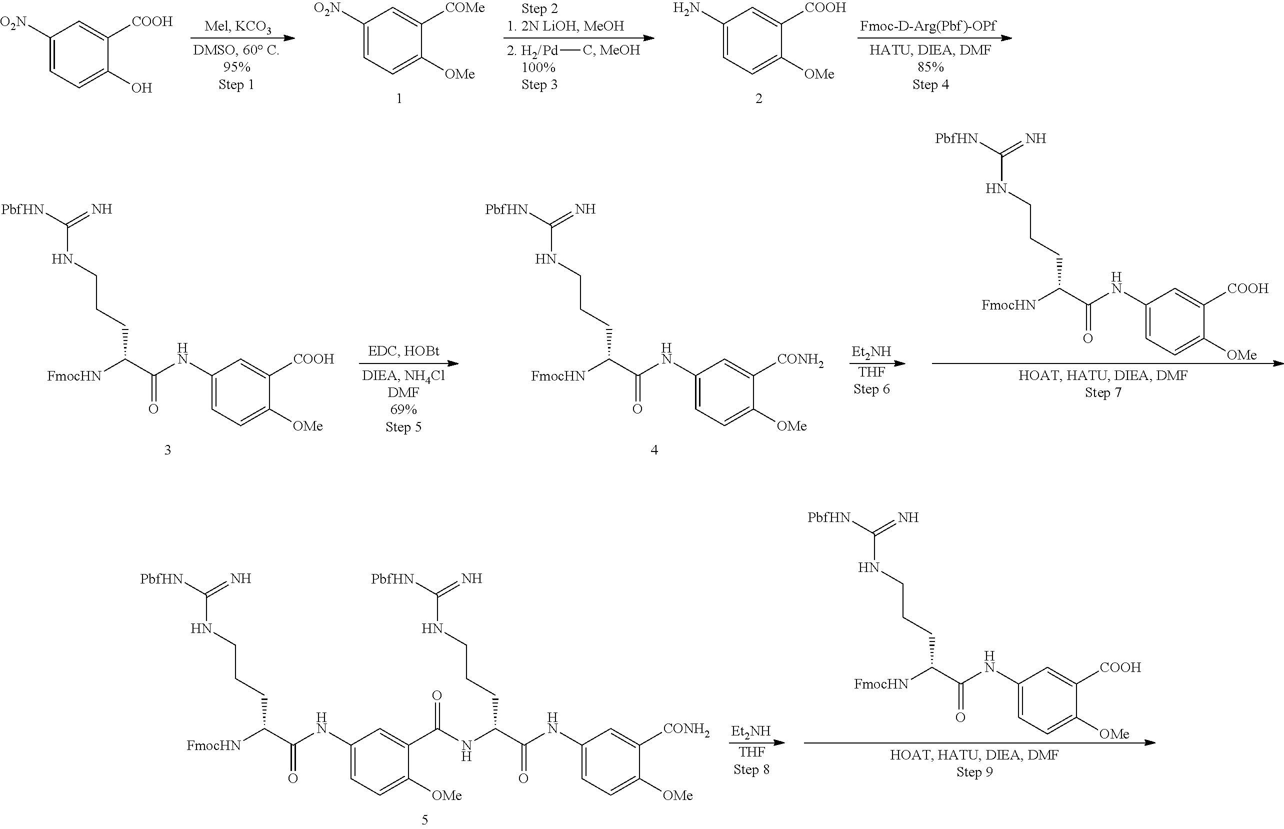

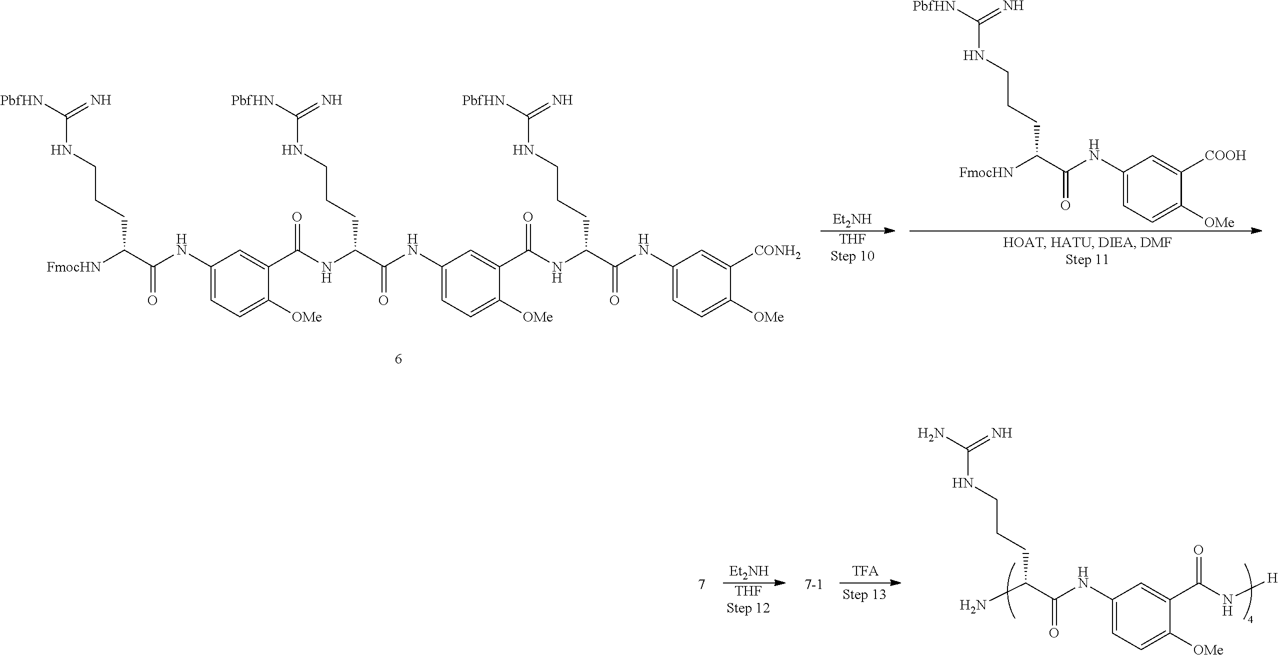

Step 1: Starting material 5-nitro salicylic acid (40 g, 0.218 mol) was dissolved in 220 mL of DMSO followed by addition of KCO₃ (151 g, 1.09 mol). Methyl iodide (136 mL, 2.18 mol) was added to the solution. The reaction mixture was heated to 60° C. and stirred (mechanical stir) overnight. Ethyl acetate (6 L) was added to the reaction mixture in 4 portions to completely dissolve the desired product. The suspension was filtered to remove solid. The organic layer was washed with 1N HCl, saturate NaCl and water, dried over Na₂SO₄. The solvent was removed by rotovap. Yield: 45.7 g, 99%.

Steps 2 and 3: To the solution of ester compound 1 (10 g, 47.36 mmol) in 4:1 methanol/acetonitrile (250 mL) there was added 2 N LiOH (47.4 mL, 94.7 mmol). The resulting solution was stirred at room temperature until no starting material remained (ca. 3 hours). The solution was then acidified to pH=4˜5 with cold HCl, extracted with EtOAc-MeOH (10% MeOH) five times. The combined organic layers were washed with brine, dried over Na₂SO₄, filtered and concentrated to give 9.5 g of the acid.

The product from the hydrolysis was dissolved 120 mL of MeOH-THF (5:1), and Pd—C (10% wt. 1.7 g 94.7 mmol) was introduced. The resulting mixture was charged hydrogen by a balloon, and stirred at room temperature overnight. The catalyst was filtered with celite and solvent was removed under reduced pressure. The product was dried under vacuum overnight. Yield: 8.3 g, 100%.

Step 4: Fmoc-D-Arg(Pbf)-Opf (25 g, 30.68 mmol), compound 2 (5.64 g, 33.75 mmol) were dissolved in anhydrous DMF (85 mL). HOAT (30.78 mmol in 61.4 mL of DMF) and DIEA (6.41 ml, 36.82 mmol) were added to the solution at 0° C. under Ar. The solution was warmed up to room temperature and stirred overnight. The solvent was removed on a rotovap. The product was purified by flash column using DCM: MeOH (25:1 to 15:1). Purification was done on a C18 reverse phase flash column as well using AcCN:water. Yield: 15.4 g, 57%.

Step 5: The Fmoc protected compound 3 (6.74 g, 8.45 mmol), EDC (3.24 g, 16.9 mmol), HOBt (2.28 g, 16.9 mmol), DIEA (4.36 g, 33.8 mmol) and NH₄Cl (0.904 g, 16.9 mmol) were mixed and dissolved in anhydrous DMF (35 mL), and stirred for 6 hours at 0° C. The solution was diluted with EtOAc and washed with 10% citric acid, sat. NaHCO₃ and NaCl. The final product was purified on a flash column with DCM:MeOH (35:1 to 20:1). Yield 3.77 g, 56%.

Steps 6 and 7:

Fmoc deprotection: The amide 4 (3.7 g, 4.6 mmol) was treated with Et₂NH (7.76 ml) in 60 mL of THF at 0° C. for 6 hours. After the liquid is removed under vacuum, the solid was redissolved in AcCN:MeOH (1:1) and the solvent was remove on a rotovap. This process was repeated two times to remove any residual Et₂NH. The resulting off-white frothy material was trituated with diethyl ether (6×40 mL) and the resulting thick liquid was dried on a vacuum pump overnight to afford the pure deprotected amine.

The deprotected amine was dissolved in 20 mL of anhydrous DMF. Compound 3 (3.69 g, 4.62 mmol), HATU (1.755 g, 4.62 mmol), HOAT (4.62 mmol) and DIEA (1.49 g, 11.57 mmol) were dissolved in 30 mL of anhydrous DMF and added to a solution of the deprotected amine in 10 mL of DMF. The reaction mixture was stirred at room temperature for 3 hours. The solution was diluted with 200 mL of DCM and washed with 10% citric acid, sat. NaHCO₃, brine and water. The organic layer was concentrated on a rotovap. Final product was purified on a C18 reverse phase column using a gradient of AcCN/water. Yield: 4.72 g, 75%.

Steps 8 and 9:

Fmoc deprotection: The amide 5 (4.5 g, 3.32 mmol) was dissolved in 23 mL of DMF and cooled to 0° C. Et₂NH (5.1 g) was added to the solution dropwise under Ar. The resulting solution was stirred at 0° C. for 3.5 hours. After the liquid is removed under vacuum, the deprotected amine was triturated and washed with EtOAc-Hexanes (3:1) three times to afford pure compound.

After the solid was dried under vacuum, it was coupled with compound 3 using HOAT, HATU, DIEA in DMF for 4 hours. (procedure and reactant are the same as the procedure for synthesize compound 5). The product was purified using a C18 reverse phase column with gradient of AcCN/water. Yield: 1.21 g, 20%

Steps 10 and 11: Compound 7 was synthesized from 0.68 mmol of 6 using the same procedures (Fmoc deprotection and coupling) to synthesize compound 6. After work up, the crude compound 7 was used for next step without purification.

Steps 12 and 13: The amide 7 (1.68 g, 70% purity) was treated with Et₂NH (0.767 g) in 10 mL of DMF at 0° C. for 1.5 hours. The deprotected amine was worked up as usual. The Pbf group was removed by a treatment of 250 mL of TFA cocktail (95% TFA, 2.5% water and 2.5% triisopropylsilane) for 1 hour. The reaction mixture was concentrated on a rotovap to its half volume and cooled with ice water bath and triturated with 400 mL of cold MTBE. The solid was washed twice with cold MTBE and dried under vacuum. The final product was purified by prep HPLC on a C4 reverse phase column using a gradient of AcCN:water (with 0.1% TFA). Yield 0.379 g, 43%.

### The Synthesis of Salicylamides: (Compounds 7-85, 89-102, 107-146)

For salicylamides with the same repeating unit, they are made using procedures that at similar to the synthesis of compound 12 and 89. For salicylamides with different building units, they were made via solid phase synthesis which is described as following:

Solid phase synthesis procedure for salicylamides: The synthesis was carried at 0.2 mmol scale using Fmoc chemistry. PAL-PEG resin was used for amide oligomers, and Wang resin was used for acid oligomers. The coupling reagents are HATU/HOAT with DIEA, solvent was DMF. Piperidine (20% in DMF) was used for Fmoc removal. The cleavage and final deprotection were performed using 95% TFA with 5% TIS. The final products were purified on RP-HPLC.

### Synthesis of Labeled Compound 121

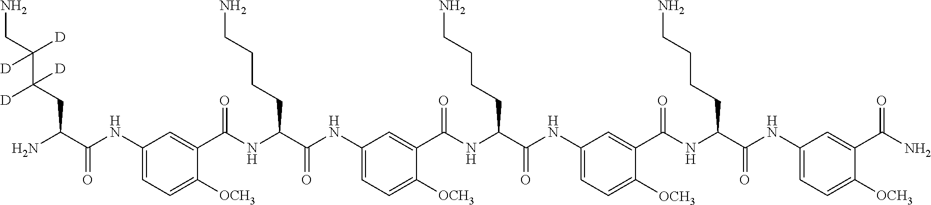

The compound was made via solid phase synthesis. The last building block for the solid phase synthesis (3) was made by the following procedure:

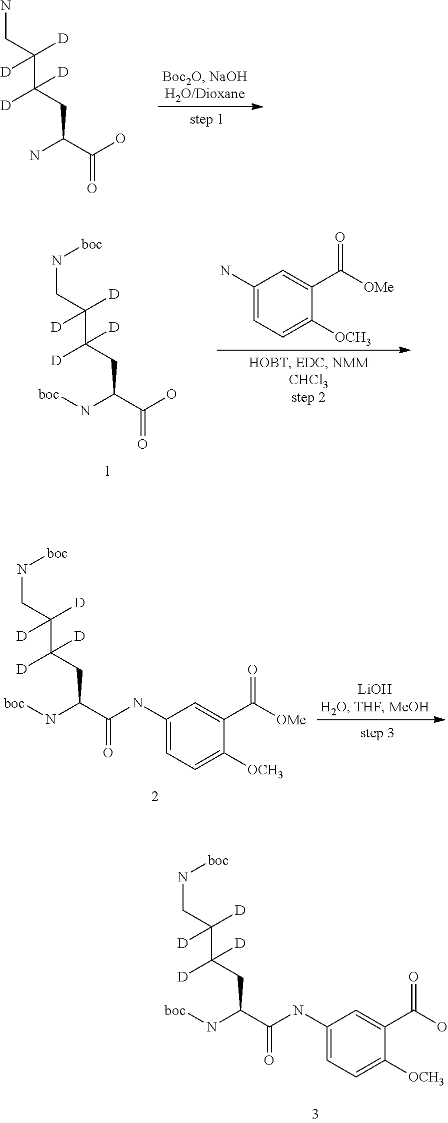

Step 1: L-D4-Lysine (12.4 mmol) was dissolved in 36 mL of water/dioxane (1:1). Boc₂O (31 mmol) was added to the solution, followed by 12.7 mL of 1N NaOH. The reaction mixture was stirred for 18 hours before more Boc₂O (9.3 mmol), 1N NaOH (6.5 mL) and dioxane (6 mL) were added. The reaction was stirred for another 18 hours. The pH of the solution was adjusted to 2-3 with KHSO₄ while cooled with ice bath. The product was extracted by EtOAc for 4 times. The organic layer was dried and concentrated to a solid. The product was used for next step without purification.

Step 2: Product from step 1 (1, 9 mmol) was dissolved in 130 mL of chloroform. To the solution were added 9 mmol of methyl 5-amino-2-methoxybenzoate, HOBT (18 mmol), EDC (10.8 mmol) and 1.5 mL of n-methyl morpholine. The reaction mixture was stirred overnight. The solution was diluted with DCM and washed with water. The aqueous layer was extracted twice with DCM. The combined organic layer was washed with sat. NaHCO₃ and brine, and dried and concentrated to a solid. The product was used for the next step without purification.

Step 3: The product from step 2 (2, 8.37 mmol) was dissolved in 50 mL of THF/33 mL of MeOH. LiOH (2N, 16.75 mL) was added to the solution. The reaction mixture was stirred overnight. While cooled with ice bath, the solution was neutralized with 1N HCl to pH 6-7. The product was extracted by EtOAc. After the solvent was removed, the product was dried under vacuum.

### Example 2

### Compounds for Evaluation as Anti-Heparin Agents

The following exemplary compounds (and/or their salts) in Table 1 were prepared by methods such as those reported in U.S. Patent Application Publication Nos. U.S. 2005/0287108, U.S. 2006/0041023, U.S. Pat. No. 7,173,102, WO 2005/123660, WO 2004/082643, WO 2006/093813, and U.S. patent application Ser. No. 12/510,593 filed Jul. 28, 2009.

TABLE 1

| Compd. | |
|---|---|
| No. | Structure |
| 1 | 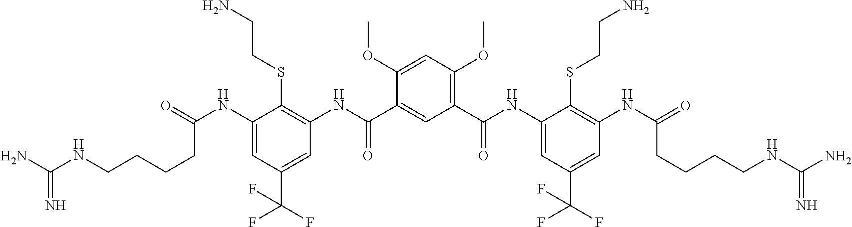 |
| 2 | 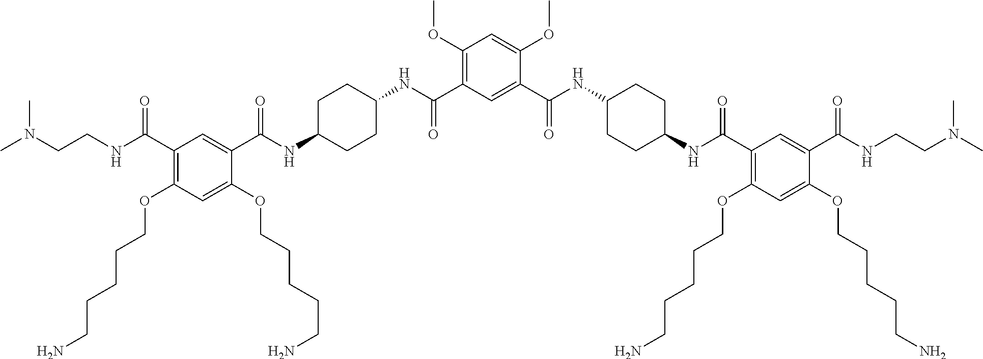 |
| 3 | 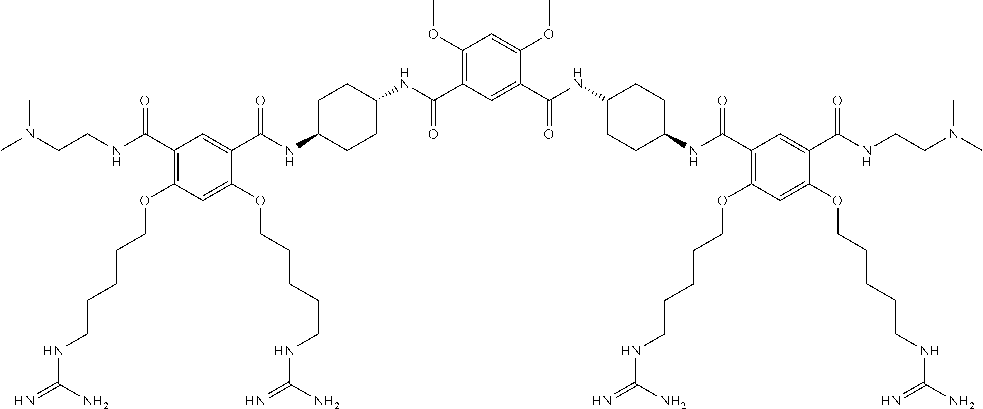 |
| 4 | 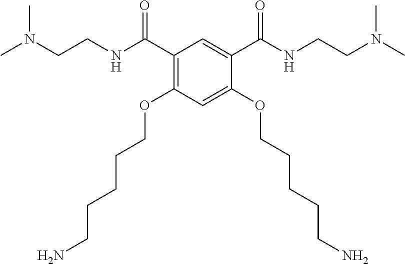 |
| 5 | 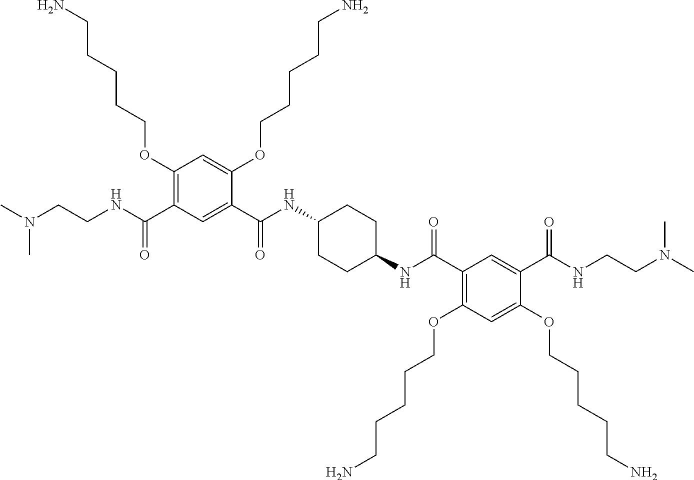 |
| 6 | 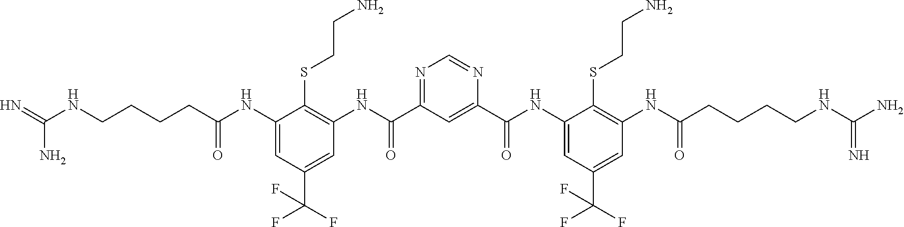 |
| 7 | 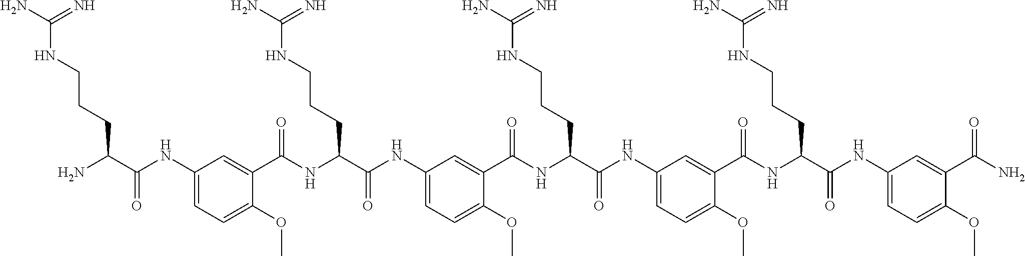 |
| 8 | 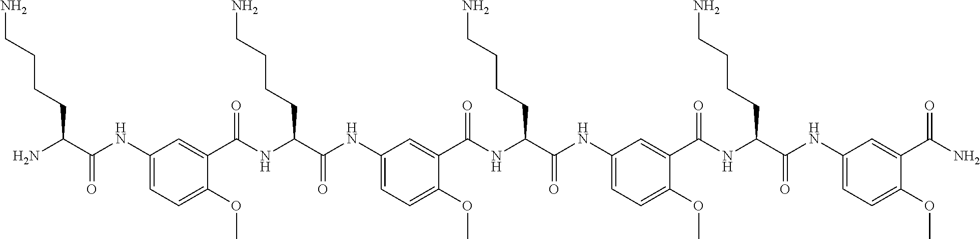 |
| 9 | 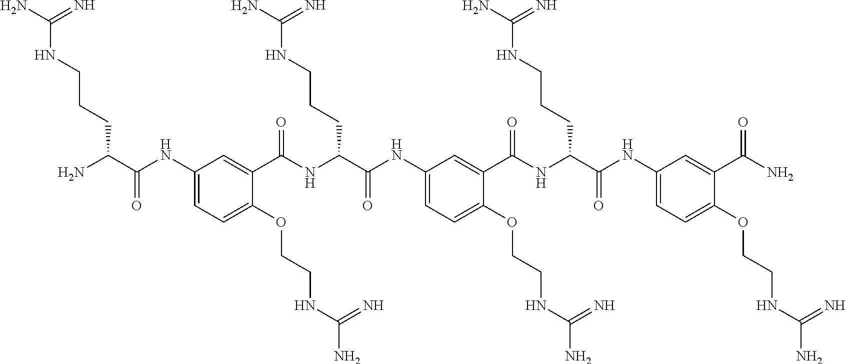 |
| 10 | 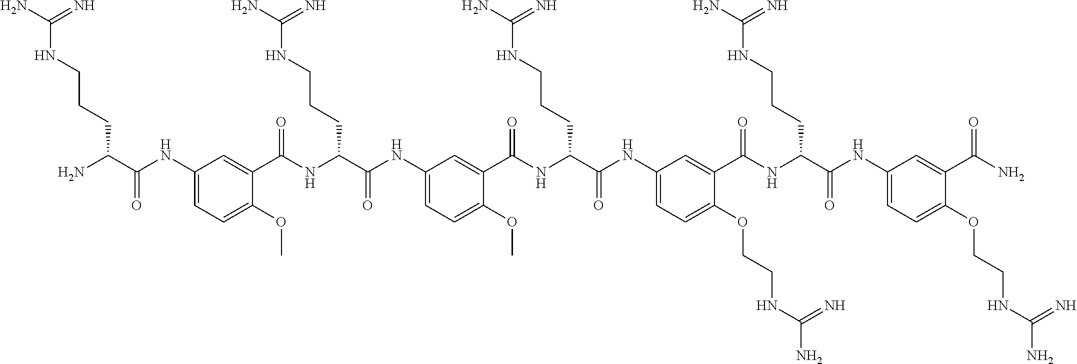 |
| 11 | 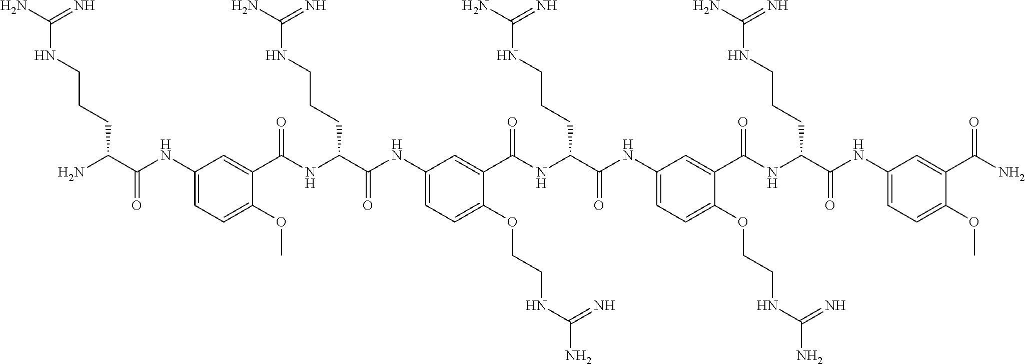 |
| 12 | 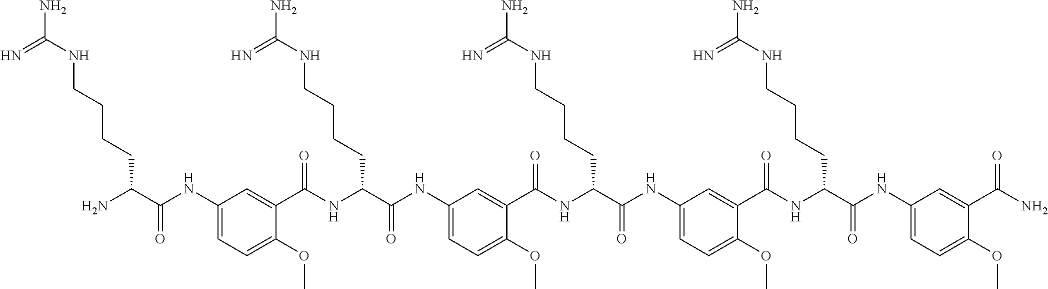 |
| 13 | 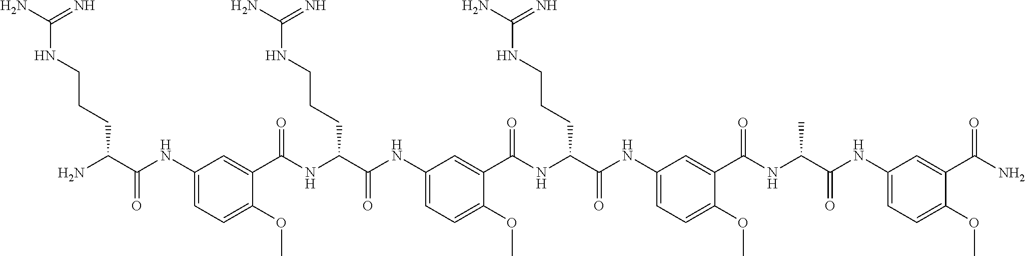 |
| 14 | 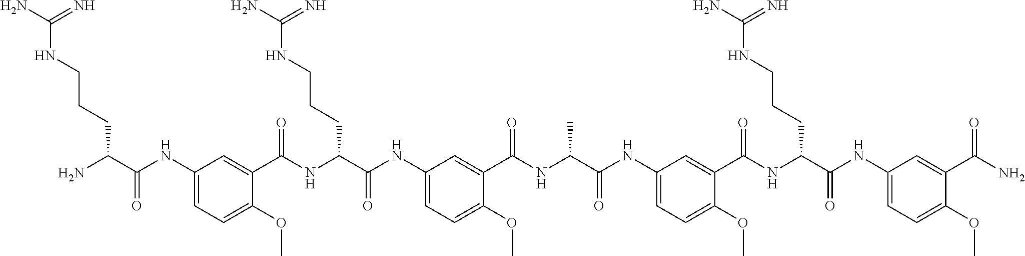 |
| 15 | 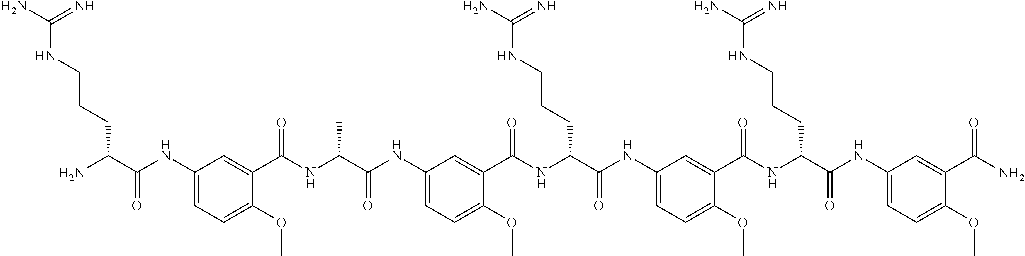 |
| 16 | 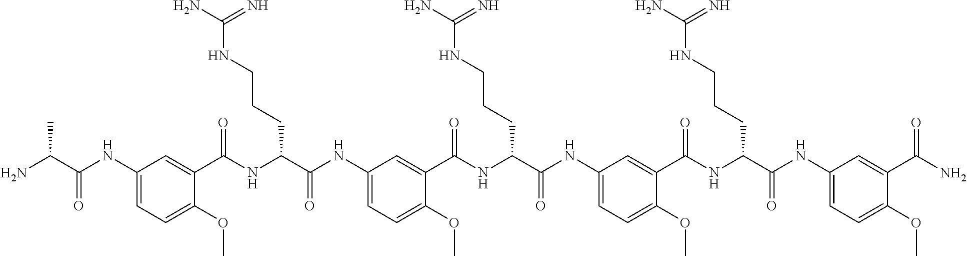 |
| 17 | 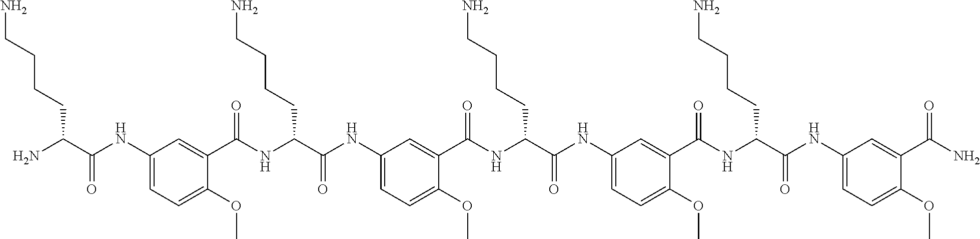 |
| 18 | 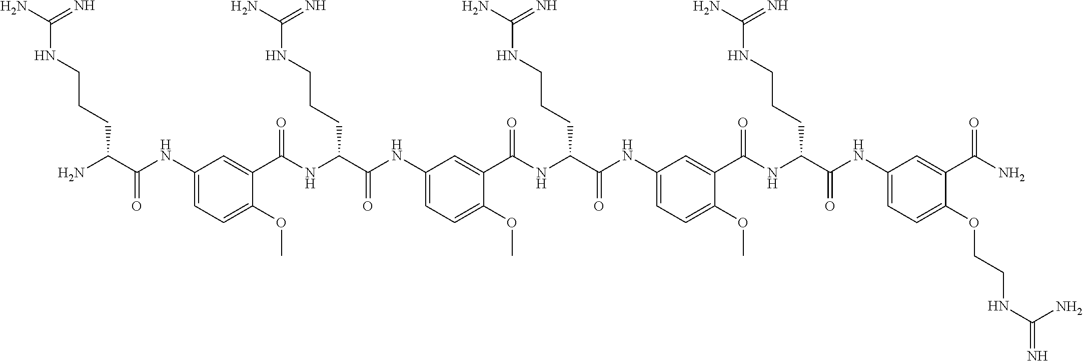 |
| 19 | 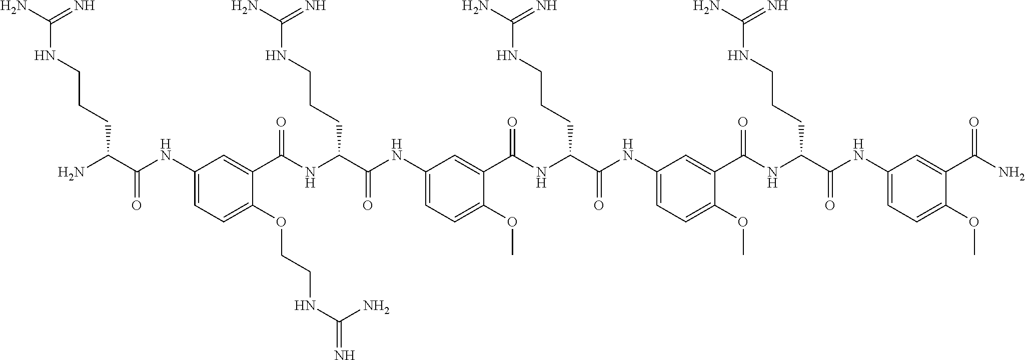 |
| 20 | 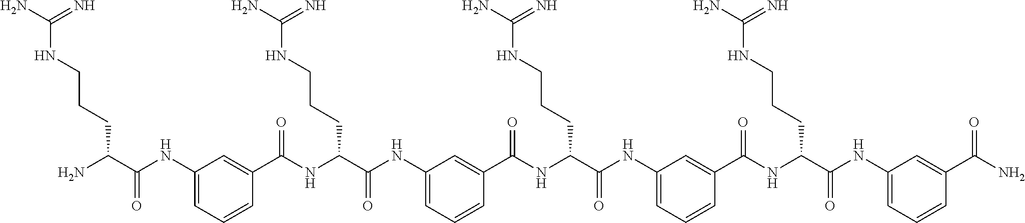 |
| 21 | 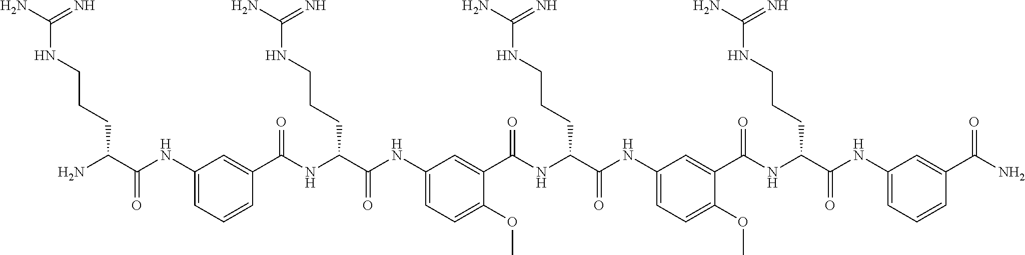 |
| 22 | 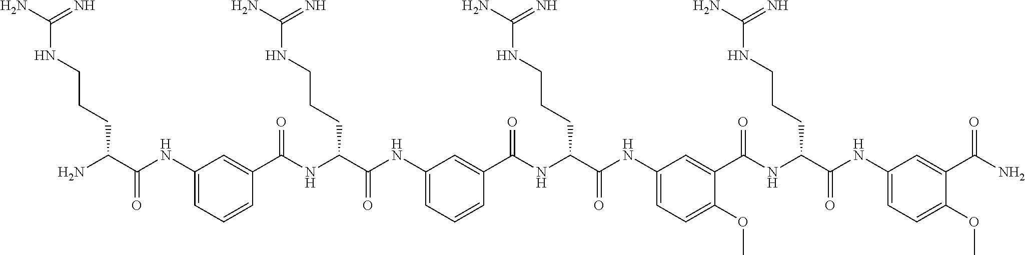 |
| 23 | 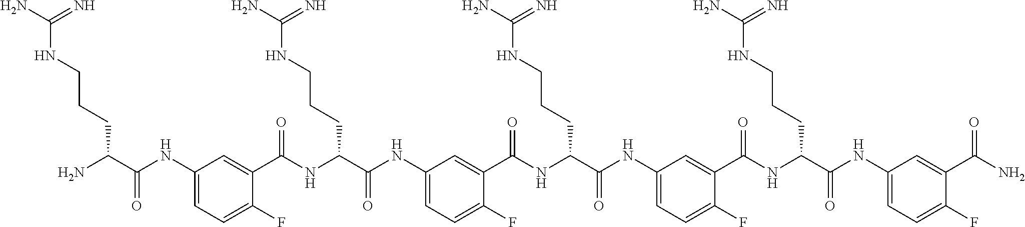 |
| 24 | 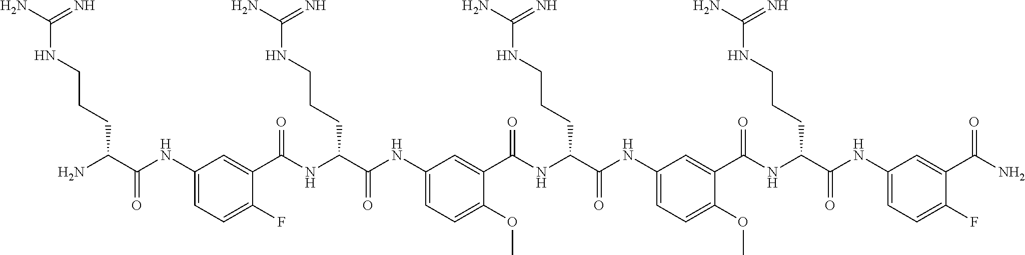 |
| 25 | 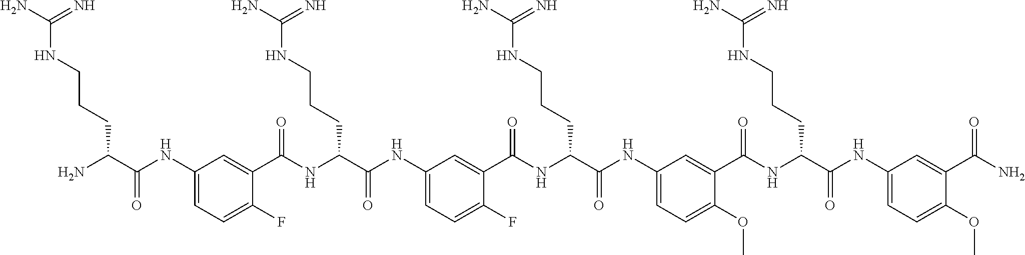 |
| 26 | 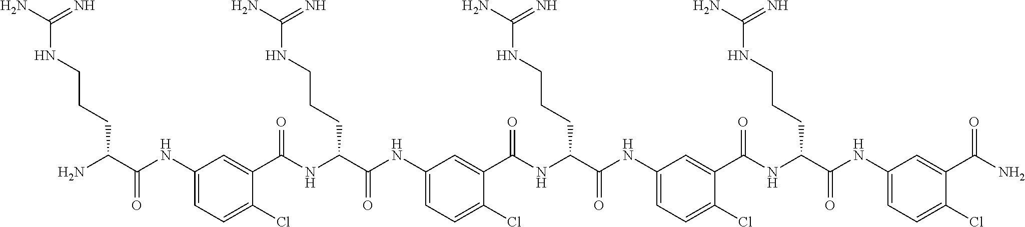 |
| 27 | 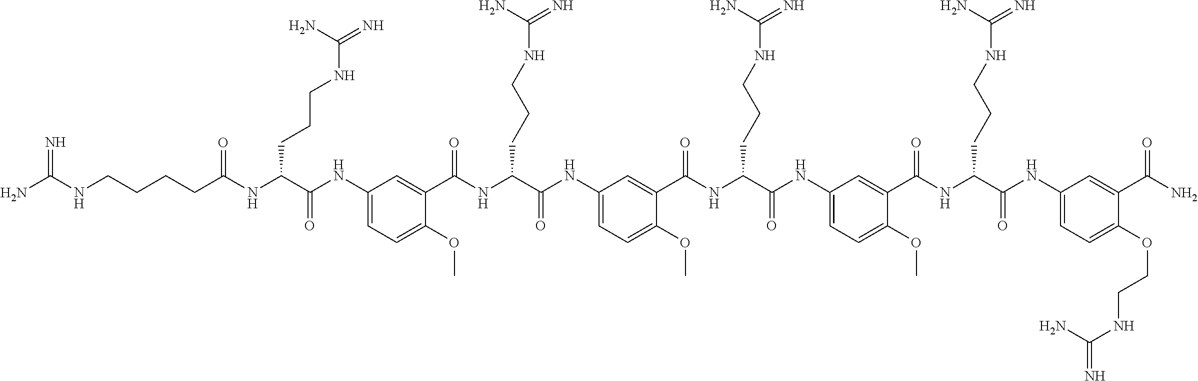 |
| 28 | 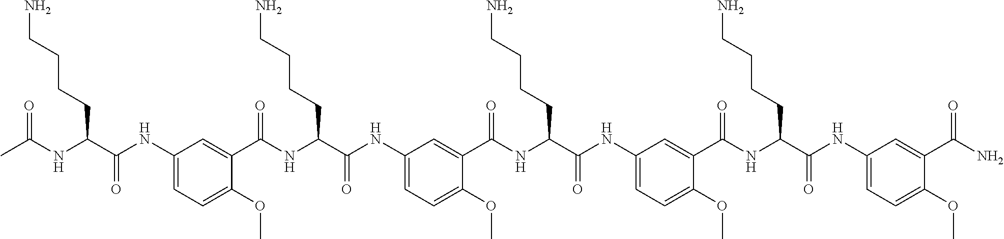 |
| 29 | 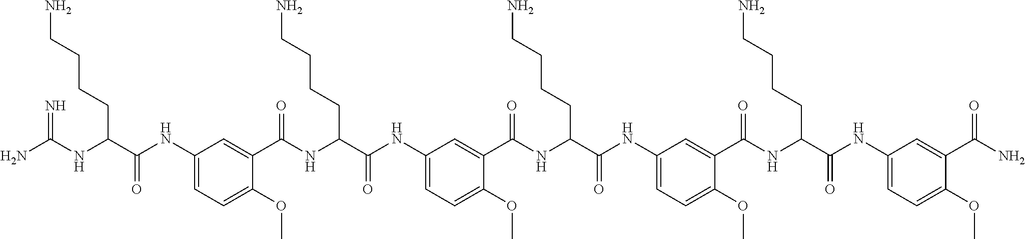 |
| 30 | 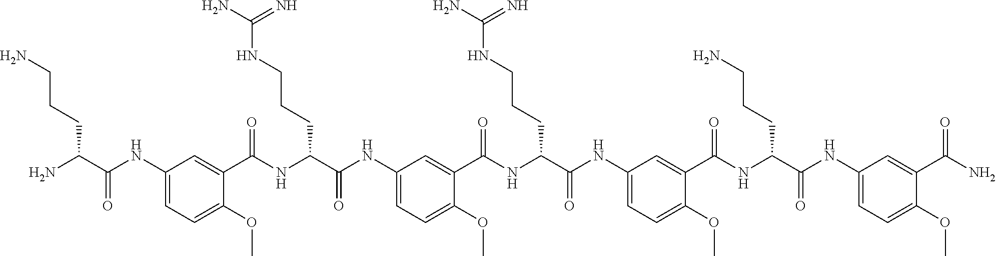 |
| 31 | 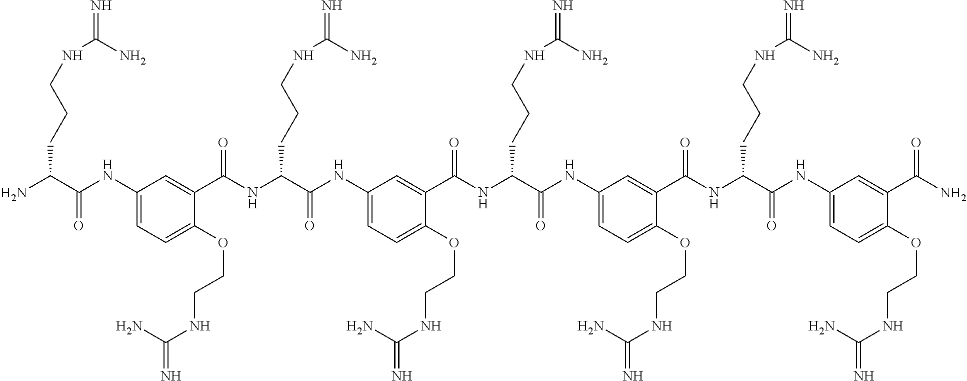 |
| 32 | 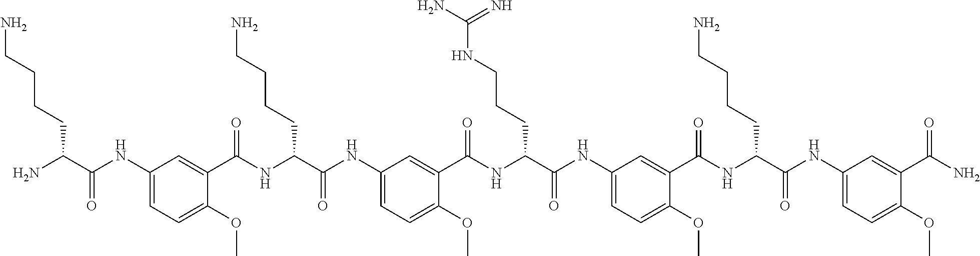 |
| 33 | 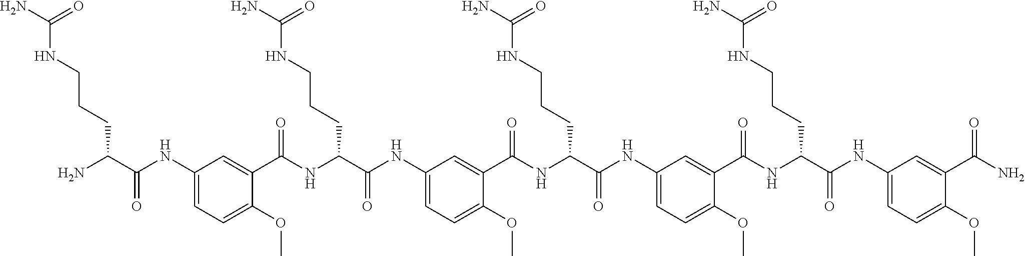 |
| 34 | 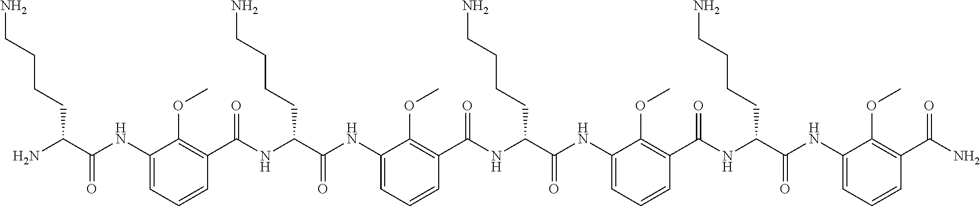 |
| 35 | 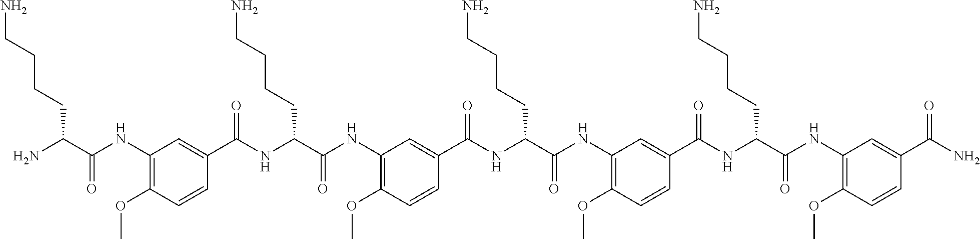 |
| 36 | 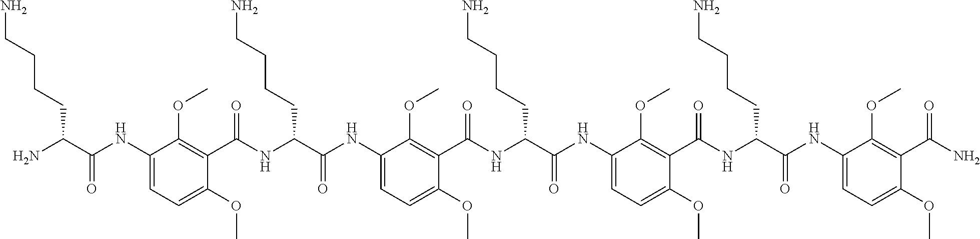 |
| 37 | 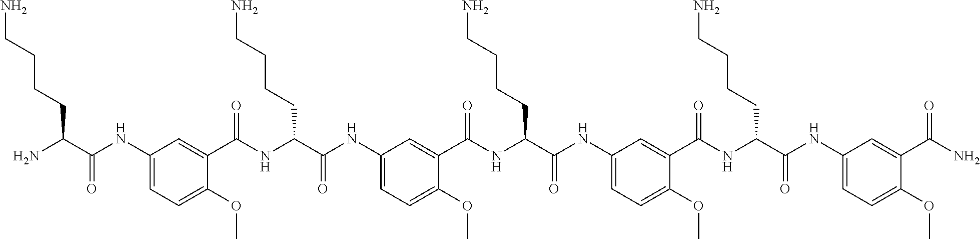 |
| 38 | 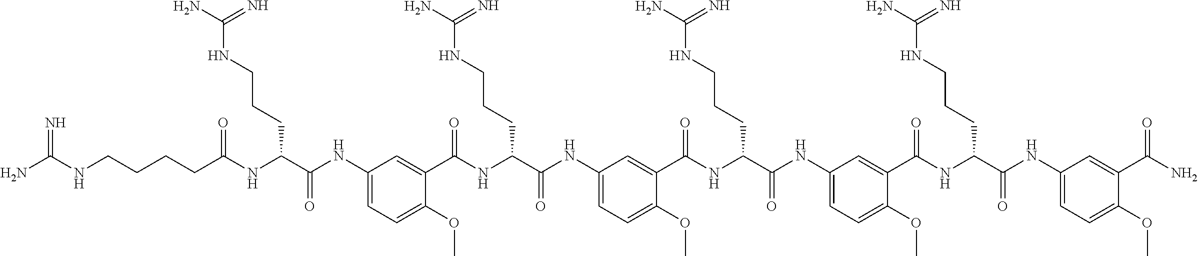 |
| 39 | 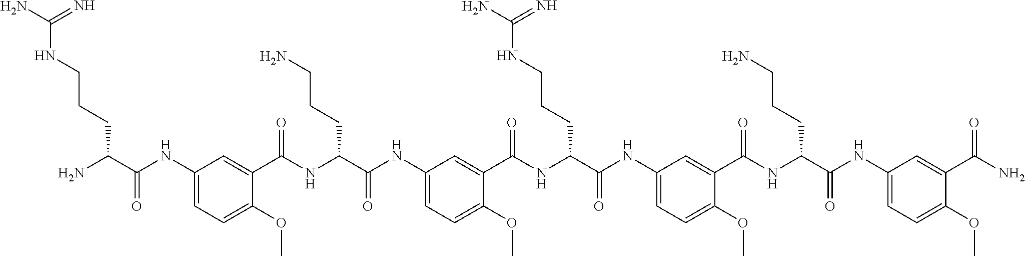 |
| 40 | 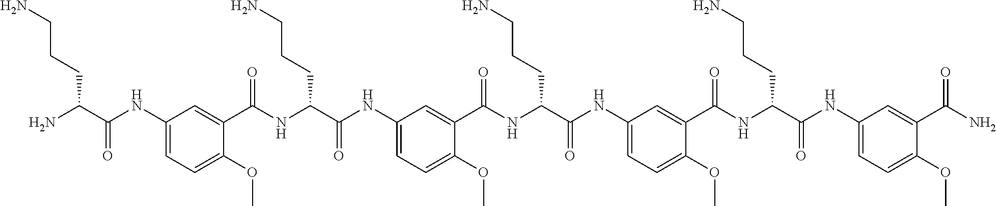 |
| 41 | 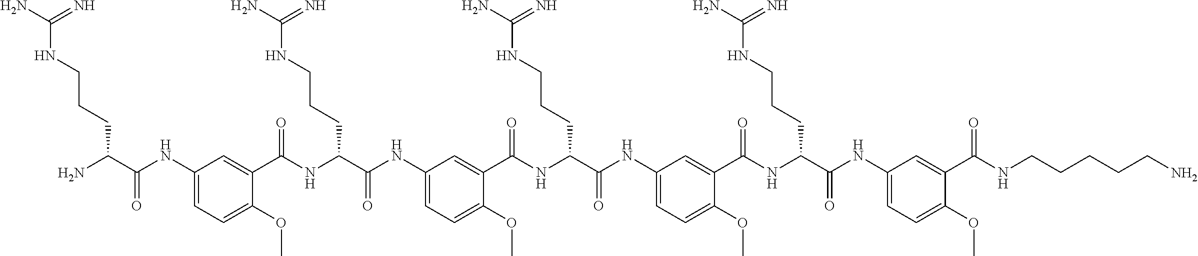 |
| 42 | 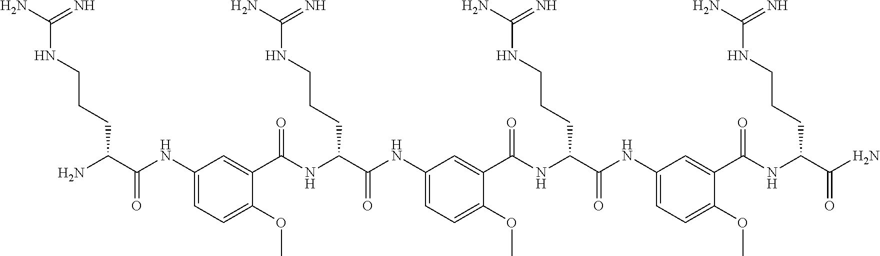 |
| 43 | 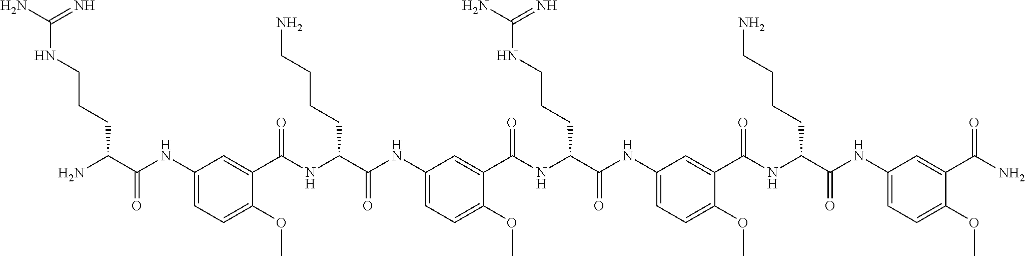 |
| 44 | 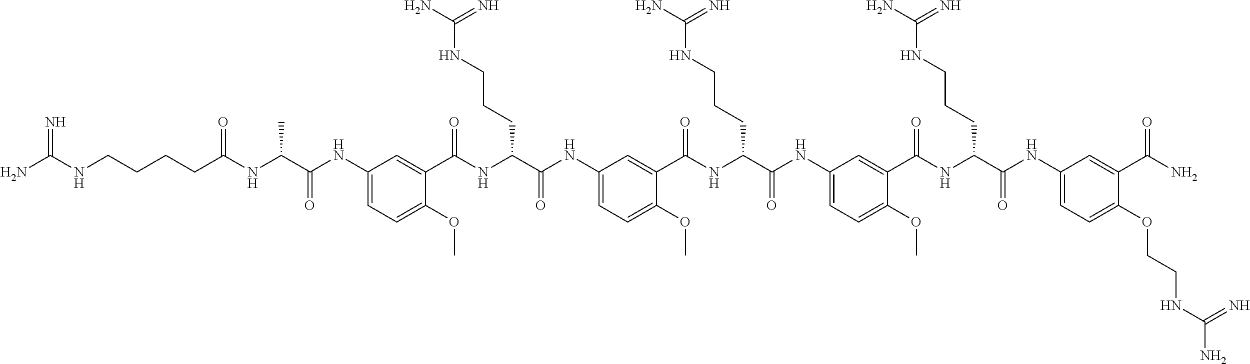 |
| 45 | 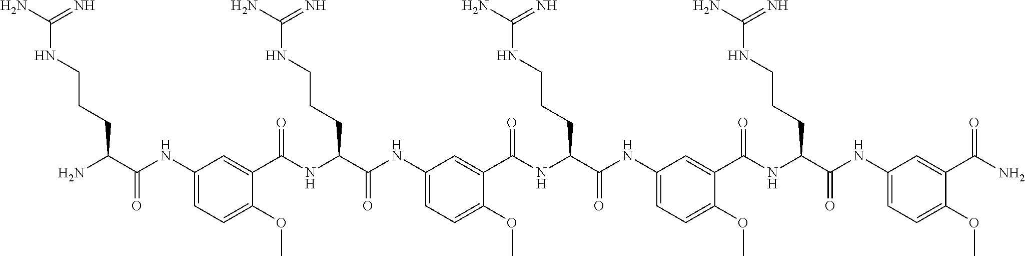 |
| 46 | 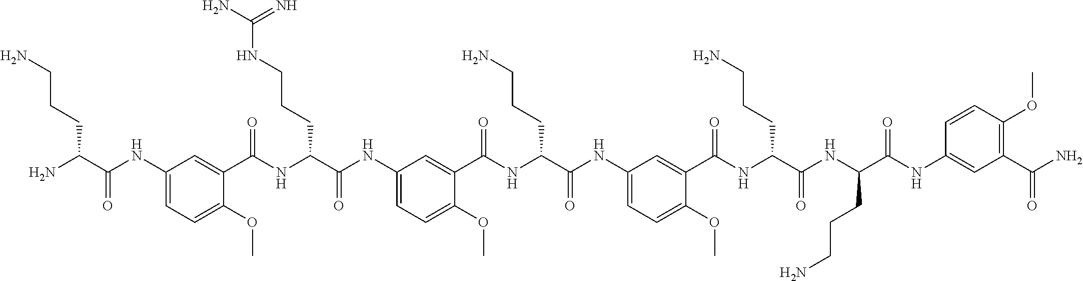 |
| 47 | 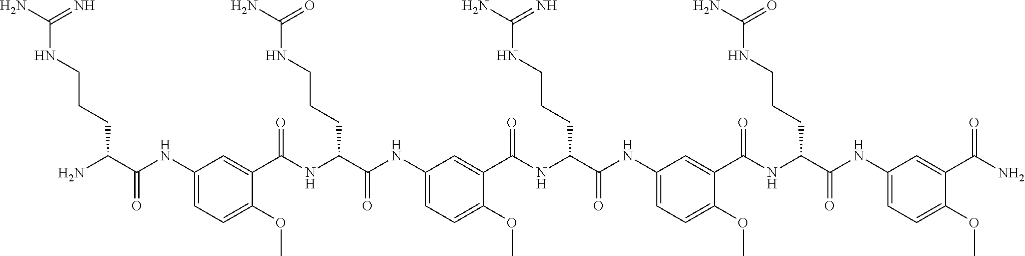 |
| 48 | 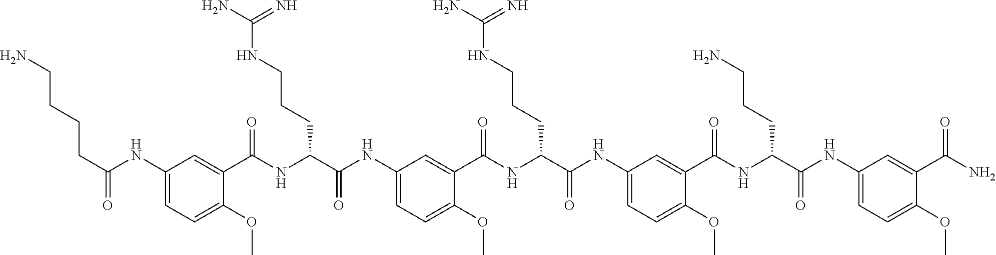 |
| 49 | 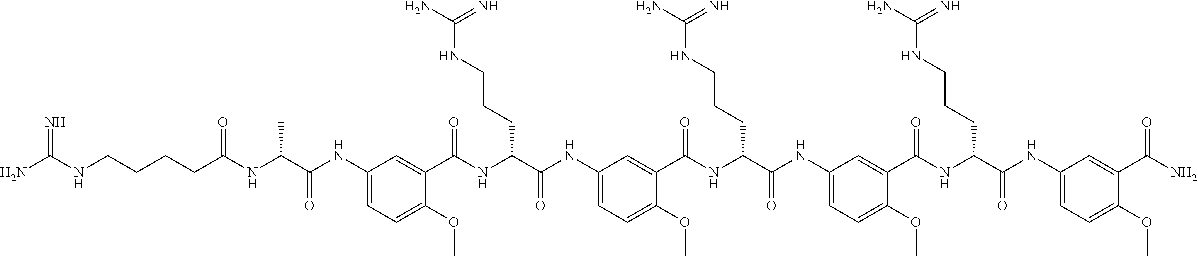 |
| 50 | 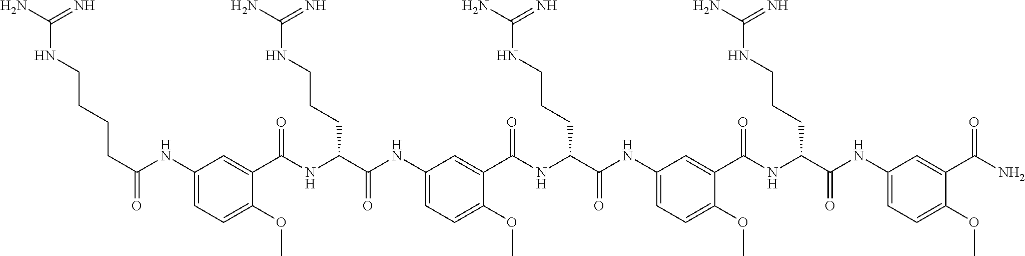 |
| 51 | 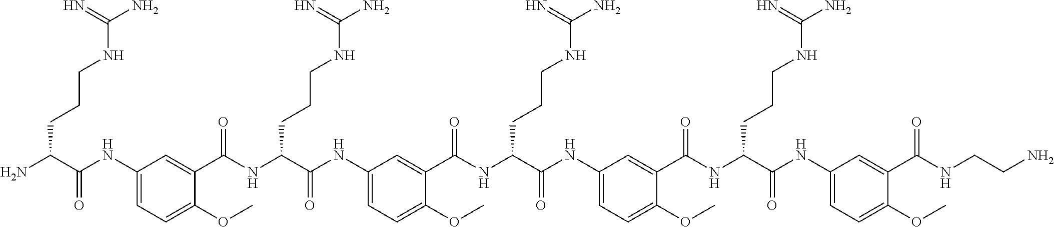 |
| 52 | 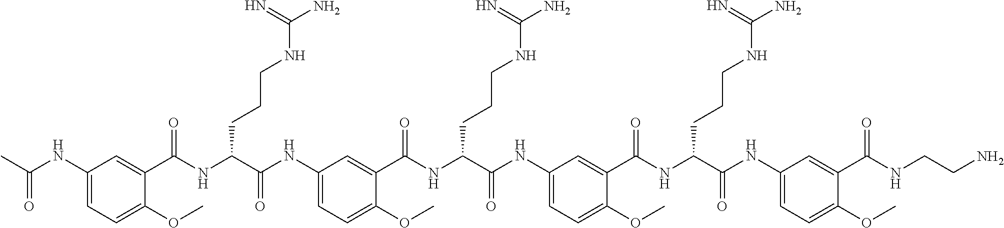 |
| 53 | 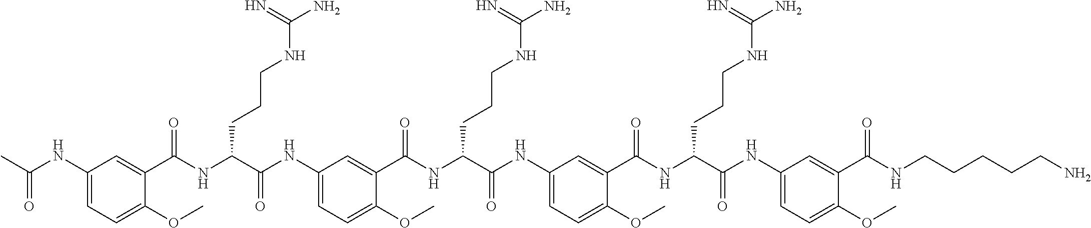 |
| 54 | 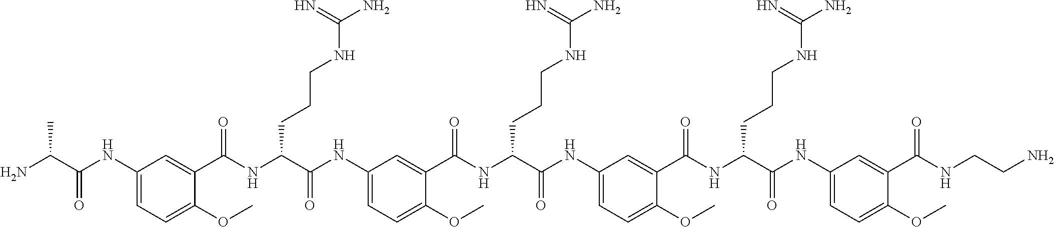 |
| 55 | 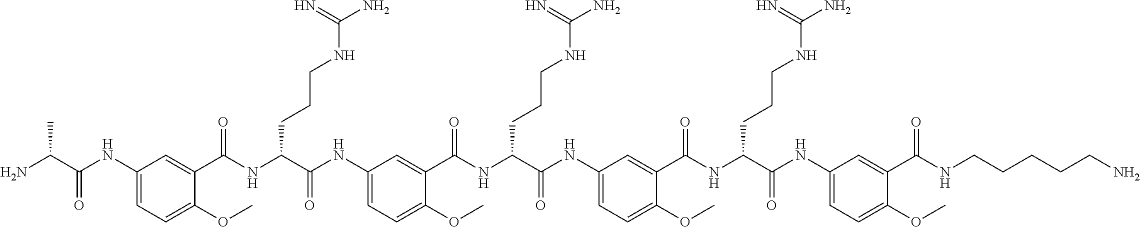 |
| 56 | 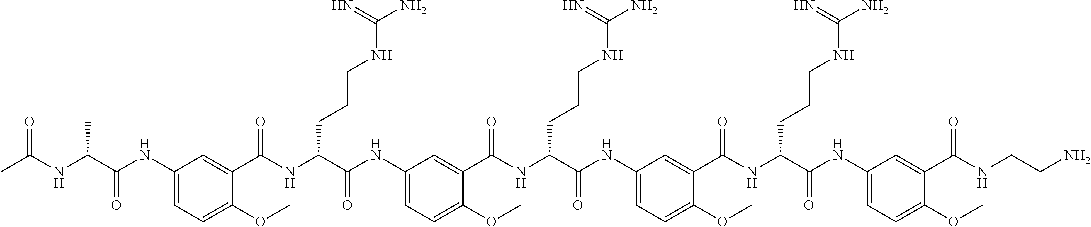 |
| 57 | 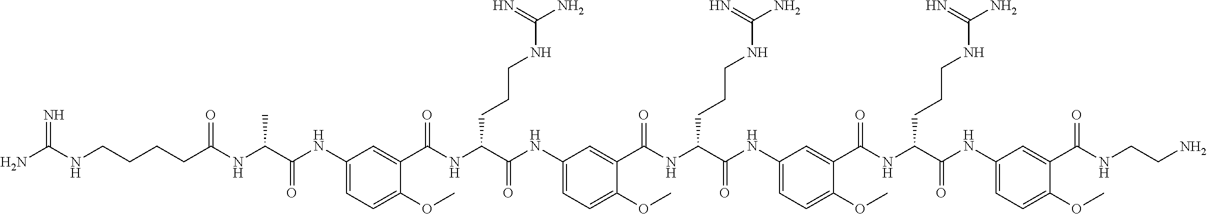 |
| 58 | 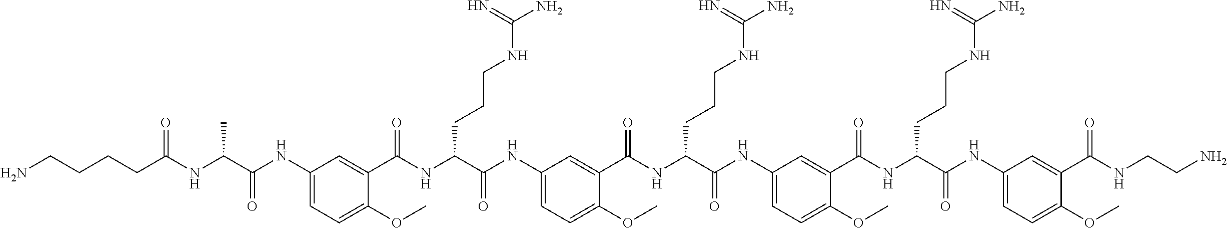 |
| 59 | 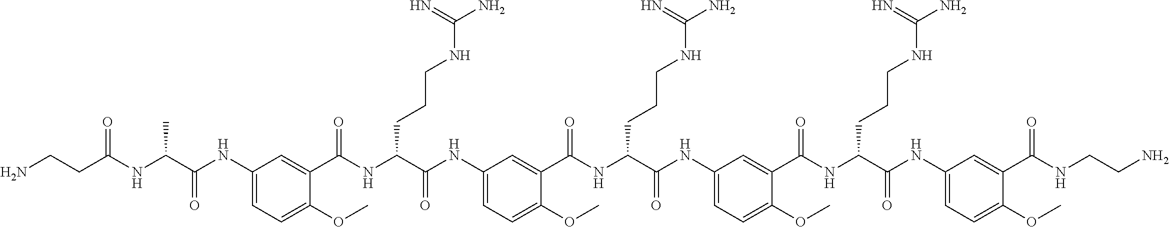 |
| 60 | 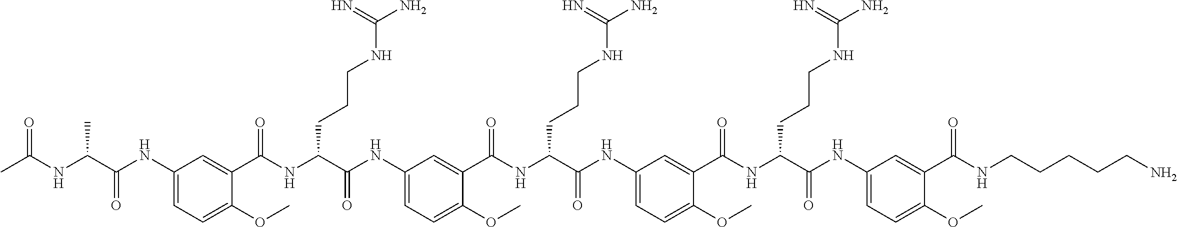 |
| 61 | 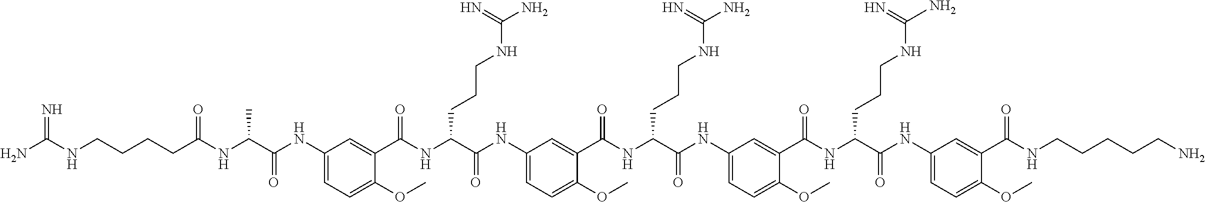 |
| 62 | 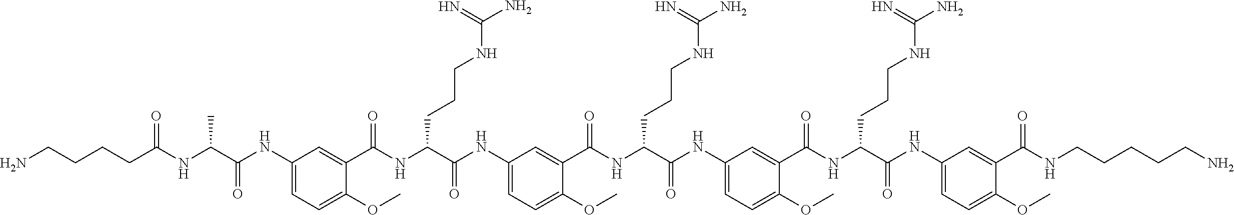 |
| 63 | 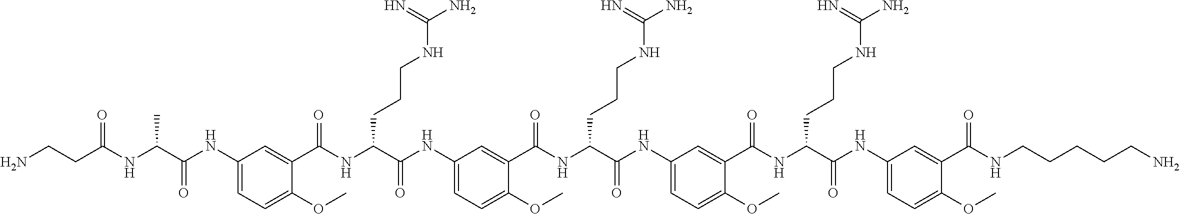 |
| 64 | 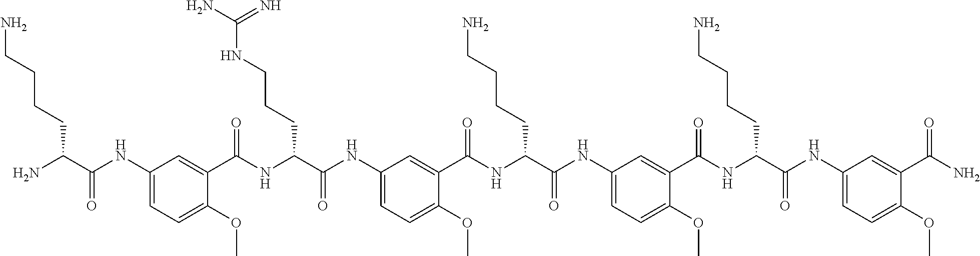 |
| 65 | 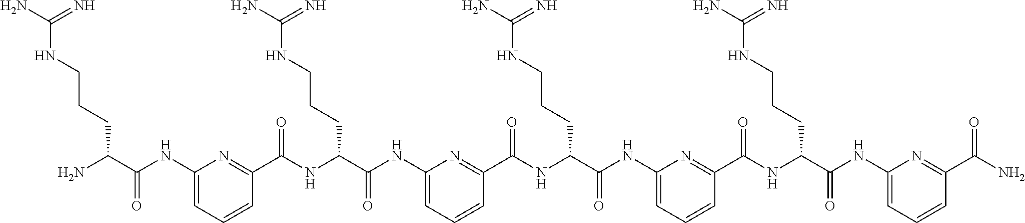 |
| 66 | 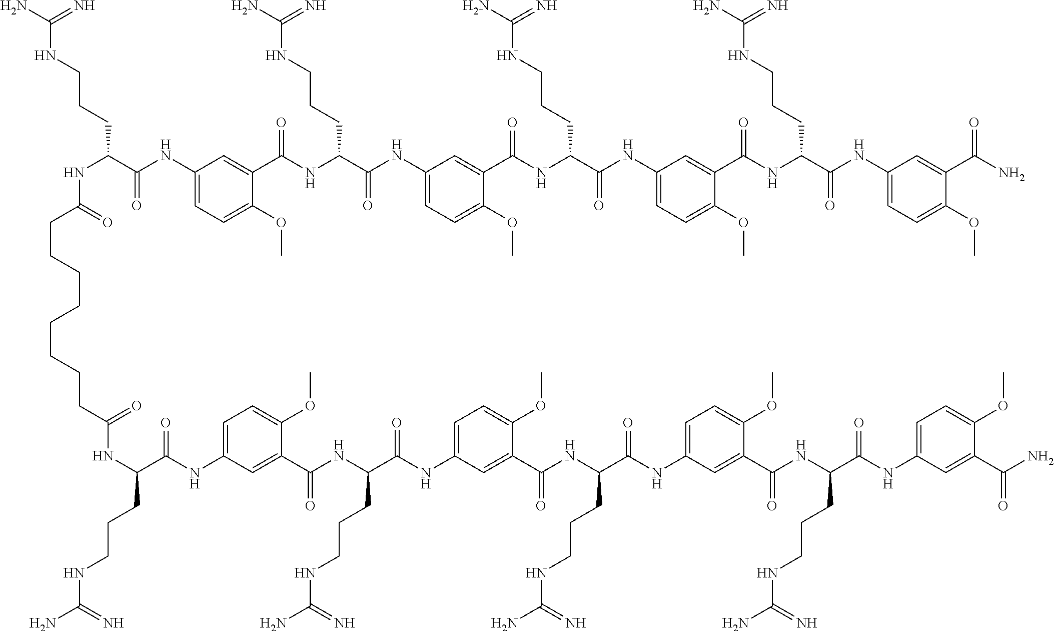 |
| 67 | 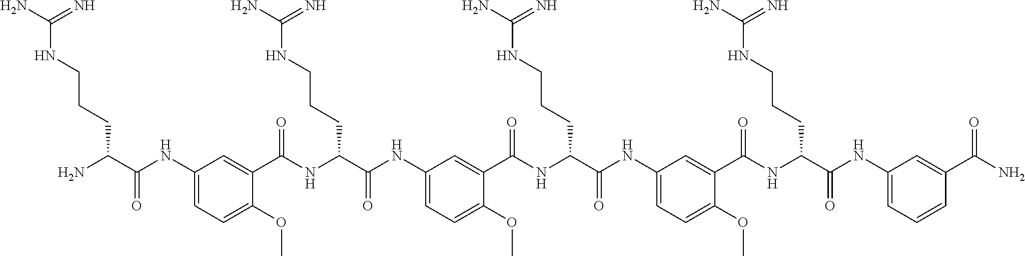 |
| 68 | 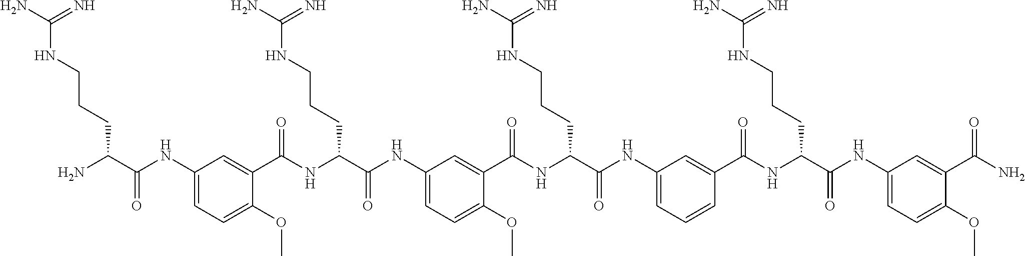 |
| 69 | 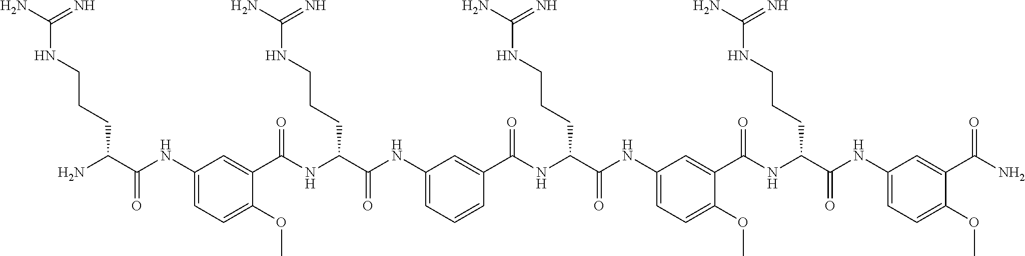 |
| 70 | 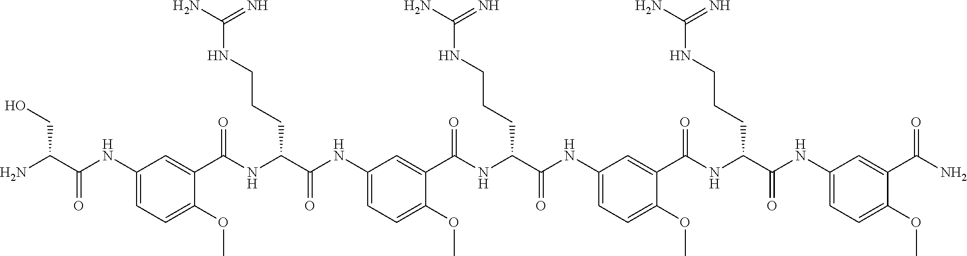 |
| 71 | 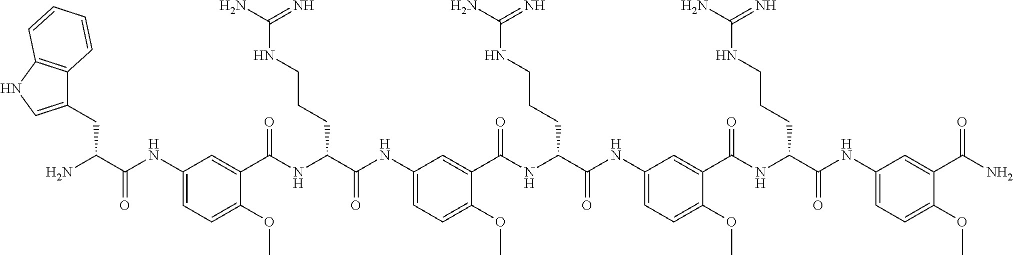 |
| 72 | 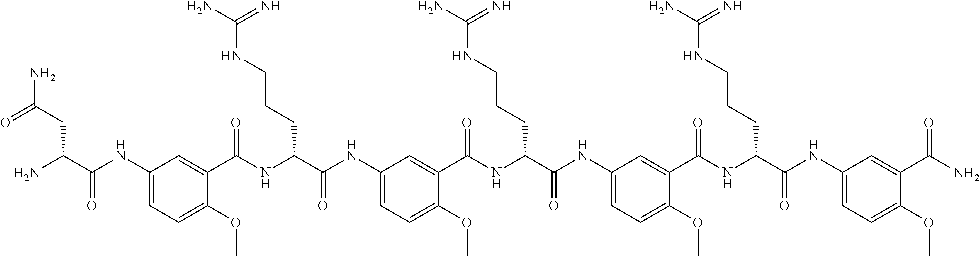 |
| 73 | 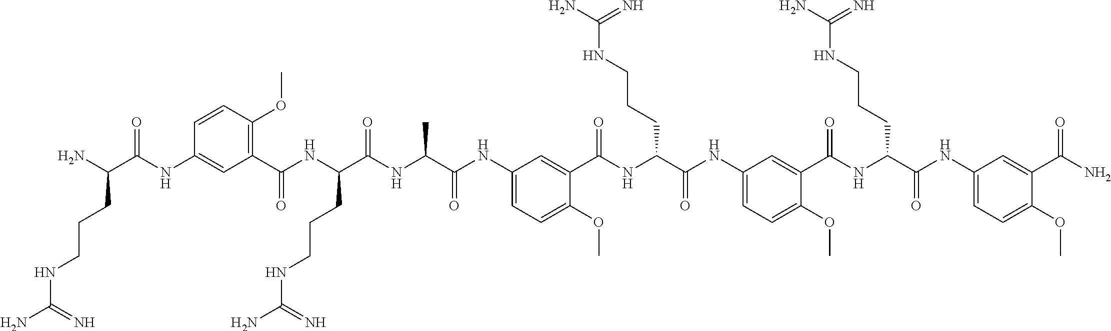 |
| 74 | 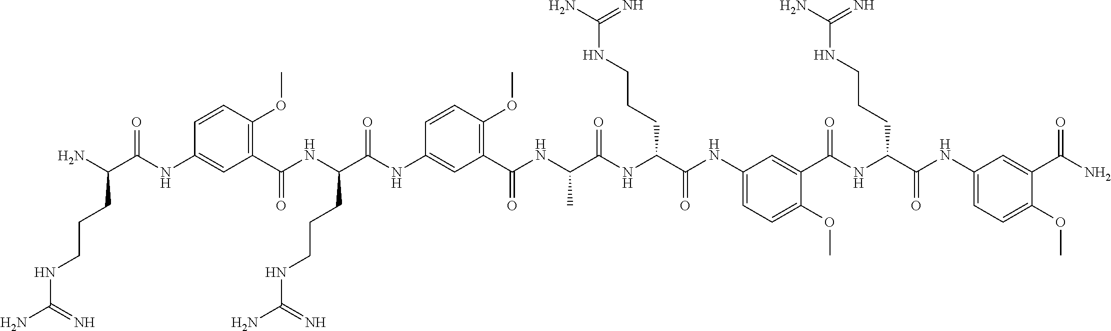 |
| 75 | 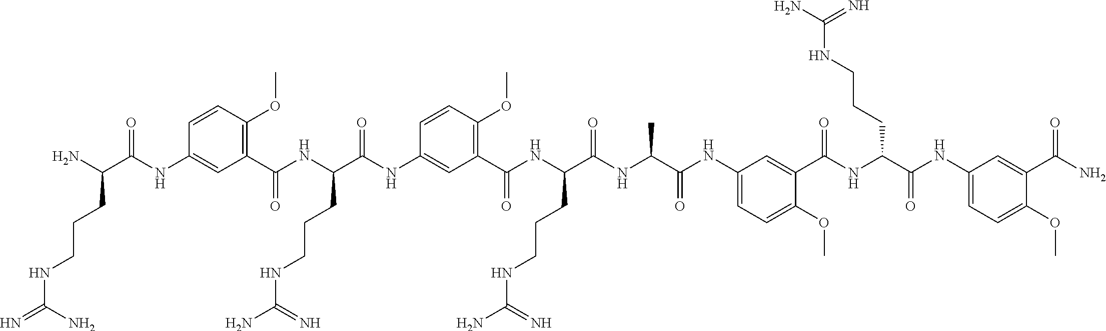 |
| 76 | 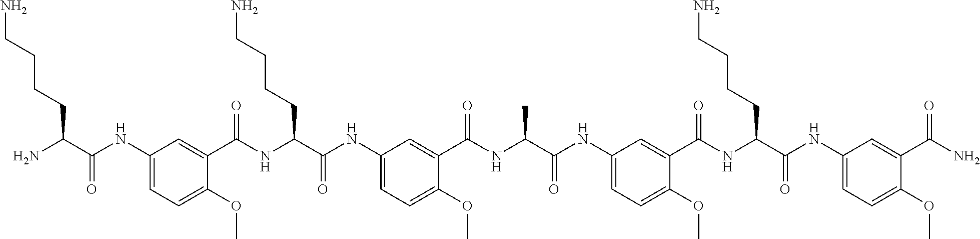 |
| 77 | 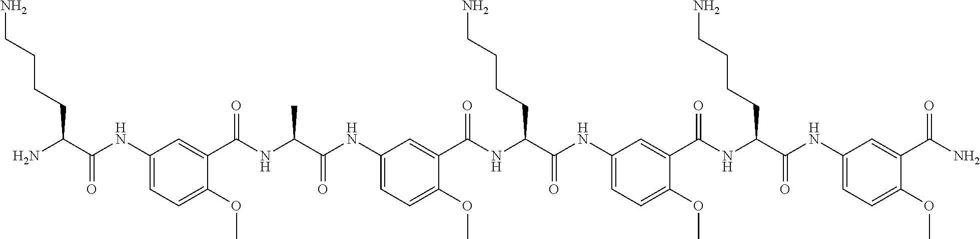 |
| 78 | 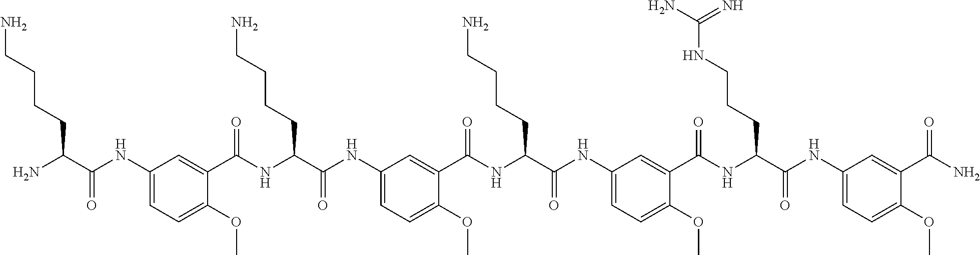 |
| 79 | 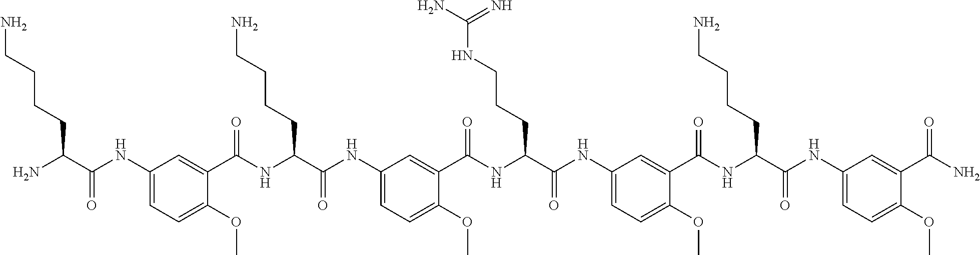 |
| 80 | 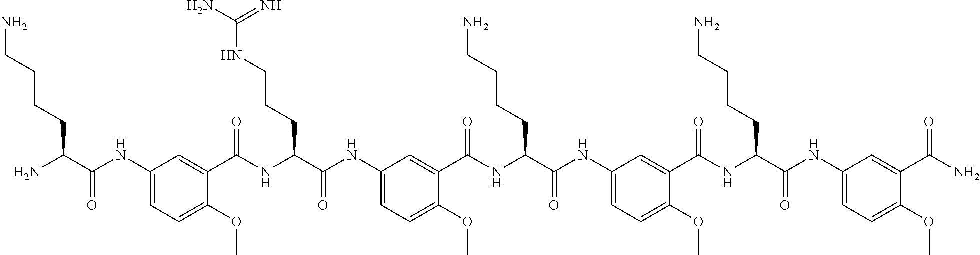 |
| 81 | 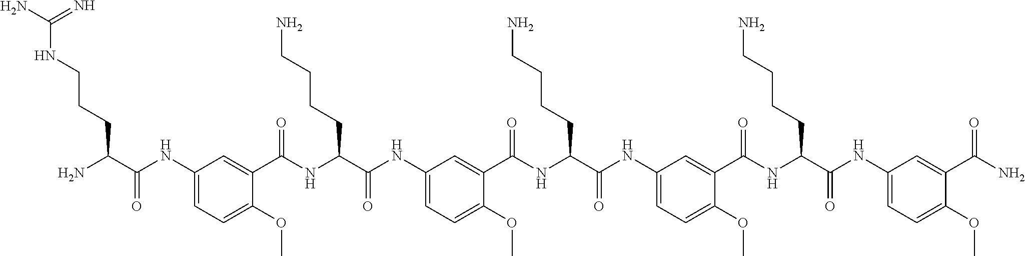 |
| 82 | 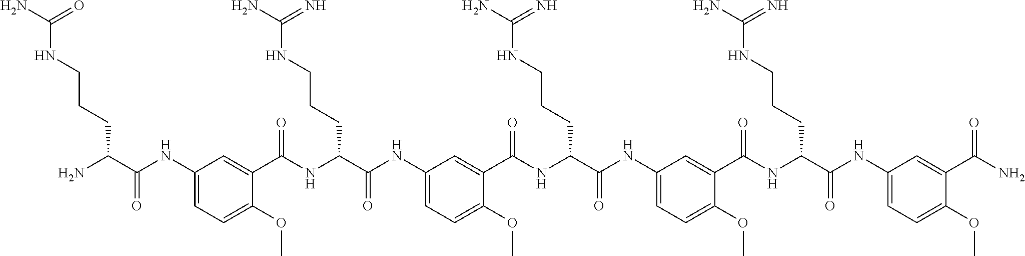 |
| 83 | 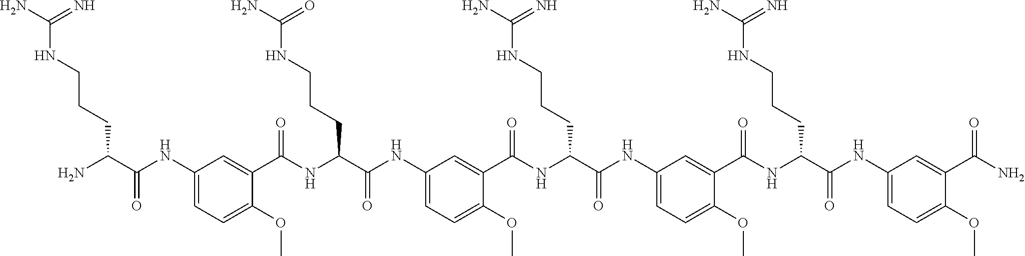 |
| 84 | 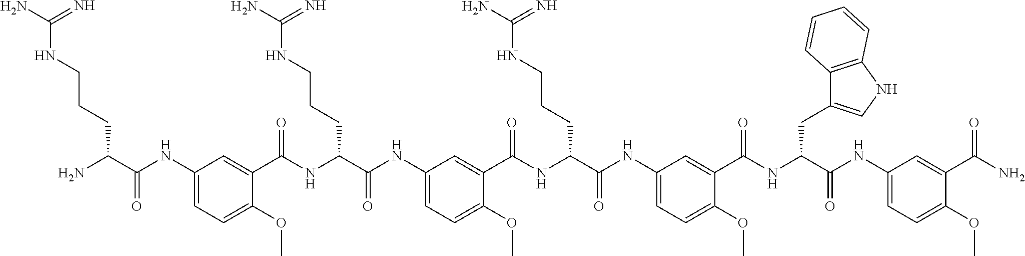 |
| 85 | 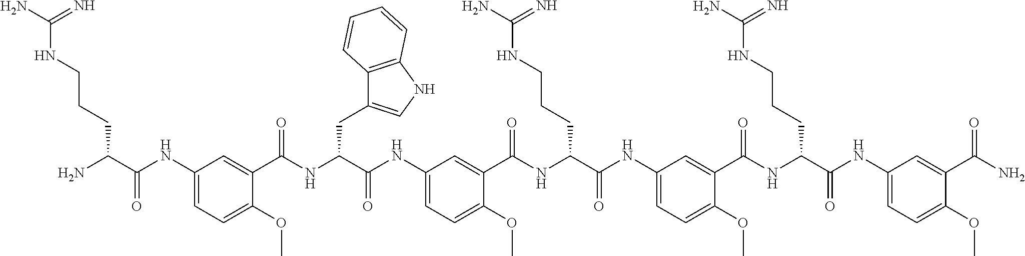 |
| 86 | 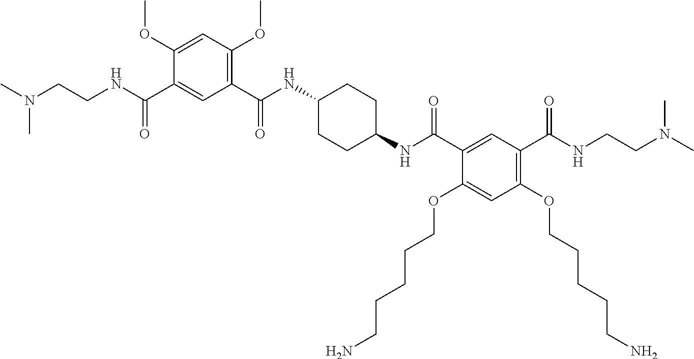 |
| 87 | 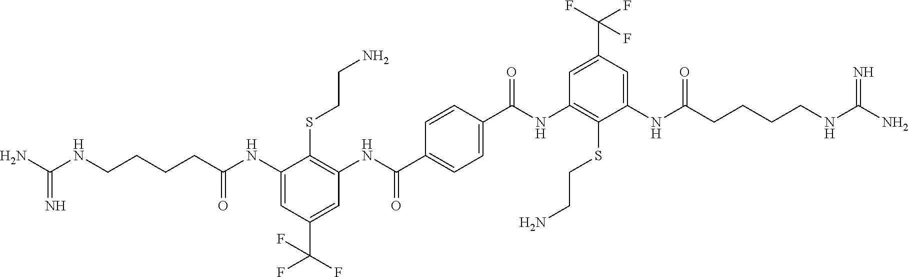 |
| 88 | 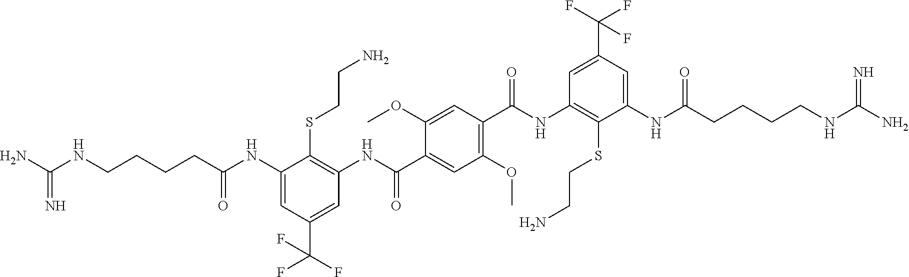 |
| 89 | 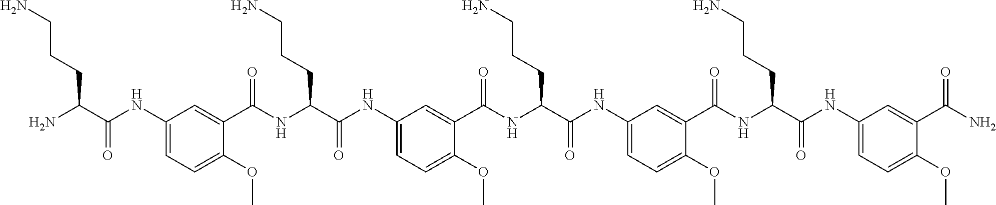 |
| 90 | 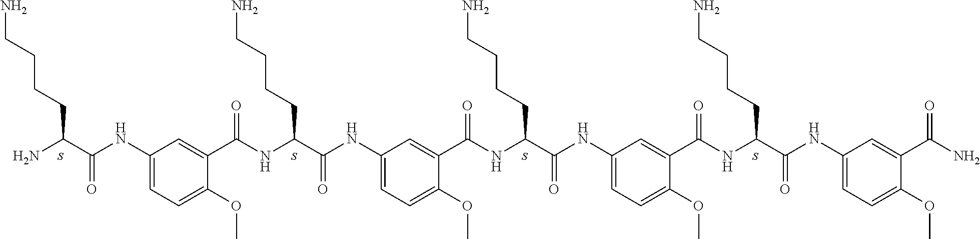 |
| 91 | 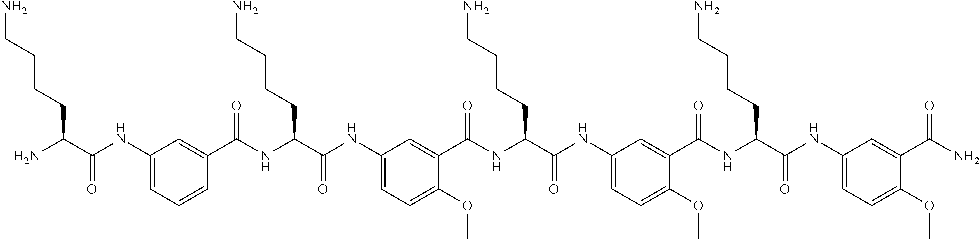 |
| 92 | 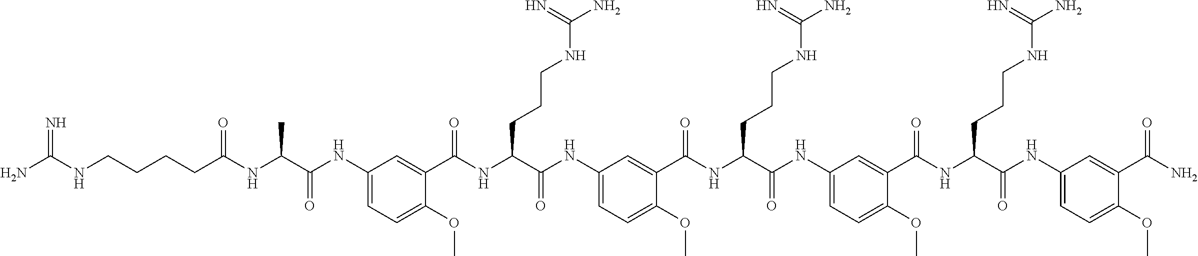 |
| 93 | 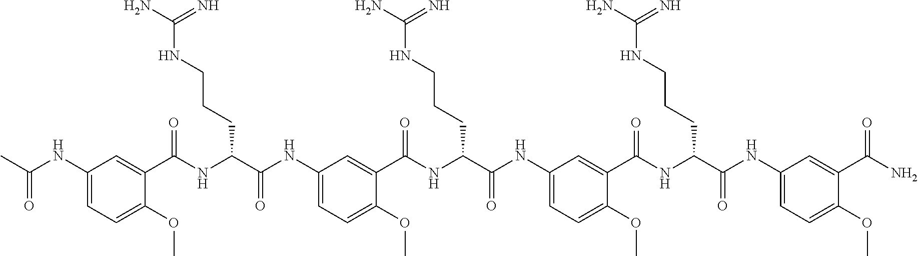 |
| 94 | 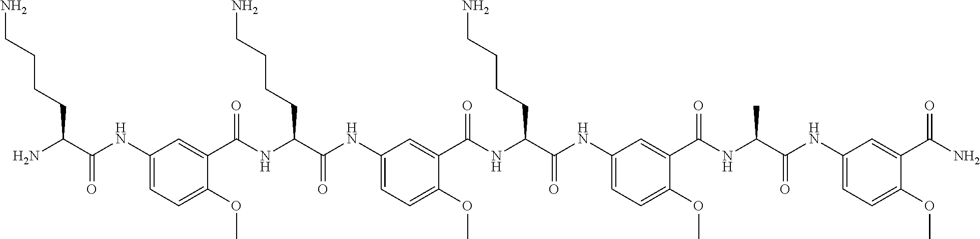 |
| 95 | 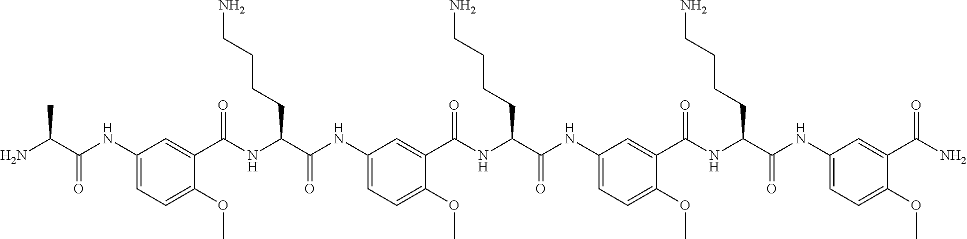 |
| 96 | 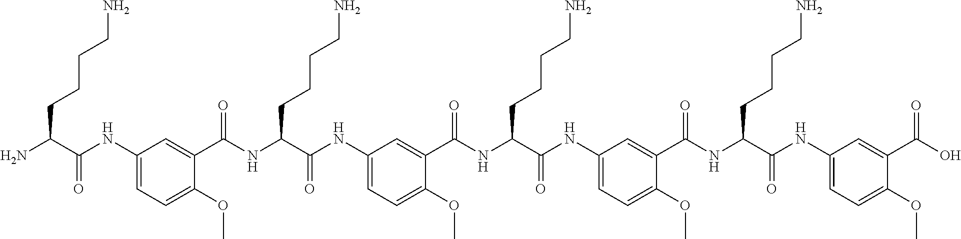 |
| 97 | 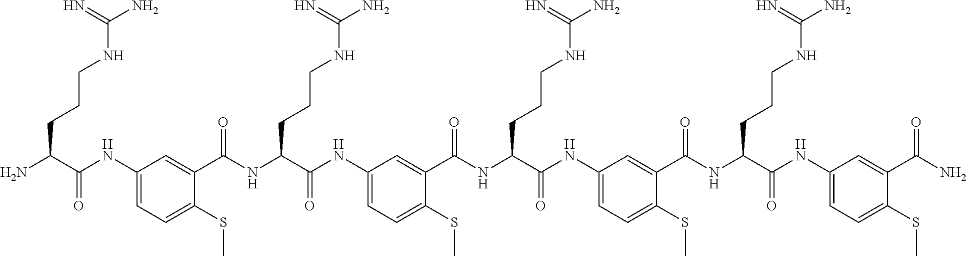 |
| 98 | 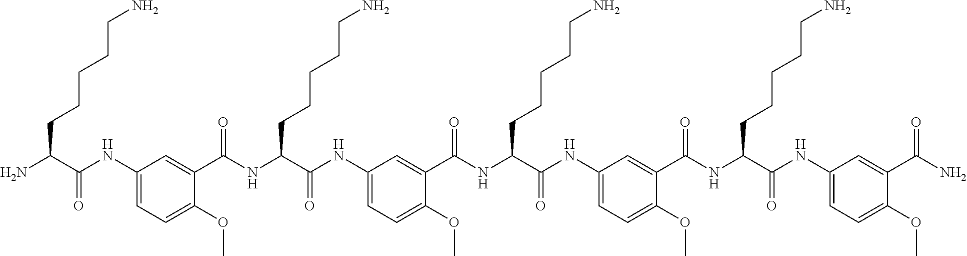 |
| 99 | 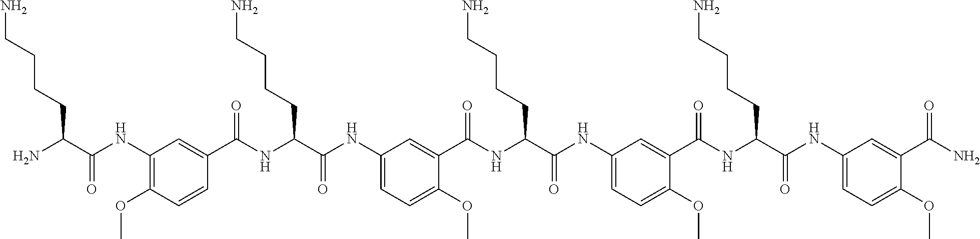 |
| 100 | 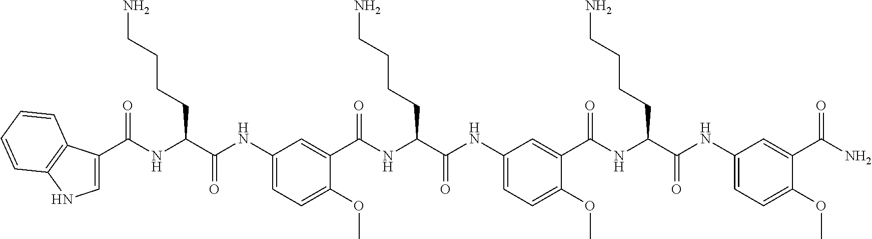 |
| 101 | 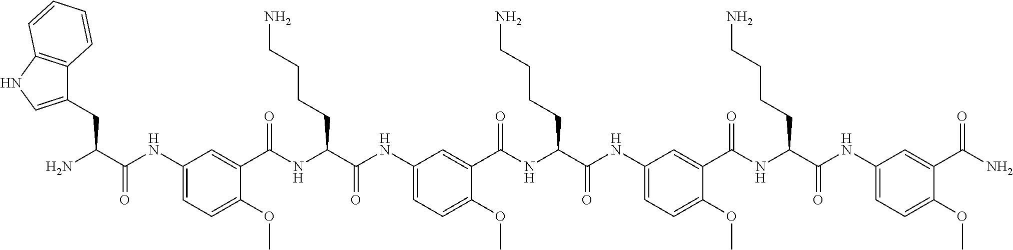 |
| 102 | 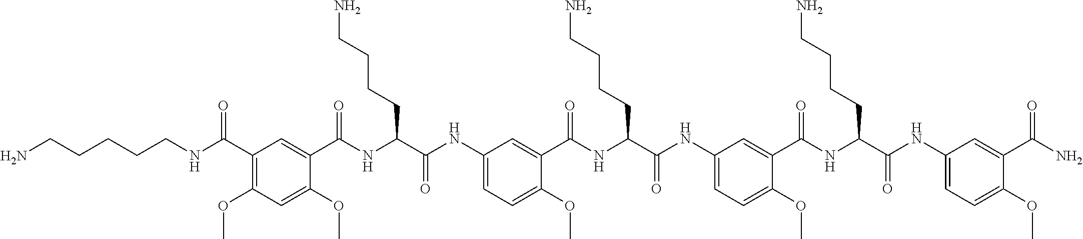 |
| 103 | 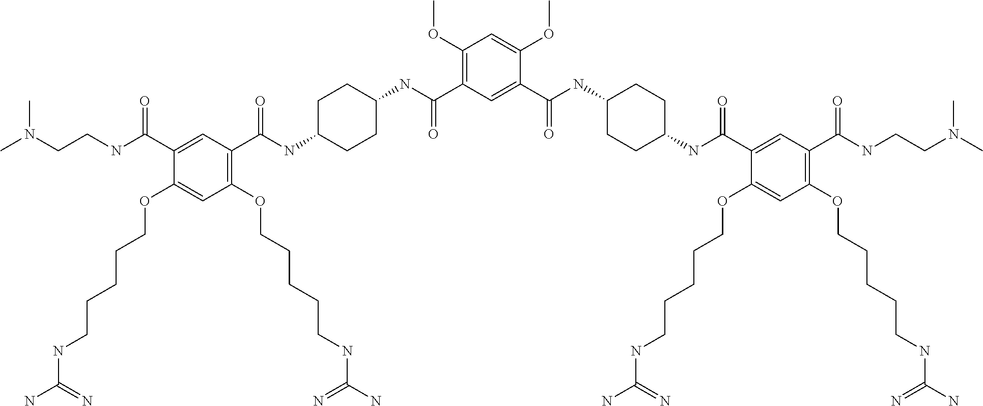 |
| 104 | 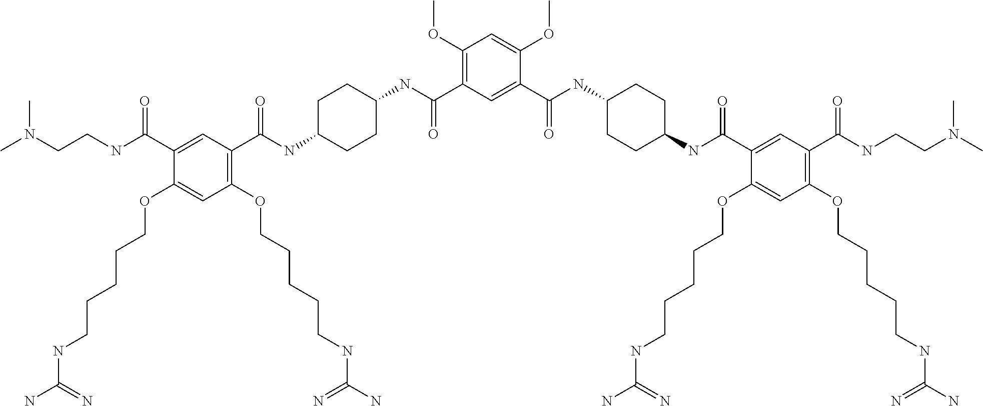 |
| 105 | 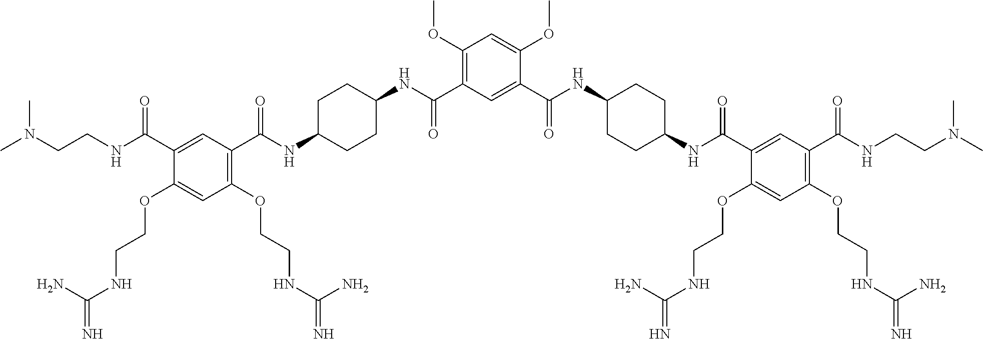 |
| 106 | 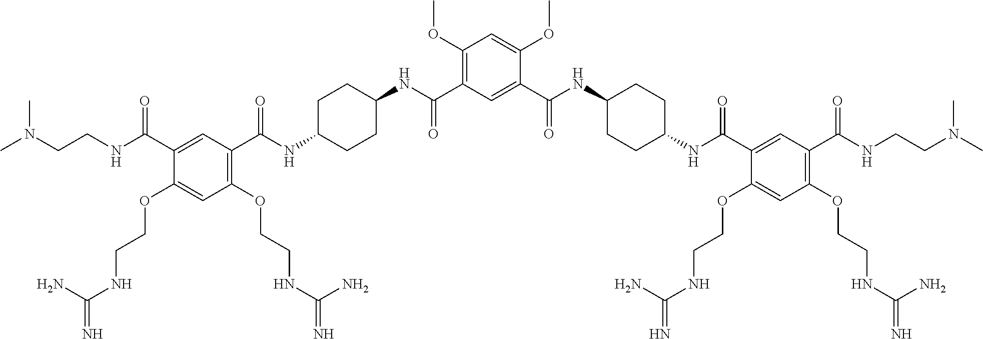 |
| 107 | 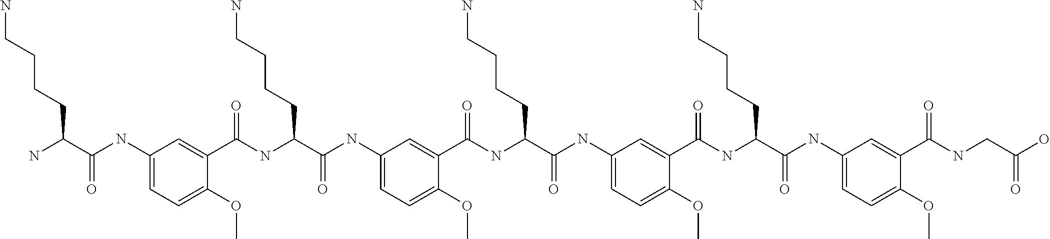 |
| 108 | 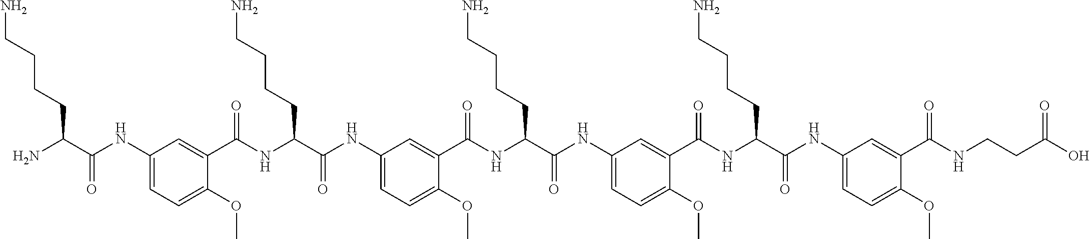 |
| 109 | 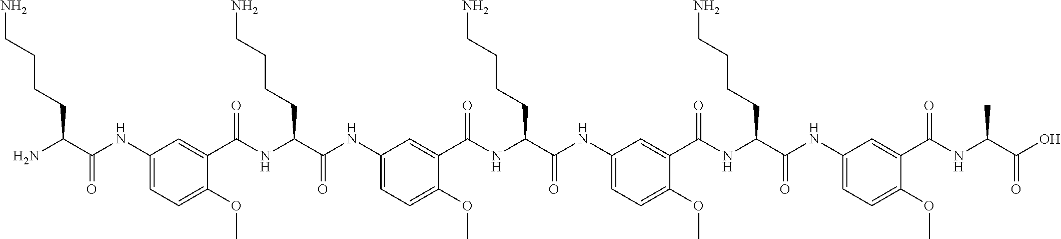 |
| 110 | 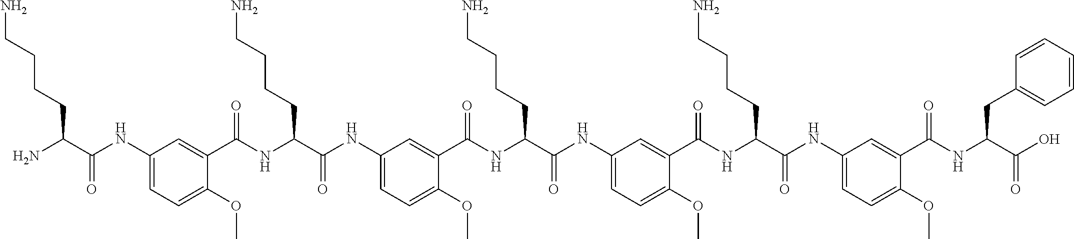 |
| 111 | 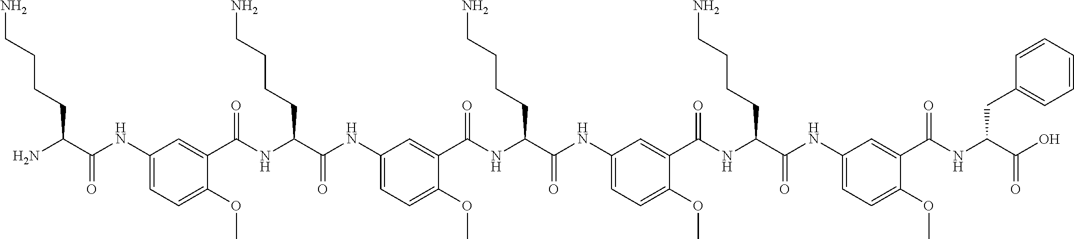 |
| 112 | 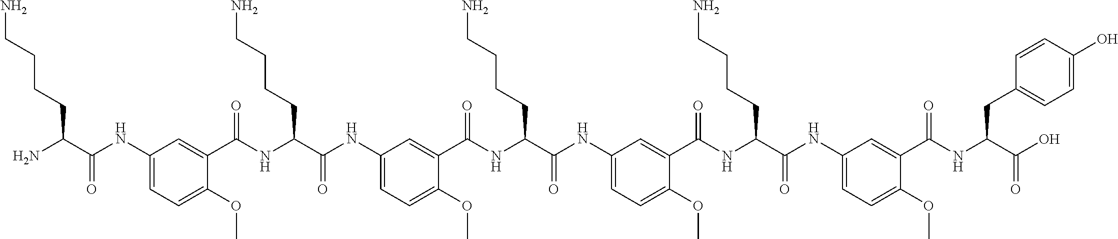 |
| 113 | 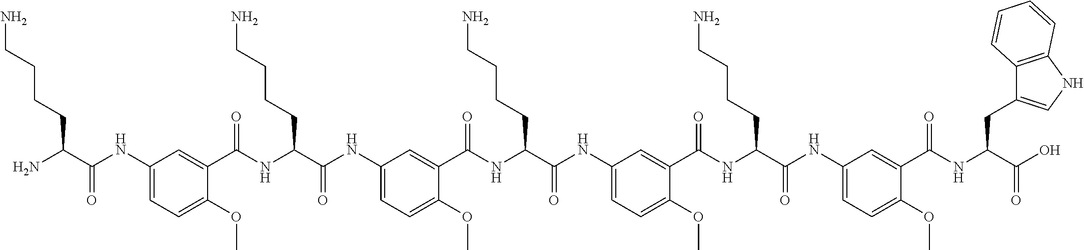 |
| 114 | 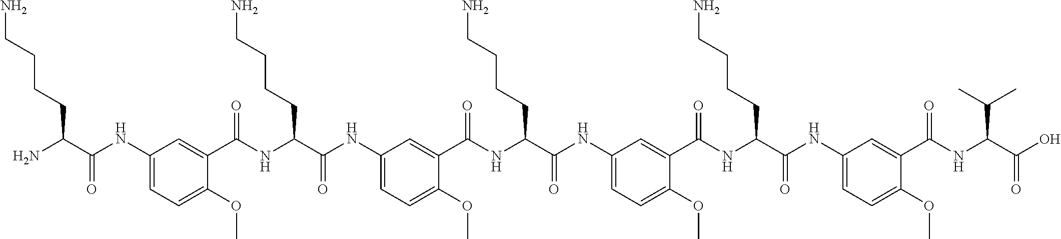 |
| 115 | 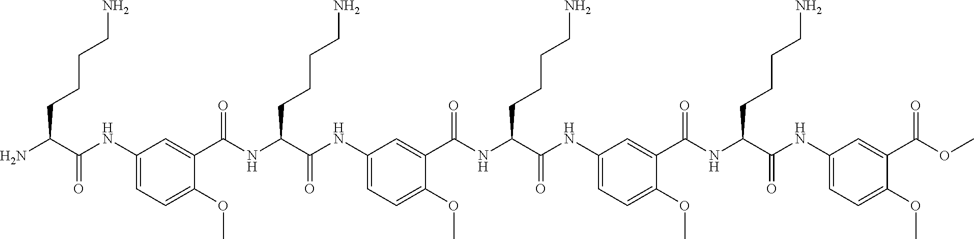 |
| 116 | 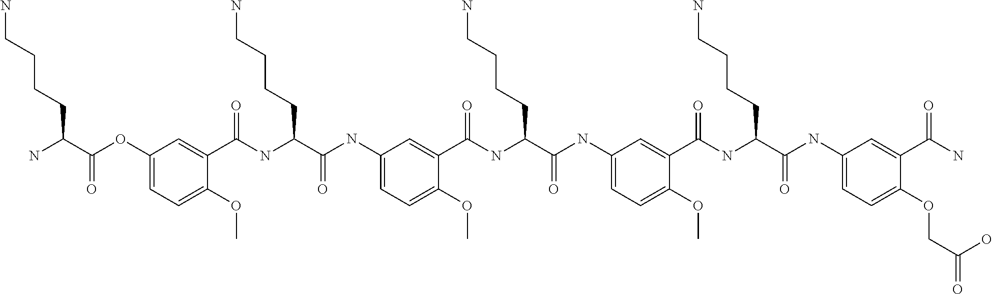 |
| 117 | 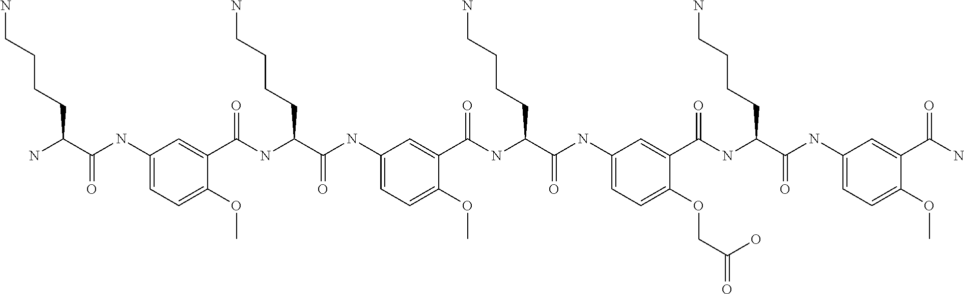 |
| 118 | 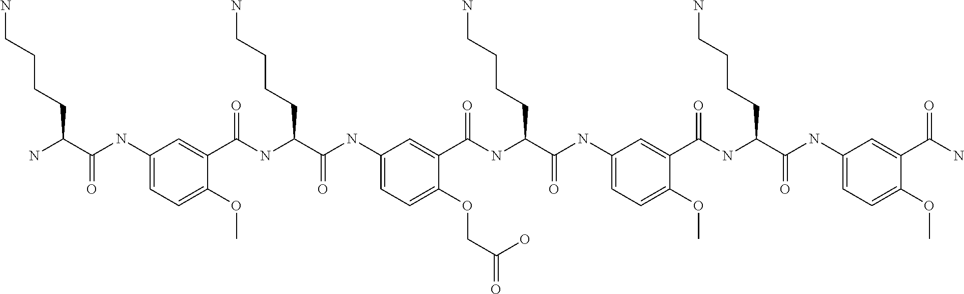 |
| 119 | 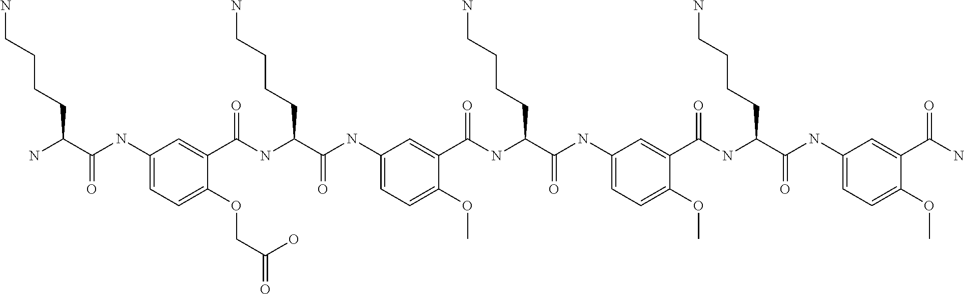 |
| 120 | 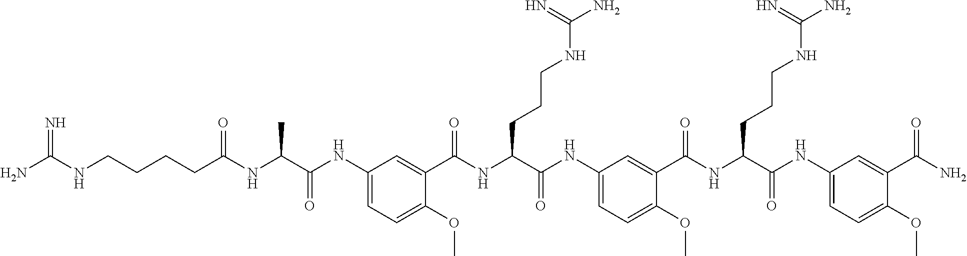 |
| 121 | 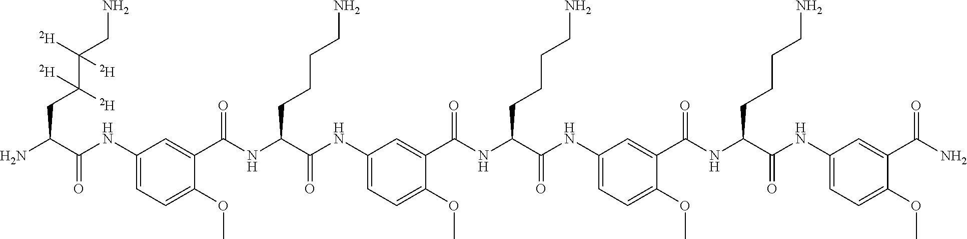 |
| 122 | 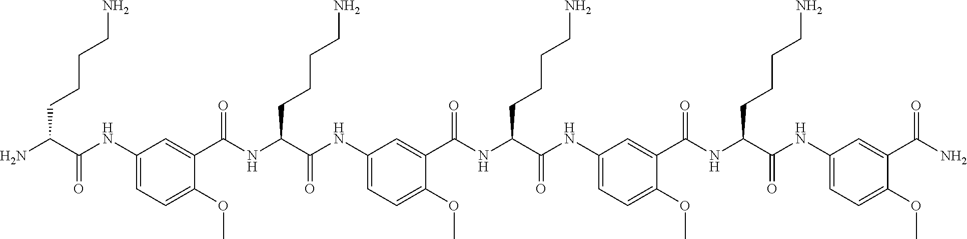 |
| 123 | 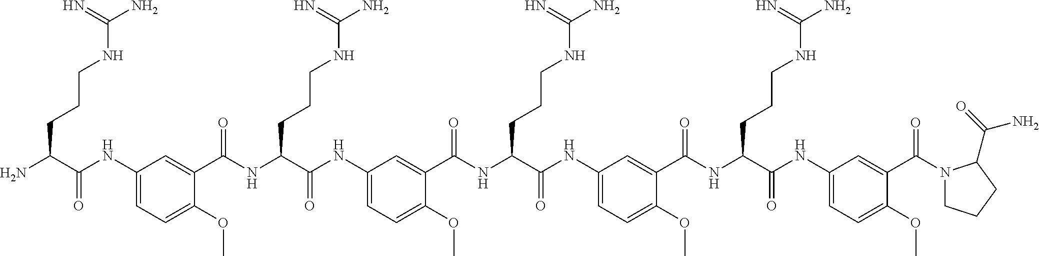 |
| 124 | 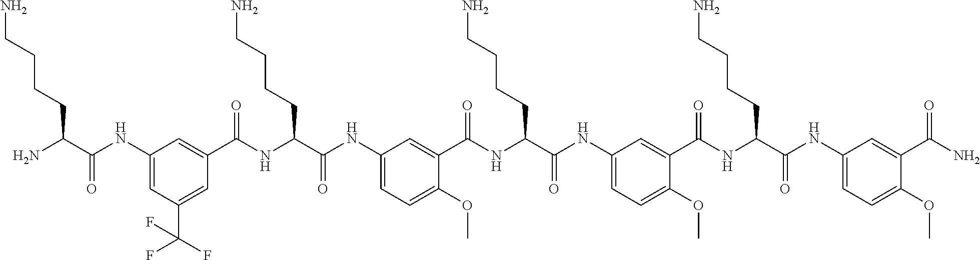 |
| 125 | 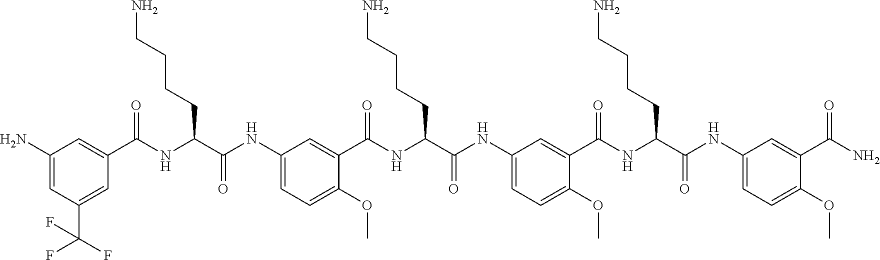 |
| 126 | 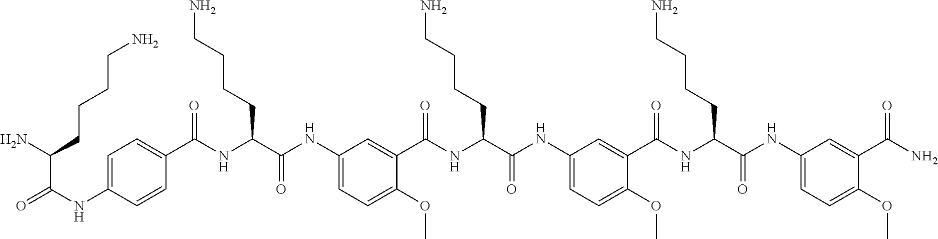 |
| 127 | 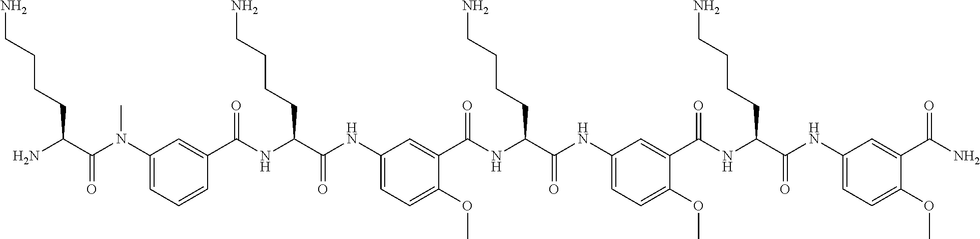 |
| 128 | 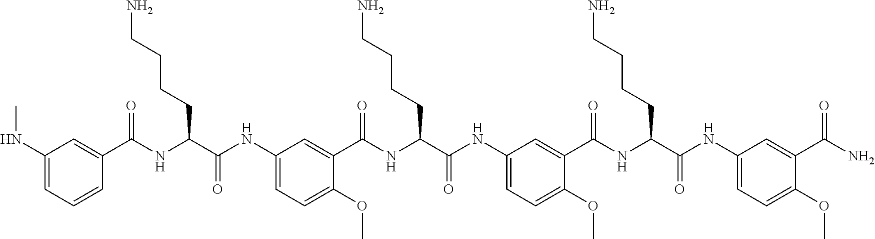 |
| 129 | 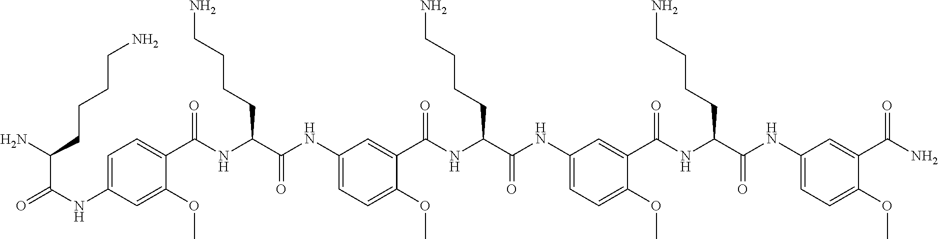 |
| 130 | 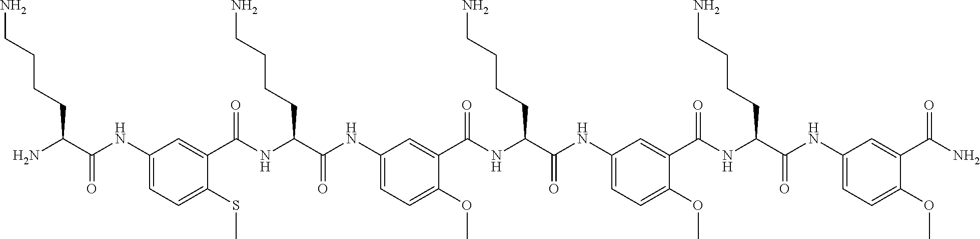 |
| 131 | 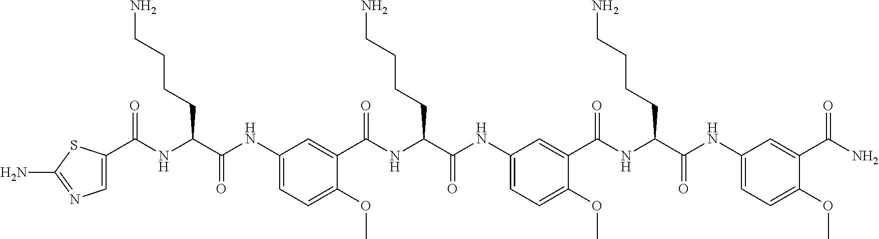 |
| 132 | 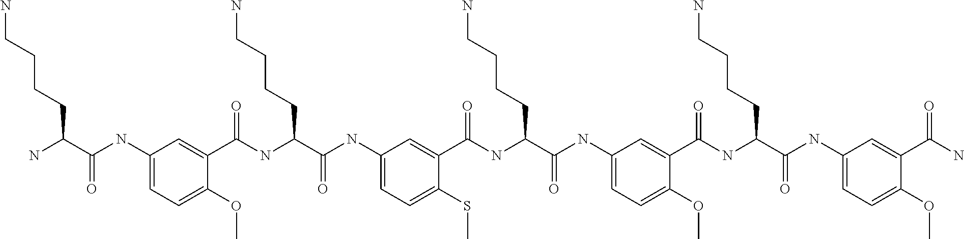 |
| 133 | 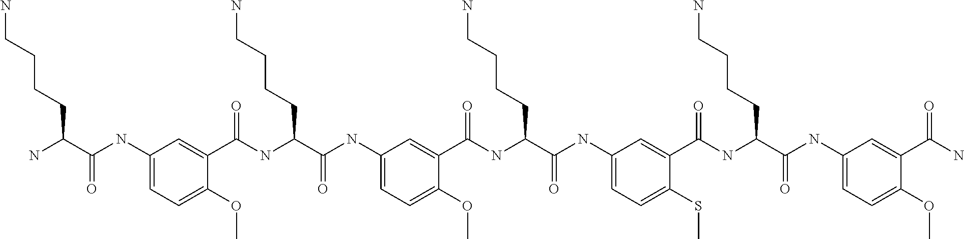 |
| 134 | 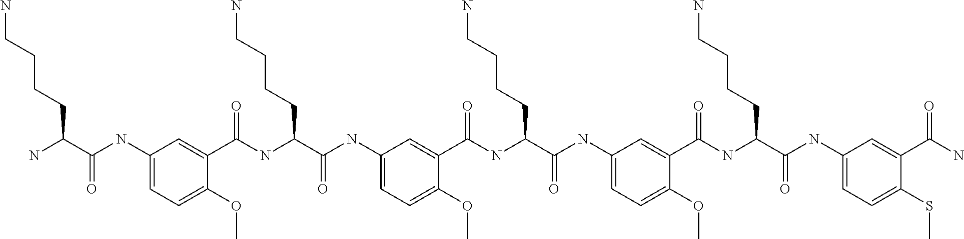 |
| 135 | 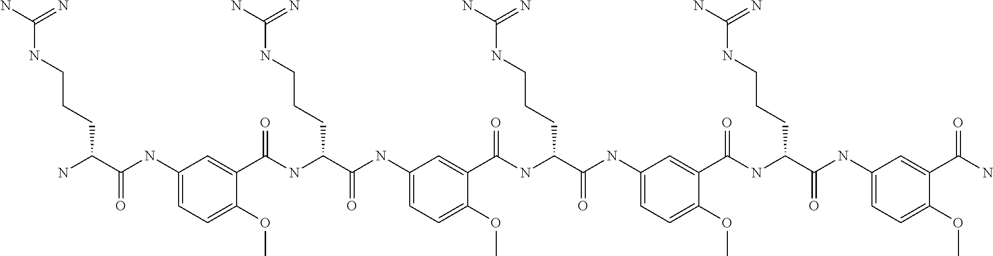 |
| 136 | 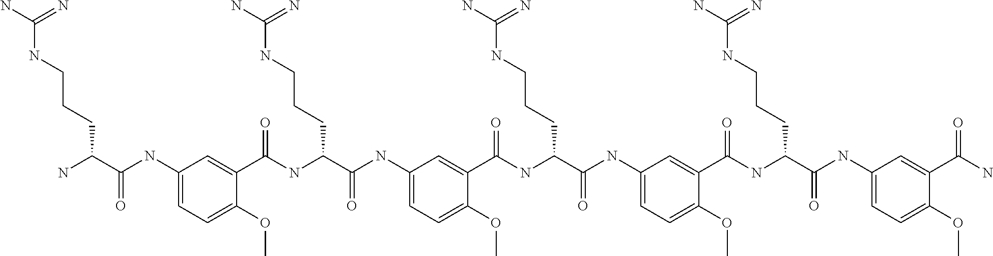 |
| 137 | 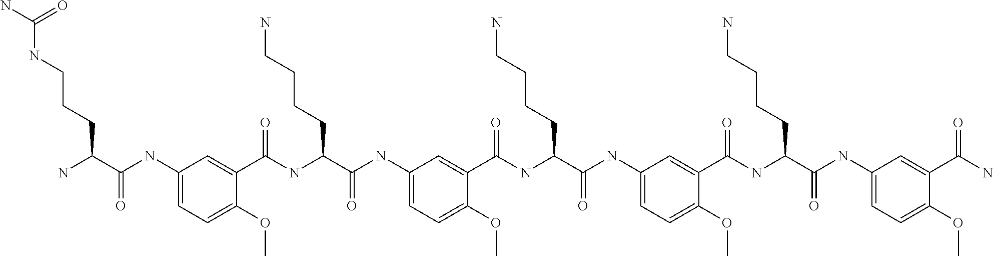 |
| 138 | 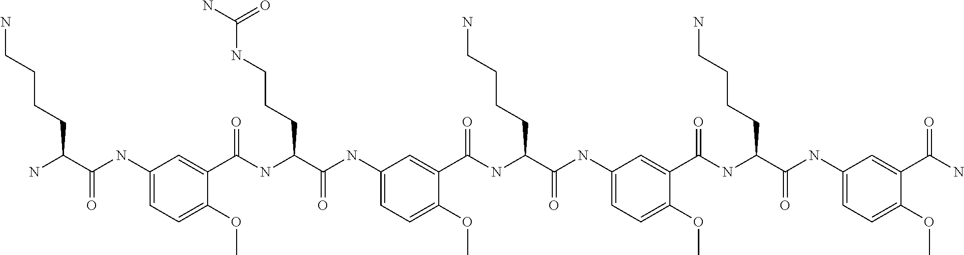 |
| 139 | 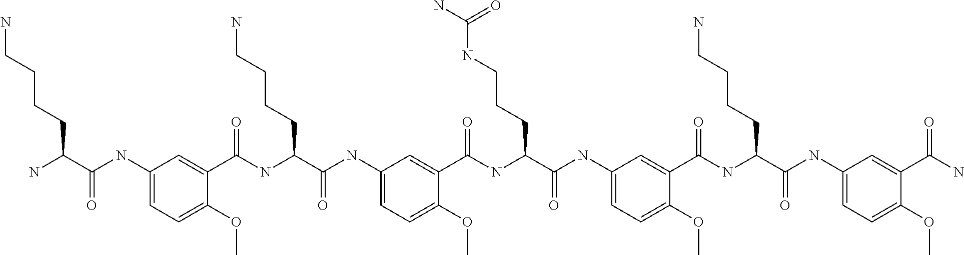 |
| 140 | 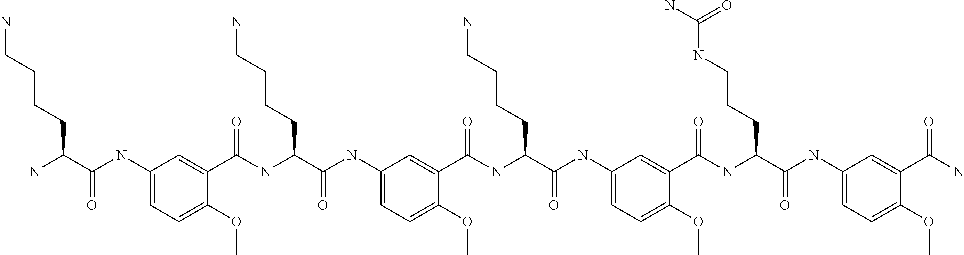 |
| 141 | 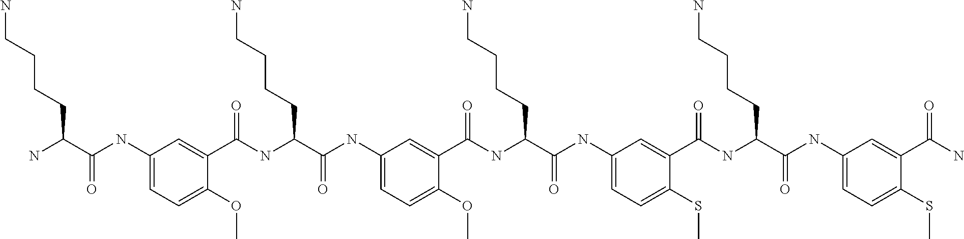 |
| 142 | 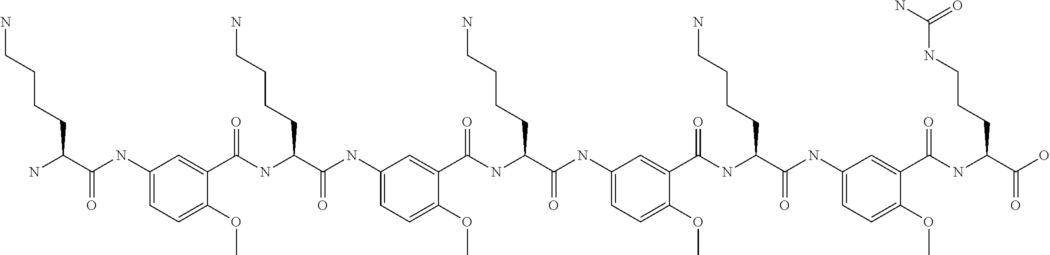 |
| 143 | 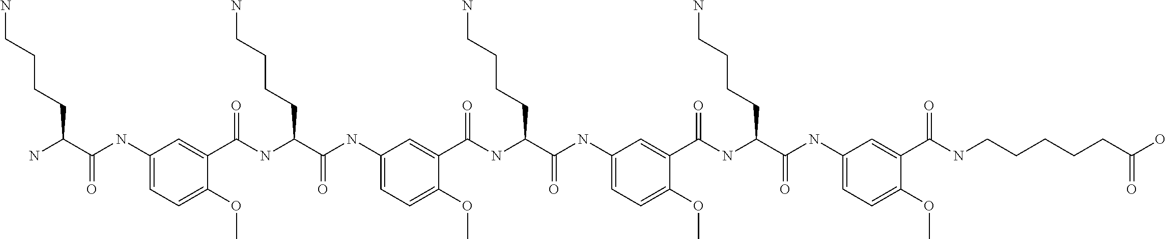 |
| 144 | 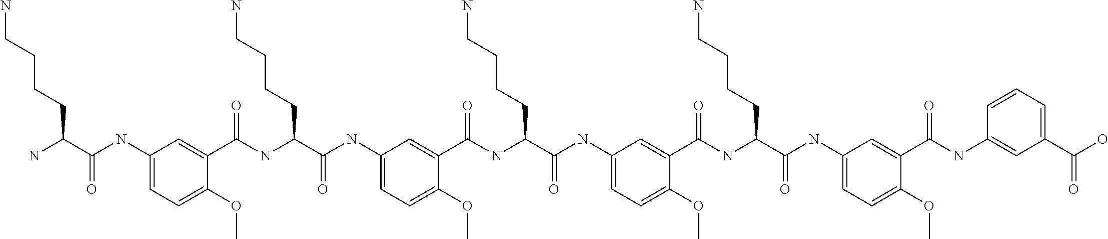 |
| 145 | 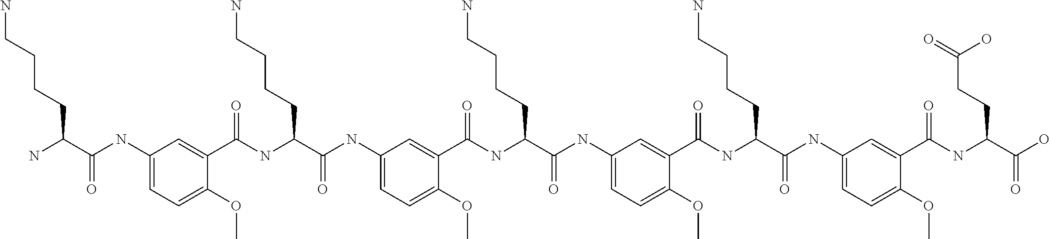 |
| 146 | 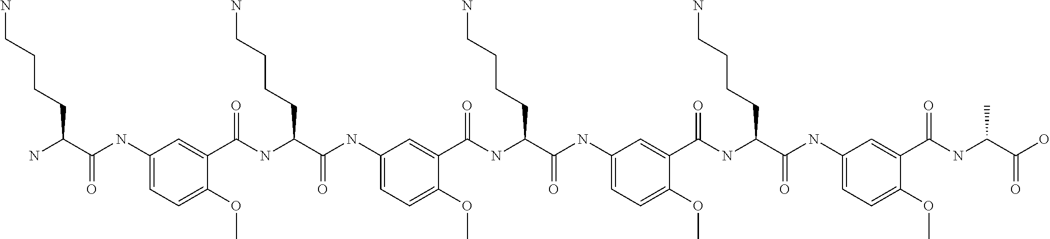 |

### Example 3

### FXa Chromogenic Assay (Absence of Plasma)

Human antithrombin was mixed with an anticoagulant agent (a LMWH or fondaparinux); final concentrations were 0.22 μg/mL for the LMWHs and 0.07 μg/mL for fondaparinux. Different concentrations of a test compound were added (typically 0.07 to 9 μg/mL range) followed by factor Xa and substrate (S-2765). Absorbance was read every 30 seconds over a 4 minute period in a SpectraMax 250 instrument (Molecular Devices, Inc.). EC₅₀ values are determined by a curve-fit program (SoftMax Pro) using the following formula:

P(Cₚ)=1/[1+(K/Cₚ)ⁿ]

### Example 4

### FIIa (Thrombin) Chromogenic Assay (Absence of Plasma)

The procedure for measuring anti-FIIa activity is similar to that for the anti-FXa assay except FIIa and S-2238 are used in place of FXa and S-2765, respectively.

### Example 5

### Clotting and Amidolytic Assays in Presence of Human Plasma

Eight parts of pooled human plasma was supplemented with 1 part LMWH or UFH at final concentrations of 4 μg/mL, or fondarinux at a final concentration of 1.25 μg/mL. One 1 μL sample of test agent was then added to 9 μL of supplemented plasma (test agent concentration ranges=0.156 to 20 μg/mL) and mixed. The supplemented plasmas were analyzed immediately in clotting and amidolytic assays as described below. All samples were performed in duplicate.

aPTT Clotting Assay. Supplemented plasma was added to aPTT reagent (activated partial thromboplastin time reagent) (activator) in fibrometer. Clotting was initiated by addition of CaCl₂ and time to clot was recorded.

All test agents/compounds showed a dose-dependent antagonism of aPTT inhibition by UFH in human plasma. For instance, each of Compounds 7, 8, 16, 45, 52, and 53 inhibited/antagonized about 50% of the anticoagulant effect of UFH (4 μg/mL) at a concentration of about 1-3 μg/mL, and inhibited about 90%-100% of the anticoagulant effect of UFH (4 μg/mL) at a concentration of about 6-16 μg/mL. Protamine showed similar dose-dependent antagonism effect to that of Compound 7.

HepTest Clotting Assay. Factor Xa was added to supplemented plasma in a fibrometer and incubated for 120 seconds. Recalmix was added and time to clot was recorded.

Thrombin time (TT) Clotting Assay. Human thrombin was added to supplemented plasma in a fibrometer and time to clot was recorded.

FXa Amidolytic Assay: Bovine factor Xa was added to supplemented plasma and incubated for 5 minutes at 37° C. Spectrozyme FXa substrate was added and the optical density change at 405 nm was measured for 30 seconds. % factor Xa inhibition is calculated using the following equation:

% Inhibition=[(OD_{baseline}−ODₛₐₘₚₗₑ)/OD_{baseline}]×100.

Test agents/compounds showed a dose-dependent antagonism. For instance, Compound 8 inhibited/antagonized about 50% of the anticoagulant effect of UFH (4 μg/mL) at a concentration of about 12-18 μg/mL, and inhibited about 90%-100% of the anticoagulant effect of UFH (4 μg/mL) at a concentration of greater than about 25 μg/mL. Protamine inhibited/antagonized about 50% of the anticoagulant effect of UFH (4 μg/mL) at a concentration of about 18-22 μg/mL, and inhibited about 80% of the anticoagulant effect of UFH (4 μg/mL) at a concentration of greater than about 25 μg/mL.

Protamine (or protamine sulfate) was ineffective in antagonizing the anticoagulant effect of enoxaparin (10 μg/mL); even at a concentration of about 50 μg/mL, it only inhibited about 20% of the anticoagulant effect of enoxaparin (10 μg/mL). Each of Compounds 7 and 38 inhibited/antagonized about 50% of the anticoagulant effect of enoxaparin (10 μg/mL) at a concentration of about 25-30 μg/mL, and inhibited about 90%-100% of the anticoagulant effect of enoxaparin (10 μg/mL) at a concentration of about 50 μg/mL. Each of Compounds 28 and 30 inhibited/antagonized about 50% of the anticoagulant effect of enoxaparin (10 μg/mL) at a concentration of about 25-30 μg/mL, and inhibited about 80%-900% of the anticoagulant effect of enoxaparin (10 μg/mL) at a concentration of about 50 μg/mL. Compound 8 inhibited about 60% of the anticoagulant effect of enoxaparin (10 μg/mL) at a concentration of about 50 μg/mL. Compound 16 inhibited about 20% of the anticoagulant effect of enoxaparin (10 μg/mL) at a concentration of about 50 μg/mL.

Protamine and Compounds 7, 8, 41, and 49 were tested for their antagonism effect against the anticoagulant effect of fondaparinux (1.25 μg/mL). Each of Compounds 7, 8, 41, and 49 had EC₅₀ ranged from about 1-3 μg/mL. The EC₅₀ of protamine was measured at greater than about 20 μg/mL.

FIIa Amidolytic Assay. Human thrombin was added to supplemented plasma and incubated for 1 minute at 37° C. Spectrozyme TH substrate was added and the optical density change at 405 nm was measured for 30 seconds in a SpectraMax 250 instrument. % factor IIa inhibition was calculated using the following equation:

% Inhibition=[(OD_{baseline}−ODₛₐₘₚₗₑ)/OD_{baseline}]×100.

Test agents/compounds showed a dose-dependent antagonism. For instance, Compound 8 inhibited/antagonized about 50% of the anticoagulant effect of UFH (4 μg/mL) at a concentration of about 14-20 μg/mL, and inhibited about 98%-100% of the anticoagulant effect of UFH (4 μg/mL) at a concentration of greater than about 25 μg/mL. Protamine inhibited/antagonized about 50% of the anticoagulant effect of UFH (4 μg/mL) at a concentration of about 18-22 μg/mL, and inhibited about 80%-90% of the anticoagulant effect of UFH (4 μg/mL) at a concentration of greater than about 25 μg/mL.

### Example 6

### Heparin-Binding Activity

The heparin (unfractionated) preparations were tyramine end-labeled and radiolabeled with ¹²⁵Iodine to a specific activity of 1-2.5×10⁷ cpm/μg. Increasing concentrations of a test agent (protamine or an exemplary compound provided herein) were added to individual wells across a 1% agarose gel in 125 mM sodium acetate, 50 mM MOPSO (3-(n-morpholino)-2-hydroxypropanesulfonic acid), pH 7.0). The radio-labeled heparin was added to a closely neighboring upper well and electrophoresed through the test agent wells. Heparin binding was visualized on the dried gel using a Phosphorimager. The dissociation constant (Kd) was calculated from the test agent concentration (n=3) at which the polysaccharide is half-shifted between its fully mobile position at low concentrations of test agent and its fully retarded position at saturating concentrations of test agent according to the methods of Lee and Lander (See Lee, M. K. and Lander, A. D., “Analysis of affinity and structural selectivity in the binding of proteins to glycosaminoglycans: development of a sensitive electrophoretic approach” Proc. Natl. Acad. Sci. USA, 1991, 88, 2768-2772). Although not wishing to be bound by any particular theory, the heparin-binding Kds were found to correlate to the EC₅₀ data for UFH antagonism (by the aPTT assay in Example 4).

TABLE 2

| | | n |
|---|---|---|
| | Kd ± S.D. | (number of |
| Compound tested | (nM) | experiments) |
| 7 | 76 ± 16 | 4 |
| 8 | 219 ± 14 | 4 |
| 34 | 11612 ± 986 | 4 |
| 35 | 2095 ± 1078 | 4 |
| Protamine | 39 ± 25 | 6 |

### Example 7

### EC₅₀ Data for the Compounds and Protamine

Table 3 shows in vitro neutralization results (EC₅₀ data) for several compounds and protamine against unfractionated heparin (UFH), the low molecular weight heparin, enoxaparin (ENOX) and the pentasaccharide, fondaparinux (FONDAPX).

FXa Chromogenic Assay (absence of plasma). Human antithrombin was mixed with an anticoagulant (a LMWH or fondaparinux); final concentrations were 0.1 μg/mL for the LMWHs and 0.02 μg/mL for fondaparinux. Different concentrations of test compound were added (typically 0.01 to 22 μg/mL range) followed by bovine factor Xa and chromogenic substrate (S-2765). Absorbance was read every 30 seconds over a 4 minute period in a SpectraMax 250 instrument (Molecular Devices Inc.™) The slope of absorbance vs time was calculated for each compound concentration. EC₅₀ values were determined using a curve fit program (GraphPad Prism 5).

aPTT clotting Assay. Unfractionated heparin was mixed with plasma at a final concentration of 0.2 U/mL. Different concentrations of test compound were added (typically 0.15 to 20 μg/mL range). The ACL Elite Hemostasis analyzer (Beckman Coulter™) was used to add aPTT reagent (HemosIL SynthASil) to supplemented plasma. Clotting was initiated by addition of CaCl₂ and time to clot was recorded. EC₅₀ values were determined using a curve fit program (GraphPad Prism 5).

TABLE 3

| | ENOX (FXa) | FONDAPX (FXa) | UFH (aPTT) |
|---|---|---|---|
| Compound tested | EC₅₀ μg/mL | EC₅₀ μg/mL | EC₅₀ μg/mL |
| 1 | 0.18 | inactive | inactive |
| 2 | 2.26 | inactive | 6.25 |
| 3 | 0.25 | 1.34 | 1.76 |
| 4 | 47.94 | 84.66 | inactive |
| 5 | 23.71 | inactive | 19.76 |
| 6 | 0.57 | inactive | 29.11 |
| 7 | 0.21 | 1.49 | 1.84 |
| 8 | 0.29 | 6.19 | 2.26 |
| 9 | 2.90 | 40.16 | 5.35 |
| 10 | 1.43 | — | — |
| 11 | 1.66 | 7.04 | 5.79 |
| 12 | 0.36 | 4.26 | — |
| 13 | 0.88 | 6.72 | — |
| 14 | 2.04 | 10.31 | — |
| 15 | 0.92 | 7.84 | — |
| 16 | 0.32 | 1.22 | 2.41 |
| 17 | 0.50 | 11.15 | 2.33 |
| 18 | 0.57 | — | — |
| 19 | 0.27 | 3.35 | 2.11 |
| 20 | 8.62 | 11.10 | — |
| 21 | 0.65 | 8.84 | — |
| 22 | 0.56 | 7.63 | — |
| 23 | 3.68 | 18.01 | — |
| 24 | 0.47 | 9.23 | — |
| 25 | 0.97 | 10.36 | — |
| 26 | 3.93 | 36.65 | 4.26 |
| 27 | 0.10 | 2.51 | 2.19 |
| 28 | 0.23 | 3.21 | 2.28 |
| 29 | 0.78 | 8.33 | — |
| 30 | 0.28 | 3.99 | 1.52 |
| 31 | 0.72 | — | — |
| 32 | 30.00 | inactive | — |
| 33 | 4.30 | inactive | — |
| 34 | 45.00 | inactive | inactive |
| 35 | 45.00 | inactive | 30.65 |
| 36 | 45.00 | inactive | — |
| 37 | 45.00 | inactive | — |
| 38 | 0.18 | 1.94 | 2.37 |
| 39 | 1.11 | 17.09 | 3.07 |
| 40 | 1.84 | >90 | 2.88 |
| 41 | 0.16 | 3.85 | 1.65 |
| 42 | 4.07 | 70.66 | 2.34 |
| 43 | 0.36 | 4.44 | 2.33 |
| 44 | 0.21 | 2.91 | 2.38 |
| 45 | 0.23 | 2.61 | 2.35 |
| 46 | 13.76 | 76.88 | inactive |
| 47 | 0.36 | 6.52 | 3.23 |
| 48 | 0.30 | 3.35 | 1.72 |
| 49 | 0.16 | 1.03 | 1.42 |
| 50 | 0.22 | 1.12 | 1.67 |
| 51 | 0.15 | 4.26 | — |
| 52 | 0.35 | 1.90 | 2.13 |
| 53 | 0.21 | 1.12 | 2.08 |
| 54 | 0.22 | 5.92 | — |
| 55 | 0.18 | 4.16 | — |
| 56 | 0.43 | 5.24 | — |
| 57 | 0.17 | 3.25 | — |
| 58 | 0.36 | 3.94 | — |
| 59 | 0.13 | 4.53 | — |
| 60 | 0.21 | 2.66 | — |
| 61 | 0.11 | 2.99 | — |
| 62 | 0.10 | 3.99 | — |
| 63 | 0.10 | 3.29 | — |
| 64 | — | — | — |
| 65 | 16.29 | — | 4.48 |
| 66 | — | — | 2.53 |
| 67 | 0.33 | 5.19 | 1.75 |
| 68 | 0.29 | 4.26 | 1.99 |
| 69 | 0.19 | 2.74 | 1.58 |
| 70 | 0.33 | 1.59 | 1.55 |
| 71 | 0.87 | 0.86 | 2.58 |
| 72 | 0.22 | 1.22 | 2.11 |
| 73 | 2.49 | 17.92 | 5.16 |
| 74 | 5.13 | 47.76 | 5.20 |
| 75 | 3.71 | 44.99 | 4.80 |
| 76 | 1.49 | 10.50 | 6.00 |
| 77 | 0.61 | 2.47 | 4.55 |
| 78 | 0.49 | 6.28 | 2.99 |
| 79 | 0.41 | 7.07 | 3.02 |
| 80 | 0.35 | 4.34 | inactive |
| 81 | 0.26 | 3.23 | 1.97 |
| 82 | 0.07 | 0.77 | — |
| 83 | 1.02 | 14.79 | 4.73 |
| 84 | 0.32 | 1.99 | 3.09 |
| 85 | 0.36 | 1.93 | — |
| 86 | — | >90 | inactive |
| 87 | 6.35 | 15.91 | >10 |
| 88 | 0.24 | 1.38 | 4.30 |
| 89 | 16.70 | — | — |
| 90 | 0.093 | 0.272 | 0.9713 |
| 91 | 0.58 | 13.11 | 2.03 |
| 92 | 0.55 | 1.40 | 7.17 |
| 93 | 0.38 | 0.42 | 2.28 |
| 94 | 1.32 | >20 | 4.94 |
| 95 | 0.5734 | 5.277 | 2.42 |
| 96 | 5.567 | inactive | 3.59 |
| 97 | 0.9842 | >20 | >10 |
| 98 | 0.1731 | inactive | 3.14 |
| 99 | 0.2998 | 6.132 | 1.54 |
| 100 | 1.12 | 2.482 | 7.13 |
| 101 | 0.8434 | 4.158 | approx 16 |
| 102 | 0.3328 | 2.938 | 3.32 |
| 103 | 0.23 | 0.64 | >4 |
| 104 | 0.36 | 2.02 | >10 |
| 105 | 2.07 | >20 | Inactive |
| 106 | 1.66 | >20 | Inactive |
| 107 | 0.498 | 6.961 | 1.34 |
| 108 | 0.6085 | 10.57 | 1.50 |
| 109 | 0.6089 | 9.853 | 2.23 |
| 110 | 0.694 | 2.951 | 2.31 |
| 111 | 1.348 | >20 | 2.54 |
| 112 | 0.257 | 0.717 | 2.71 |
| 113 | 0.2306 | 0.4025 | 4.89 |
| 114 | 0.9758 | 12.56 | 3.00 |
| 115 | 0.7967 | >20 | 2.44 |
| 116 | | | 3.10 |
| 117 | 0.9281 | 7.052 | 1.94 |
| 118 | | | 3.14 |
| 119 | 0.9421 | 13.39 | 11.56 |
| 120 | 1.36 | 5.96 | 9.62 |
| 121 | 0.4289 | 8.293 | 7.48 |
| 122 | 0.3254 | 6.037 | 3.91 |
| 123 | 1.934 | inactive | >10 |
| 124 | 0.418 | 6.94 | 7.39 |
| 125 | 0.9448 | 3.114 | >10 |
| 126 | 0.7187 | >20 | 9.07 |
| 127 | >15 | inactive | 2.33 |
| 128 | 1.651 | 8.495 | 4.17 |
| 129 | 0.3888 | 7.996 | 2.47 |
| 130 | 0.3835 | 5.47 | 2.50 |
| 131 | 4.499 | >20 | 3.68 |
| 132 | >20 | inactive | >10 |
| 133 | >20 | inactive | >10 |
| 134 | 1.829 | inactive | 2.84 |
| 135 | 0.1581 | 0.7596 | 2.79 |
| 136 | 0.194 | 0.8018 | 2.91 |
| 137 | 0.3761 | 4.025 | 2.00 |
| 138 | 0.3298 | 2.332 | 2.15 |
| 139 | 0.3196 | 2.485 | 2.46 |
| 140 | 0.4309 | 4.636 | 2.52 |
| 141 | inactive | inactive | inactive |
| 142 | 0.592 | 7.045 | 2.88 |
| 143 | 1.011 | 12.700 | 2.96 |
| 144 | 0.244 | 1.051 | 3.13 |
| 145 | 1.141 | 9.463 | 4.26 |
| 146 | 1.027 | 19.030 | 2.74 |
| Protamine CG | 0.34 | 3.73 | 1.28 |
| Protamine Rx | 0.26 | 3.88 | 1.08 |

a. when the experiment limit is set as “a” and the EC₅₀ measurement of the example compound exceeds the limit, then the EC₅₀ data is shown as “> a”
b. “—”: data not available (no measurement performed)

### Example 8

### Compounds for Evaluation as Anti-Heparin Agents

The following exemplary compounds (and/or their salts) in Table 4 were prepared by methods such as those reported in U.S. Patent Application Publication Nos. U.S. 2005/0287108, U.S. 2006/0041023, U.S. Pat. No. 7,173,102, WO 2005/123660, WO 2004/082643, WO 2006/093813, and U.S. patent application Ser. No. 12/510,593 filed Jul. 28, 2009.

TABLE 4

| Compound | |
|---|---|
| Number | Compound Structure |
| 201 | 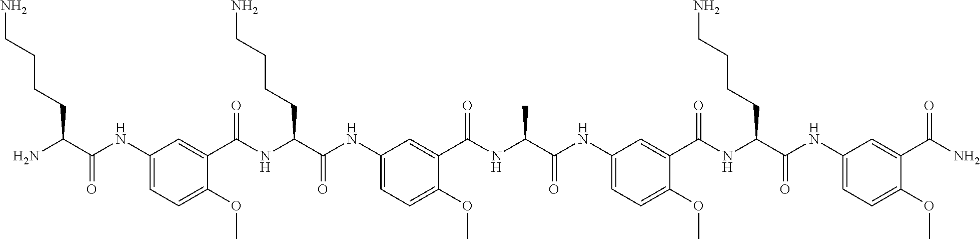 |
| 202 | 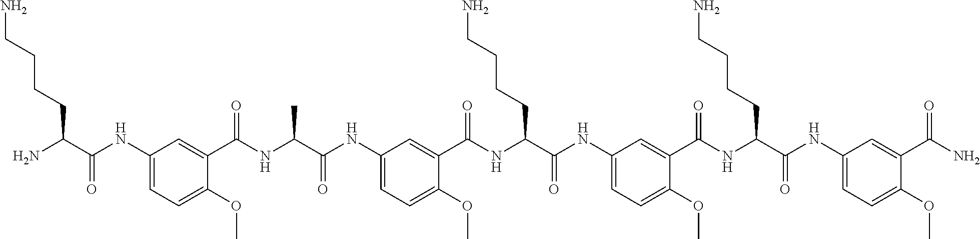 |
| 203 | 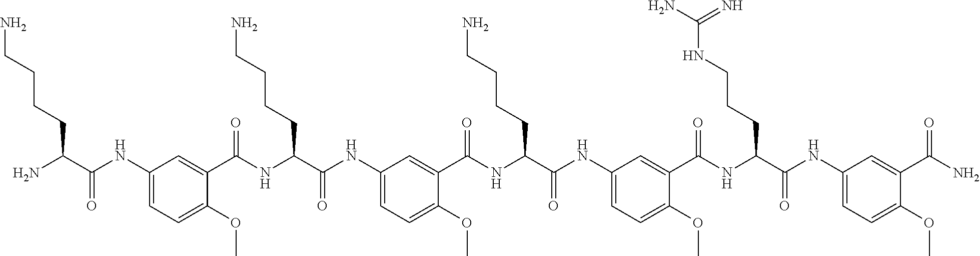 |
| 204 | 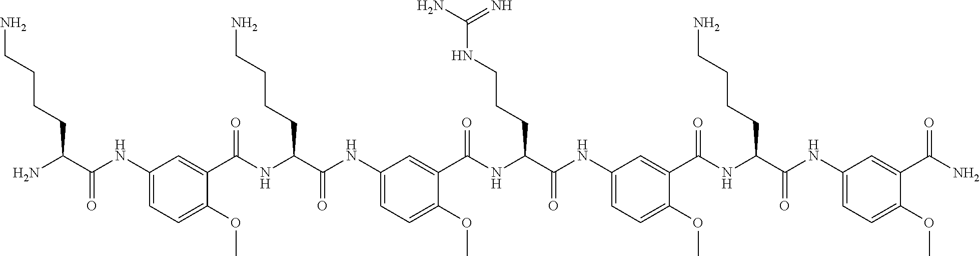 |
| 205 | 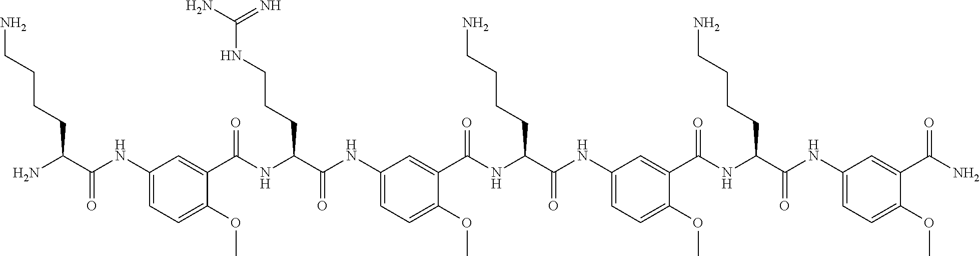 |
| 206 | 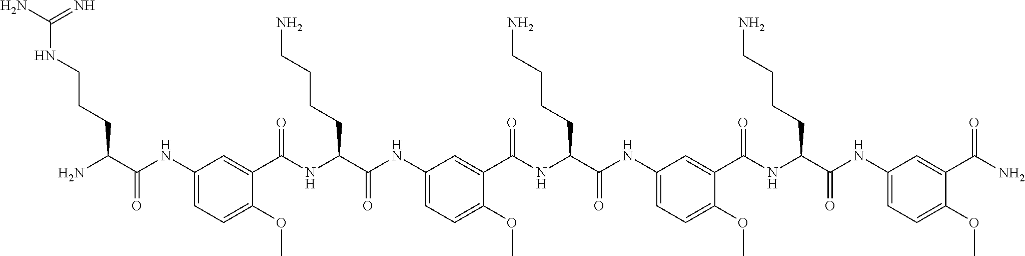 |
| 207 | 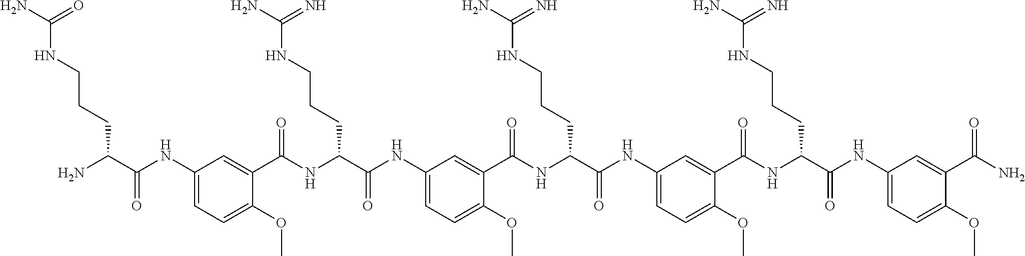 |
| 208 | 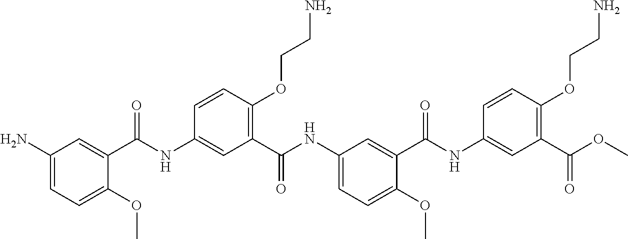 |
| 209 | 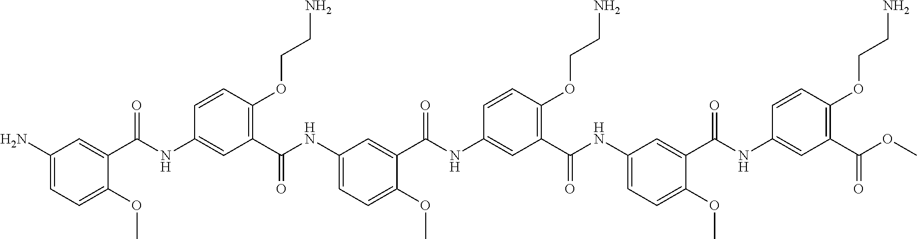 |
| 210 | 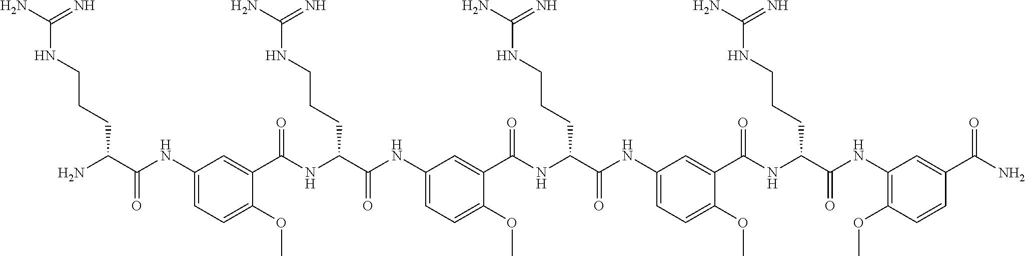 |
| 211 | 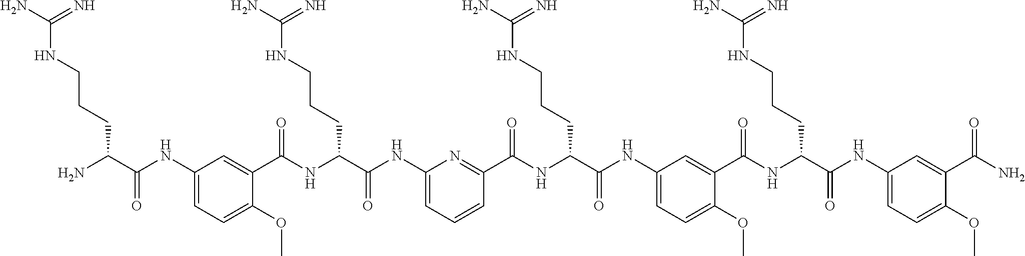 |
| 212 | 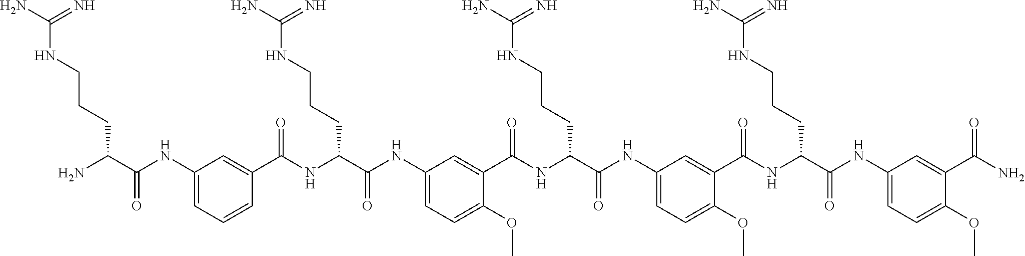 |
| 213 | 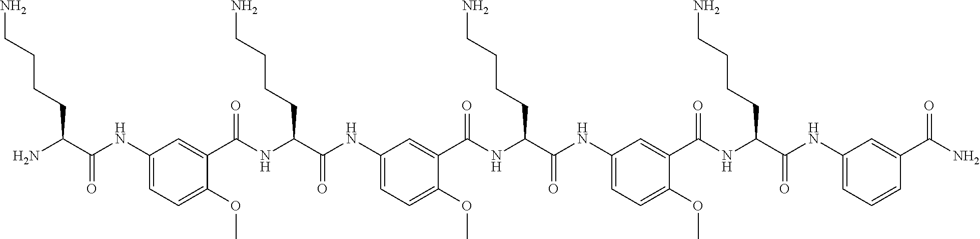 |
| 214 | 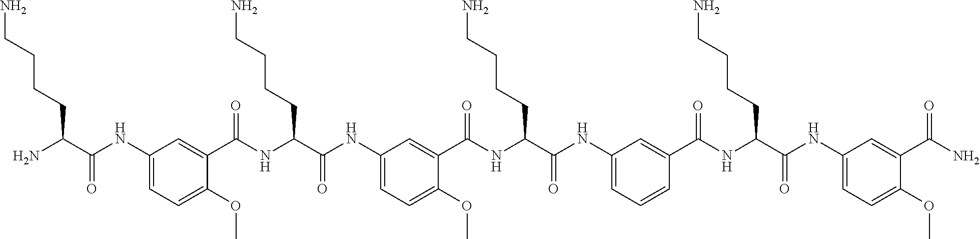 |
| 215 | 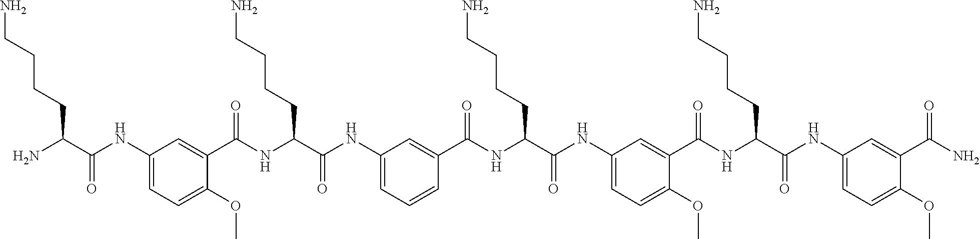 |
| 216 | 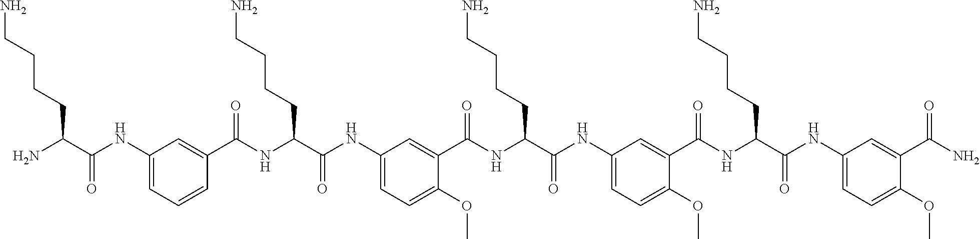 |
| 217 | 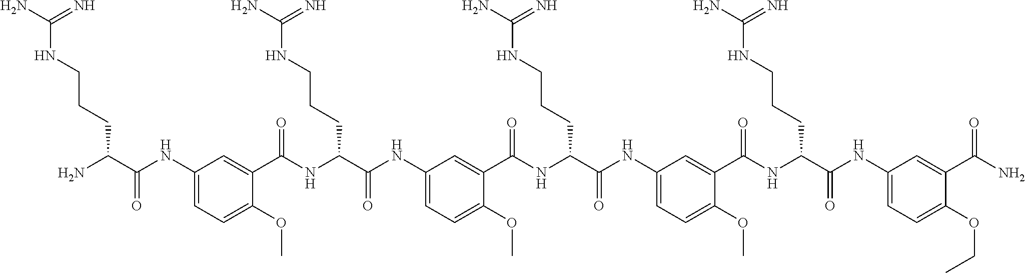 |
| 218 | 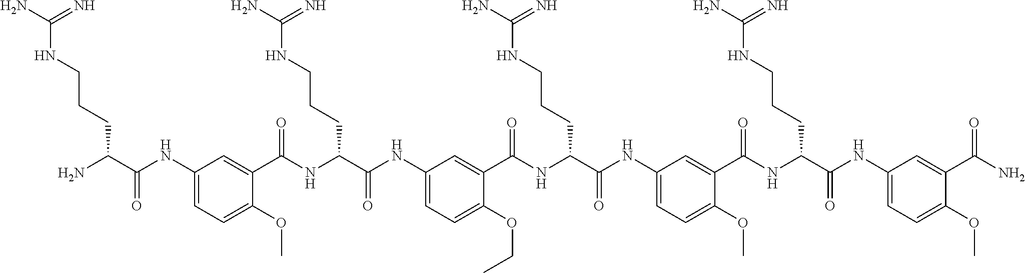 |
| 219 | 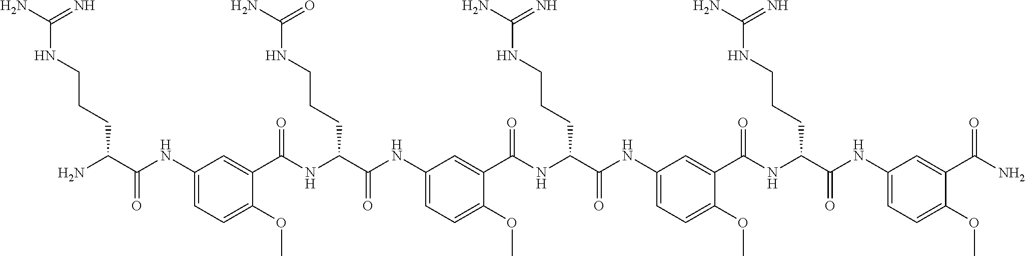 |
| 220 | 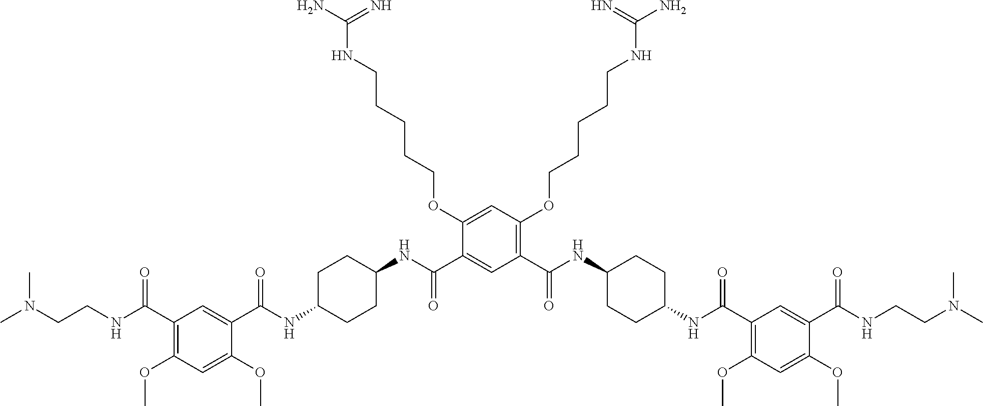 |
| 221 | 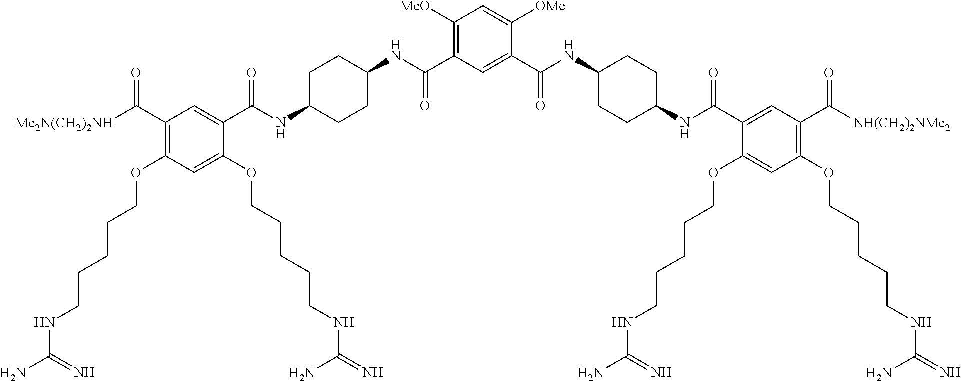 |
| 222 | 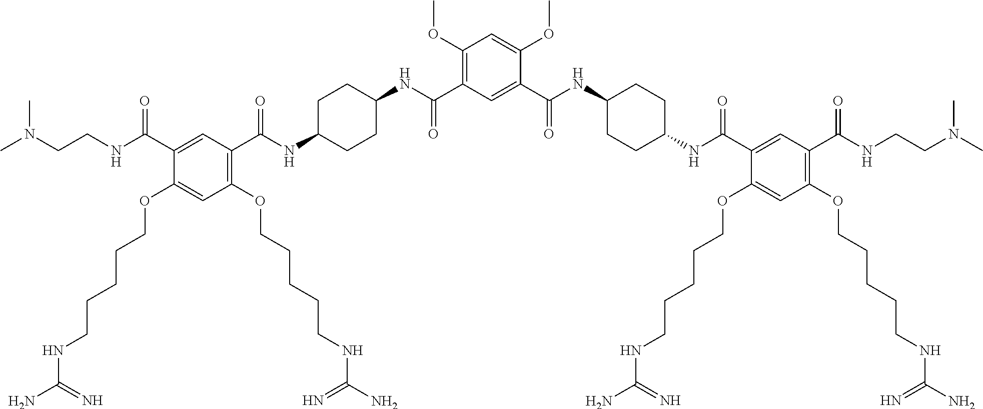 |
| 223 | 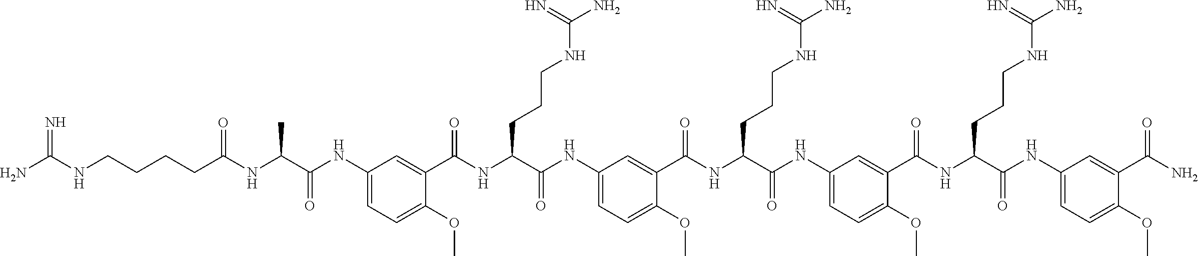 |
| 224 | 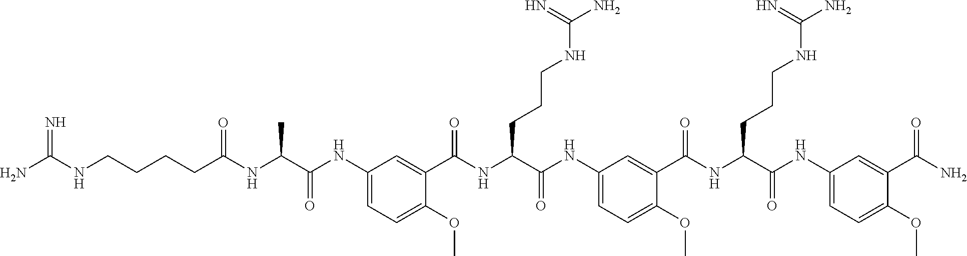 |
| 225 | 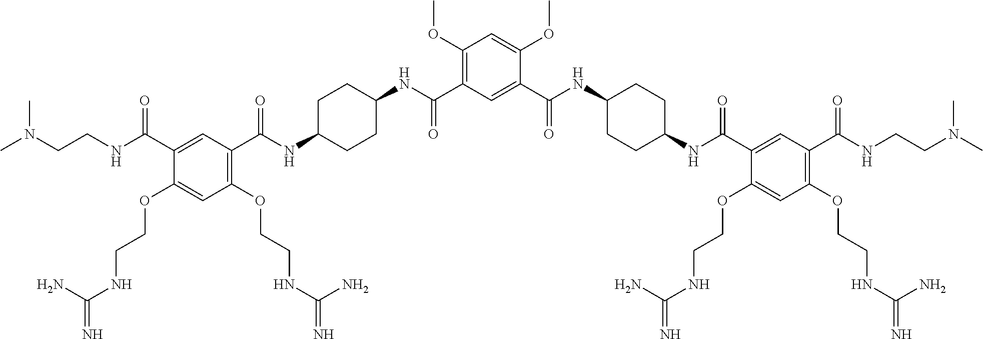 |
| 226 | 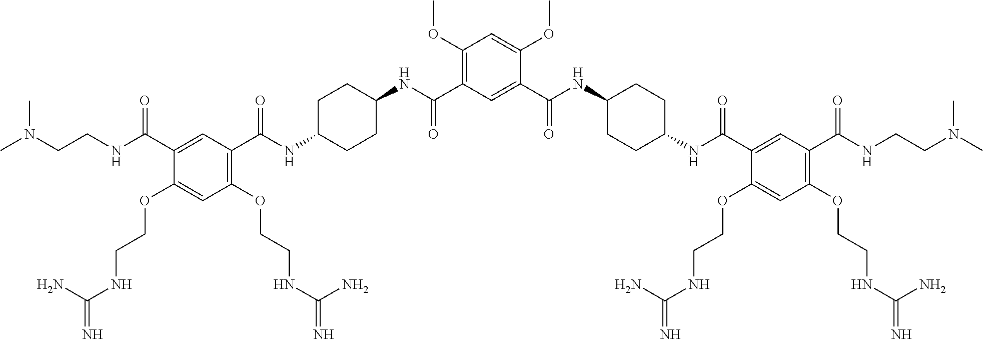 |
| 227 | 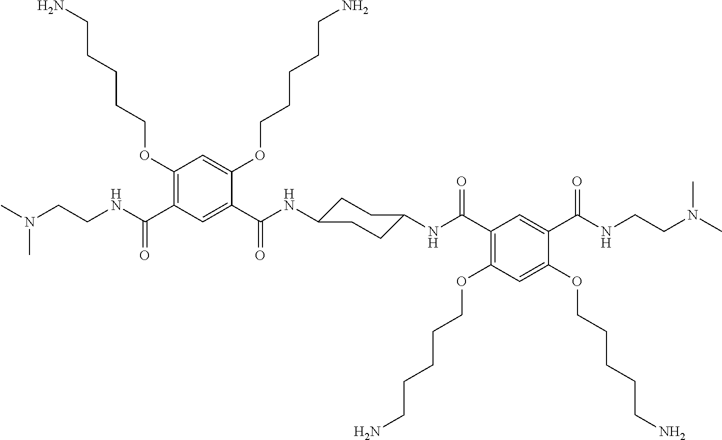 |
| 228 | 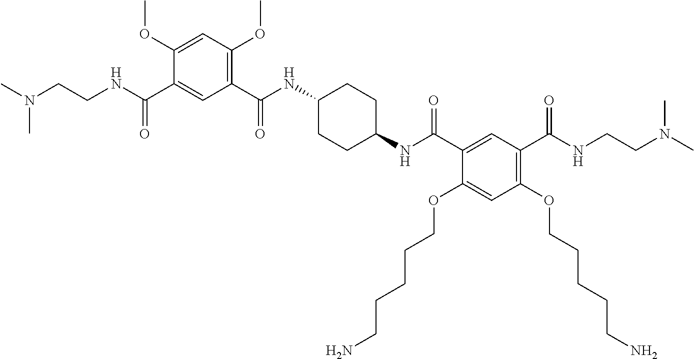 |
| 229 | 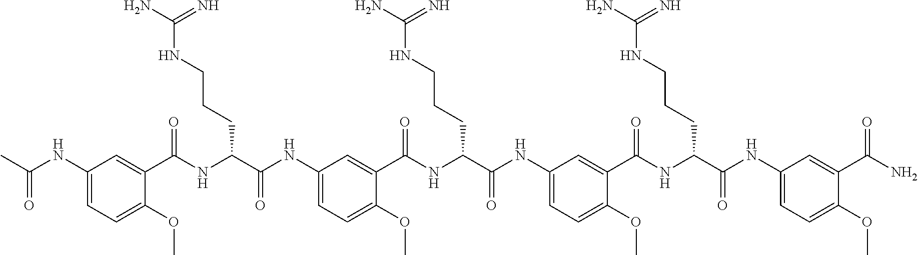 |
| 230 | 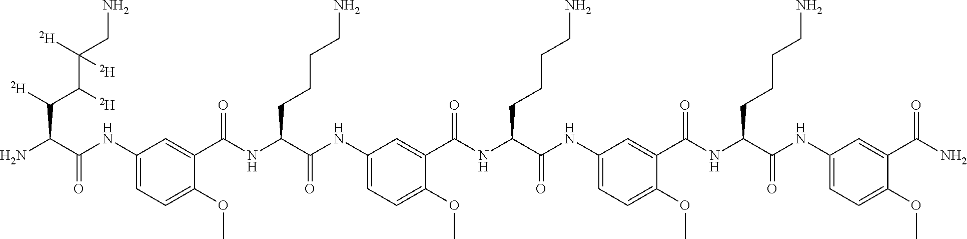 |
| 231 | 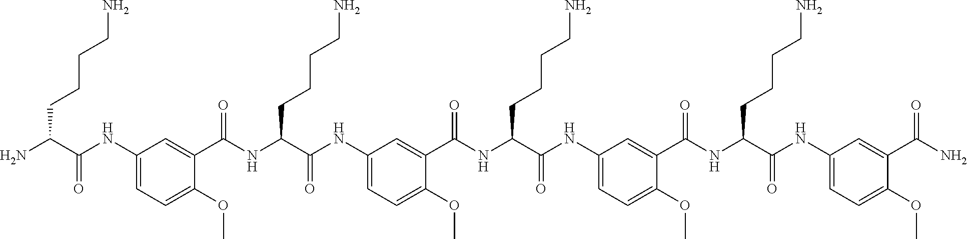 |
| 232 | 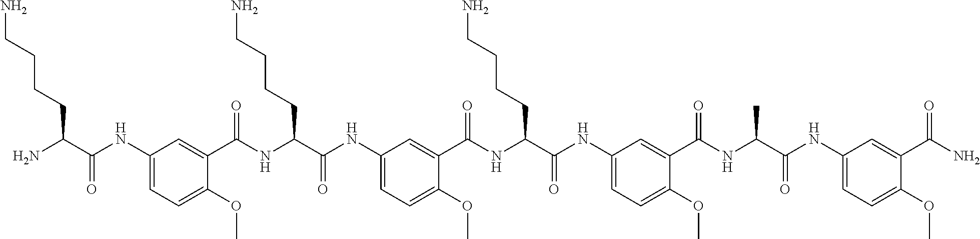 |
| 233 | 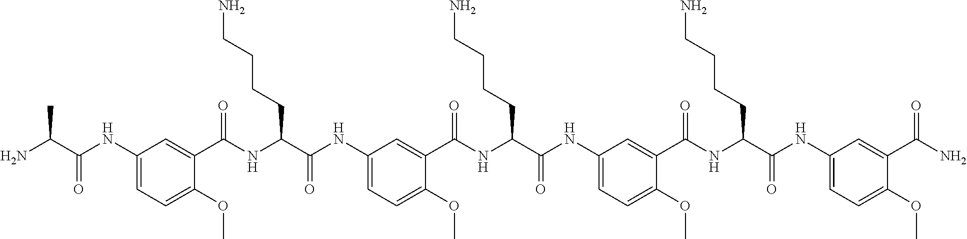 |
| 234 | 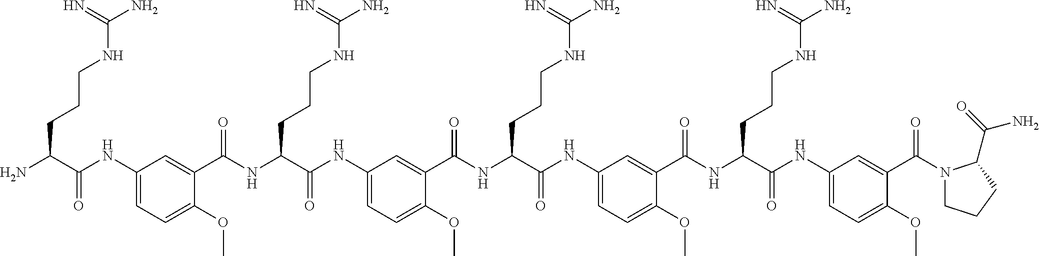 |
| 235 | 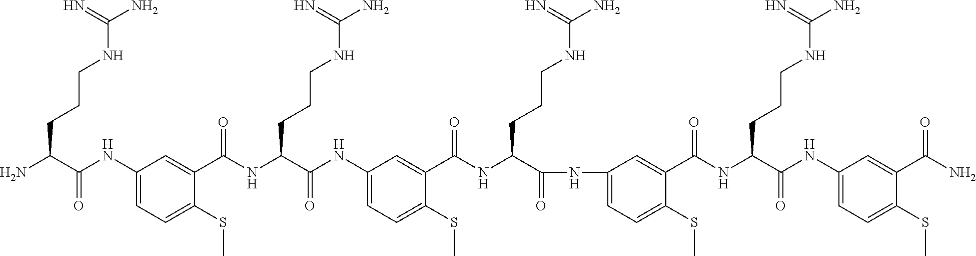 |
| 236 | 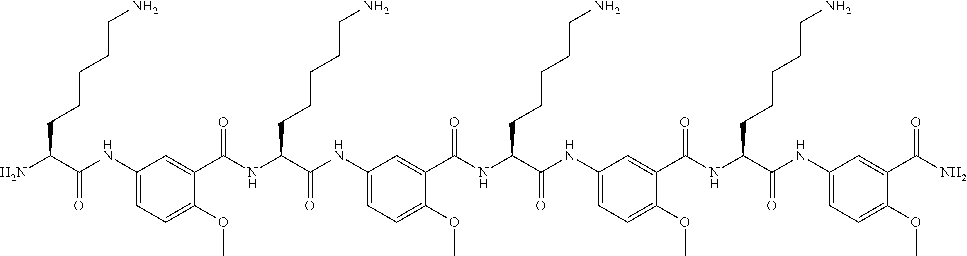 |
| 237 | 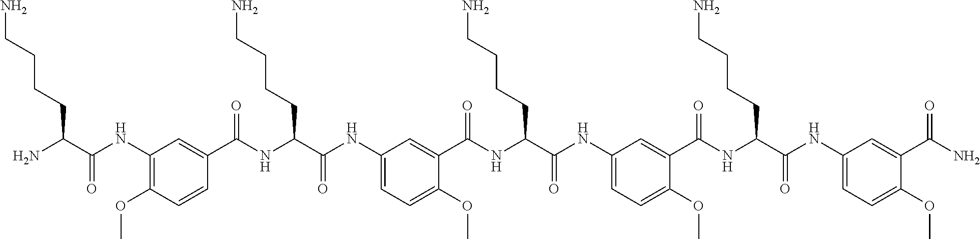 |
| 238 | 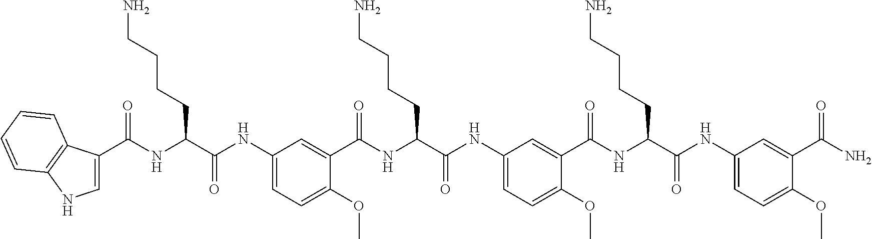 |
| 239 | 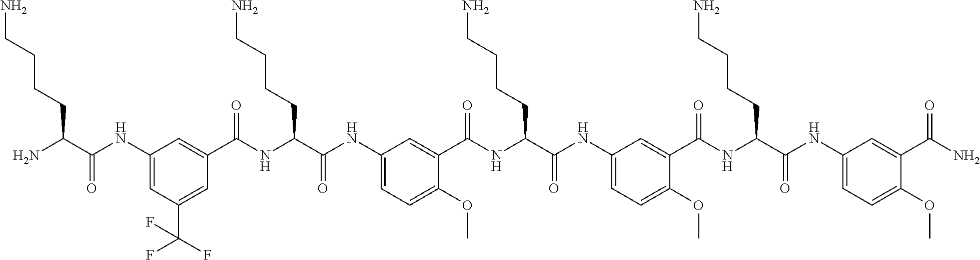 |
| 240 | 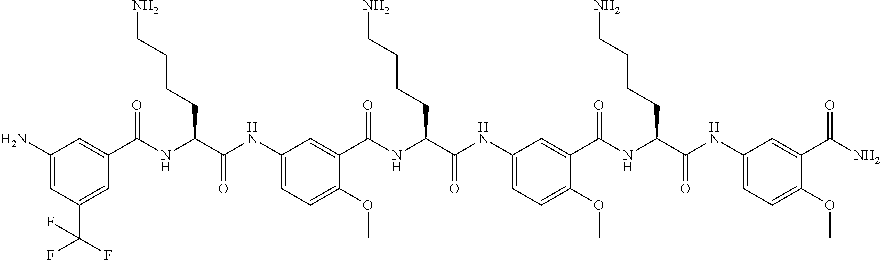 |
| 241 | 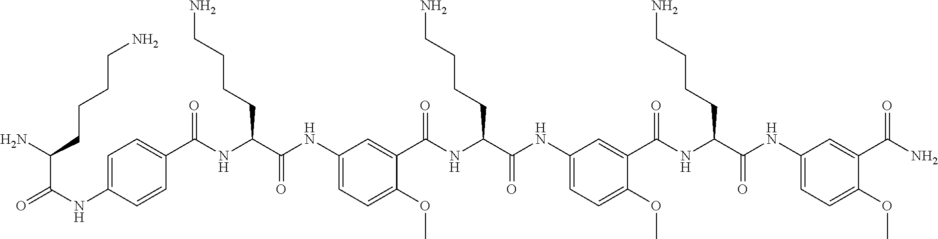 |
| 242 | 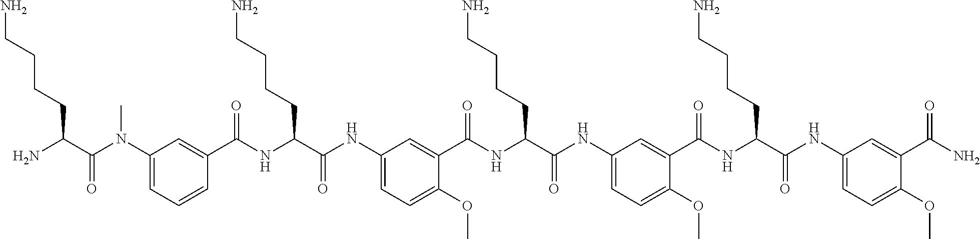 |
| 243 | 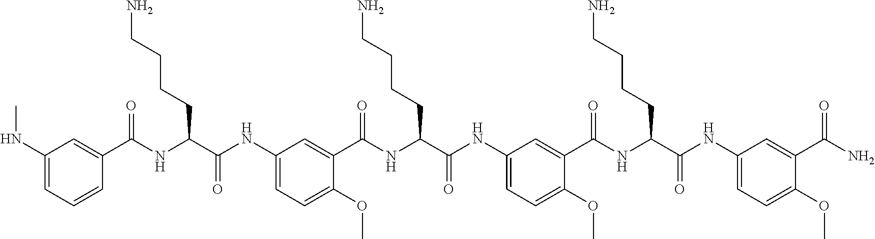 |
| 244 | 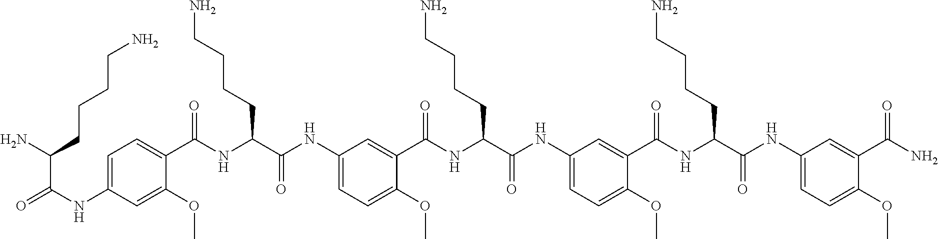 |
| 245 | 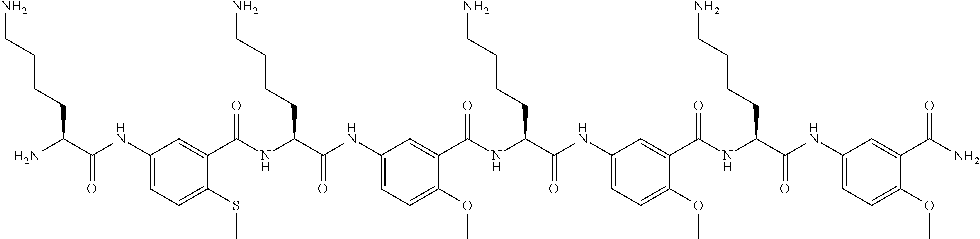 |
| 246 | 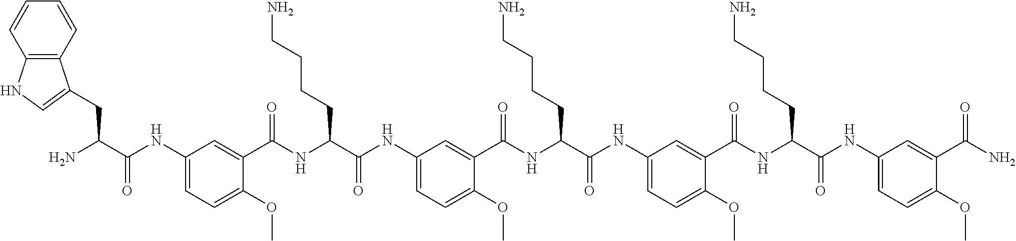 |
| 247 | 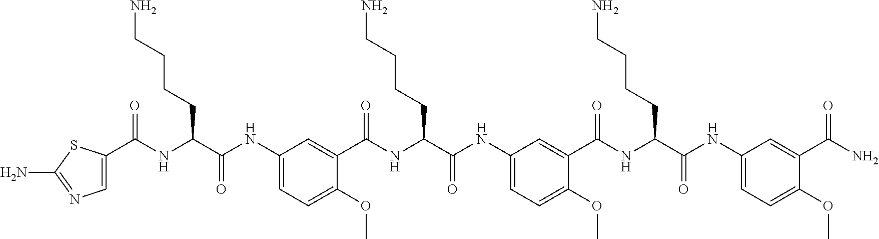 |
| 248 | 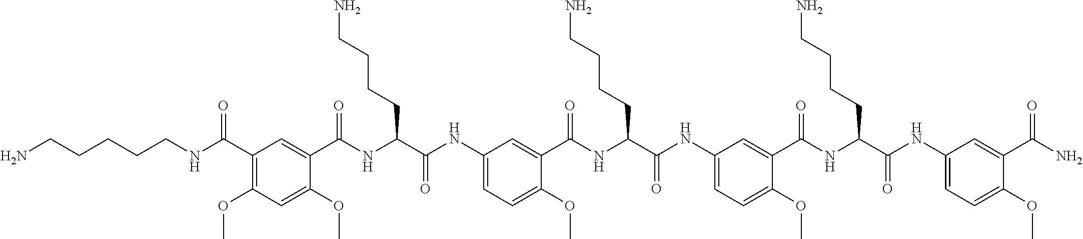 |
| 249 | 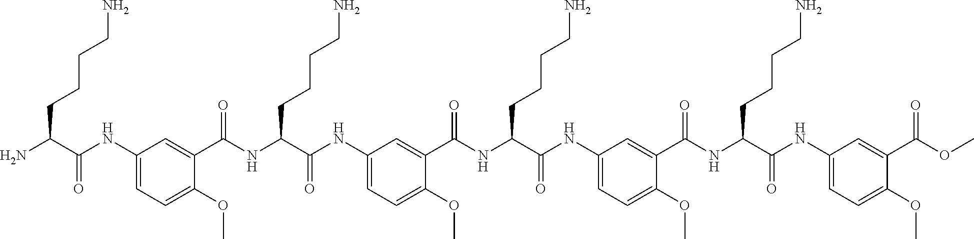 |
| 250 | 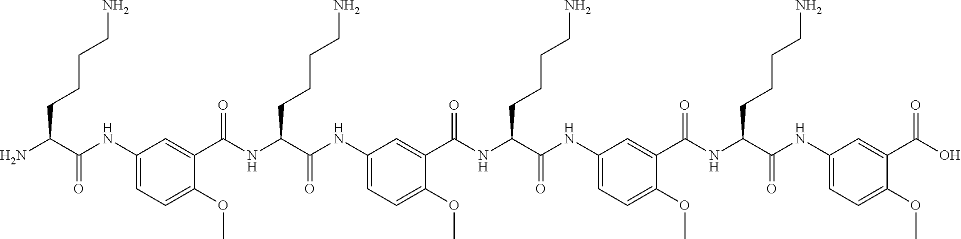 |
| 251 | 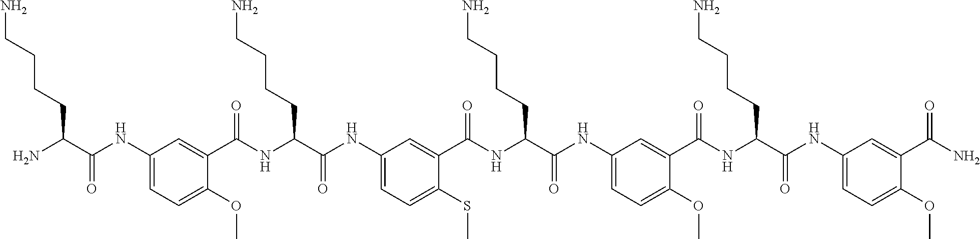 |
| 252 | 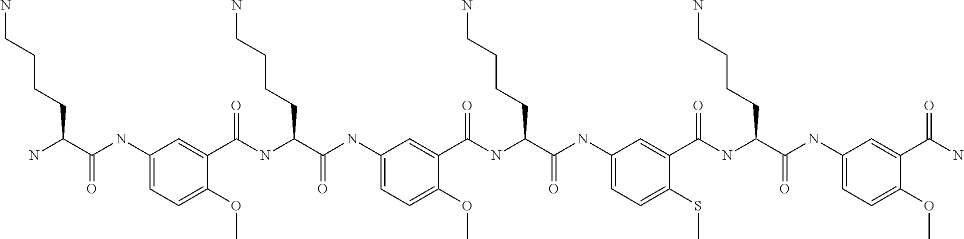 |
| 253 | 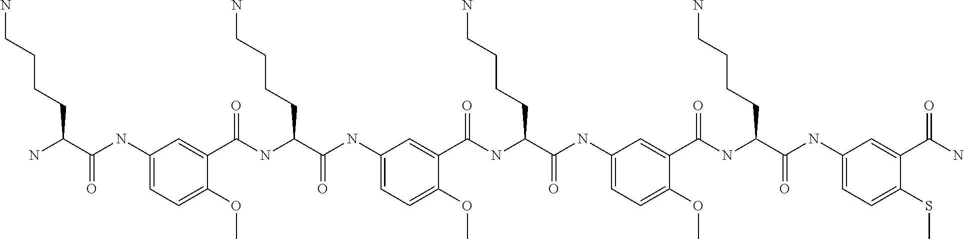 |
| 254 | 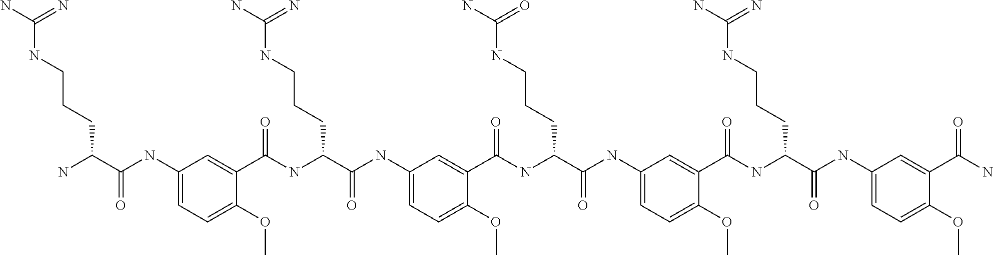 |
| 255 | 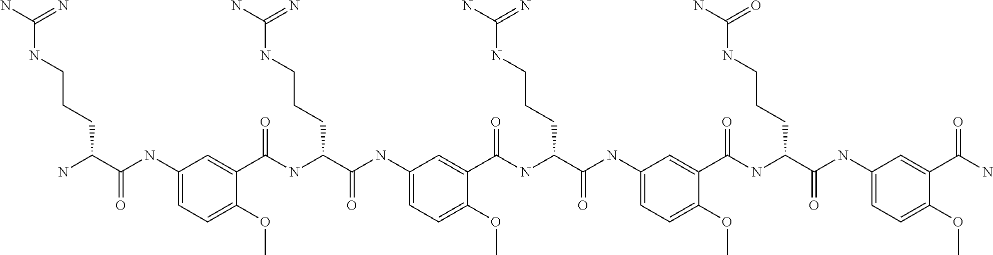 |
| 256 | 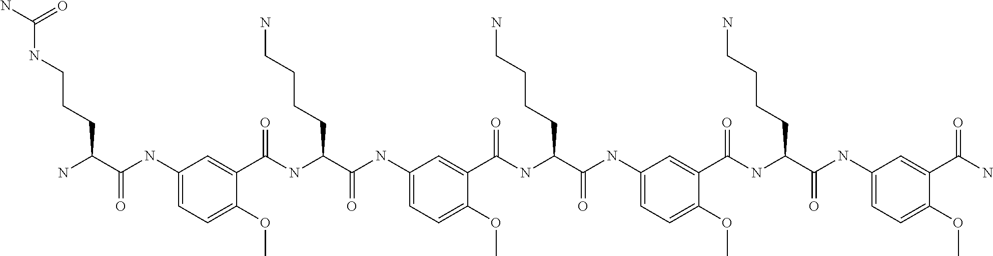 |
| 257 | 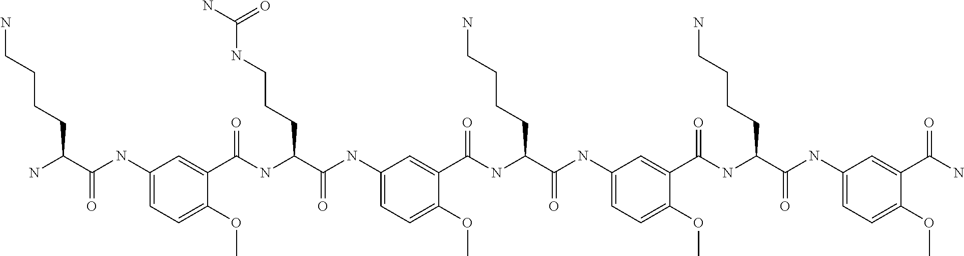 |
| 258 | 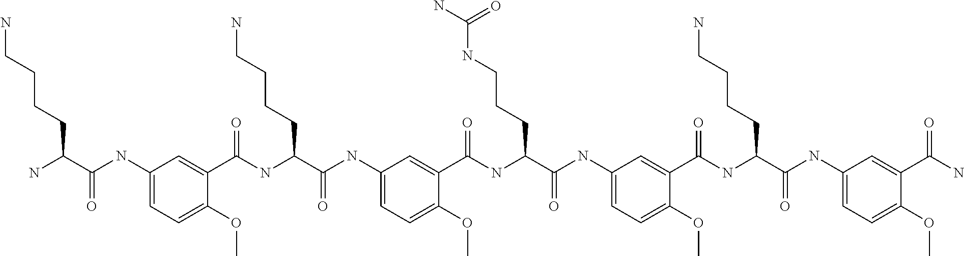 |
| 259 | 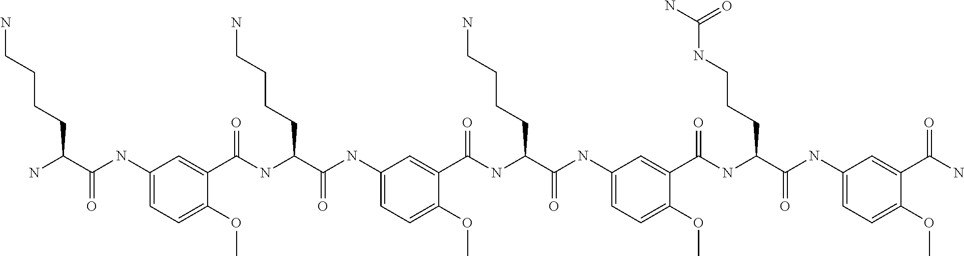 |
| 260 | 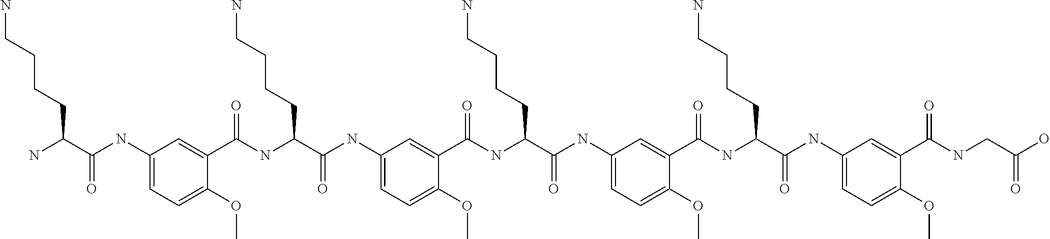 |
| 261 | 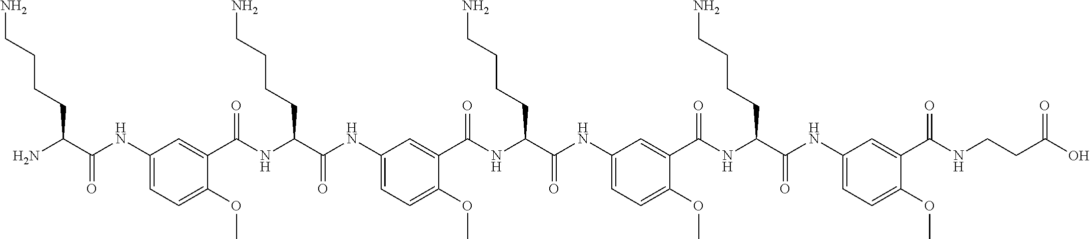 |
| 262 | 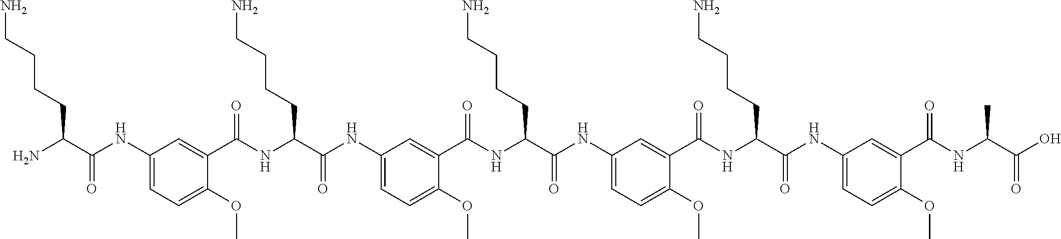 |
| 263 | 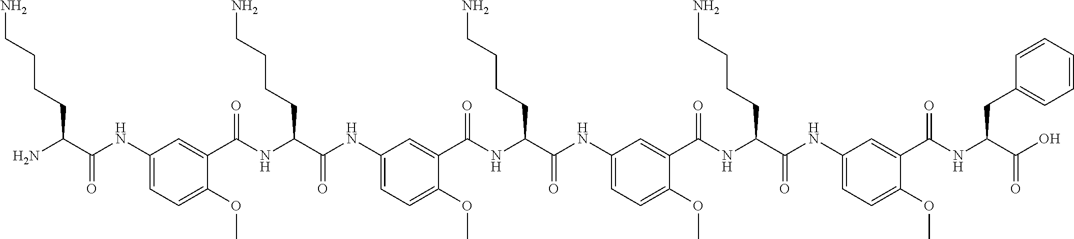 |
| 264 | 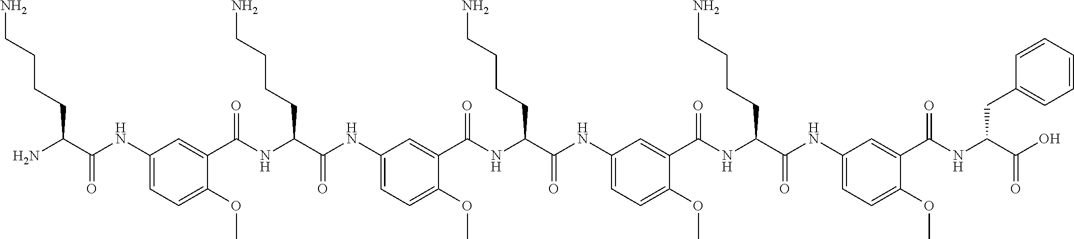 |
| 265 | 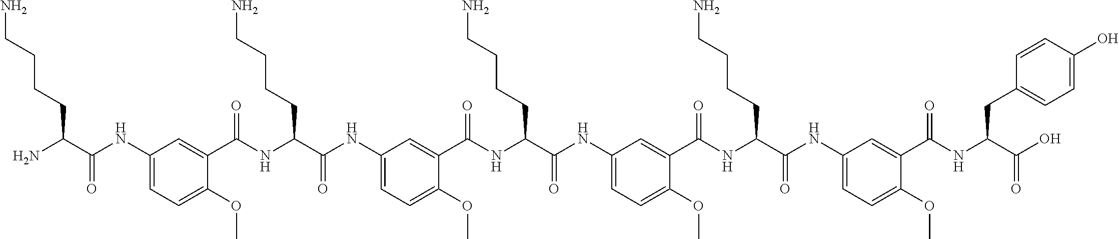 |
| 266 | 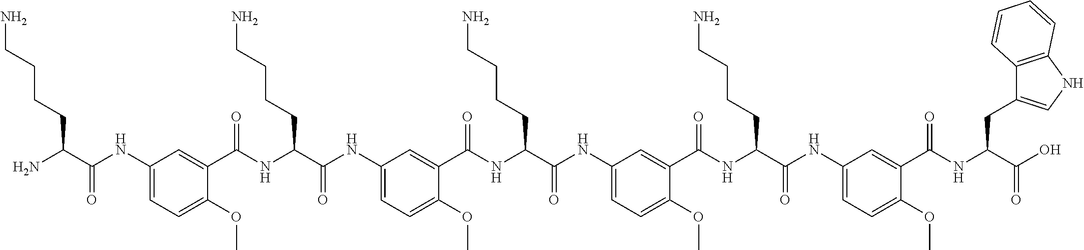 |
| 267 | 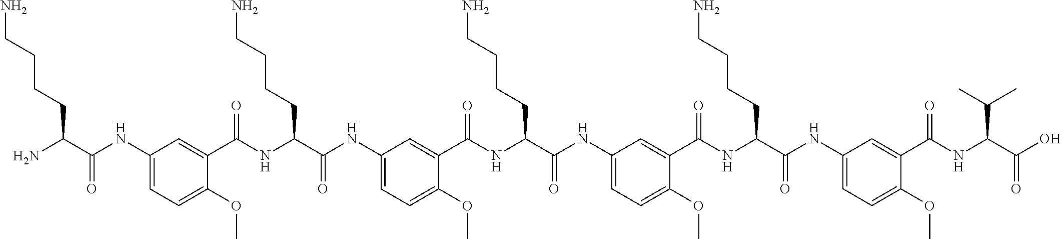 |
| 268 | 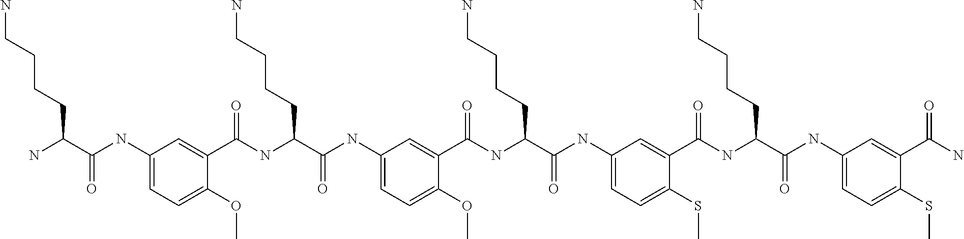 |
| 269 | 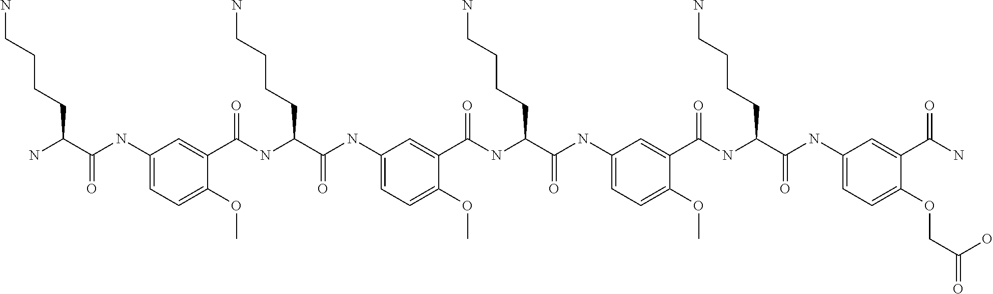 |
| 270 | 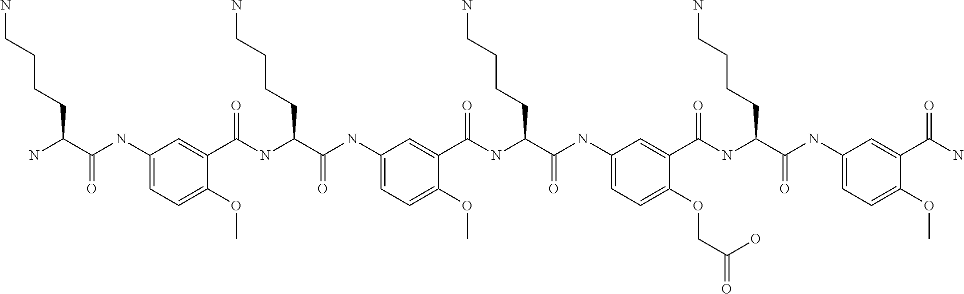 |
| 271 | 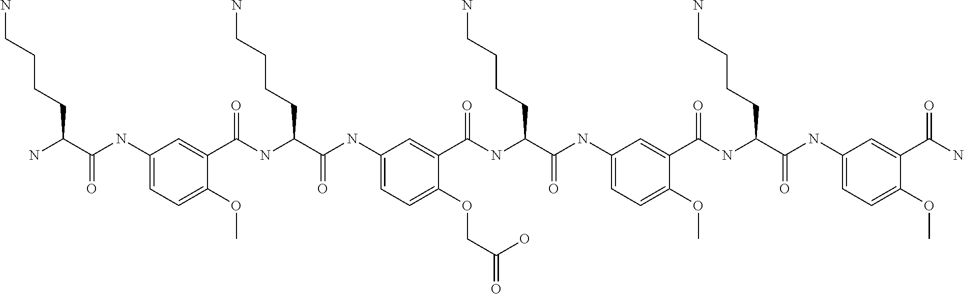 |
| 272 | 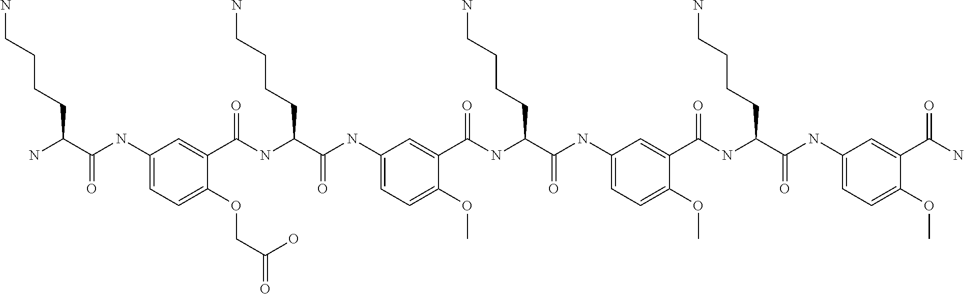 |
| 273 | 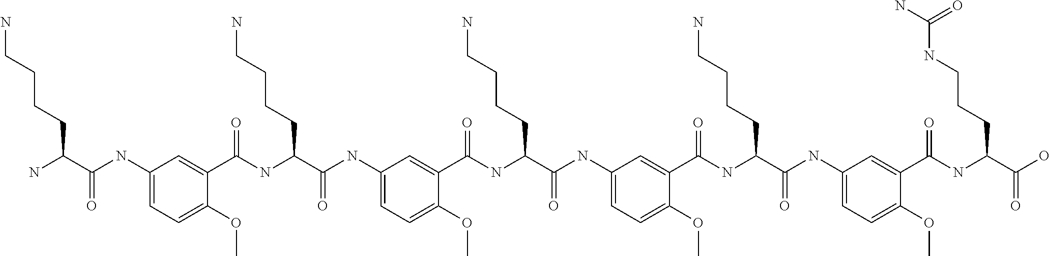 |
| 274 | 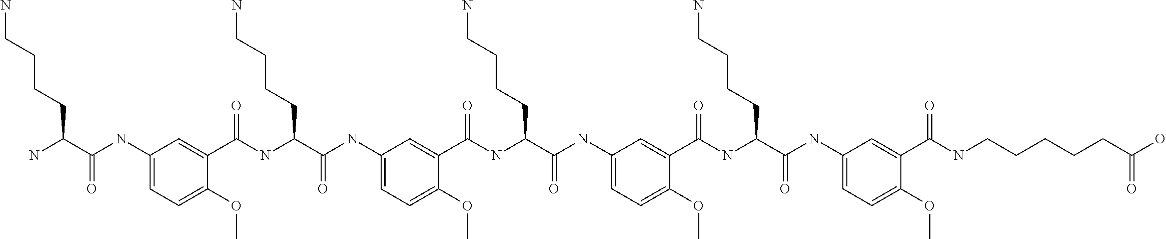 |
| 275 | 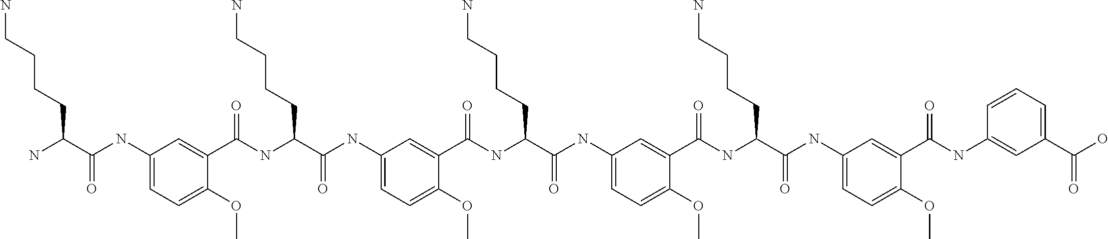 |
| 276 | 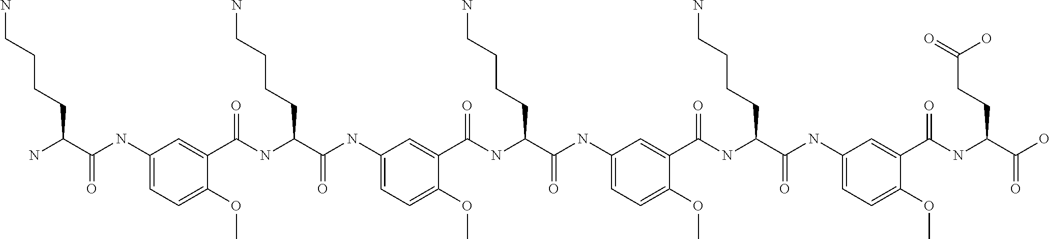 |
| 277 | 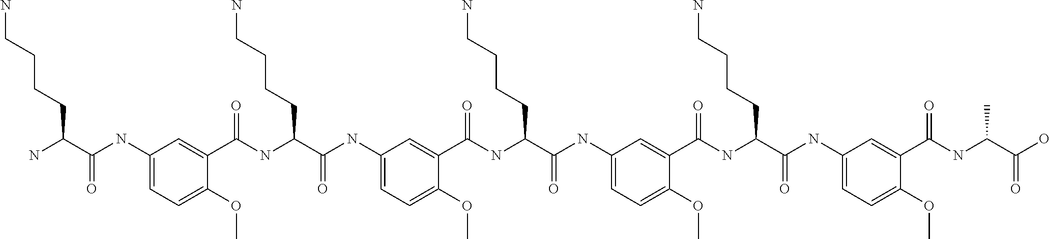 |
| 278 | 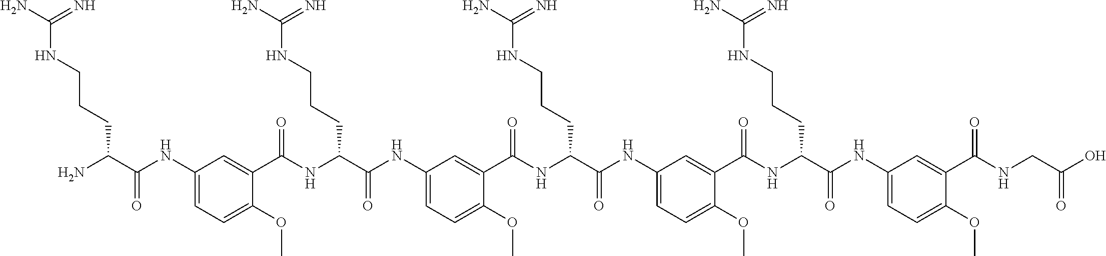 |
| 279 | 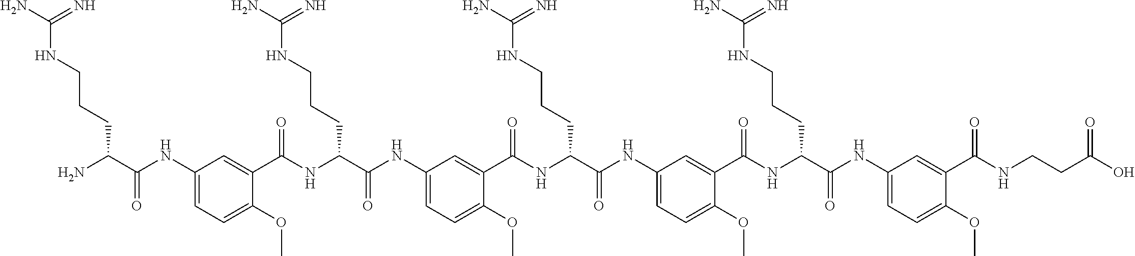 |
| 280 | 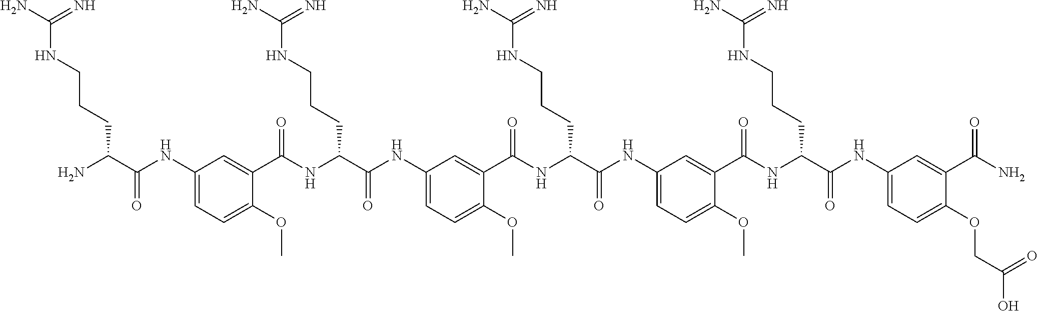 |
| 281 | 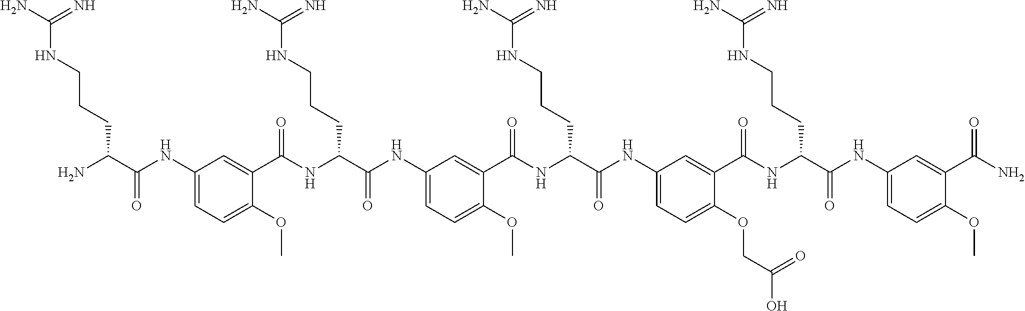 |
| 282 | 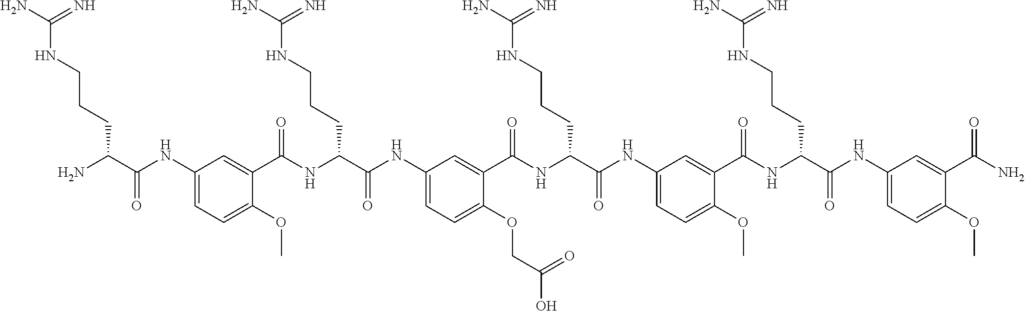 |
| 283 | 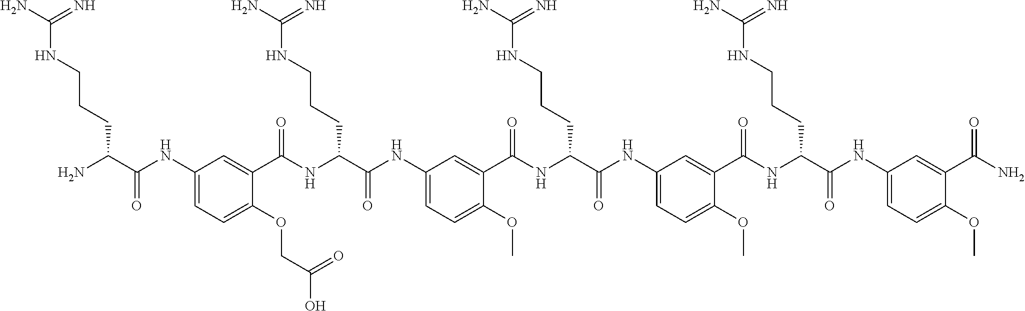 |
| 284 | 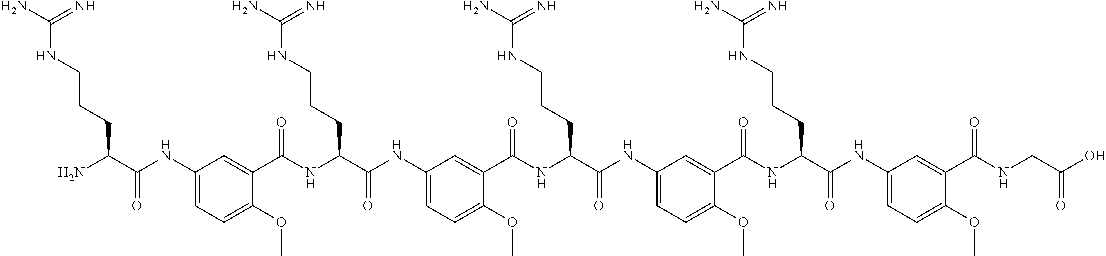 |
| 285 | 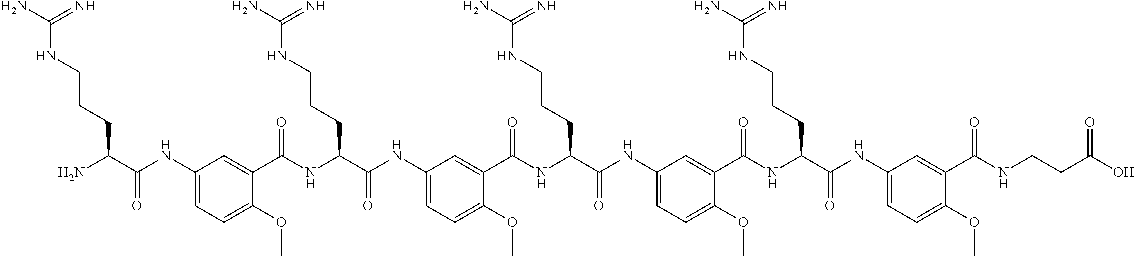 |
| 286 | 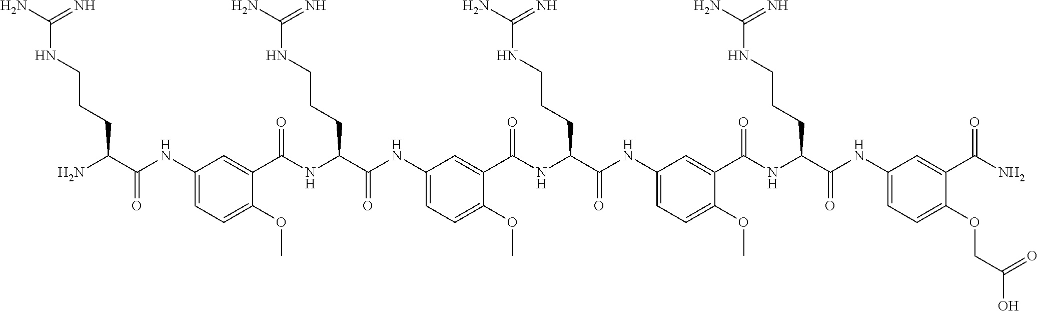 |
| 287 | 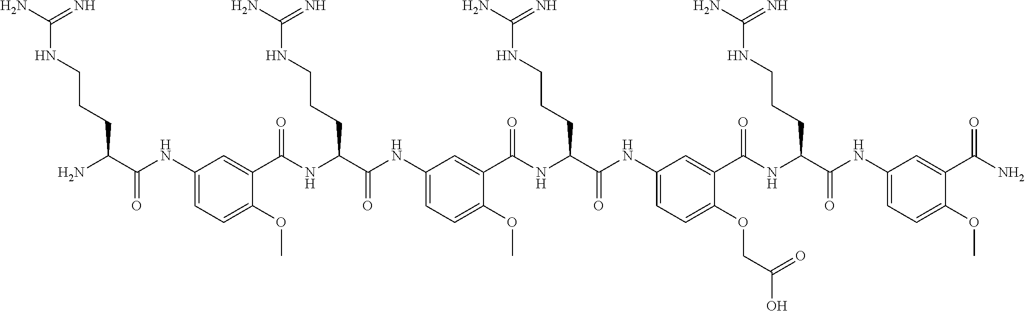 |
| 288 | 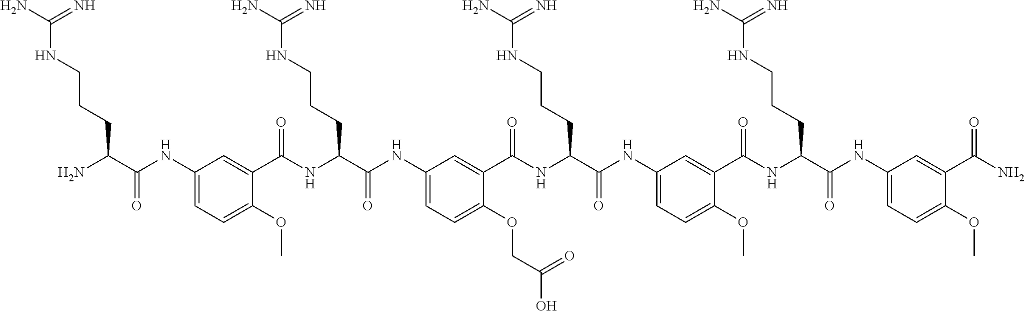 |
| 289 | 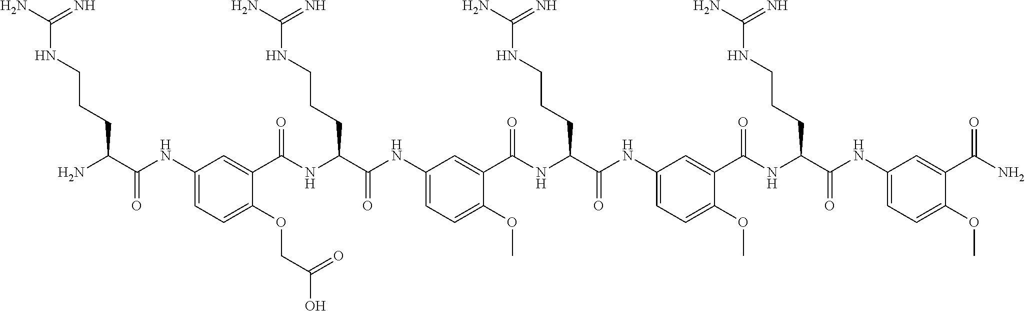 |
| 290 | 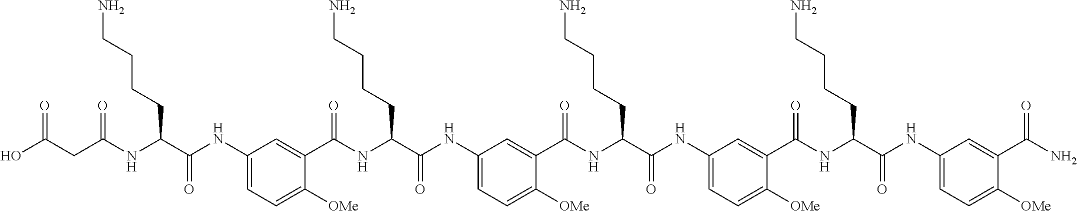 |
| 291 | 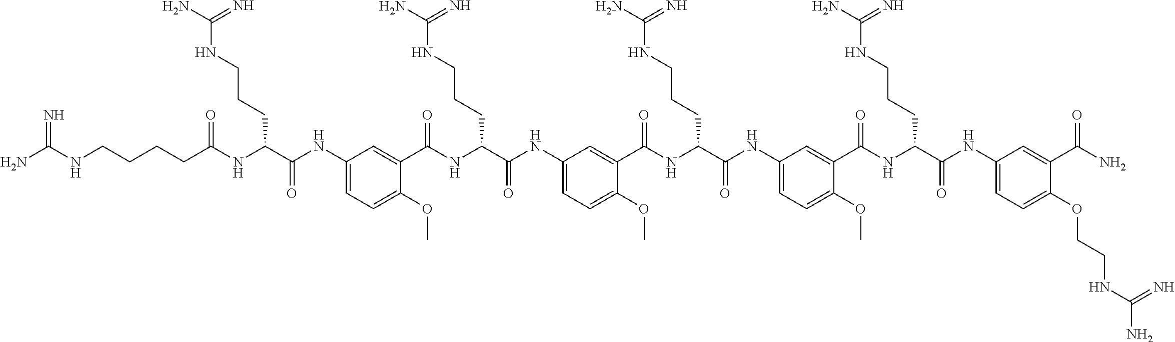 |
| 292 | 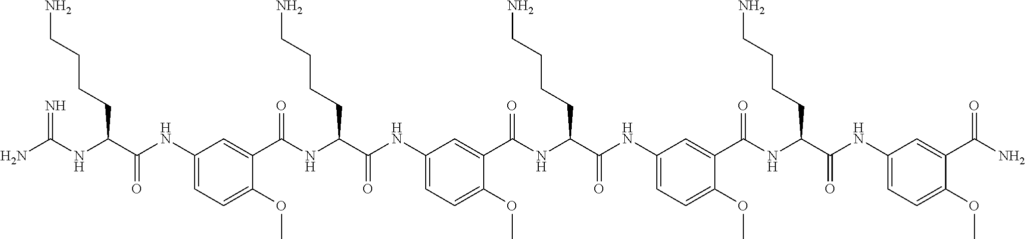 |
| 293 | 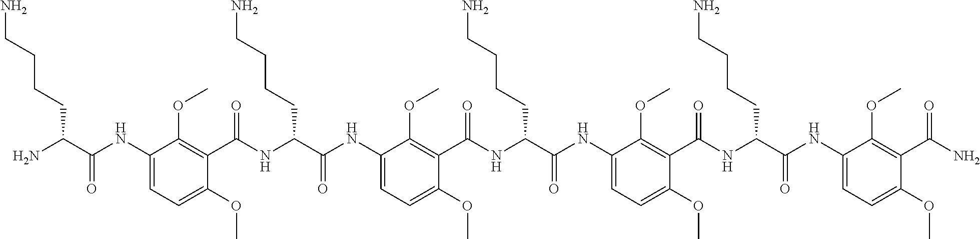 |
| 294 | 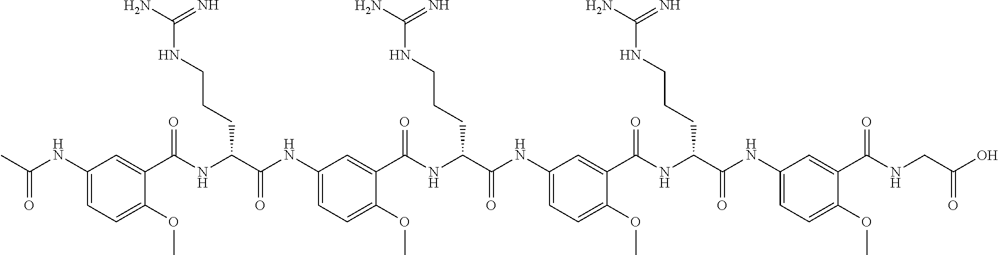 |
| 295 | 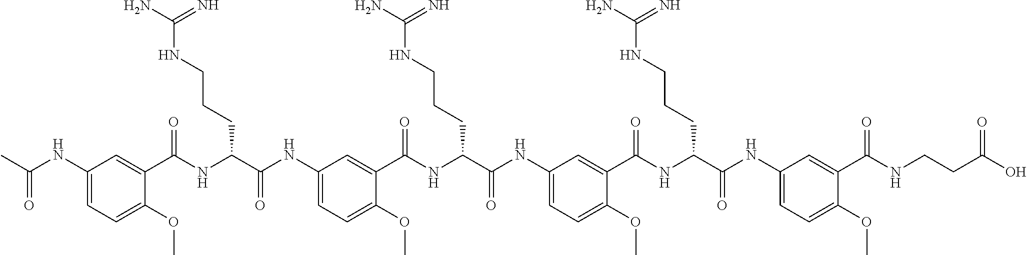 |
| 296 | 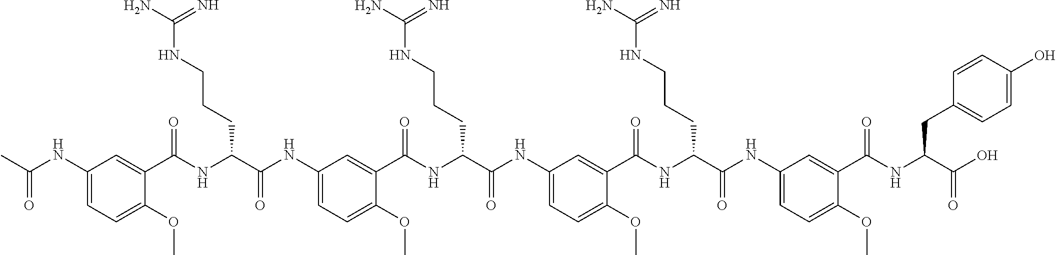 |
| 297 | 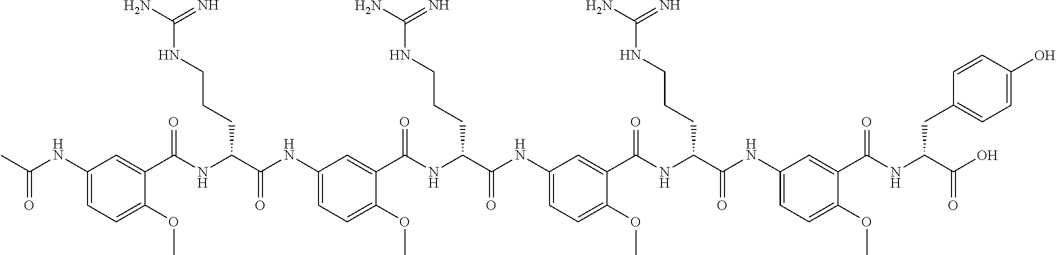 |
| 298 | 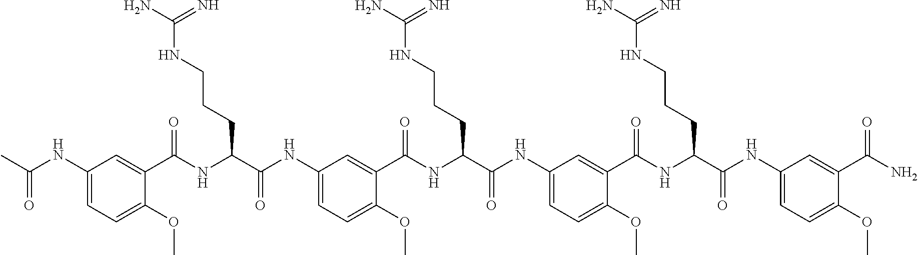 |
| 299 | 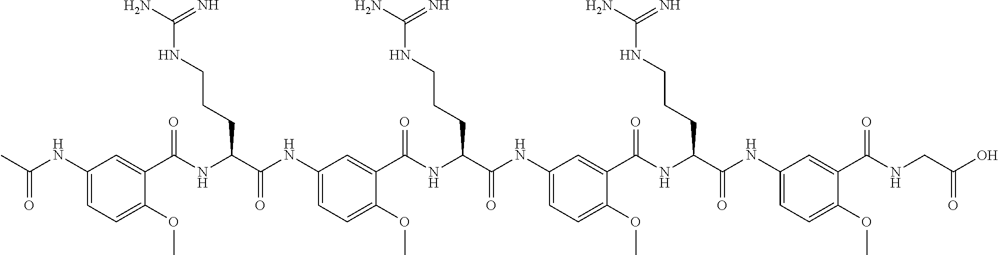 |
| 300 | 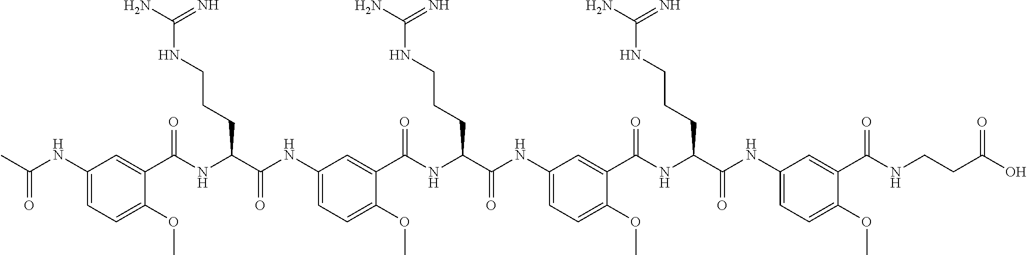 |
| 301 | 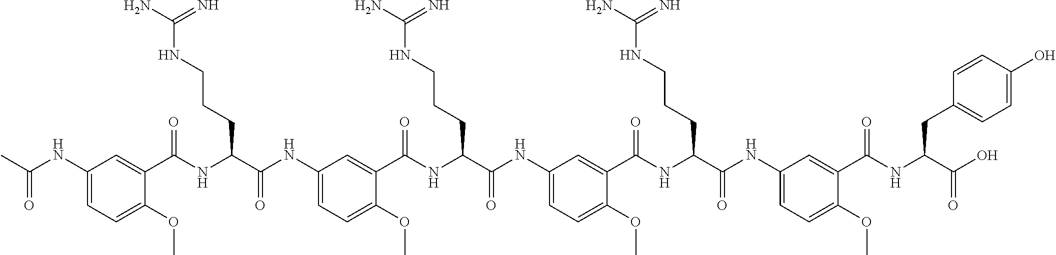 |
| 302 | 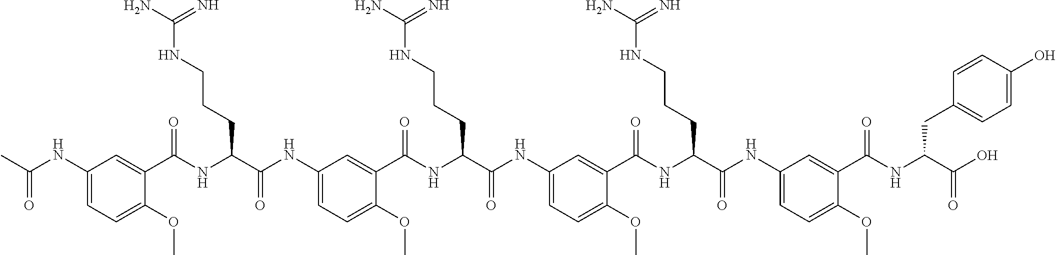 |
| 303 | 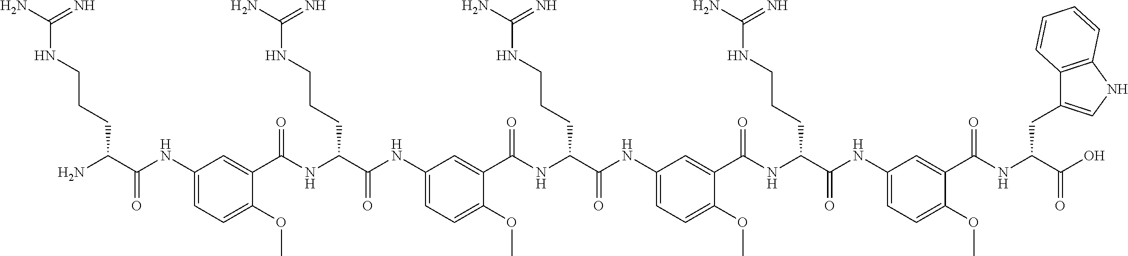 |
| 304 | 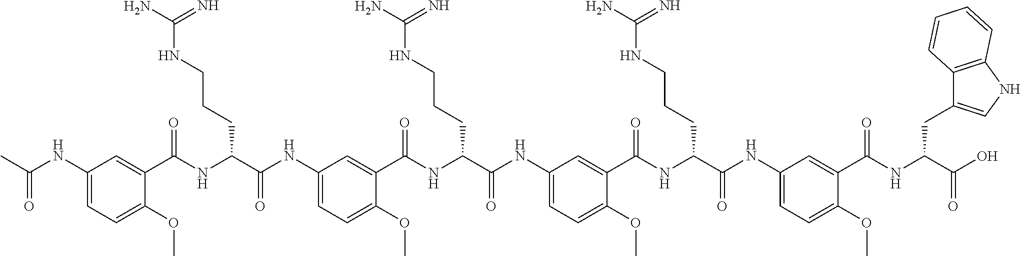 |
| 305 | 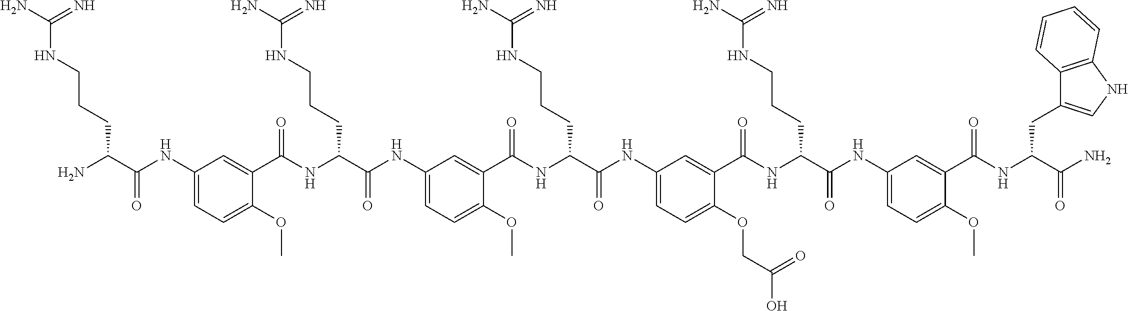 |
| 306 | 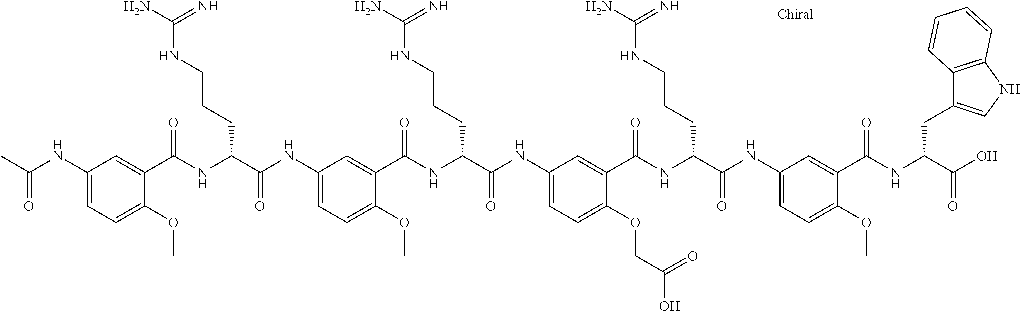 |
| 307 | 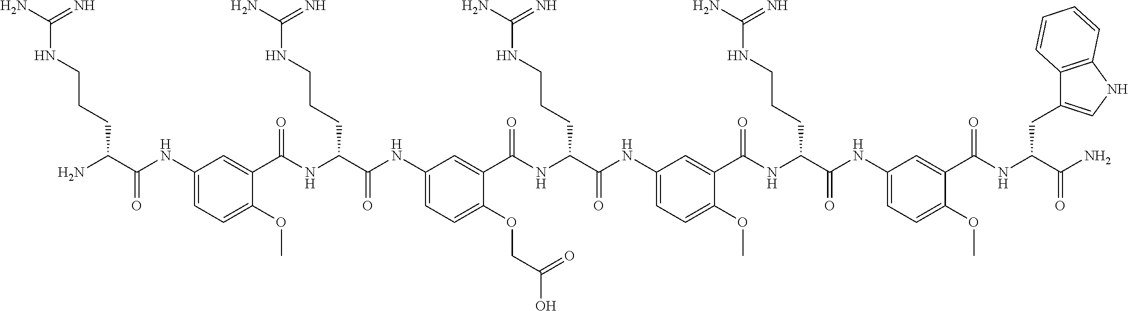 |
| 308 | 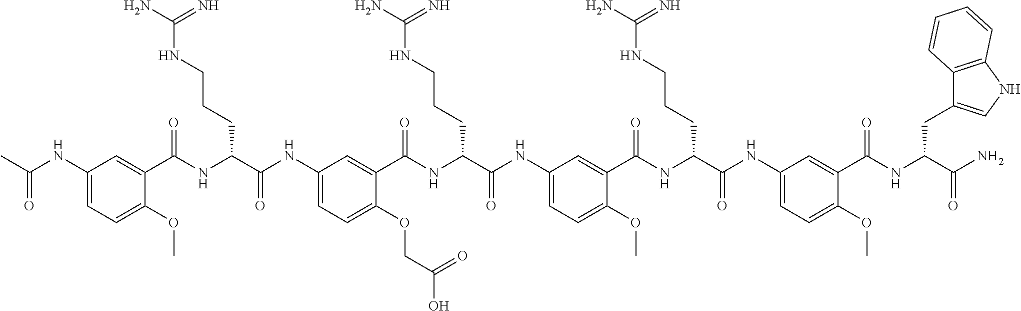 |
| 309 | 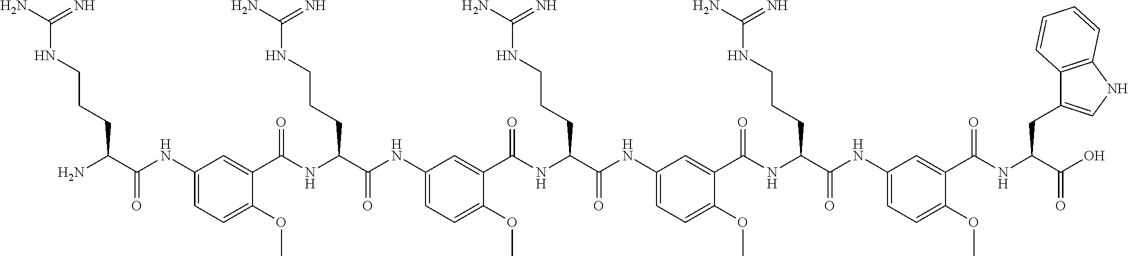 |
| 310 | 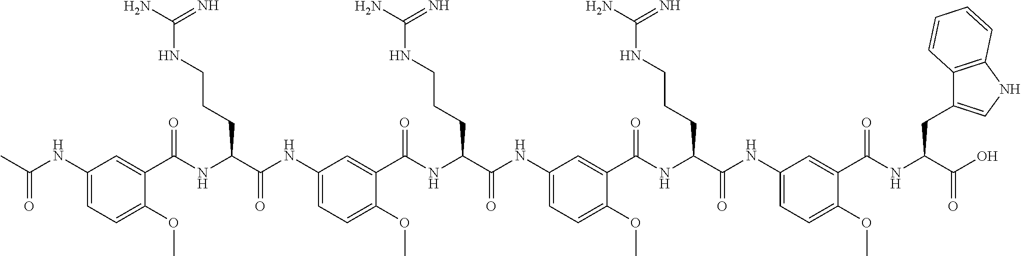 |
| 311 | 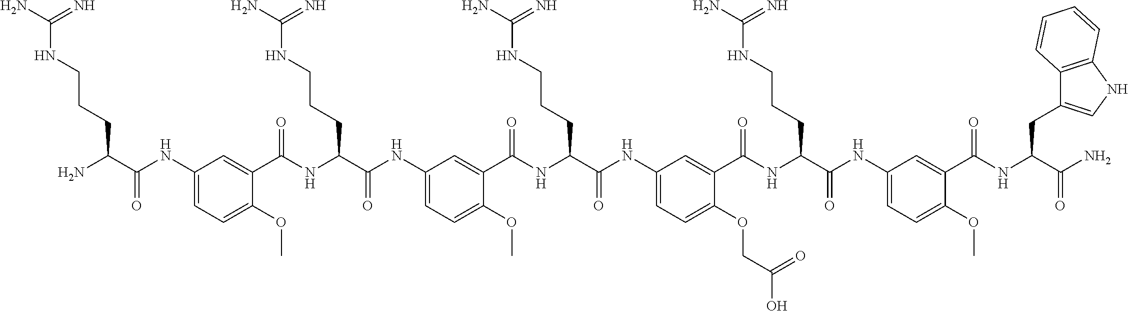 |
| 312 | 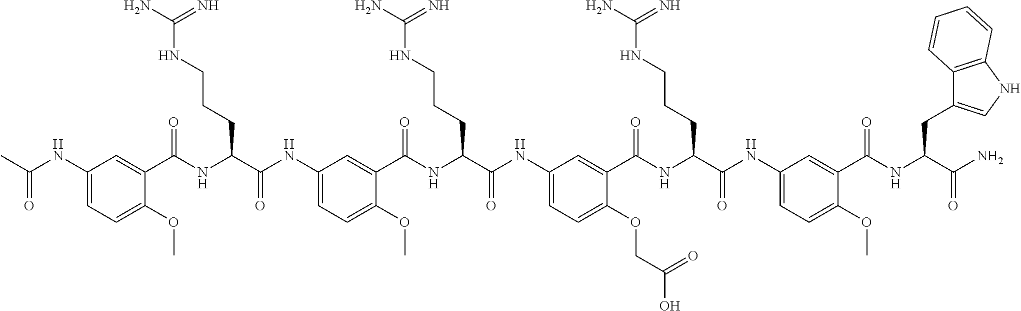 |
| 313 | 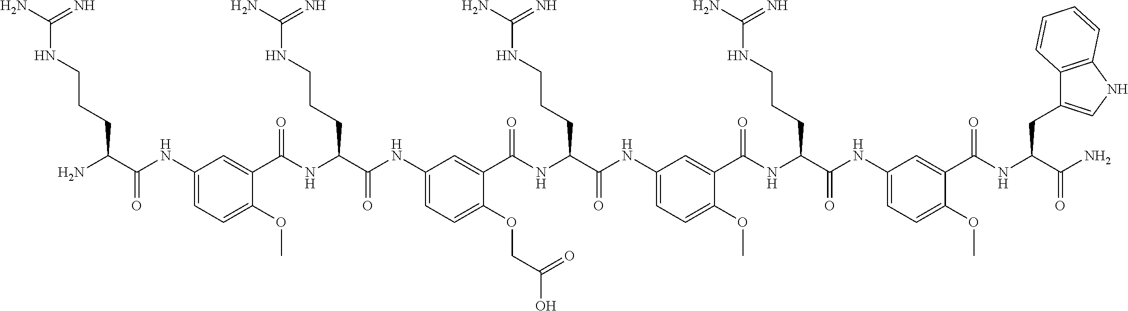 |
| 314 | 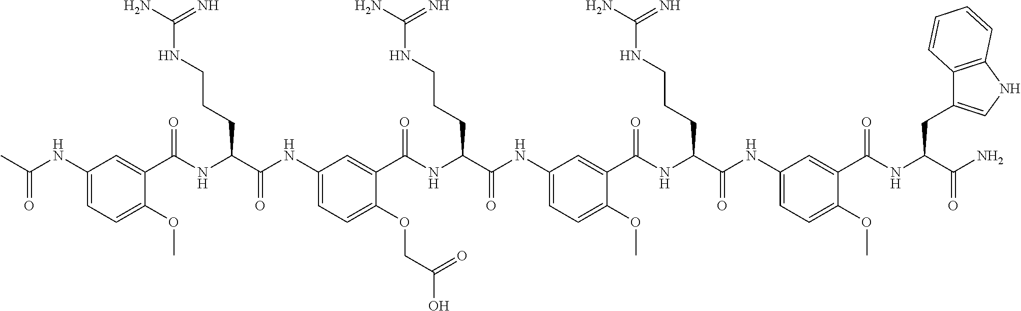 |
| 315 | 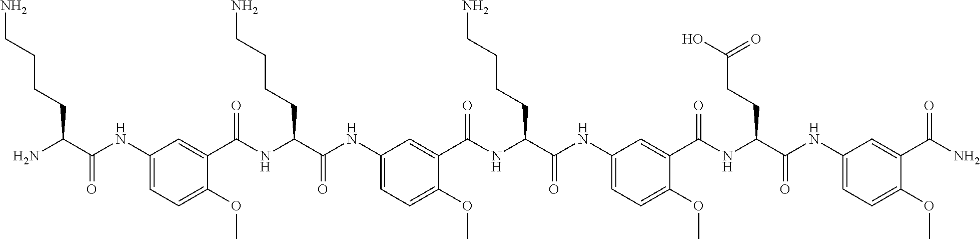 |
| 316 | 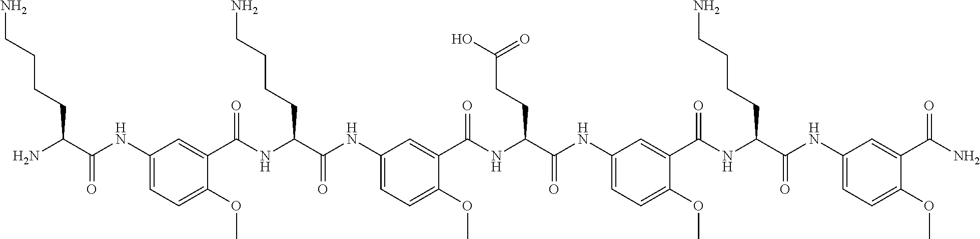 |
| 317 | 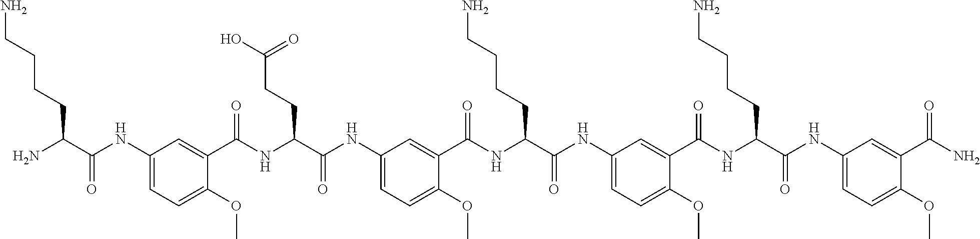 |
| 318 | 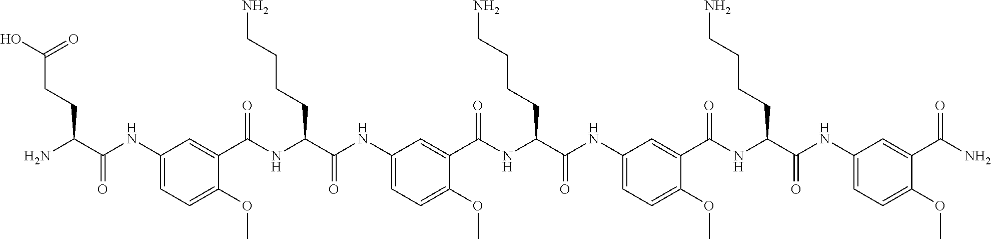 |
| 319 | 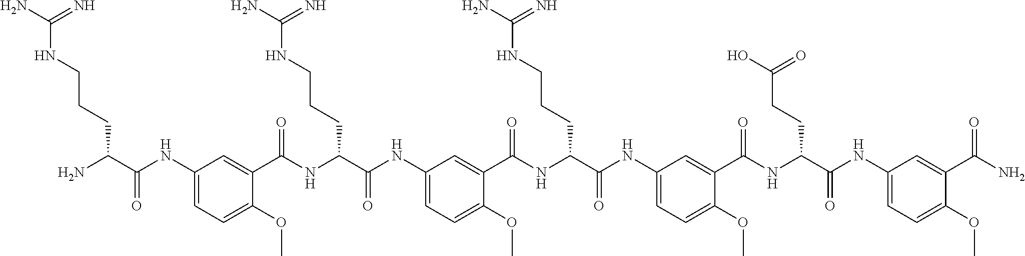 |
| 320 | 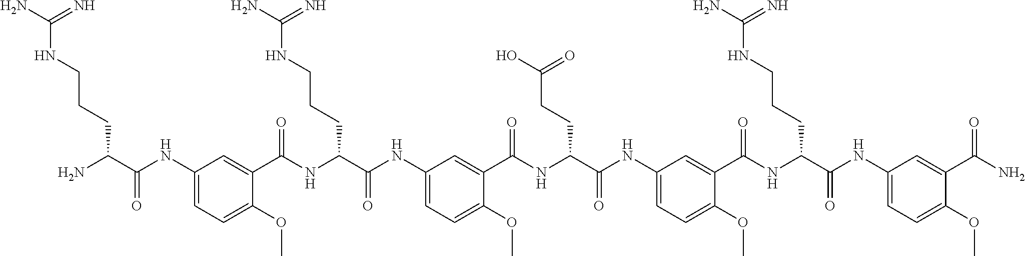 |
| 321 | 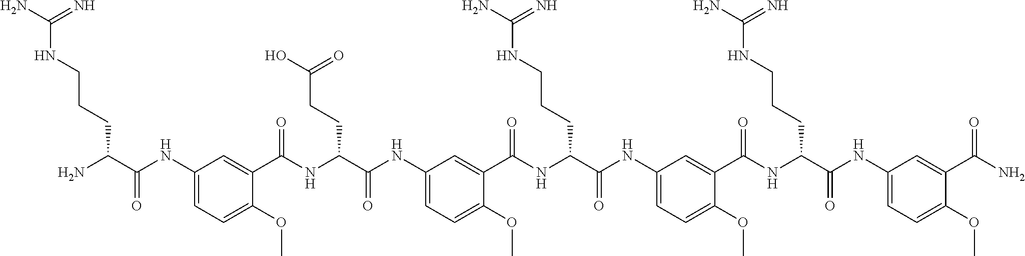 |
| 322 | 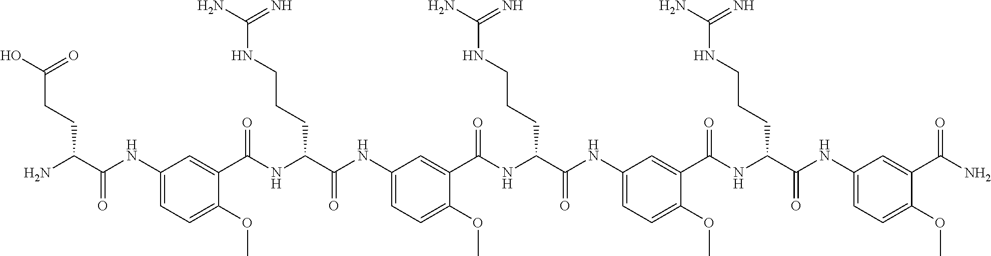 |
| 323 | 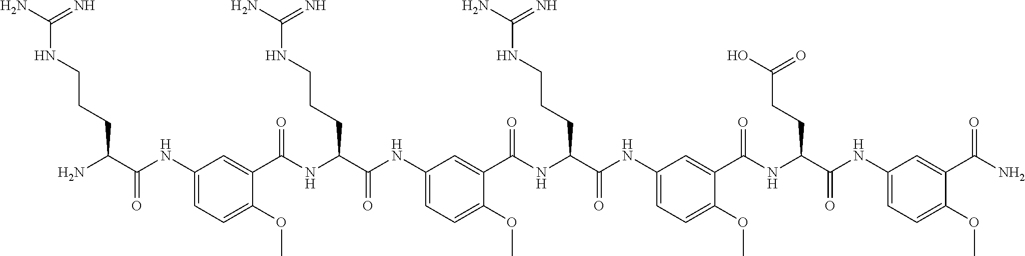 |
| 324 | 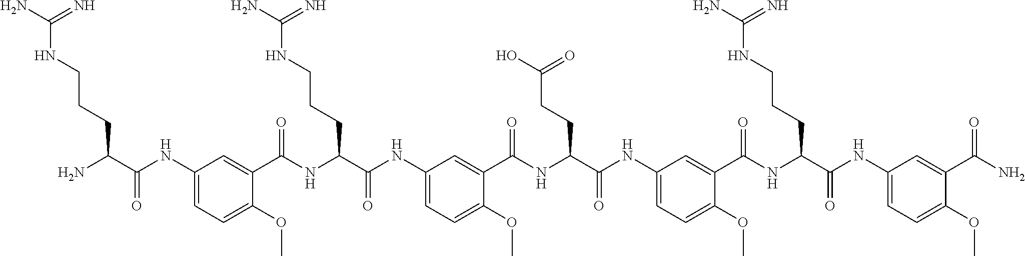 |
| 325 | 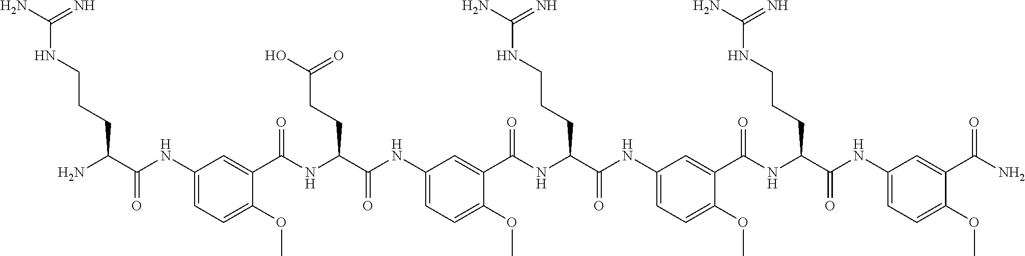 |
| 326 | 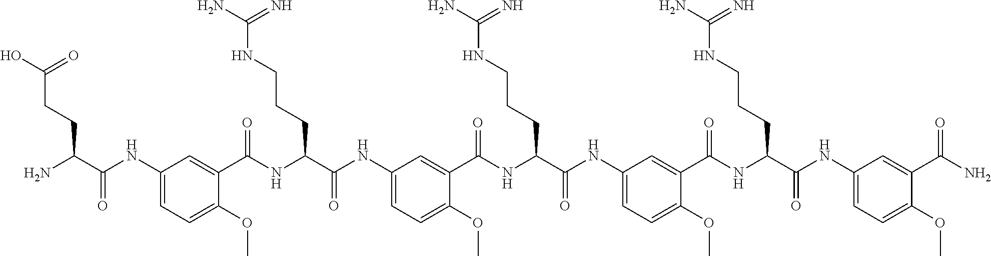 |
| 327 | 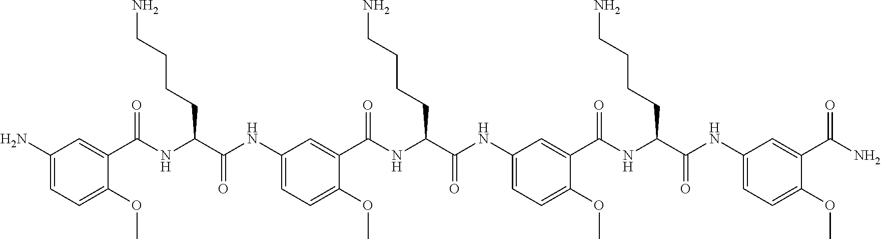 |
| 328 | 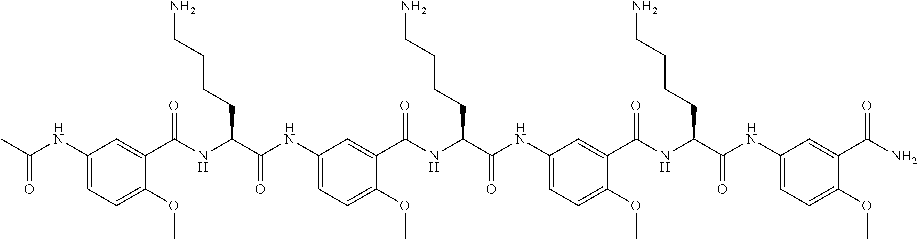 |
| 329 | 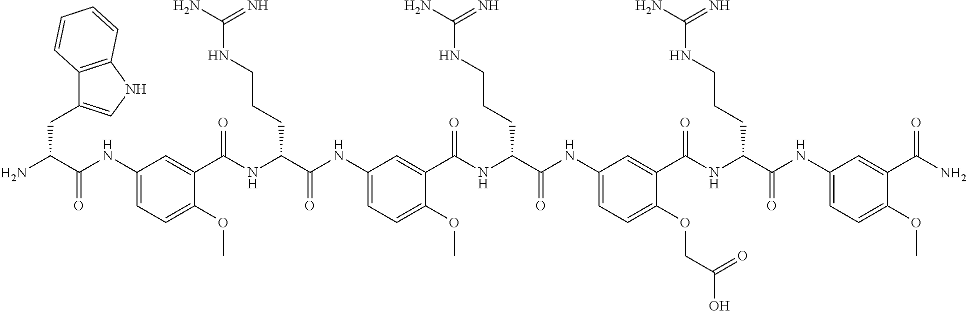 |
| 330 | 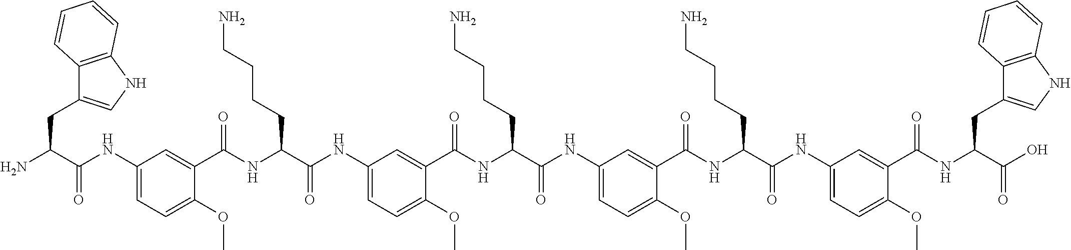 |
| 331 | 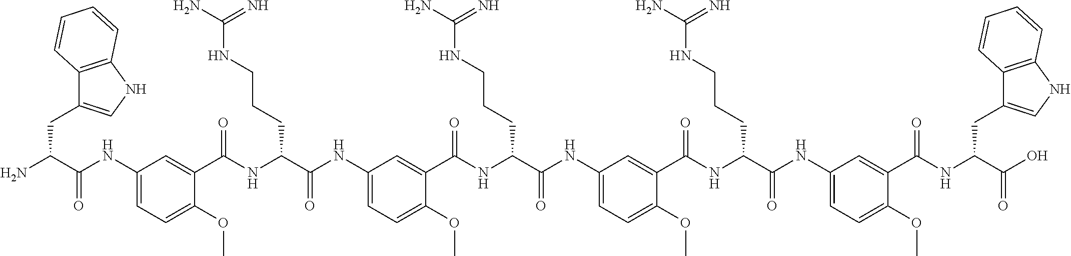 |
| 332 | 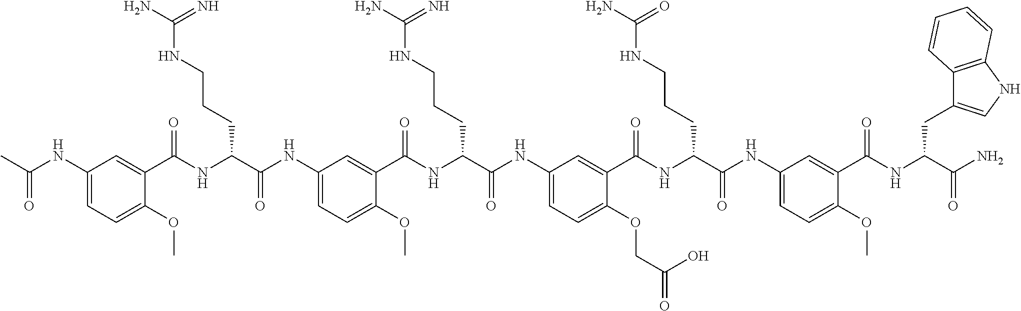 |
| 333 | 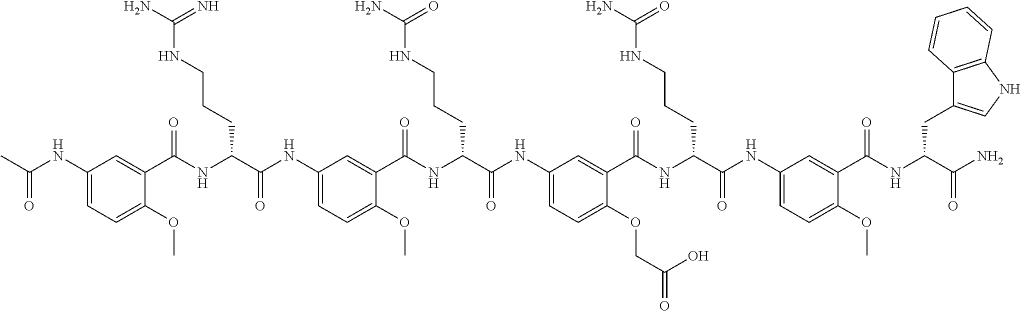 |
| 334 | 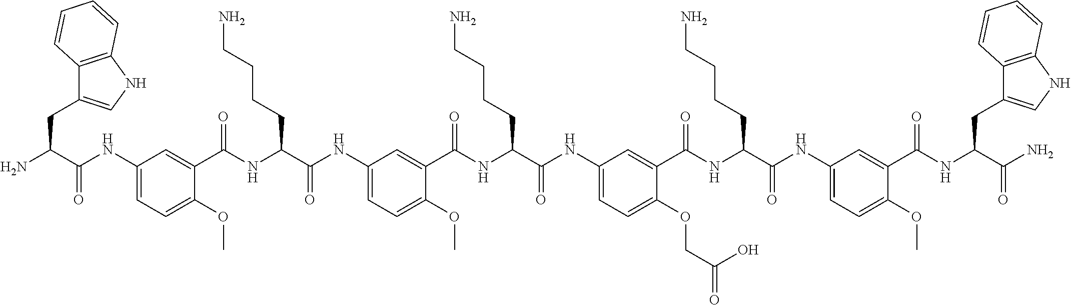 |
| 335 | 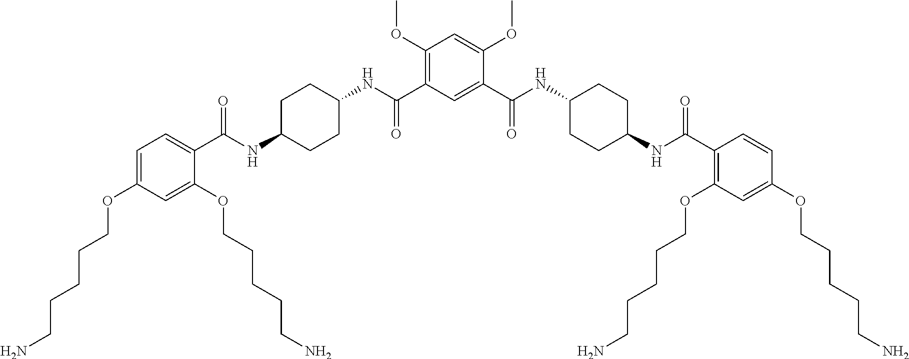 |
| 336 | 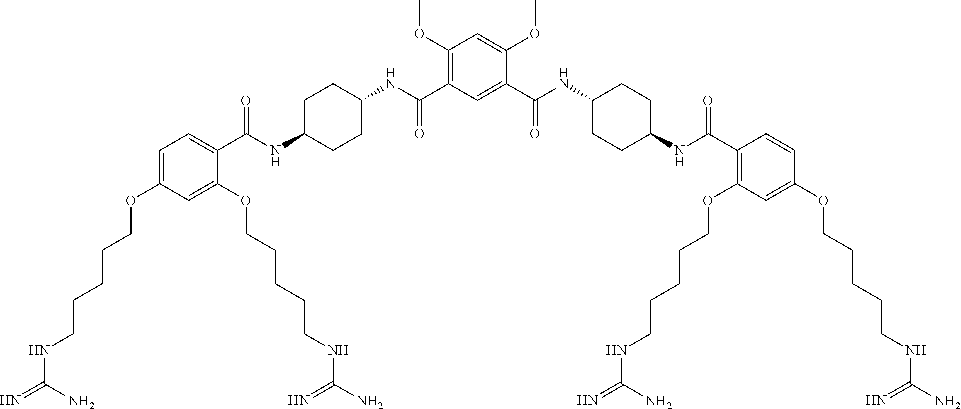 |
| 337 | 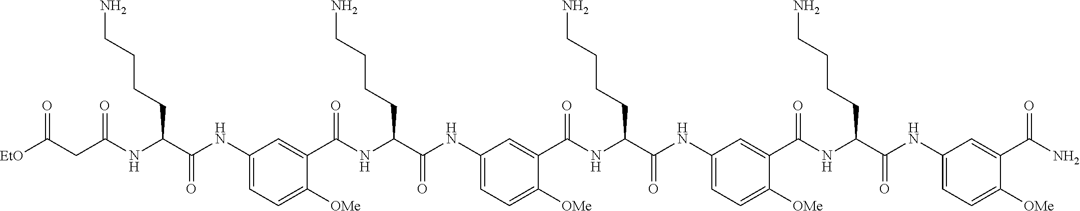 |
| 338 | 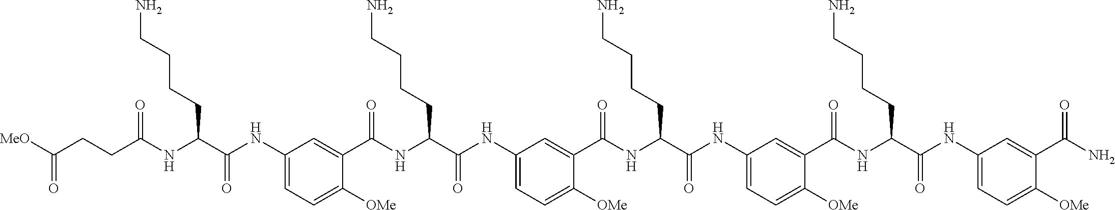 |
| 339 | 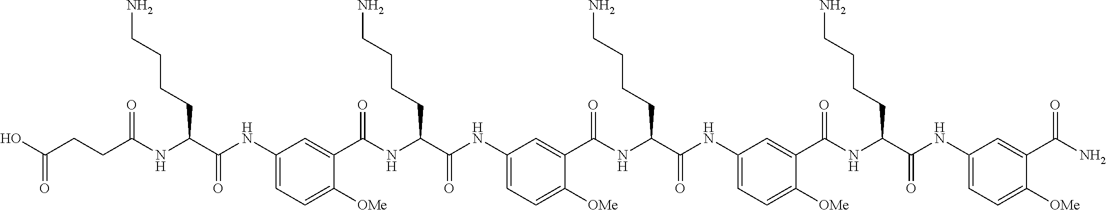 |
| 340 | 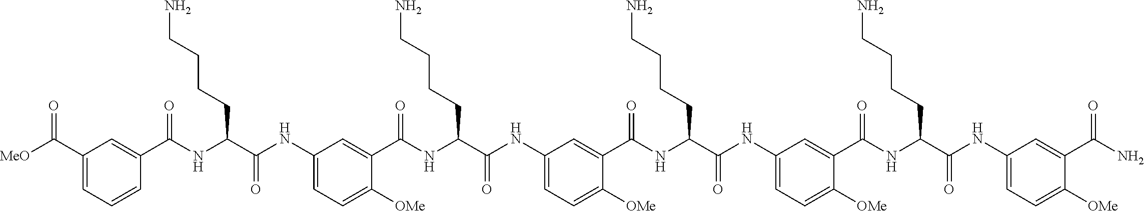 |
| 341 | 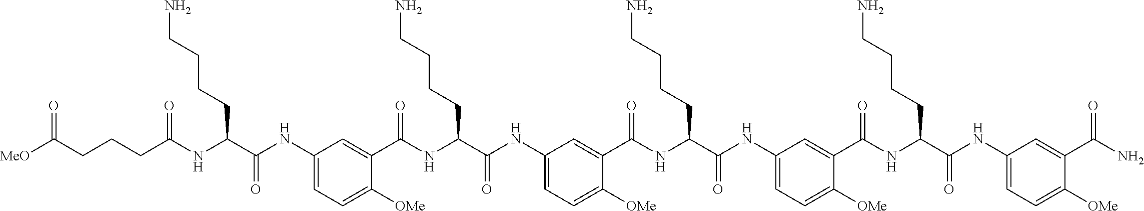 |
| 342 | 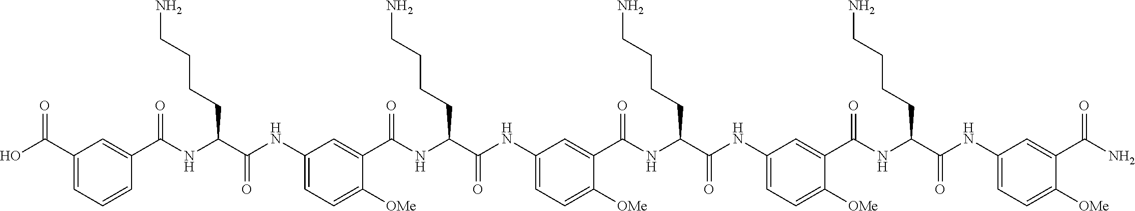 |
| 343 | 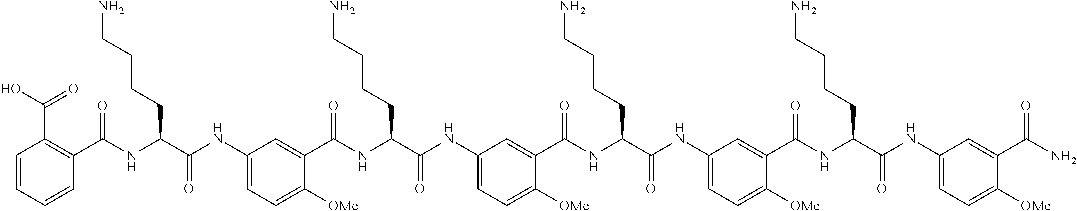 |
| 344 | 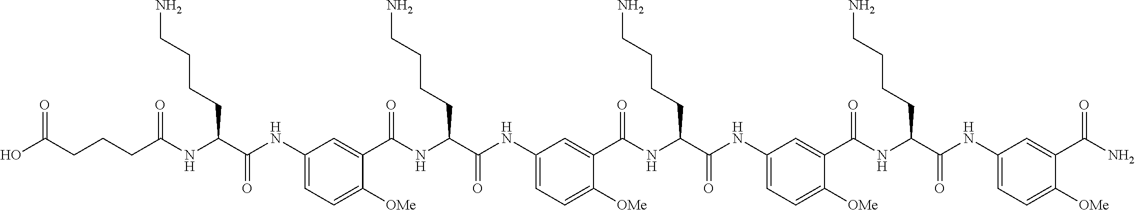 |
| 345 | 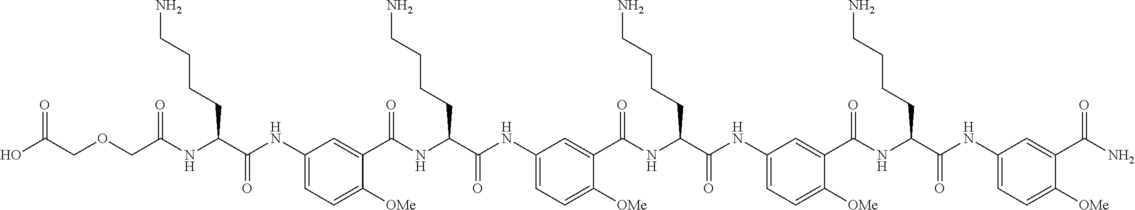 |
| 346 | 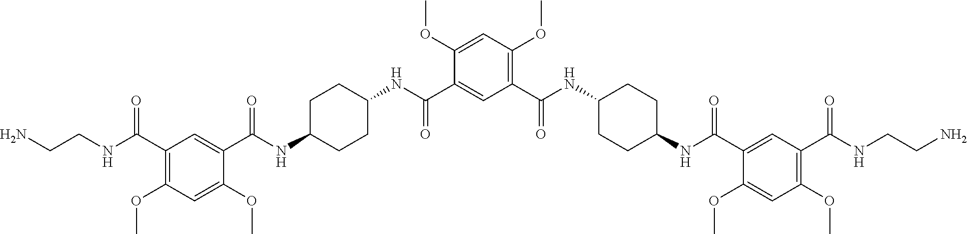 |
| 347 | 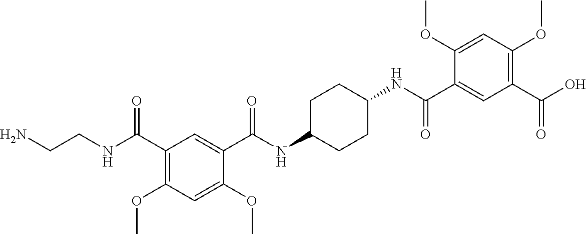 |
| 348 | 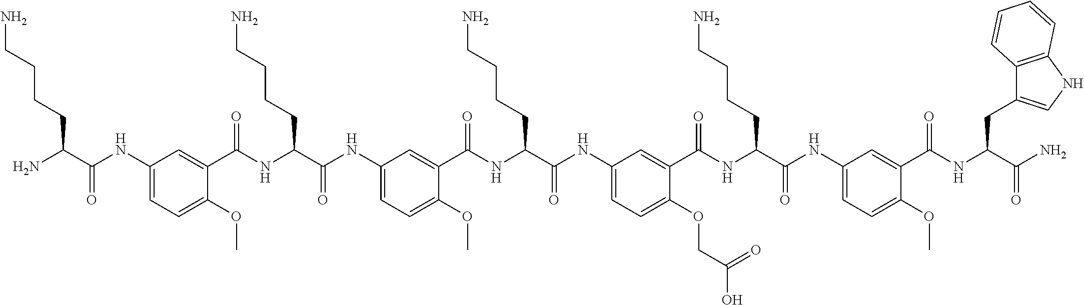 |
| 349 | 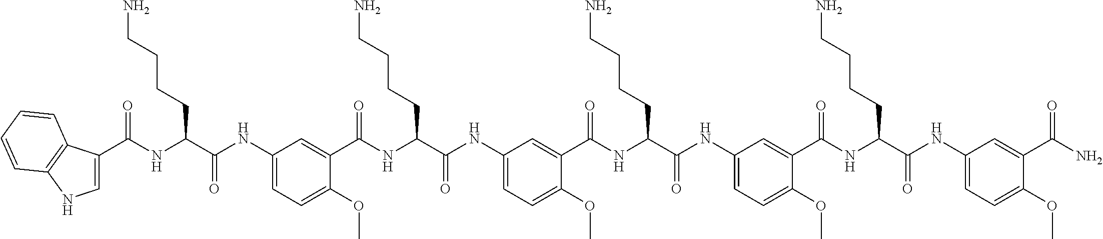 |
| 350 | 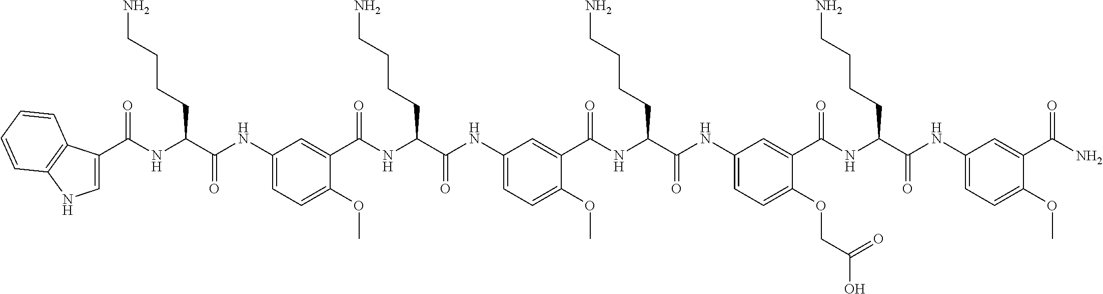 |
| 351 | 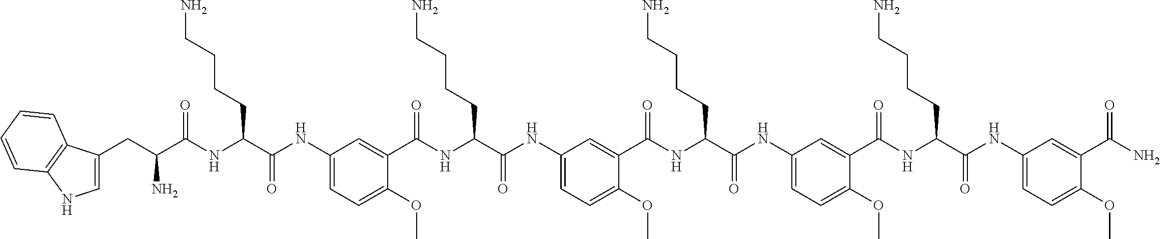 |
| 352 | 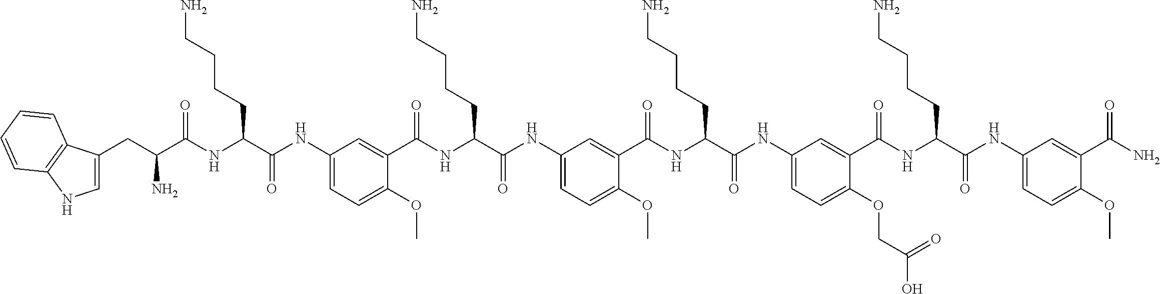 |
| 353 | 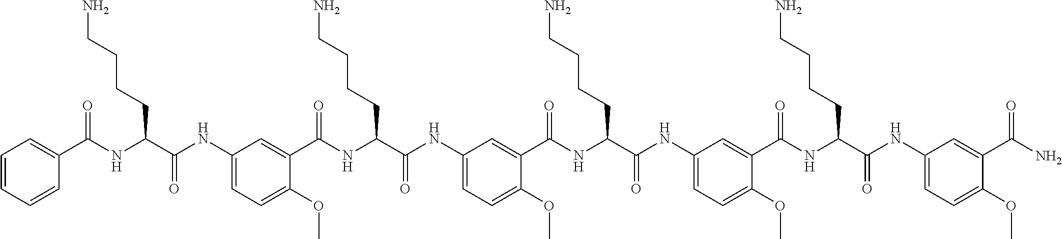 |
| 354 | 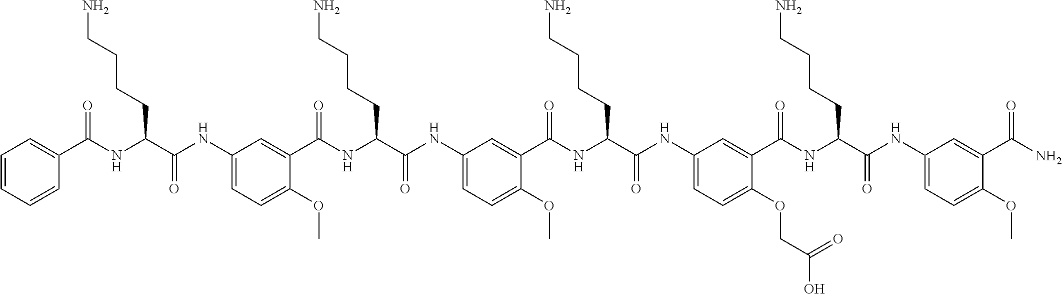 |
| 355 | 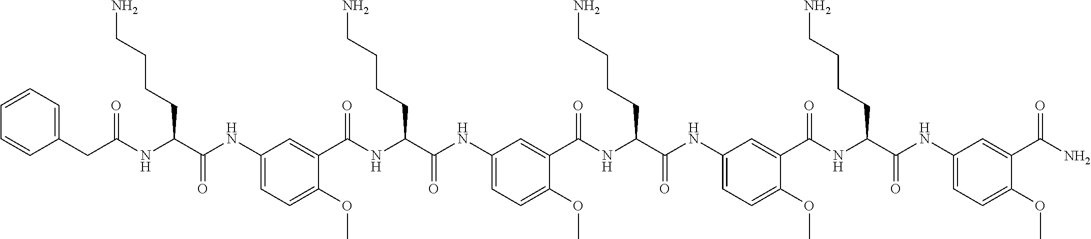 |
| 356 | 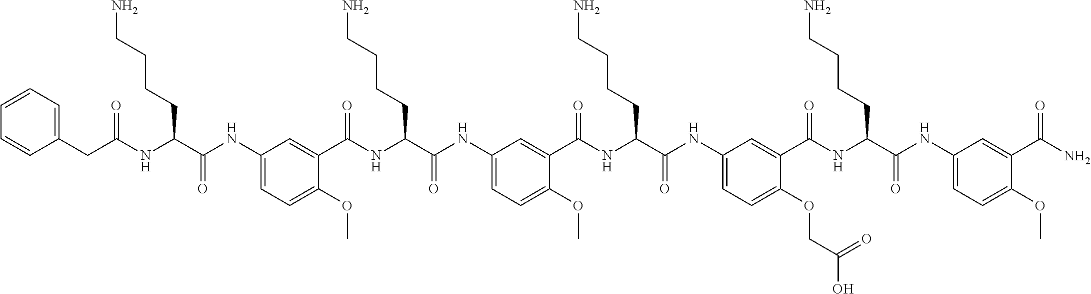 |
| 357 | 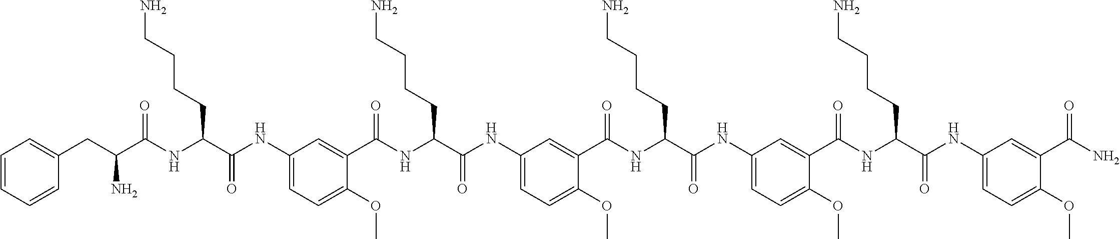 |
| 358 | 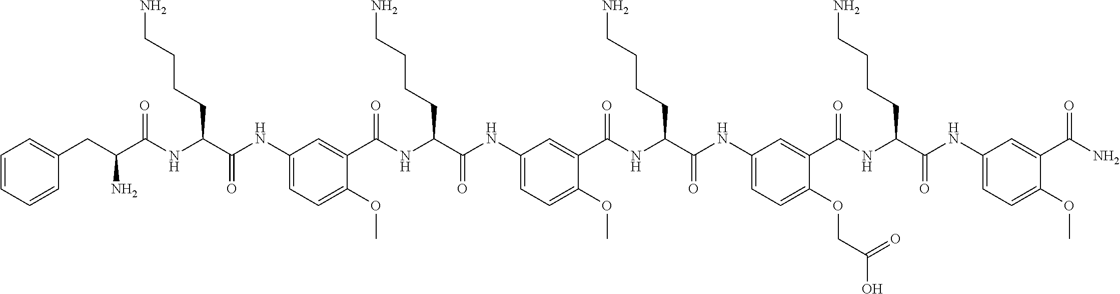 |
| 359 | 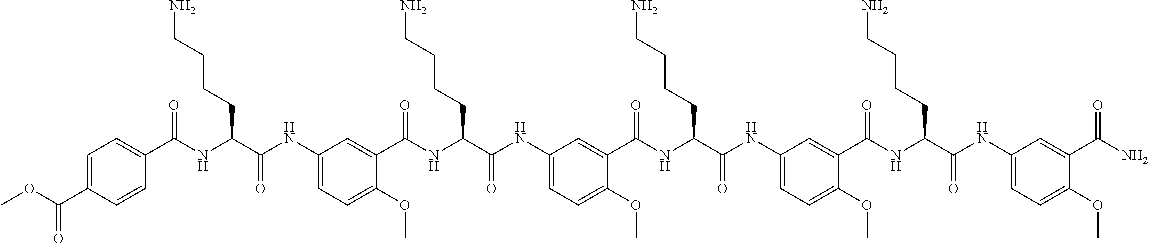 |
| 360 | 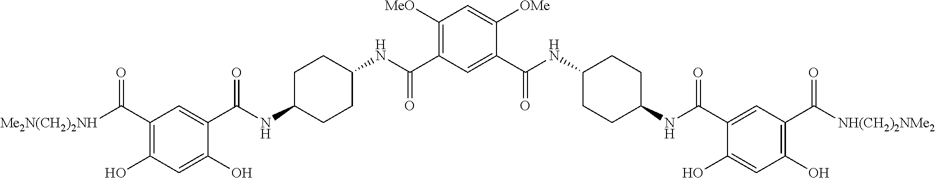 |
| 361 | 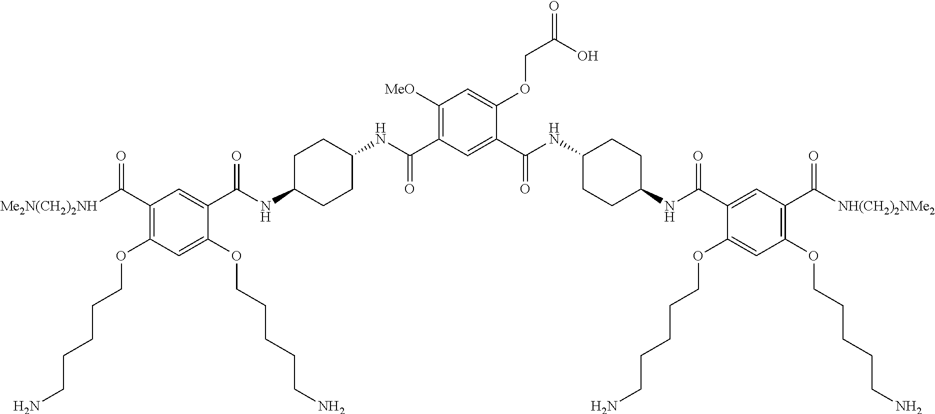 |
| 362 | 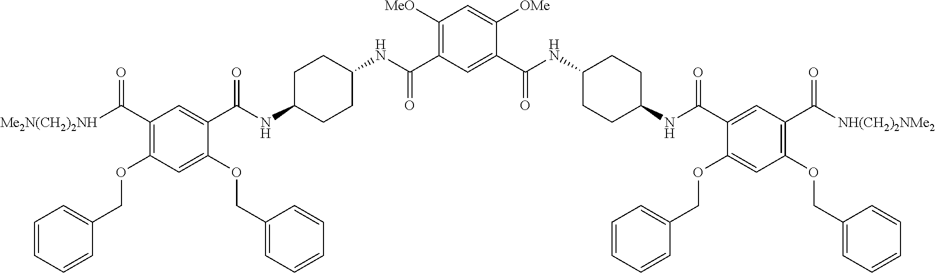 |
| 363 | 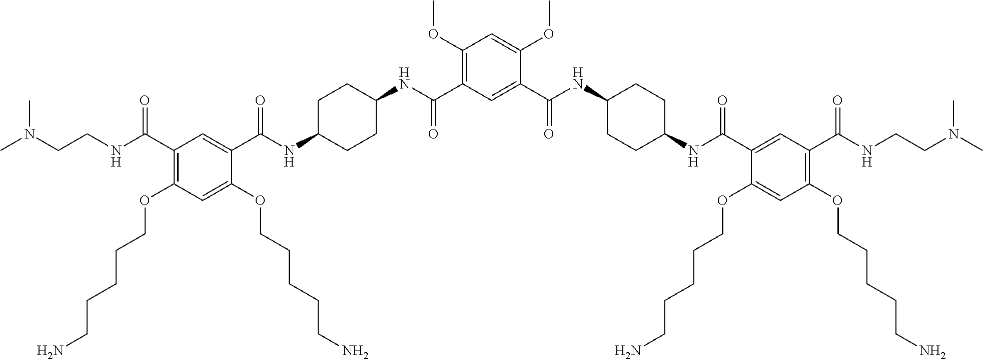 |
| 364 | 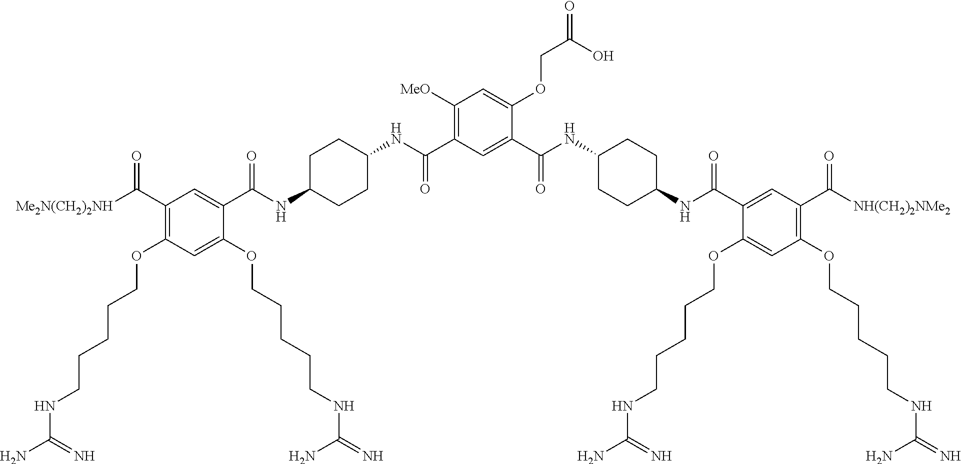 |
| 365 | 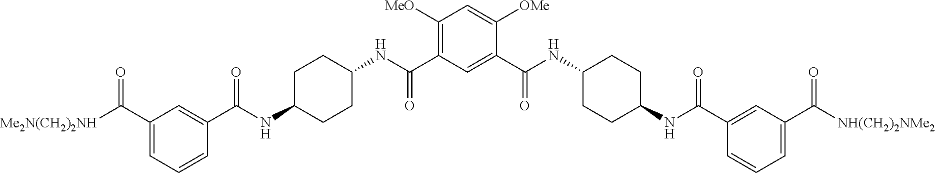 |
| 366 | 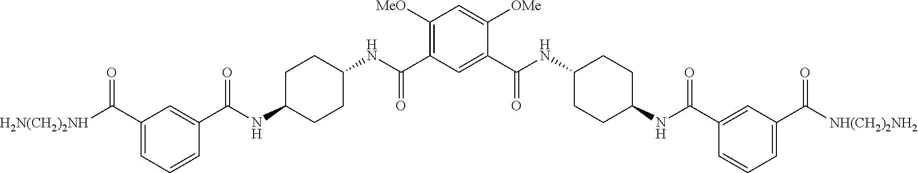 |
| 367 | 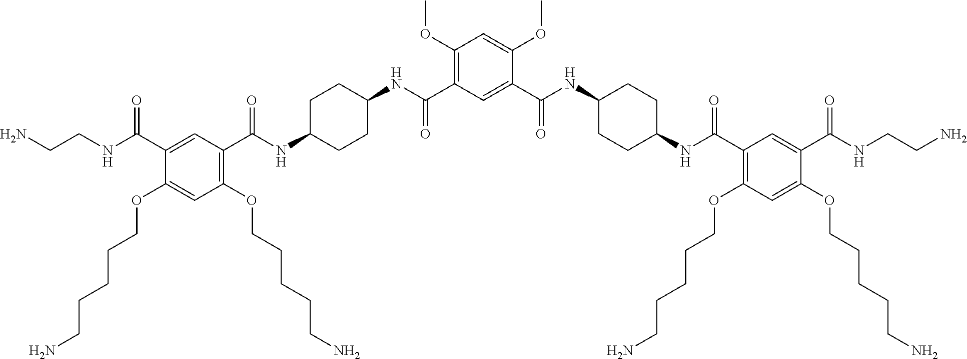 |
| 368 | 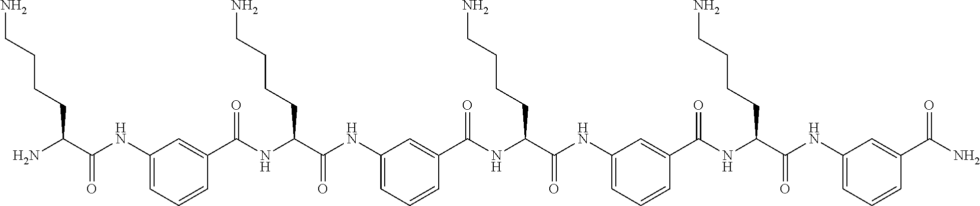 |
| 369 | 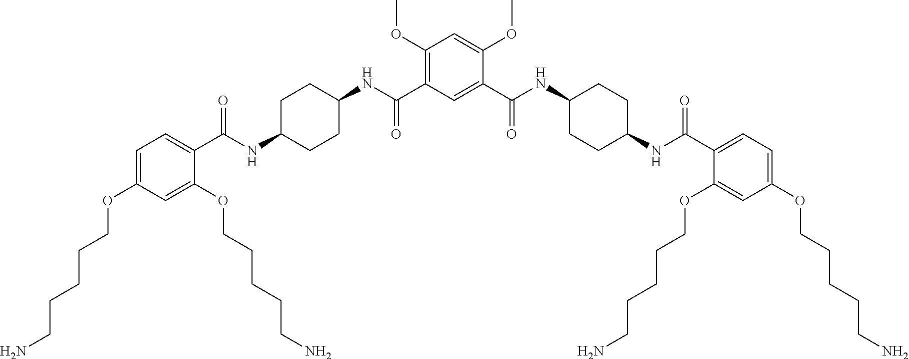 |
| 370 | 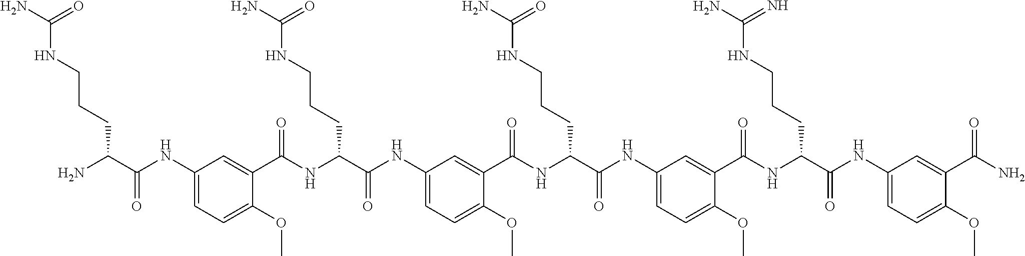 |
| 371 | 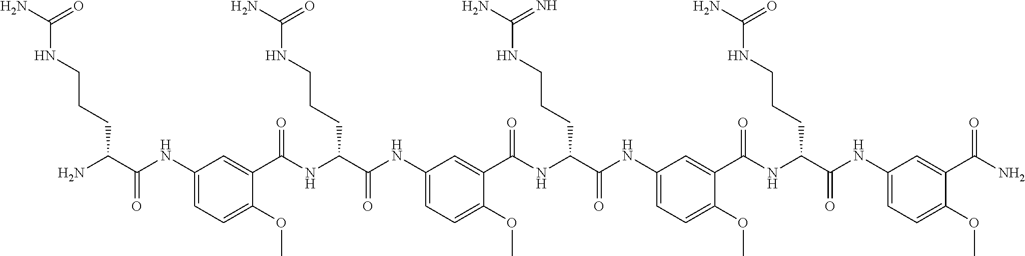 |
| 372 | 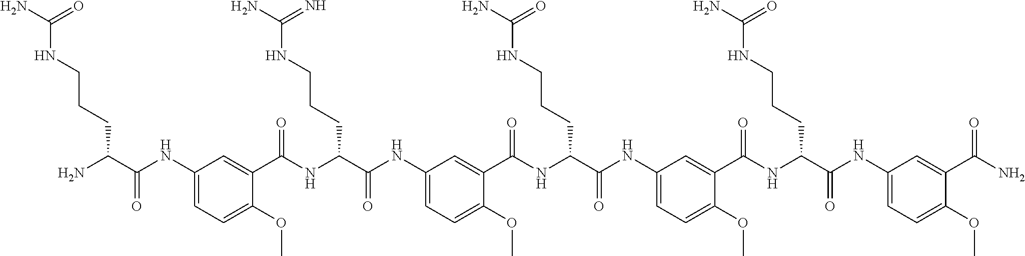 |
| 373 | 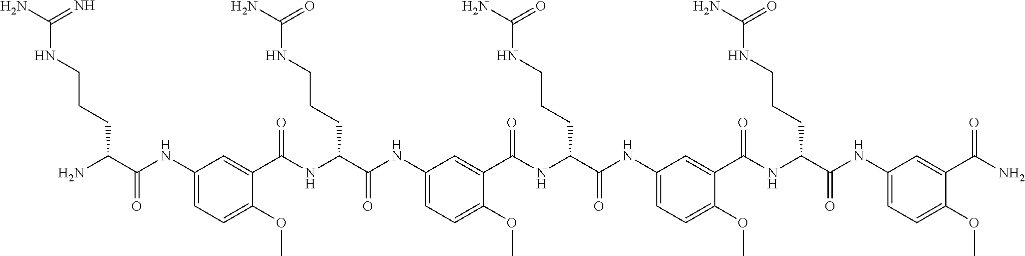 |
| 374 | 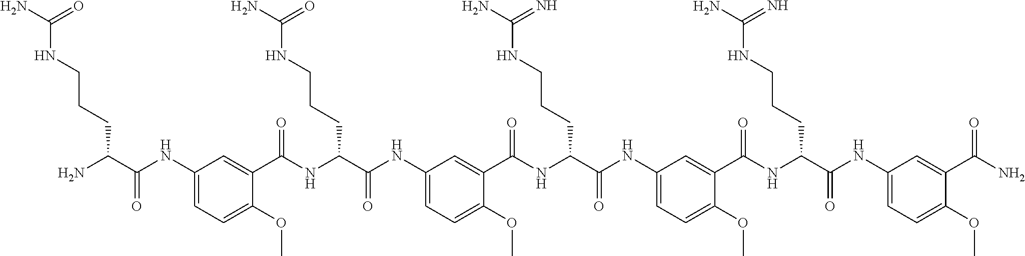 |
| 375 | 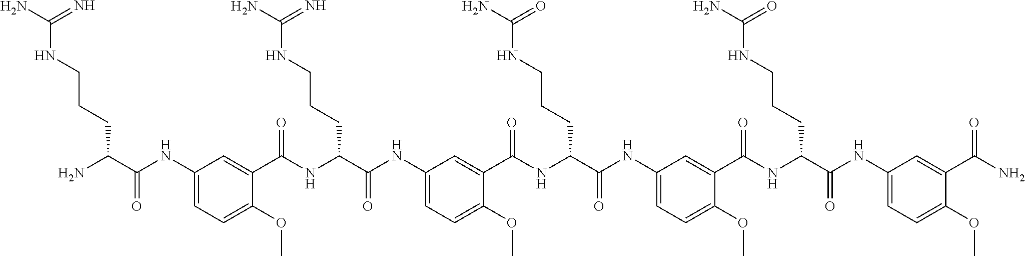 |
| 376 | 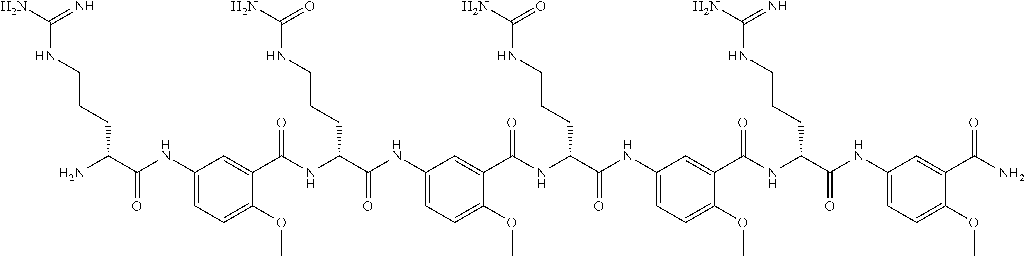 |
| 377 | 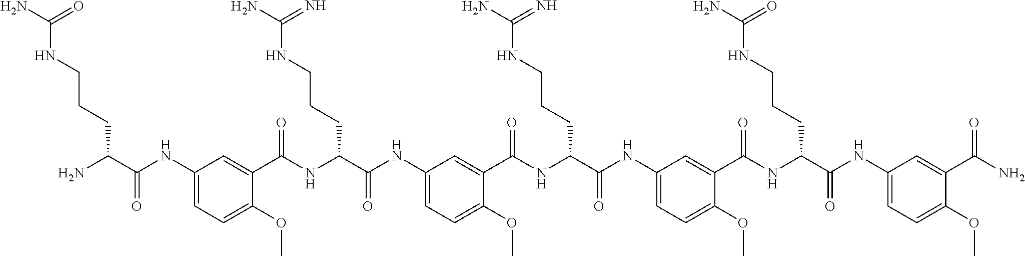 |
| 378 | 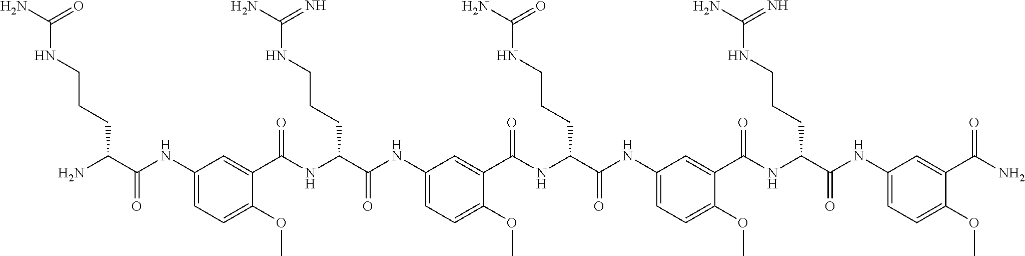 |
| 379 | 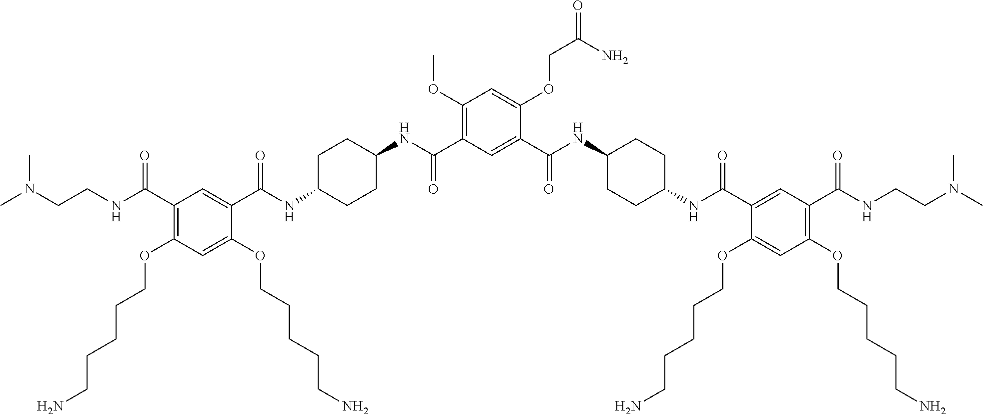 |
| 380 | 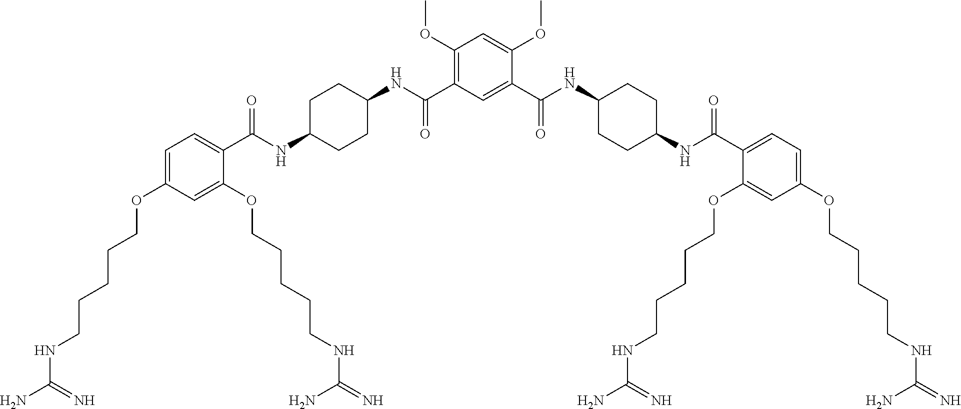 |
| 381 | 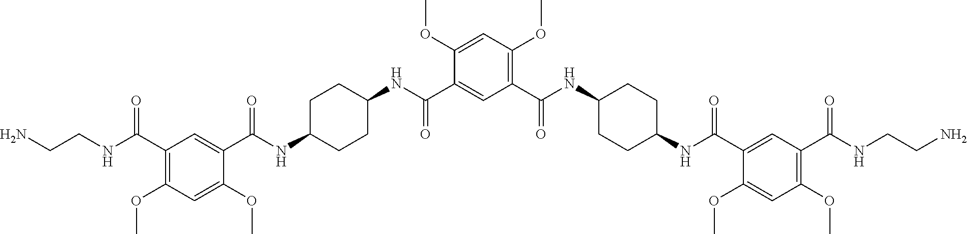 |
| 382 | 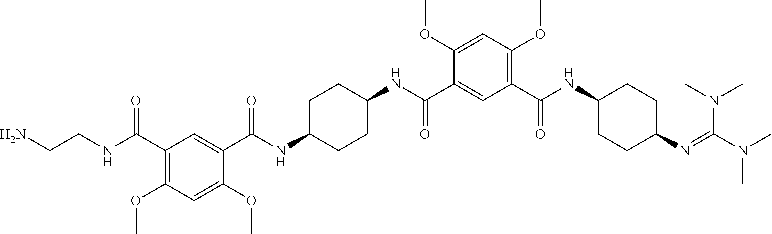 |
| 383 | 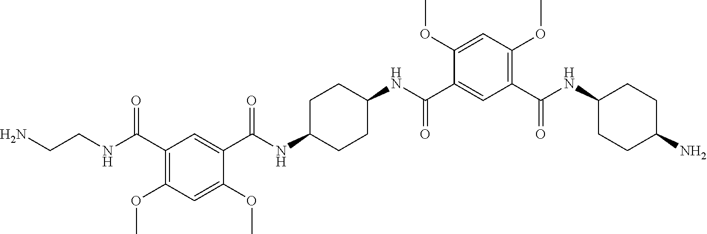 |
| 384 | 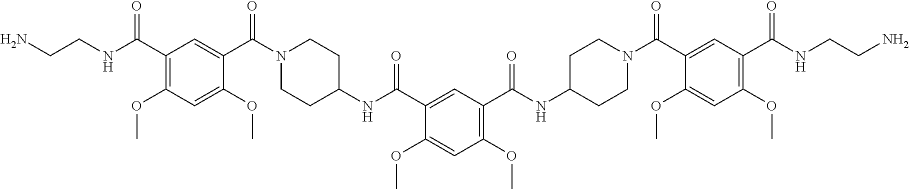 |
| 385 | 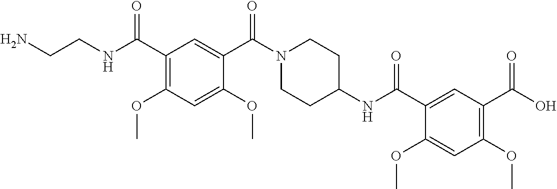 |
| 386 | 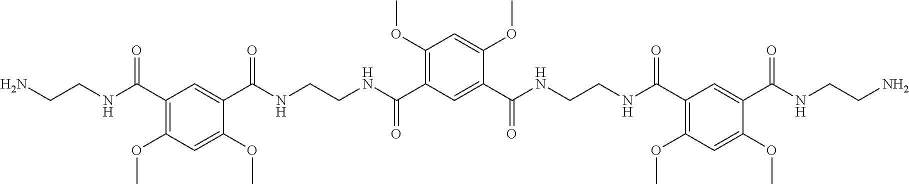 |
| 387 | 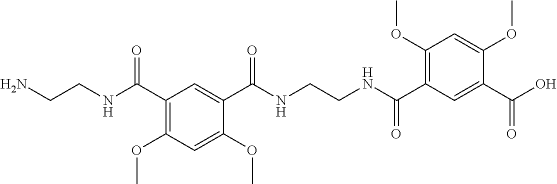 |
| 388 | 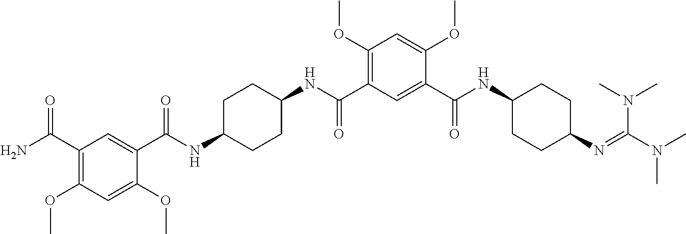 |
| 389 | 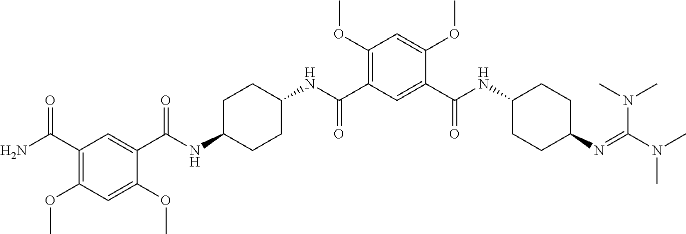 |
| 390 | 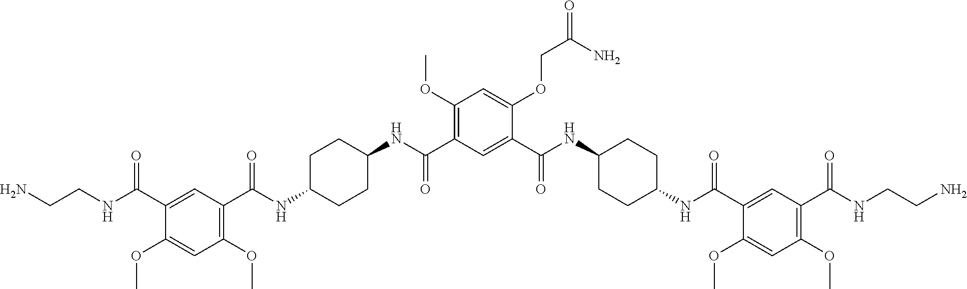 |
| 391 | 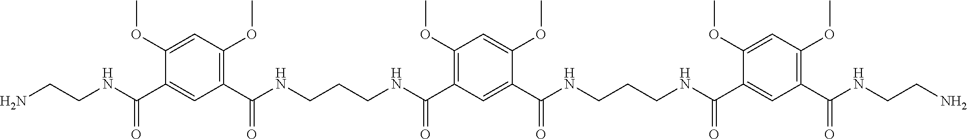 |
| 392 | 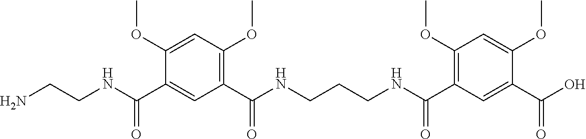 |
| 393 | 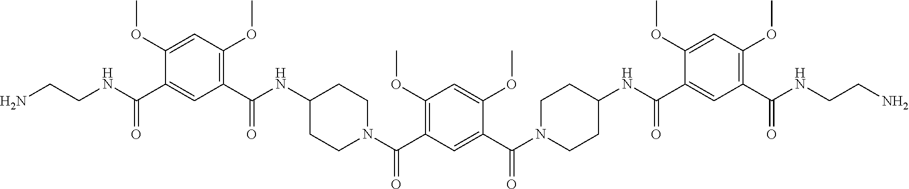 |
| 394 | 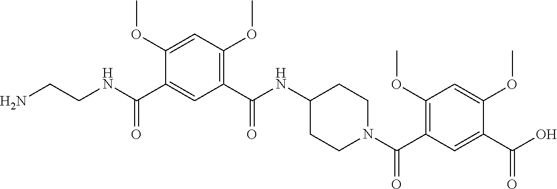 |
| 395 | 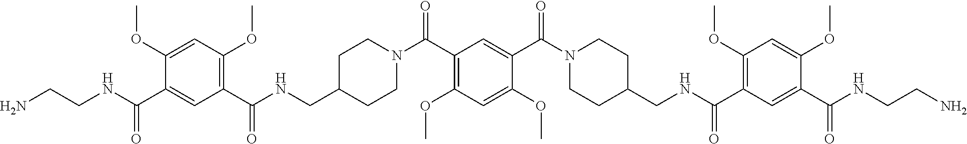 |
| 396 | 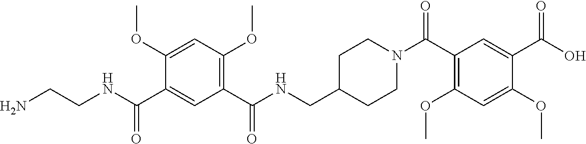 |
| 397 | 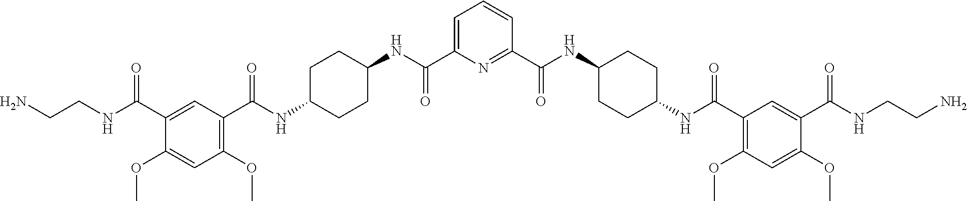 |
| 398 | 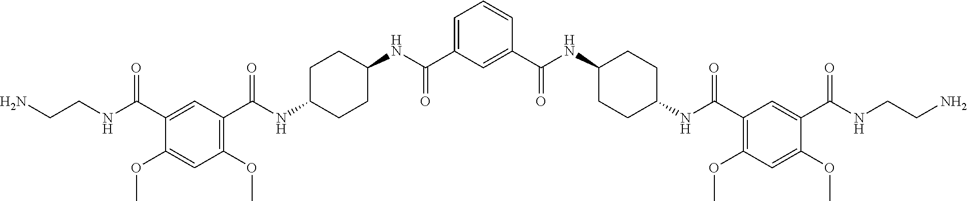 |
| 399 | 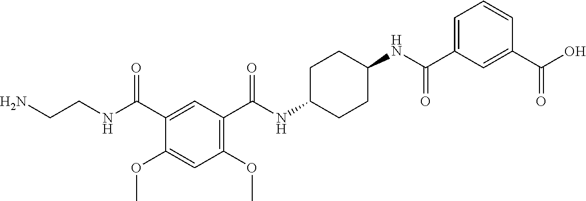 |
| 400 | 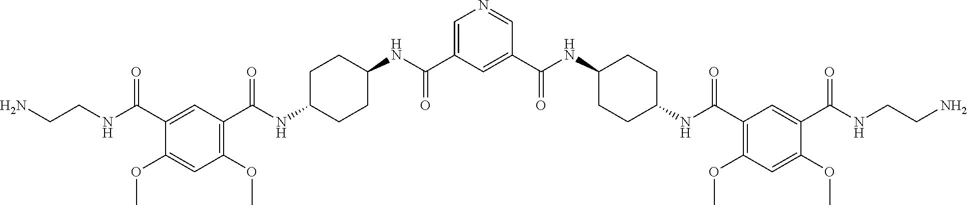 |
| 401 | 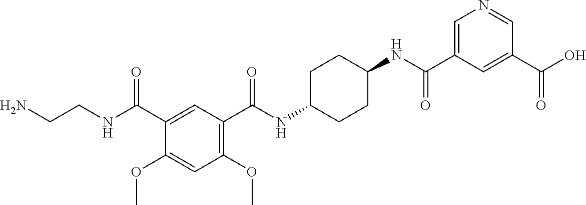 |
| 402 | 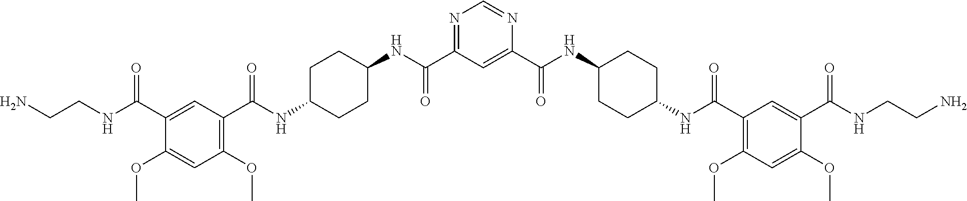 |
| 403 | 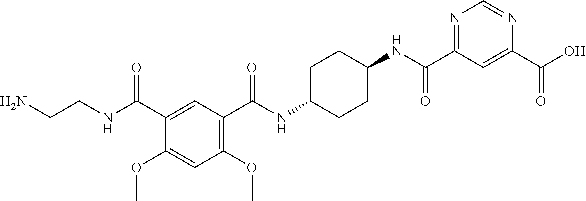 |
| 404 | 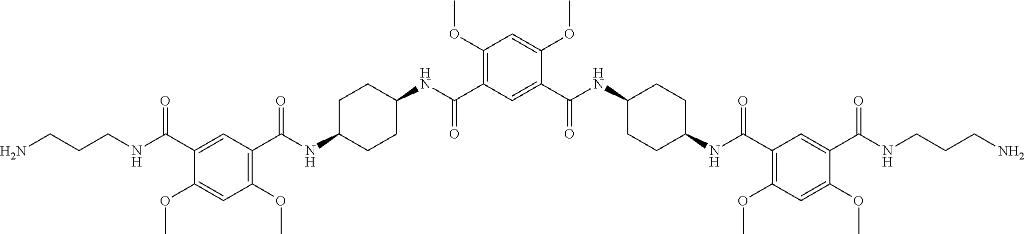 |
| 405 | 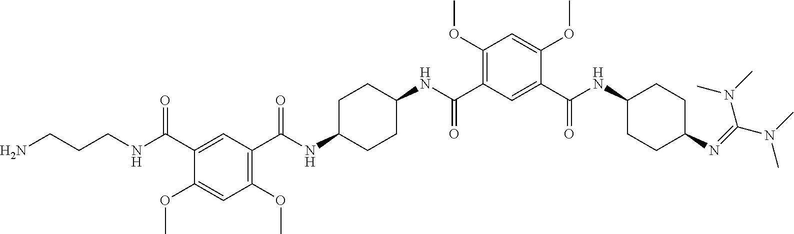 |
| 406 | 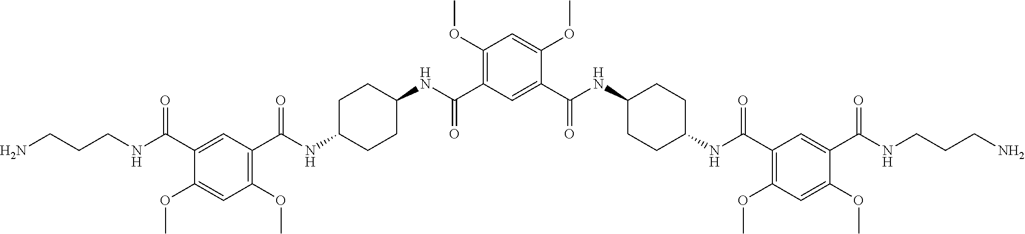 |
| 407 | 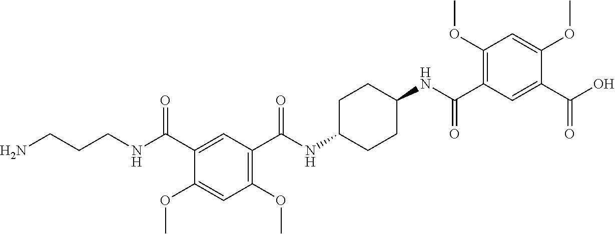 |
| 408 | 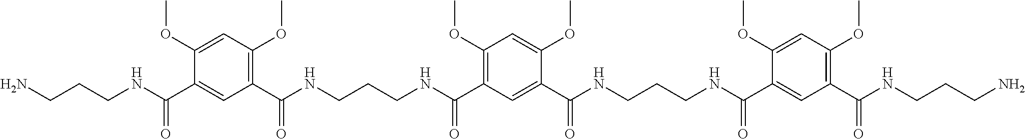 |
| 409 | 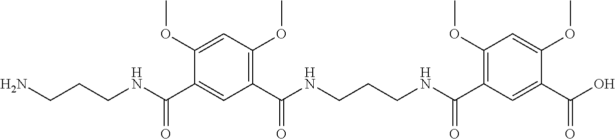 |
| 410 | 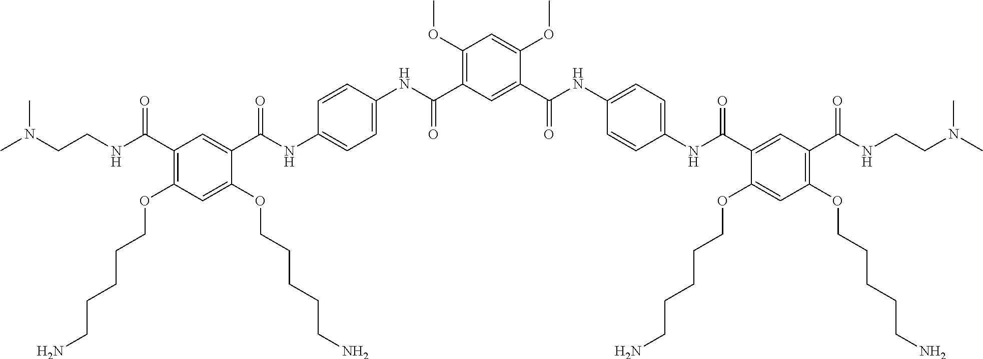 |
| 411 | 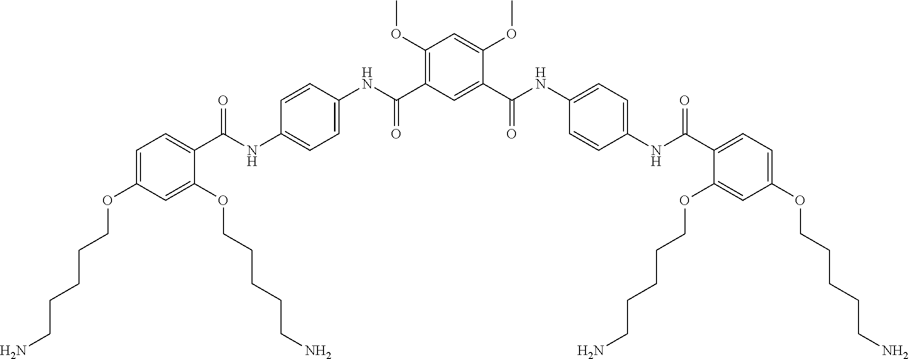 |
| 412 | 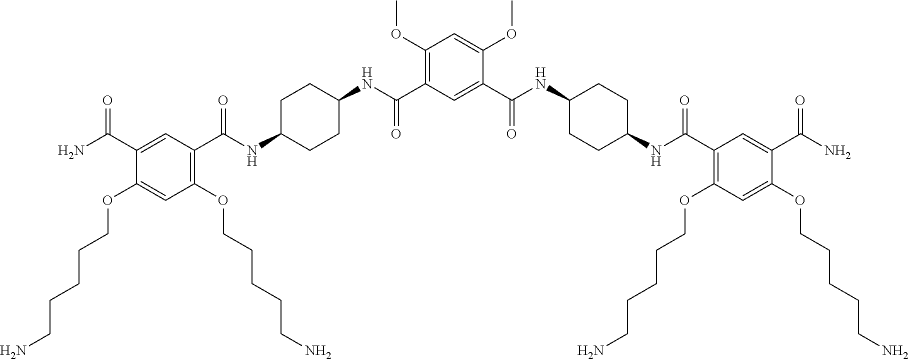 |
| 413 | 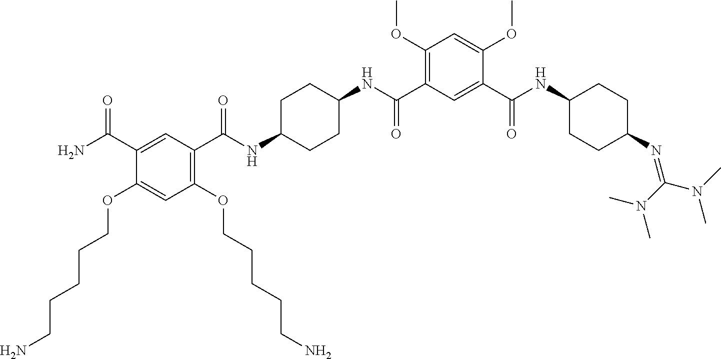 |
| 414 | 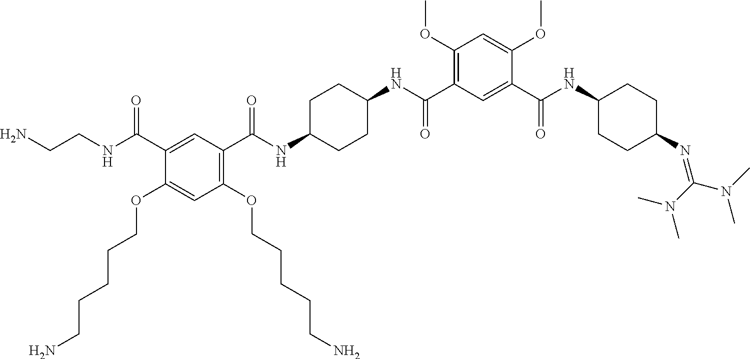 |
| 415 | 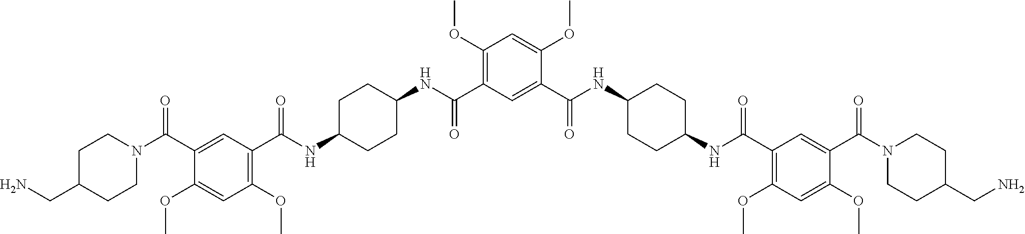 |
| 416 | 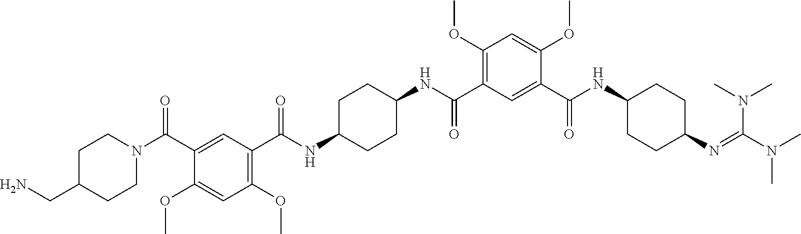 |
| 417 | 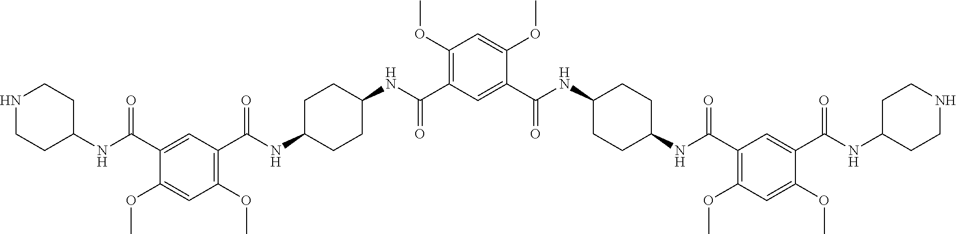 |
| 418 | 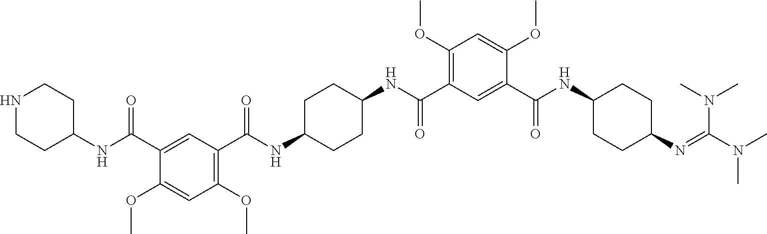 |
| 419 | 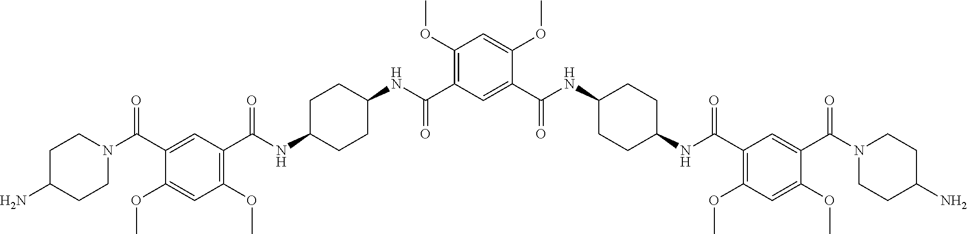 |
| 420 | 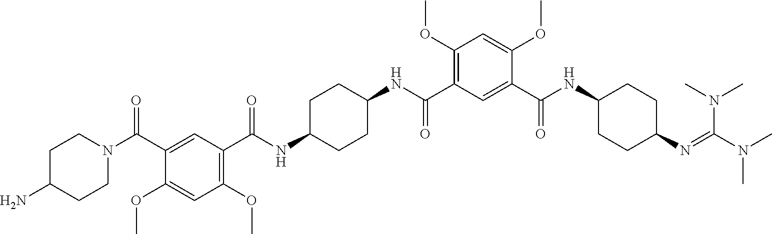 |
| 421 | 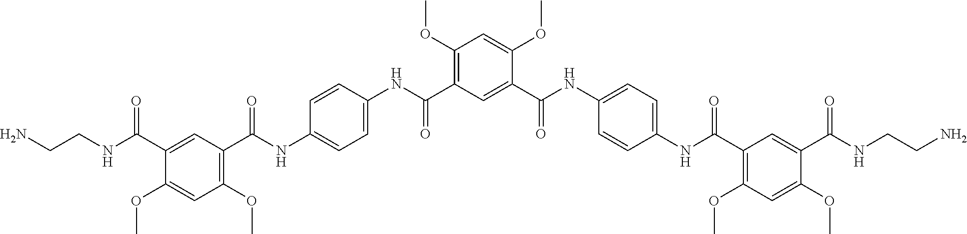 |
| 422 | 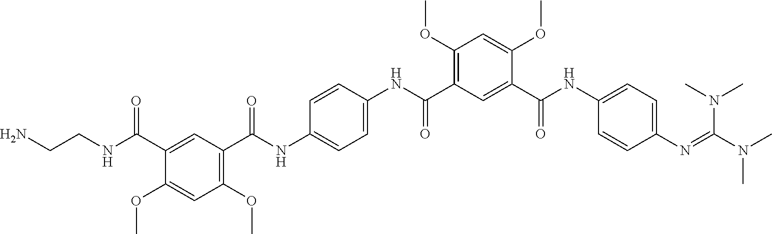 |
| 423 | 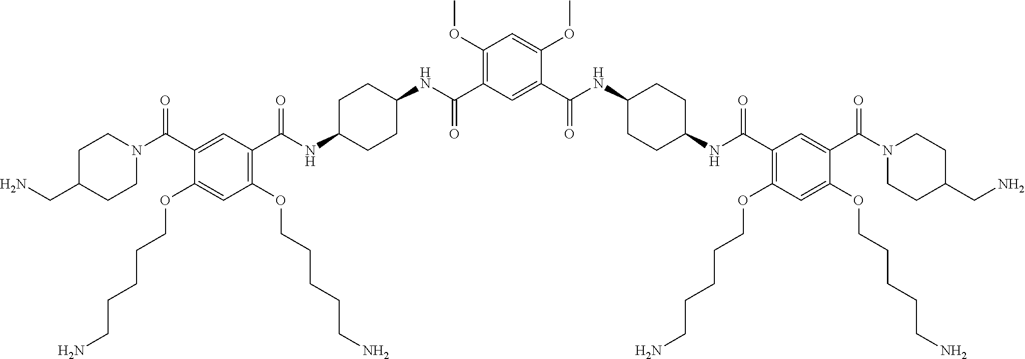 |
| 424 | 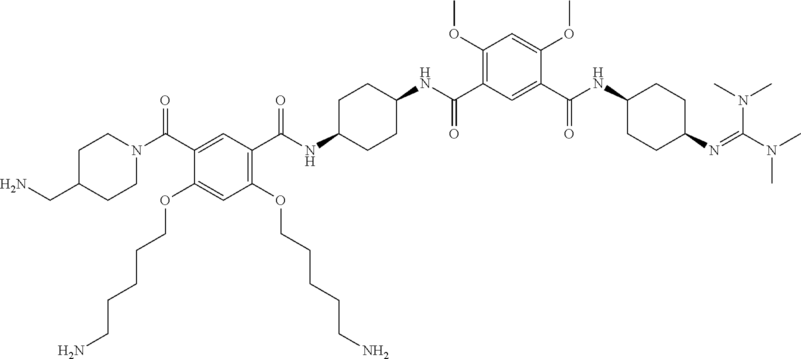 |
| 425 | 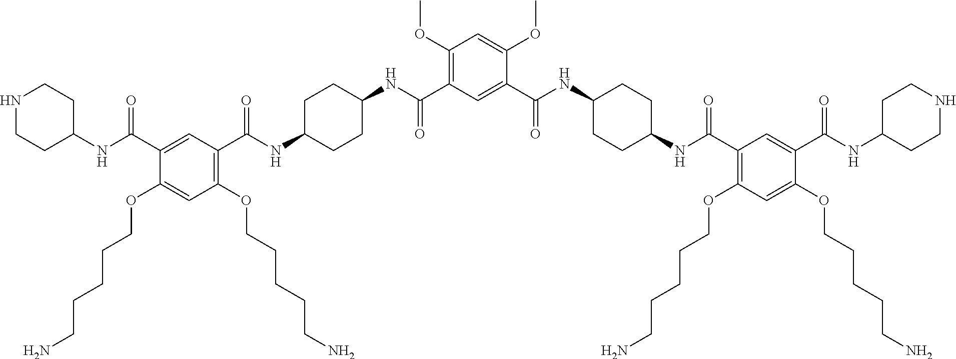 |
| 426 | 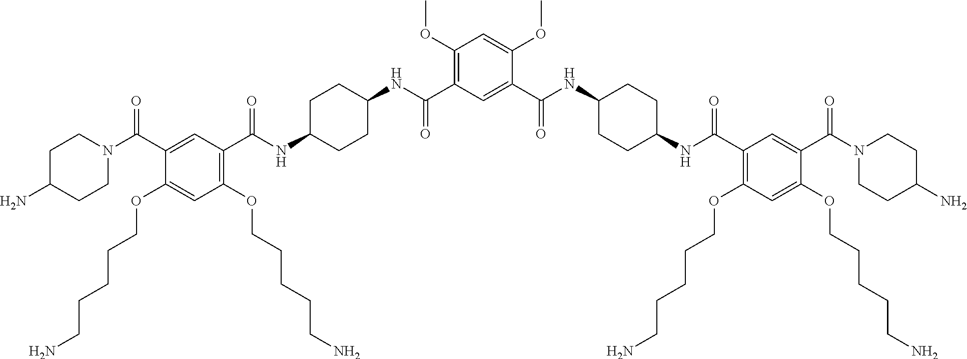 |
| 427 | 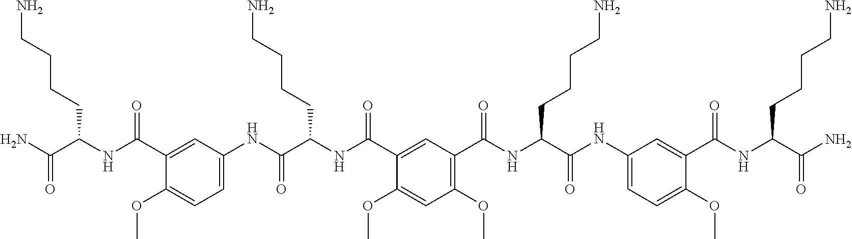 |

### Example 9

### Cytotoxicity

Colorimetric assay: Cytotoxicity was evaluated in a colorimetric assay using a transformed human liver cell line (HepG2, HB-8065) and an embryonic mouse cell line (NIH/3T3 cells, CRL-1658). This assay measures the bioreduction of a novel tetrazolium compound to a soluble formazan product by viable cells. HepG2 cells were seeded in 96 well plates at 3×10⁴ cells/well in MEM medium with 10% fetal bovine serum (FBS) 24 hours prior to use. NIH/3T3 cells were seeded in 96 well plates at 2×10⁴ cells/well in DMEM medium with 10% bovine calf serum (BCS) 24 hours prior to use. Cell monolayers were rinsed in serum-free media and incubated for one hour with test agent in serum-free media. After incubation, the media was replaced with serum supplemented media and live cells were measured using the Cell Titer 96 Aqueous Non-Proliferation Assay kit (Promega, Madison, Wis.). EC₅₀ values were determined using a four parameter logistic equation: Y=Bottom+(Top-Bottom)/(1+10̂((LogEC50-X)*HillSlope)). Results for some compounds are shown in Table 2.

Hemolysis assay: Cytotoxicity was also evaluated in a hemolysis assay using isolated human erythrocytes. Pooled whole human blood was centrifuged to separate the red blood cells (RBC). The isolated RBCs were rinsed and diluted in Tris-buffered saline (TBS buffer, pH 7.4) to obtain a 0.22% RBC stock suspension. Serial two-fold dilutions of test agent were assayed over a concentration range of 1000 to 0.48 μg/ml with shaking for 1 hour at 37° C. At the conclusion of the incubation time, samples were centrifuged and 30 μl of the supernatant was added to 100 μl H₂O. OD₄₀₅ measurements were read for hemoglobin concentration. The bee venom peptide melittin was used as a positive control. EC₅₀ values were determined using a four parameter logistic equation: Y=Bottom+(Top-Bottom)/(1+10̂((LogEC50-X)*HillSlope)). Results for some compounds are shown in Table 5.

TABLE 5

| | Hemolysis | Cytotoxicity | Cytotoxicity |
|---|---|---|---|
| Compound # | EC₅₀ (μg/mL) | HepG2 EC₅₀ (μg/mL) | 3T3 EC₅₀ (μg/mL) |
| protamine sulfate | >1000 | >1000 | >1000 |
| 221 | 936 | 108.4 | 84.6 |
| 229 | ~500 | >1000 | — |
| 246 | >1000 | 60 (80%) | — |
| 250 | — | >1000 | >1000 |
| 261 | — | >1000 | >1000 |
| 263 | >1000 | 255 (60%) | — |
| 265 | no significant decrease | — | — |
| 266 | >1000 | 534 | 838 |
| | — | >1000 | — |
| | — | 534 | 848 |
| 275 | — | 1000 (50%) | — |
| 278 | no significant decrease | — | — |
| 281 | >1000 | 1000 (50%) | — |
| 283 | >1000 | 1000 | 1000 |
| | >1000 | >1000 | >1000 |
| | — | — | — |
| 284 | 1000 (50%) | — | — |
| 287 | no significant decrease | 1000 (30%) | 1000 (50%) |
| 288 | no significant decrease | — | — |
| 297 | >1000 | 300 (60%) | — |
| 298 | no significant decrease | — | — |
| 303 | ~500 | — | — |
| 304 | ~500 | 220 (50%) | — |
| | — | >1000 | — |
| 305 | ~20 | 1000 (50%) | 1000 (50%) |
| | >1000 | >1000 | >1000 |
| 306 | ~1000 | ~1000 | — |
| 307 | ~50 | — | — |
| 308 | ~500 | — | — |
| 309 | ~100 | 1000 (50%) | 1000 (50%) |
| 310 | ~1000 | — | — |
| 311 | ~20 | 1000 (50%) | 1000 (50%) |
| | >1000 | >1000 | >1000 |
| 312 | 1000 (25%) | — | — |
| 313 | ~100 (20%) | — | — |
| 314 | 1000 (25%) | — | — |
| 317 | >1000 | — | — |
| 321 | >1000 | — | — |
| 322 | 1000 (50%) | — | — |
| 325 | >1000 | — | — |
| 329 | 18 (50%) | — | — |
| 330 | 5.4 (75%) | — | — |
| 331 | ~100 (50%) | — | — |
| 332 | 1000 | — | — |
| 333 | 1000 | — | — |
| 334 | ~15 | — | — |
| 335 | >1000 | 700 | 120 |
| 336 | >1000 | 505 | 390 |
| 340 | — | 1000 (60%) | 1000 (60%) |
| 346 | >1000 | >1000 | >1000 |
| 347 | — | >1000 | >1000 |
| 348 | >1000 | >1000 | >1000 |
| 349 | >1000 | 236 | 108 |
| 350 | >1000 | 425 | 150 |
| 351 | >1000 | — | — |
| 352 | >1000 | 758 | >1000 |
| 353 | >1000 | >1000 | ~100 |
| 354 | >1000 | >1000 | >1000 |
| 355 | >1000 | 133 | 52 |
| 356 | >1000 | — | — |
| 357 | >1000 | — | — |
| 358 | >1000 | — | — |
| 359 | >1000 | >1000 | >1000 |
| 360 | 973 | >1000 | >1000 |
| 361 | 1311 | 593 | 400.6 |
| 363 | >1000 | >1000 | 500 |
| 364 | 852 | 179 | 147 |
| 369 | >1000 | 280 | 100 |
| 379 | >1000 | >1000 | 650 |
| 380 | >1000 | 81.3 | 43.23 |
| 381 | 22.41 | 1129 | 1083 |
| 390 | >1000 | 891.6 | 985.6 |
| 397 | >1000 | 616.5 | 408.5 |
| 398 | >1000 | 1091 | >1000 |
| 400 | >1000 | 1304 | >1000 |
| 402 | >1000 | 863.5 | 753.3 |
| 404 | — | >1000 | 1096 |
| 406 | 12.12 | 208.8 | 215.3 |
| 410 | >1000 | 249.9 | 97.45 |
| 411 | >1000 | 590 | 86 |

### Example 10

### Clotting and Amidolytic Assays

aPTT clotting Assay: Unfractionated heparin was mixed with plasma at a final concentration of 0.4 U/mL (or concentration which increases aPTT time to between 120 and 300 seconds). Different concentrations of test compound were added (typically 0.15 to 20 μg/mL range). The ACL Elite Hemostasis analyzer (Beckman Coulter™) was used to add aPTT reagent (HemosIL SynthASil) to supplemented plasma. Clotting was initiated by addition of CaCl₂ and time to clot was recorded. EC₅₀ values were determined using a curve fit program (GraphPad Prism 5). Results for numerous compounds are shown in Table 6.

FXa Amidolytic Assay: LMWH (enoxaparin or tinzaparin) at final concentrations of 0.1 ug/ml, UFH at final concentrations of 0.03 units/mL, or fondaparinux at a final concentration of 0.02 μg/mL (or concentration which fully inhibits factor Xa) is combined with human antithrombin at a final concentration of 0.036 units/ml. Two μl of test agent are added (range between 0.01 and 23 ug/ml) and incubated for 5 minutes at 23° C. Bovine Factor Xa was added to a final concentration of 0.636 nkat/mL and incubated for a further 10 minutes at 23° C. Using a SpectraMax 250 (Molecular Devices, Inc.) and SoftMax Pro V.5 software, the plate is read every 30 seconds for 4 minutes, with a 10 second shaking before first read and maximum interval shaking Fit curve to report an EC₅₀ (50% reversal of anticoagulant effects) value for each compound: P(Cₚ)=1/[1+(K/Cₚ)ⁿ]. Results for numerous compounds are shown in Table 6.

TABLE 6

| | | | Tinzap | Fondap | Heparin |
|---|---|---|---|---|---|
| | aPTT | Enox FXa | FXa | FXa | FXa uM |
| Compound # | EC₅₀ (μM) | EC₅₀ (μM) | EC₅₀ (μM) | EC₅₀ (μM) | EC₅₀ (μM) |
| 201 | 6.000 | 0.974 | — | — | — |
| 202 | 4.550 | 0.398 | — | — | — |
| 203 | 2.987 | 0.283 | — | — | — |
| 204 | 3.016 | 0.239 | — | — | — |
| 205 | — | 0.202 | — | — | — |
| 206 | 1.965 | 0.148 | — | — | — |
| 207 | — | 0.039 | — | — | — |
| 208 | — | — | — | — | — |
| 209 | — | — | — | — | — |
| 210 | 2.552 | 0.082 | — | 1.403 | — |
| 211 | 5.904 | 0.137 | — | 2.866 | — |
| 212 | 0.883 | 0.073 | — | 0.259 | — |
| 213 | 0.893 | 0.078 | — | 0.354 | — |
| 214 | 1.907 | 0.087 | — | 0.432 | — |
| 215 | 2.852 | 0.073 | — | 0.288 | — |
| 216 | 1.220 | 0.347 | 0.191 | 7.867 | — |
| 217 | 1.117 | 0.074 | — | 0.252 | — |
| 218 | 0.853 | 0.074 | — | 0.176 | — |
| 219 | 1.170 | 0.095 | 0.187 | 0.731 | 0.153 |
| 220 | 5.452 | 0.125 | — | — | — |
| 221 | 1.173 | 0.107 | 0.095 | 0.304 | — |
| | 1.010 | 0.048 | 0.104 | 0.210 | 0.155 |
| 222 | 2.590 | 0.173 | 0.115 | 0.955 | — |
| 223 | 4.090 | 0.316 | 0.248 | 0.802 | — |
| 224 | 7.230 | 1.024 | 0.792 | 4.447 | — |
| 225 | 12.070 | 1.064 | 0.359 | — | — |
| 226 | 12.930 | 0.852 | 0.343 | — | — |
| 227 | >10 | — | — | — | — |
| 228 | 1.108 | 0.281 | >5 | >10 | — |
| 229 | 1.850 | 0.215 | 0.167 | 0.587 | — |
| | 2.270 | 0.207 | 0.164 | 0.322 | — |
| 230 | 2.780 | 0.252 | 0.147 | 4.877 | — |
| 231 | 2.310 | 0.192 | 0.099 | 3.559 | — |
| 232 | 3.240 | 0.862 | 0.536 | >10 | — |
| 233 | 1.590 | 0.376 | 0.268 | 3.460 | — |
| 234 | >10 | 1.015 | 0.446 | >10 | — |
| 235 | >10 | 0.497 | 0.433 | 10.680 | — |
| 236 | 1.930 | 0.099 | 0.396 | >12.6 | 0.364 |
| 237 | 1.140 | 0.177 | 0.109 | 3.615 | — |
| | — | 0.341 | 0.121 | 3.733 | 0.199 |
| 238 | 5.340 | 0.839 | 0.581 | 1.860 | — |
| 239 | 4.260 | 0.241 | 0.124 | 4.001 | — |
| 240 | >10 | 0.633 | 0.401 | 2.087 | — |
| 241 | 5.440 | 0.431 | 0.167 | >10 | — |
| 242 | 1.390 | >5 | >5 | >10 | — |
| 243 | 2.900 | 1.148 | 0.752 | 5.907 | — |
| 244 | 1.460 | 0.229 | 0.113 | 4.713 | — |
| 245 | 1.310 | 0.256 | 0.118 | 4.013 | — |
| | 1.460 | 0.224 | 0.125 | 3.194 | — |
| 246 | 2.370 | 0.514 | 0.368 | 2.535 | 0.110 |
| 247 | 2.570 | 3.143 | 1.351 | >10 | — |
| 248 | 2.080 | 0.208 | 0.158 | 1.839 | — |
| 249 | 1.420 | 0.466 | 0.215 | 10.576 | — |
| 250 | 2.730 | 3.280 | 1.287 | >10 | — |
| 251 | >10 | >5 | >5 | >10 | — |
| 252 | >10 | >5 | >5 | >10 | — |
| 253 | 1.660 | 1.068 | 0.472 | >10 | — |
| 254 | 1.640 | 0.093 | 0.097 | 0.448 | — |
| 255 | 1.720 | 0.114 | 0.119 | 0.473 | — |
| 256 | 1.450 | 0.233 | 0.151 | 2.498 | — |
| 257 | 1.330 | 0.205 | 0.130 | 1.447 | — |
| 258 | 1.520 | 0.198 | 0.132 | 1.542 | — |
| 259 | 1.570 | 0.267 | 0.180 | 2.877 | — |
| 260 | 1.660 | 0.399 | 0.247 | 5.085 | — |
| | 1.410 | 0.284 | 0.215 | 3.968 | — |
| 261 | 1.530 | 0.344 | 0.232 | 5.977 | — |
| | 2.150 | 0.294 | 0.214 | 6.255 | — |
| 262 | 1.260 | 0.344 | 0.213 | 5.571 | — |
| 263 | 1.250 | 0.376 | 0.352 | 1.600 | 0.179 |
| 264 | 1.380 | 0.731 | 0.365 | >10 | — |
| 265 | 1.450 | 0.138 | 0.120 | 0.385 | — |
| 266 | 2.440 | 0.122 | 0.122 | 0.214 | 0.210 |
| | 1.840 | 0.162 | 0.151 | 0.265 | — |
| | 3.480 | 0.164 | 0.121 | 0.368 | 0.247 |
| 267 | 1.670 | 0.543 | 0.313 | 6.991 | — |
| 268 | >10 | >5 | >5 | >10 | — |
| 269 | 1.780 | 0.247 | 0.165 | 5.108 | — |
| 270 | 1.250 | 0.533 | 0.349 | 4.052 | — |
| 271 | 1.640 | 0.219 | 0.135 | 3.116 | — |
| 272 | 3.090 | 0.541 | 0.240 | 7.694 | — |
| 273 | 1.550 | 0.319 | 0.202 | 3.799 | — |
| 274 | 1.630 | 0.558 | 0.299 | 7.015 | — |
| 275 | 1.720 | 0.134 | 0.113 | 0.579 | — |
| 276 | 2.330 | 0.625 | 0.363 | 5.181 | — |
| 277 | 1.550 | 0.581 | 0.285 | 10.761 | — |
| 278 | 1.990 | 0.150 | 0.132 | 0.408 | — |
| 279 | 1.950 | 0.154 | 0.134 | 0.775 | — |
| 280 | 1.630 | 0.133 | 0.120 | 0.581 | — |
| 281 | 1.810 | 0.129 | 0.111 | 0.441 | 0.108 |
| 282 | 2.000 | 0.121 | 0.114 | 0.424 | — |
| 283 | 1.390 | 0.146 | 0.124 | 0.575 | 0.101 |
| | 1.390 | 0.119 | 0.096 | 0.530 | — |
| | 1.840 | 0.070 | 0.096 | 0.452 | — |
| 284 | 1.560 | 0.144 | 0.138 | 0.390 | — |
| 285 | 2.150 | 0.169 | 0.151 | 0.799 | — |
| 286 | 1.900 | 0.118 | 0.104 | 0.526 | — |
| 287 | 1.980 | 0.114 | 0.108 | 0.402 | — |
| 288 | 2.020 | 0.119 | 0.102 | 0.390 | — |
| 289 | 1.760 | 0.133 | 0.108 | 0.508 | — |
| 290 | 2.270 | 0.318 | 0.212 | 1.878 | — |
| 291 | 2.485 | 0.047 | — | 1.178 | — |
| 292 | — | 0.450 | — | 4.789 | — |
| 293 | — | >10 | — | >10 | — |
| 294 | 2.740 | 0.264 | 0.264 | 0.244 | — |
| 295 | 3.990 | 0.312 | 0.325 | 0.387 | — |
| 296 | 15.260 | 0.286 | 0.296 | 0.437 | — |
| 297 | 7.120 | 0.293 | 0.318 | 0.225 | 0.215 |
| 298 | 2.060 | 0.201 | 0.167 | 0.334 | — |
| 299 | 2.900 | 0.243 | 0.242 | 0.265 | — |
| 300 | 3.430 | 0.272 | 0.311 | 0.462 | — |
| 301 | 6.080 | 0.276 | 0.278 | 0.295 | — |
| 302 | 10.630 | 0.298 | 0.278 | 0.491 | — |
| 303 | >10 | 0.192 | 0.189 | 0.146 | — |
| 304 | >10 | 0.256 | 0.257 | 0.209 | 0.242 |
| 305 | 5.020 | 0.186 | 0.192 | 0.153 | 0.343 |
| | 1.480 | 0.186 | 0.202 | 0.218 | — |
| | 4.360 | 0.059 | 0.242 | 0.058 | — |
| | — | 0.094 | 0.101 | 0.091 | — |
| 306 | >10 | 0.198 | 0.199 | 0.134 | 0.236 |
| | >10 | 0.338 | 0.367 | 0.295 | — |
| 307 | 3.780 | 0.120 | 0.118 | 0.118 | — |
| 308 | >10 | 0.157 | 0.136 | 0.192 | — |
| 309 | 2.270 | 0.131 | 0.137 | 0.120 | 0.301 |
| 310 | >10 | 0.119 | 0.141 | 0.098 | 0.242 |
| 311 | 2.540 | 0.101 | 0.108 | 0.090 | 0.245 |
| | 1.780 | 0.140 | 0.150 | 0.154 | — |
| 312 | >10 | 0.152 | 0.169 | 0.120 | 0.295 |
| 313 | 3.810 | 0.107 | 0.115 | 0.117 | 0.196 |
| 314 | >10 | 0.208 | 0.199 | 0.265 | 0.295 |
| 315 | 2.050 | 1.791 | 0.492 | >10 | — |
| 316 | 2.040 | 0.329 | 0.223 | 1.749 | — |
| 317 | 1.610 | 0.293 | 0.178 | 0.873 | — |
| 318 | 1.410 | 0.407 | 0.261 | 3.051 | — |
| 319 | 2.440 | 0.276 | 0.178 | 1.051 | — |
| 320 | 2.420 | 0.901 | 0.285 | 4.891 | — |
| 321 | 1.820 | 0.441 | 0.225 | 2.692 | — |
| 322 | 2.110 | 0.195 | 0.158 | 0.417 | — |
| 323 | 2.960 | 0.326 | 0.250 | 1.158 | — |
| 324 | 2.930 | 0.784 | 0.438 | 3.841 | — |
| 325 | 2.700 | 0.505 | 0.213 | 2.227 | — |
| 326 | 1.990 | 0.289 | 0.234 | 0.549 | — |
| 327 | 1.930 | 1.032 | 0.470 | 6.773 | — |
| 328 | 1.880 | 0.448 | 0.257 | 2.330 | — |
| 329 | 7.910 | 0.202 | 0.203 | 0.371 | — |
| 330 | >10 | 0.169 | 0.182 | 0.218 | — |
| 331 | >10 | 0.155 | 0.184 | 0.161 | — |
| 332 | >10 | >5 | >5 | >5 | >5 |
| 333 | >10 | >5 | >5 | >5 | >5 |
| 334 | >10 | 0.249 | 0.281 | 0.224 | — |
| 335 | 1.700 | 0.185 | 0.148 | 1.563 | 0.151 |
| | 1.409 | 0.201 | 0.120 | 1.392 | — |
| 336 | 1.427 | 0.079 | 0.133 | 0.091 | — |
| | 1.225 | 0.091 | 0.084 | 0.063 | — |
| 337 | — | 0.225 | 0.174 | 1.832 | — |
| 338 | — | 0.241 | 0.176 | 2.750 | — |
| 339 | 0.910 | 0.272 | 0.235 | 1.900 | — |
| 340 | 0.720 | 0.119 | 0.115 | 0.208 | 0.292 |
| 341 | — | 0.198 | 0.138 | 1.576 | — |
| 342 | 2.350 | 0.153 | 0.147 | 0.167 | — |
| 343 | — | 0.235 | 0.201 | 0.932 | — |
| 344 | 0.680 | 0.392 | 0.244 | 2.062 | — |
| 345 | 2.990 | 0.233 | 0.205 | 0.911 | — |
| 346 | 8.810 | 0.198 | 0.410 | 0.146 | 0.772 |
| 347 | >10 | >5 | >5 | >10 | >20 |
| 348 | 2.400 | 0.120 | 0.067 | 0.263 | 0.153 |
| 349 | 2.020 | 0.108 | 0.090 | 0.129 | 0.213 |
| 350 | 0.627 | 0.147 | 0.128 | 0.146 | 0.291 |
| 351 | 1.930 | 0.214 | 0.114 | 1.106 | 0.194 |
| 352 | 2.270 | 0.299 | 0.162 | 0.934 | 0.287 |
| 353 | 1.630 | 0.123 | 0.104 | 0.407 | 0.198 |
| 354 | 2.200 | 0.117 | 0.109 | 0.202 | 0.214 |
| 355 | 1.410 | 0.095 | 0.067 | 0.800 | 0.147 |
| 356 | 1.890 | 0.127 | 0.100 | 0.512 | 0.204 |
| 357 | 1.420 | 0.248 | 0.118 | 2.220 | 0.187 |
| 358 | 1.930 | 0.324 | 0.184 | 1.601 | 0.314 |
| 359 | 1.390 | 0.090 | 0.073 | 0.115 | 0.189 |
| 360 | >10 | >5 | >5 | >10 | >20 |
| 361 | 9.850 | 0.766 | 0.126 | >10 | 0.202 |
| 362 | >10 | 0.197 | 0.805 | >10 | 0.558 |
| 363 | 1.030 | 0.067 | 0.113 | 1.065 | 0.134 |
| 364 | 1.250 | 0.109 | 0.135 | 0.806 | 0.000 |
| 365 | >10 | >5 | >5 | >10 | >20 |
| 366 | >10 | >5 | >5 | >10 | 15.502 |
| 367 | 1.270 | 0.042 | 0.106 | 0.514 | 0.085 |
| 368 | 9.250 | 10.956 | 3.388 | >10 | 3.688 |
| 369 | 0.350 | 0.143 | 0.176 | 3.865 | 0.411 |
| 370 | 1.460 | 0.463 | 0.259 | >10 | 12.475 |
| 371 | 3.260 | 0.252 | 0.197 | >10 | >10 |
| 372 | 2.970 | 0.225 | 0.150 | 0.544 | >10 |
| 373 | 2.360 | 0.123 | 0.123 | >10 | >10 |
| 374 | 2.420 | 0.157 | 0.114 | 1.815 | 0.677 |
| 375 | 2.690 | 0.113 | 0.076 | 1.259 | 0.681 |
| 376 | 1.890 | 0.191 | 0.114 | 1.670 | 0.416 |
| 377 | 2.090 | 0.149 | 0.092 | 1.435 | 0.660 |
| 378 | 2.01 | 0.110 | 0.073 | 2.328 | 0.490 |
| 379 | 1.62 | 0.464 | 0.095 | >10 | 0.130 |
| 380 | 1.690 | 0.089 | 0.071 | 0.180 | 0.164 |
| 381 | 2.690 | 0.289 | 0.274 | 0.493 | 0.777 |
| 382 | >10 | >5 | >5 | >10 | >20 |
| 383 | >10 | >5 | >5 | >10 | >20 |
| 384 | >10 | >5 | >5 | >10 | >20 |
| 385 | >10 | >5 | >5 | >10 | >20 |
| 386 | — | >5 | 7.239 | >10 | 19.357 |
| 387 | >10 | >5 | >5 | >10 | >20 |
| 388 | >10 | >5 | >5 | >10 | >20 |
| 389 | >10 | 5.608 | 6.729 | >10 | >10 |
| 390 | 2.080 | 0.707 | 0.708 | 1.281 | — |
| 391 | >10 | >5 | >5 | >10 | — |
| 392 | >10 | >5 | >5 | >10 | — |
| 393 | >10 | >5 | >5 | >10 | — |
| 394 | >10 | >5 | >5 | >10 | — |
| 395 | >10 | >5 | >5 | >10 | — |
| 396 | >10 | >5 | >5 | >10 | — |
| 397 | 1.150 | 0.985 | 0.955 | 2.627 | — |
| 398 | 4.770 | 2.534 | 2.035 | 13.919 | — |
| 399 | >10 | >5 | >5 | >10 | — |
| 400 | 5.090 | 1.218 | 1.183 | 5.513 | — |
| 401 | >10 | >5 | >5 | >10 | — |
| 402 | 5.520 | 5.812 | 3.184 | 5.856 | — |
| 403 | >10 | >5 | >5 | >10 | — |
| 404 | 2.570 | 0.350 | 0.445 | 0.325 | — |
| 405 | >10 | >5 | >5 | >10 | — |
| 406 | 2.460 | 0.299 | 0.436 | 0.113 | — |
| 407 | >10 | >5 | >5 | >10 | — |
| 408 | >10 | >5 | >5 | >10 | — |
| 409 | >10 | >5 | >5 | >10 | — |
| 410 | 0.640 | 0.056 | 0.068 | 0.041 | — |
| 411 | 1.150 | 0.096 | 0.123 | 0.061 | — |
| 412 | 0.687 | 0.125 | 0.113 | 0.197 | — |
| 413 | 7.063 | 4.650 | 2.765 | 14.791 | — |
| 414 | >10 | 3.317 | 2.055 | >10 | — |
| 415 | >10 | >5 | >5 | >10 | — |
| 416 | >10 | >5 | >5 | >10 | — |
| 417 | 2.221 | 0.495 | 0.200 | 1.108 | — |
| 418 | >10 | >5 | >5 | >10 | — |
| 419 | >10 | >5 | >5 | >10 | — |
| 420 | >10 | >5 | >5 | >10 | — |
| 421 | 1.204 | 0.238 | 0.220 | 0.159 | — |
| 422 | 1.247 | 0.608 | 0.426 | 1.887 | — |
| 423 | 3.152 | 1.177 | 0.296 | >10 | — |
| 424 | >10 | >5 | >5 | >10 | — |
| 425 | 0.429 | 0.088 | 0.062 | 0.502 | — |
| 426 | 11.319 | 2.750 | 0.530 | >10 | — |
| 427 | >10 | >5 | >5 | >10 | — |

### Example 11

### In Vivo Neutralization of Unfractionated Heparin in the Rat

The male Sprague-Dawley rats used in this study were obtained from Charles River Laboratories, Raleigh. They were nine-weeks-old at the start of the study and their weights ranged from 279-334 g. Rats were pre-treated with UFH administered by IV injection in a tail vein at 100 U/kg in a dose volume of 1 mL/kg. The rats were then treated with a single IV injection of saline, protamine or the appropriate test compound at doses of 0.25, 0.5 and 1.0 mg/kg. All treatments were dosed in a volume of 1 mL/kg. Blood was collected via the orbital sinus from three rats per group at the following time points after treatment: predose, 1, 3, 10, 30 and 60. At each time point, 1 mL of blood was collected from each animal into a single tube. The blood was analyzed using an AMEX Destiny Plus Coagulation Analyzer for activated partial thromboplastintime (APTT) and anti-Factor Xa.

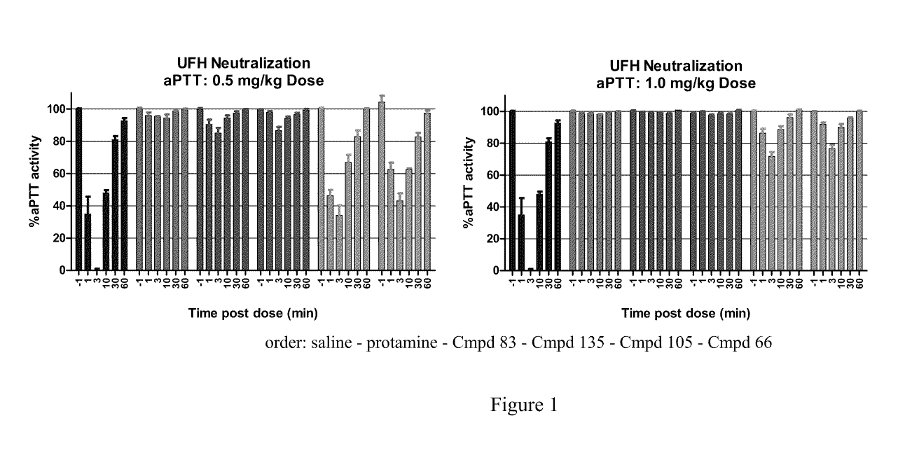

Two compounds, Compound 283 (a salicylamide) and Compound 335 (an arylamide) show complete neutralization of UFH activity in aPTT assays at a 1 mg/kg dose and are as efficacious as protamine. Results are shown in FIG. 1.

### Example 12

### In Vivo Neutralization of Enoxaparin in the Rat

Compounds were tested for their ability to neutralize enoxaparin coagulation inhibition in rats. Male Sprague-Dawley rats were used in this study (Charles River Laboratories). They were ten-weeks-old at the start of the study and their weights ranged from 319-362 g. Enoxaparin (2 mg/kg) was administered by IV injection to groups of six rats. After 3 min, saline, protamine or a test compound was administered by IV injection. Blood was collected before dosing with enoxaparin, and at 1, 3, 10, 30 and 60 min after dosing with the standard and test compounds. All treatments were dosed in a volume of 1 mL/kg. Blood was collected via the orbital sinus from three rats per group. At each time point, 1 mL of blood was collected from each animal into a single tube. The blood was analyzed using an AMEX Destiny Plus Coagulation Analyzer for activated partial thromboplastin time (APTT) and anti-Factor Xa (low-molecular weight).

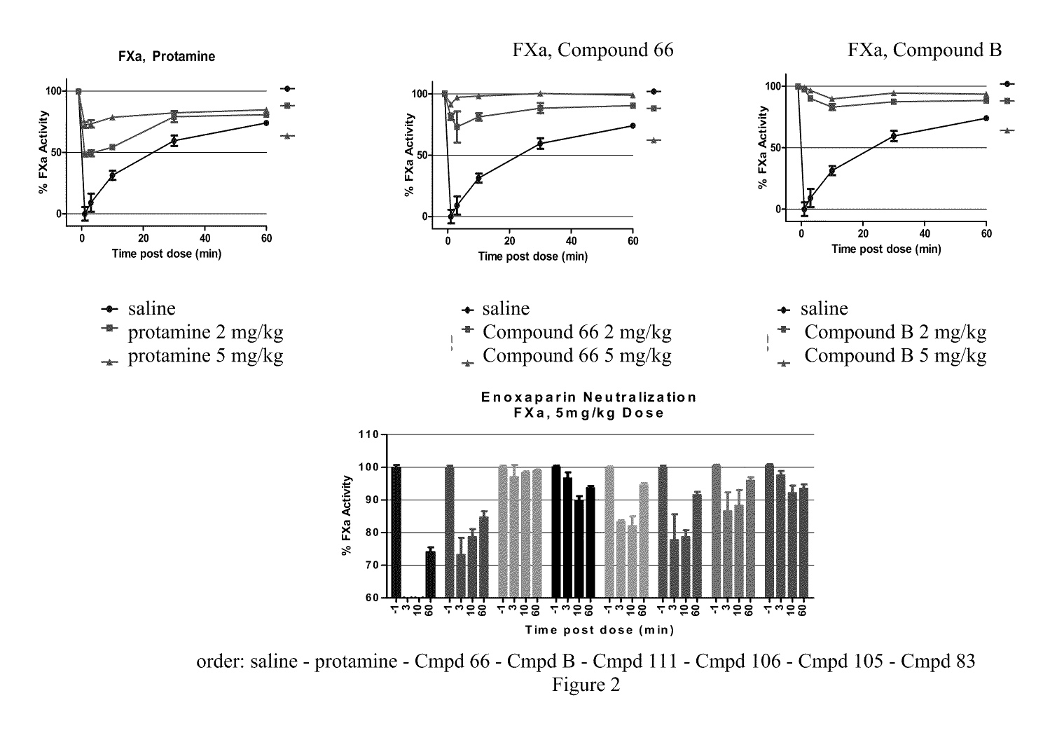

All test compounds were found to be more efficacious at neutralizing enoxaparin than protamine in both aPTT and factor Xa assays (FIG. 2). Compound 266, a Lys derivative, was the most efficacious of all compounds tested. Protamine only partially reverses enoxaparin activity, returning FXa activity to approximately 80% of pretreatment levels (blue bars), while 5 mg/kg Compound 266 completely neutralizes enoxaparin within minutes (green bars). Both Compound B (arylamide) and Compound 283 (Arg derivative) rapidly returned FXa levels to greater than 90% of pretreatment levels. The 4-Arg containing compounds (Compound 305, Compound 311) are marginally more efficacious than the 3-Arg derivative (Compound 306). Results are shown in FIG. 2.

Compound B has the following formula:

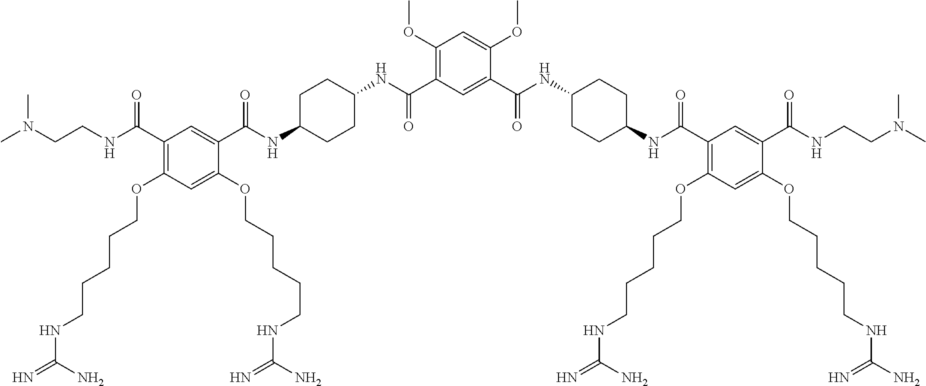

### Example 13

### Normalization of Enoxaparin-Extended Bleeding Times in a Rat Tail Transection Model

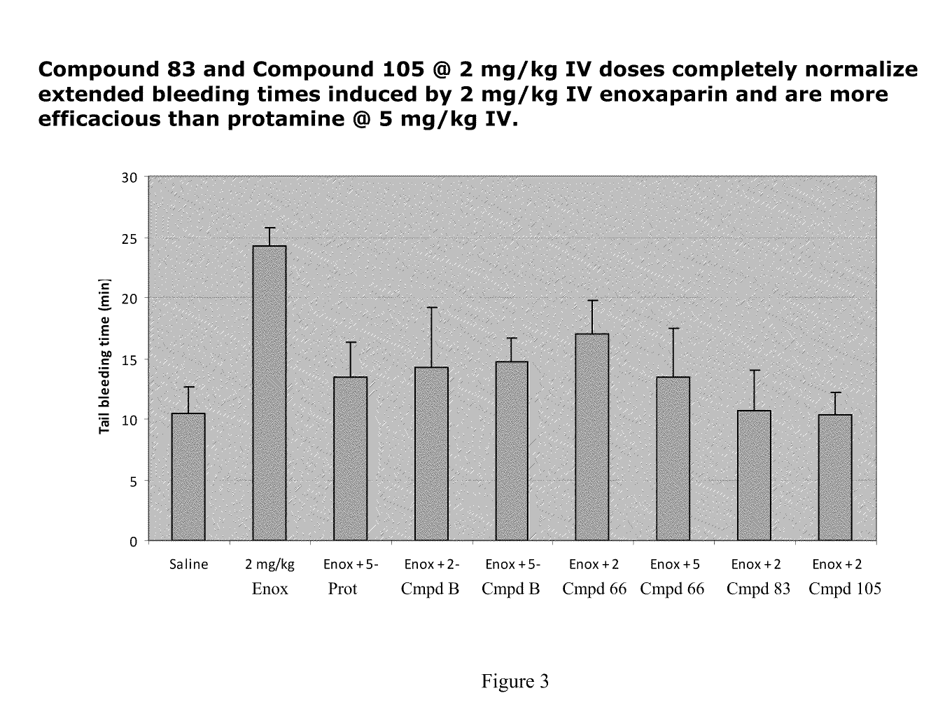

Studies have now been done to examine effects on extended bleeding times caused by enoxaparin treatment. Male Sprague Dawley rats (Charles River) were administered 2 mg/kg enoxaparin by IV injection in the tail vein, followed 3 minutes later by test agent (IV, tail vein) at 2 and 5 mg/kg doses. Tails were then rapidly transected and bleeding time onto an absorbant pad was determined. Enoxaparin alone (no antagonist) extends bleeding time from 11 to 24 seconds. Compound 283 and Compound 305 (2 mg/kg) completely neutralized the prolonged bleeding times. Compound 266 was not as effective showing incomplete neutralization even at 5 mg/kg. The arylamide Compound B was tested in this model and also found to not completely reverse the effect of 2 mg/kg enoxaparin when administered at 5 mg/kg. Significantly, as opposed to Compound 283 and Compound 305, only partial restoration to normal bleeding time was obtained with protamine at a 5 mg/kg dosage. Therefore, both Compound 283 and Compound 305 are superior to protamine in neutralization of anti-FXa and extended bleeding times caused by enoxaparin. Results are shown in FIG. 3.

### Example 14

### In Vivo Neutralization of Fondaparinux in the Rat

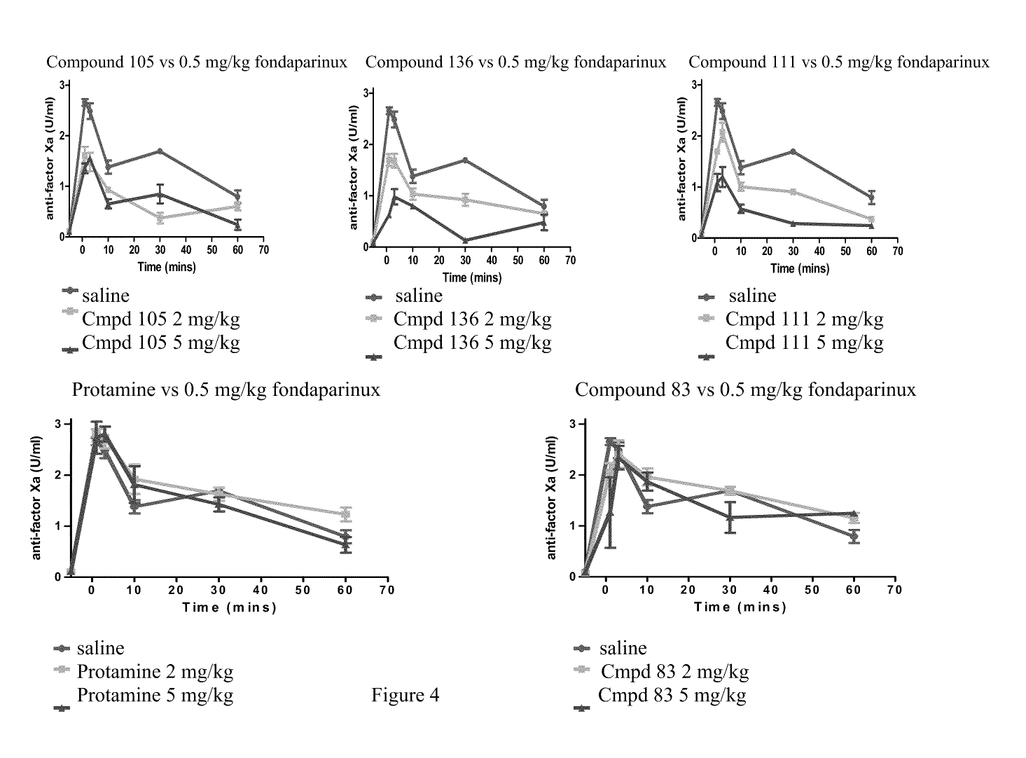

Compounds were selected to test fondaparinux neutralization in vivo. Rats were pre-treated with fondaparinux administered by IV injection at 0.5 mg/kg. The rats were then treated with a single IV injection of saline, protamine or the PMX compound. Blood was collected via the orbital sinus from three rats per group at the following time points: pre-dose, 1, 3, 10, 30 and 60 min. Plasma samples were prepared for analysis of anti-factor Xa activity using an AMEX Destiny Plus Coagulation Analyzer. Results are shown in FIG. 4.

Protamine administered at 2 and 5 mg/kg did not reduce Factor Xa levels in treated rats. The same lack of effectiveness was also observed for Compound B and Compound 283.

Compound 311 exhibited significant anti-Factor Xa activity. At 1 min after dosing, when Factor Xa activity was at its peak, the levels in the rats treated with 2 and 5 mg/kg were already markedly reduced. The levels of Factor Xa activity at the two dosages were significantly lower at the 10 and 30 min, and then returned to approximate baseline levels by 60 min.

Compound 305 was effective in reducing Factor Xa activity. At 1 min after dosing, Factor Xa levels in the groups treated with 2 and 5 mg/kg were noticeably reduced. Interestingly, the level for both doses was about the same. The levels at 10 and 30 min were also significantly lower than the saline controls, and then returned to approximate baseline levels by 60 min.

Compound 266 was effective at the higher 5 mg/kg dose.

Compound 336 was also effective. At both dose levels, significant reductions in anti-Factor Xa activity were observed.

There is excellent correlation in the rank order of anti-fondaparinux activity in vitro with efficacy in vivo; compounds with EC₅₀ values ≦0.2 μM versus fondaparinux in in vitro fXa assays are efficacious in vivo. This is strong validation of the in vitro assay and sets the target activity for new compound synthesis.

### Example 15

### Mitigation of Hemodynmic Responses in the Anesthetized Rat

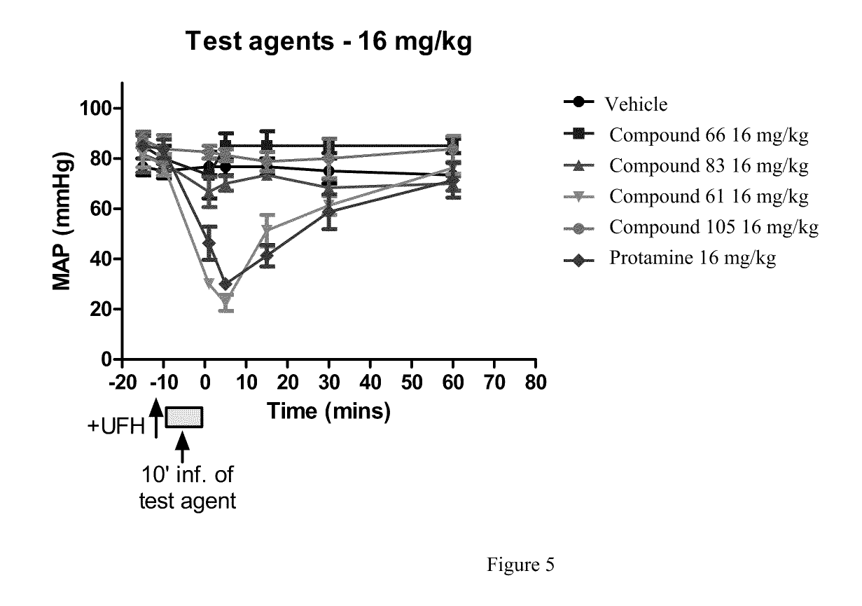

As a key safety issue for cationic compounds is reduction in blood pressure shortly after administration. To address this hemodynamic issue, a medicinal chemistry strategy with literature precedence of introducing carboxylic acid functionality was applied to the salicylamide series of compounds. Many of the compounds prepared to test this approach displayed significant in vitro activity for neutralizing the effect of heparin and/or low molecular weight heparins and four compounds were selected for safety studies measuring hemodynamic effects. Surgically prepared animals were purchased from Charles River Laboratories, Raleigh, N.C. Animals were anesthetized on the day of experiment with isoflurane (1.8-4%). Blood pressure and heart rate data were collected on a Grass Polygraph recorder. The test articles, vehicle or protamine dosing preparations were administered once to each rat by a 10 minute intravenous infusion three minutes following a single intravenous injection of heparin (50 U/kg). Each animal received a dose volume of 2.0 mL/kg. Blood pressure was recorded prior to treatment for approximately 1 minute and immediately following heparin, immediately following vehicle, test articles or protamine and at 5, 15, 30, and 60 minutes following dosing. The doses of test agent were either 8 mg/kg or 16 mg/kg. The differences in hemodynamic effect at 16 mg/kg were especially profound (FIG. 5). Three compounds that were highly efficacious in vitro and in vivo (Compound 266, Compound 283, and Compound 305) were all found to possess a hemodynamic response profile superior to protamine. The location of the carboxylic acid functionality in the molecule is important as Compound 261 did not show an improved hemodynamic effect profile however. Results are shown in FIG. 5.

The following observations have been made:

For salicylamide compounds, incorporation of a terminal aromatic moiety dramatically increases activity against fondaparinux. D-Arg and L-Arg analogs further increase activity against fondaparinux. Thus, in some embodiments of the invention, the salicylamide compounds described herein may be modified by incorporation of a terminal aromatic moiety, or D-Arg, or L-Arg.

For salicylamide compounds, addition of an acidic group mitigates some of the adverse hemodynamic side-effect. Thus, in some embodiments of the invention, the salicylamide compounds described herein may be modified by addition of an acidic group.

Potent LMWH-antagonists with superior anti-fondaparinux activity include the following salicylamides: compound 305, compound 311, compound 266, compound 348, compound 354, compound 283; and the following arylamides: compound B, compound 336, compound 411, and compound 363.

Identified UFH- and LMWH-antagonists with reduced hemodynamic liabilities include the following salicylamides: compound 283, compound 305, and compound 266; and the following arylamides: compound B.

Compounds with in vivo efficacy versus fondaparinux include the following salicylamides: compound 305 and compound 311; and the following arylamides: compound 336.

Compounds with in vivo efficacy versus LMWH superior to protamine by anti-factor Xa activity include the following salicylamides: compound 266, compound 305, compound 283, and compound 348; and the following arylamides: compound B, compound 369, and compound 363; and bleeding time include the following salicylamides: compound 305 and compound 283.

Salicylamide compounds 283, 305, and 266 and arylamides compound B demonstrate the following activities: potent anti-UFH and anti-LMWH activity in vitro; potent anti-UFH activity in vivo; potent anti-LMWH activity in vivo (improved over protamine); non-hemolytic and low cytotoxicity; normal coagulation properties at fully efficacious doses via ROTEM (improved over protamine); reduced hemodynamic liability (improved over protamine); Compound 305 and Compound 283 restore normal bleeding time versus enoxaparin (improved over protamine); and single dose toxicity (MTDs)≧30 mg/kg.

Salicylamide compounds 305 and 311 demonstrate the following activities: potent anti-LMWH and anti-fondaparinux activity in vitro (improved over protamine); potent anti-LMWH and anti-fondaparinux activity in vivo (improved over protamine); non-hemolytic and low cytotoxicity; normal coagulation properties at fully efficacious doses via ROTEM (improved over protamine); reduced hemodynamic liability for compound 305 (improved over protamine); Compound 305 restores normal bleeding time versus enoxaparin (improved over protamine); and single dose toxicity (MTDs)≧30 mg/kg.

Various modifications of the invention, in addition to those described herein, will be apparent to those skilled in the art from the foregoing description. Such modifications are also intended to fall within the scope of the appended claims. Each reference (including, but not limited to, journal articles, U.S. and non-U.S. patents, patent application publications, international patent application publications, gene bank accession numbers, and the like) cited in the present application is herein incorporated by reference in its entirety.

What is claimed is:

1. A compound of Formula I:
R¹—[—X-A₁-Y—X-A₂-Y—]ₘ—R² I
or pharmaceutically acceptable salt thereof, wherein:
each X is, independently, NR⁸;
each Y is C═O;
each R⁸ is, independently, hydrogen or alkyl;
each A₂ is optionally substituted arylene or optionally substituted heteroarylene, and each A₁ is —(CH₂)_{q}—, wherein q is 1 to 7, wherein A₁ and A₂ are each, independently, optionally substituted with one or more PL group(s), one or more NPL group(s), or a combination of one or more PL group(s) and one or more NPL group(s);
R¹ is hydrogen, a PL group, or an NPL group, and R² is —X-A₁-Y—R¹¹, wherein R¹¹ is hydrogen, a PL group, or an NPL group; or
R¹ and R² are each, independently, hydrogen, a PL group, or an NPL group; or
R¹ and R² together are a single bond; or
R¹ is —Y-A₂-X—R¹², wherein R¹² is hydrogen, a PL group, or an NPL group, and R² is hydrogen, a PL group, or an NPL group;
each NPL group is, independently, —B(OR⁴)₂ or —(NR^{3′})_{q1NPL}—U^{NPL}-LK^{NPL}—(NR^{3″})_{q2NPL}—R^{4′}, wherein:
R³, R^{3′}, and R^{3″} are each, independently, hydrogen, alkyl, or alkoxy;
R⁴ and R^{4′} are each, independently, hydrogen, alkyl, alkenyl, alkynyl, cycloalkyl, aryl, or heteroaryl, wherein each of the alkyl, alkenyl, alkynyl, cycloalkyl, aryl, and heteroaryl is optionally substituted with one or more substitutents, wherein each substituent is, independently, alkyl, halo, or haloalkyl;
each U^{NPL} is, independently, absent or O, S, S(═O), S(═O)₂, NR³, —C(═O)—, —C(═O)—NR³—, —C(═O)—N═N—NR³—, —C(═O)—NR³—N═N—, —N═N—NR³—, —C(═N—N(R³)₂)—, —C(═NR³)—, —C(═O)O—, —C(═O)S—, —C(═S)—, —O—P(═O)₂O—, —S—C═N—, or —C(═O)—NR³—O—, wherein groups with two chemically nonequivalent termini can adopt both possible orientations;
each LK^{NPL} is, independently, —(CH₂)_{pNPL}— and C₂₋₈ alkenylenyl, wherein each of the —(CH₂)_{pNPL} and C₂₋₈ alkenylenyl is optionally substituted with one or more substituents, wherein each substituent is, independently, amino, hydroxyl, aminoalkyl, hydroxylalkyl, or alkyl;
each pNPL is, independently, an integer from 0 to 8;
q1NPL and q2NPL are each, independently, 0, 1, or 2;
each PL group is, independently, halo, hydroxyethoxymethyl, methoxyethoxymethyl, polyoxyethylene, or —(NR^{5′})_{q1PL}—U^{PL}-LK^{PL}—(NR^{5″})_{q2PL}—V, wherein:
R⁵, R^{5′}, and R^{5″} are each, independently, hydrogen, alkyl, or alkoxy;
each U^{PL} is, independently, absent or O, S, S(═O), S(═O)₂, NR⁵, —C(═O)—, —C(═O)—NR⁵—, —C(═O)—N═N—NR⁵—, —C(═O)—NR⁵—N═N—, —N═N—NR⁵—, —C(═N—N(R⁵)₂)—, —C(═NR⁵)—, —C(═O)O—, —C(═O)S—, —C(═S)—, —O—P(═O)₂O—, —S—C═N—, or —C(═O)—NR⁵—O—, wherein groups with two chemically nonequivalent termini can adopt either of the two possible orientations;
each V is, independently, nitro, cyano, amino, halo, hydroxy, alkoxy, alkylthio, alkylamino, dialkylamino, —NH(CH₂)ₚNH₂ wherein p is 1 to 5, —C(═O)NH(CH₂)ₚNH₂ wherein p is 1 to 5, —C(═O)NH(CH₂)ₚNHC(═NH)NH₂ wherein p is 1 to 5, —C(═O)NH(CH₂)ₚNHC(═O)NH₂ wherein p is 1 to 5, —NHC(═O)-alkyl, —N(CH₂CH₂NH₂)₂, diazamino, amidino, guanidino, ureido, carbamoyl, —C(═O)OH, —C(═O)OR^{c}, —C(═O)NH—OH, —O—NH—C(═NH)NH₂, —NH—S(═O)₂OH, S(═O)₂OH, NR^{d}R^{e}, semicarbazone, aryl, cycloalkyl, heterocycloalkyl, or heteroaryl, wherein each of the aryl and cycloalkyl is substituted with one or more substitutents, wherein each of the heterocycloalkyl and heteroaryl is optionally substituted with one or more substituents, and wherein each of the substituents for the aryl, cycloalkyl, heterocycloalkyl, and heteroaryl is, independently, nitro, cyano, amino, halo, hydroxy, alkoxy, alkylthio, alkylamino, dialkylamino, —NH(CH₂)ₚNH₂ wherein p is 1 to 5, —N(CH₂CH₂NH₂)₂, diazamino, amidino, guanidino, ureido, carbamoyl, —C(═O)OH, —C(═O)OR^{c}, —C(═O)NH—OH, —O—NH—C(═NH)NH₂, —NH—S(═O)₂OH, S(═O)₂OH, NR^{d}R^{e}, semicarbazone, aminosulfonyl, aminoalkoxy, aminoalkylhio, lower acylamino, or benzyloxycarbonyl;
each R^{c} is, independently, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl, or heterocycloalkylalkyl, each optionally substituted by one or more substitutents, wherein each substituent is, independently, OH, amino, halo, C₁₋₆ alkyl, C₁₋₆ haloalkyl, aryl, arylalkyl, heteroaryl, heteroarylalkyl, cycloalkyl, or heterocycloalkyl;
R^{d} and R^{e} are, independently, H, C₁₋₆ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl, or heterocycloalkylalkyl, wherein each of the C₁₋₆ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl and heterocycloalkylalkyl is optionally substituted by OH, amino, halo, C₁₋₆ alkyl, C₁₋₆ haloalkyl, C₁₋₆ haloalkyl, aryl, arylalkyl, heteroaryl, heteroarylalkyl, cycloalkyl, or heterocycloalkyl;
or R^{d} and R^{e} together with the N atom to which they are attached form a 4-, 5-, 6-, 7-, or 8-membered heterocycloalkyl;
each LK^{PL} is, independently, —(CH₂)_{pPL}— or C₂₋₈ alkenylenyl, wherein each of the —(CH₂)_{pNPL}— and C₂₋₈ alkenylenyl is optionally substituted with one or more substituents, wherein each substituent is, independently, amino, hydroxyl, aminoalkyl, hydroxylalkyl, or alkyl;
each pPL is, independently, an integer from 0 to 8;
q1PL and q2PL are each, independently, 0, 1, or 2;
m is an integer from 1 to about 20; and
at least one of A₁ is a —(CH₂)_{q}— group substituted with one substituent, wherein the substituent is (CH₂)—V¹, (CH₂)₂—V¹, —(CH₂)₃—V¹, —(CH₂)₄—V¹, or —(CH₂)₅—V¹, wherein V¹ is indolyl.

2. The compound of claim 1, or pharmaceutically acceptable salt thereof, wherein:
each X is NH;
each A₂ is, independently, phenyl optionally substituted with one or more substituents, wherein each substituent is, independently, O—(CH₃), halo, or O—(CH₂)₂—V;
each A₁ is, independently, a —(CH₂)— group optionally substituted with one substituent, wherein the substituent is CH₃, —(CH₂)—V, —(CH₂)₂—V, —(CH₂)₃—V, —(CH₂)₄—V, or —(CH₂)₅—V;
each V is, independently, hydroxyl, amino, heteroarylamino, ureido, guanidino, carbamoyl, C(═O)OH, —C(═O)OR^{c}, —C(═O)NH—OH, —O—NH—C(═NH)NH₂, —NH—S(═O)₂OH, S(═O)₂OH, aziridinyl, azetidinyl, pyrrolidinyl, piperidinyl, piperazinyl, morpholino, azepanyl, azocanyl, tetrazolyl, 1,2,4-oxadiazolyl, 1,3,4-oxadiazolyl, imidazolyl, pyridinyl, indolyl, or a substituted phenyl, wherein the substituted phenyl is substituted with one or more substituents, wherein each substituent is, independently, OH or amino; and
at least one of A₁ is a —(CH₂)— group substituted with one substituent, wherein the substituent is (CH₂)—V¹, (CH₂)₂—V¹, —(CH₂)₃—V¹, —(CH₂)₄—V¹, or —(CH₂)₅—V¹, wherein V¹ is indolyl.

3. A pharmaceutical composition comprising a compound of claim 1, or pharmaceutically acceptable salt thereof, and a pharmaceutically acceptable carrier.

4. A method for antagonizing unfractionated heparin, low molecular weight heparin, or heparin/low molecular weight heparin derivative comprising administering to a mammal a compound of Formula I:
R¹—[—X-A₁-Y—X-A₂-Y—]ₘ—R² I
or pharmaceutically acceptable salt thereof, wherein:
each X is, independently, NR⁸, —N(R⁸)N(R⁸)—, O, or S;
each Y is, independently, C═O, C═S, O═S═O, —C(═O)C(═O)—, or —CR^{a}R^{b}—;
R^{a} and R^{b} are each, independently, hydrogen, a PL group, or an NPL group;
each R⁸ is, independently, hydrogen or alkyl;
A₁ and A₂ are each, independently, optionally substituted arylene or optionally substituted heteroarylene, wherein A₁ and A₂ are, independently, optionally substituted with one or more PL group(s), one or more NPL group(s), or a combination of one or more PL group(s) and one or more NPL group(s); or
each A₁ is independently optionally substituted arylene or optionally substituted heteroarylene and each A₂ is a C₃ to C₈ cycloalkyl or —(CH₂)_{q}—, wherein q is 1 to 7, wherein A₁ and A₂ are, independently, optionally substituted with one or more PL group(s), one or more NPL group(s), or a combination of one or more PL group(s) and one or more NPL group(s); or
each A₂ is optionally substituted arylene or optionally substituted heteroarylene, and each A₁ is a C₃ to C₈ cycloalkyl or —(CH₂)_{q}—, wherein q is 1 to 7, wherein A₁ and A₂ are each, independently, optionally substituted with one or more PL group(s), one or more NPL group(s), or a combination of one or more PL group(s) and one or more NPL group(s);
R¹ is hydrogen, a PL group, or an NPL group, and R² is —X-A₁-Y—R¹¹, wherein R^{H} is hydrogen, a PL group, or an NPL group; or
R¹ and R² are each, independently, hydrogen, a PL group, or an NPL group; or
R¹ and R² together are a single bond; or
R¹ is —Y-A₂-X—R¹², wherein R¹² is hydrogen, a PL group, or an NPL group, and R² is hydrogen, a PL group, or an NPL group;
each NPL group is, independently, —B(OR⁴)₂ or —(NR^{3′})_{q1NPL}—U^{NPL}-LK^{NPL}—(NR^{3″})_{q2NPL}—R^{4′}, wherein:
R³, R^{3′}, and R^{3″} are each, independently, hydrogen, alkyl, or alkoxy;
R⁴ and R^{4′} are each, independently, hydrogen, alkyl, alkenyl, alkynyl, cycloalkyl, aryl, or heteroaryl, wherein each of the alkyl, alkenyl, alkynyl, cycloalkyl, aryl, and heteroaryl is optionally substituted with one or more substitutents, wherein the substituent is alkyl, halo, or haloalkyl;
each U^{NPL} is, independently, absent or O, S, S(═O), S(═O)₂, NR³, —C(═O)—, —C(═O)—NR³—, —C(═O)—N═N—NR³—, —C(═O)—NR³—N═N—, —N═N—NR³—, —C(═N—N(R³)₂)—, —C(═NR³)—, —C(═O)O—, —C(═O)S—, —C(═S)—, —O—P(═O)₂O—, —S—C═N—, or —C(═O)—NR³—O—, wherein groups with two chemically nonequivalent termini can adopt both possible orientations;
each LK^{NPL} is, independently, —(CH₂)_{pNPL}— or C₂₋₈ alkenylenyl, wherein each of the —(CH₂)_{pNPL} and C₂₋₈ alkenylenyl is optionally substituted with one or more substituents, wherein each substituent is, independently, amino, hydroxyl, aminoalkyl, hydroxylalkyl, or alkyl;
each pNPL is, independently, an integer from 0 to 8;
q1NPL and q2NPL are each, independently, 0, 1, or 2;
each PL group is, independently, halo, hydroxyethoxymethyl, methoxyethoxymethyl, polyoxyethylene, or —(NR^{5′})_{q1PL}—U^{PL}-LK^{PL}—(NR^{5″})_{q2PL}—V, wherein:
R⁵, R^{5′}, and R^{5″} are each, independently, hydrogen, alkyl, or alkoxy;
each U^{PL} is, independently, absent or O, S, S(═O), S(═O)₂, NR⁵, —C(═O)—, —C(═O)—NR⁵—, —C(═O)—N═N—NR⁵—, —C(═O)—NR⁵—N═N—, —N═N—NR⁵—, —C(═N—N(R⁵)₂)—, —C(═NR⁵)—, —C(═O)O—, —C(═O)S—, —C(═S)—, —O—P(═O)₂O—, —S—C═N—, or —C(═O)—NR⁵—O—, wherein groups with two chemically nonequivalent termini can adopt either of the two possible orientations;
each V is, independently, nitro, cyano, amino, halo, hydroxy, alkoxy, alkylthio, alkylamino, dialkylamino, —NH(CH₂)ₚNH₂ wherein p is 1 to 5, —C(═O)NH(CH₂)ₚNH₂ wherein p is 1 to 5, —C(═O)NH(CH₂)ₚNHC(═NH)NH₂ wherein p is 1 to 5, —C(═O)NH(CH₂)ₚNHC(═O)NH₂ wherein p is 1 to 5, —NHC(═O)-alkyl, —N(CH₂CH₂NH₂)₂, diazamino, amidino, guanidino, ureido, carbamoyl, —C(═O)OH, —C(═O)OR^{c}, —C(═O)NH—OH, —O—NH—C(═NH)NH₂, —NH—S(═O)₂OH, S(═O)₂OH, NR^{d}R^{e}, semicarbazone, aryl, cycloalkyl, heterocycloalkyl, or heteroaryl, wherein each of the aryl and cycloalkyl is substituted with one or more substitutents, wherein each of the heterocycloalkyl and heteroaryl is optionally substituted with one or more substituents, and wherein each of the substituents for the aryl, cycloalkyl, heterocycloalkyl, and heteroaryl is, independently, nitro, cyano, amino, halo, hydroxy, alkoxy, alkylthio, alkylamino, dialkylamino, —NH(CH₂)ₚNH₂ wherein p is 1 to 5, —N(CH₂CH₂NH₂)₂, diazamino, amidino, guanidino, ureido, carbamoyl, —C(═O)OH, —C(═O)OR^{c}, —C(═O)NH—OH, —O—NH—C(═NH)NH₂, —NH—S(═O)₂OH, S(═O)₂OH, NR^{d}R^{e}, semicarbazone, aminosulfonyl, aminoalkoxy, aminoalkylhio, lower acylamino, or benzyloxycarbonyl;
each R^{c} is, independently, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl, or heterocycloalkylalkyl, each optionally substituted by one or more substitutents, wherein the substituent is, independently, OH, amino, halo, C₁₋₆ alkyl, C₁₋₆ haloalkyl, aryl, arylalkyl, heteroaryl, heteroarylalkyl, cycloalkyl, or heterocycloalkyl;
R^{d} and R^{e} are, independently, H, C₁₋₆ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl, or heterocycloalkylalkyl, wherein each of the C₁₋₆ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl and heterocycloalkylalkyl is optionally substituted by OH, amino, halo, C₁₋₆ alkyl, C₁₋₆ haloalkyl, C₁₋₆ haloalkyl, aryl, arylalkyl, heteroaryl, heteroarylalkyl, cycloalkyl, or heterocycloalkyl;
or R^{d} and R^{e} together with the N atom to which they are attached form a 4-, 5-, 6-, 7-, or 8-membered heterocycloalkyl;
each LK^{PL} is, independently, —(CH₂)_{pPL}— or C₂₋₈ alkenylenyl, wherein each of the —(CH₂)_{pNPL}— and C₂₋₈ alkenylenyl is optionally substituted with one or more substituents, wherein the substituent is, independently, amino, hydroxyl, aminoalkyl, hydroxylalkyl, or alkyl;
each pPL is, independently, an integer from 0 to 8;
q1PL and q2PL are each, independently, 0, 1, or 2; and
m is an integer from 1 to about 20.

5. The method of claim 4 wherein the low molecular weight heparin is enoxaparin, reviparin, or tinzaparin, and the heparin/low molecular weight heparin derivative is fondaparinux.

6. A method for antagonizing unfractionated heparin, low molecular weight heparin, or heparin/low molecular weight heparin derivative comprising administering to a mammal a compound of Formula II:
R¹—[—X-A₁-X—Y-A₂-Y—]ₘ—R² II
or pharmaceutically acceptable salt thereof, wherein:
each X is, independently, NR⁸, O, S, —N(R⁸)N(R⁸)—, —N(R⁸)—(N═N)—, —(N═N)—N(R⁸)—, —C(R⁷R⁷)NR⁸—, —C(R⁷R⁷)O—, or —C(R⁷R⁷)S—;
each Y is, independently, C═O, C═S, O═S═O, —C(═O)C(═O)—, C(R⁶R^{6′})C═O, or C(R⁶R^{6′})C═S;
each R⁸ is, independently, hydrogen or alkyl;
each R⁷ and each R^{7′} are, independently, hydrogen or alkyl; or R⁷ and R^{7′} together form —(CH₂)ₚ—, wherein p is 4 to 8;
each R⁶ and each R^{6′} are, independently, hydrogen or alkyl; or R⁶ and R^{6′} together form —(CH₂)₂NR¹²(CH₂)₂—, wherein R¹² is hydrogen, —C(═N)CH₃, or —C(═NH)—NH₂;
A₁ and A₂ are each, independently, optionally substituted arylene or optionally substituted heteroarylene, wherein A₁ and A₂ are each, independently, optionally substituted with one or more PL group(s), one or more NPL group(s), or a combination of one or more PL group(s) and one or more NPL group(s);
or each A₂ is, independently, optionally substituted arylene or optionally substituted heteroarylene, and each A₁ is, independently, optionally substituted C₃ to C₈ cycloalkyl, wherein A₁ and A₂ are each, independently, optionally substituted with one or more PL group(s), one or more NPL group(s), or a combination of one or more PL group(s) and one or more NPL group(s);
R¹ is hydrogen, a PL group, or an NPL group, and R² is —X-A₁-X—R¹, wherein A₁ is as defined above and is optionally substituted with one or more PL group(s), one or more NPL group(s), or a combination of one or more PL group(s) and one or more NPL group(s); or
R¹ is hydrogen, a PL group, or an NPL group, and R² is —X-A′-X—R¹, wherein A′ is C₃ to C₈ cycloalkyl, aryl, or heteroaryl and is optionally substituted with one or more PL group(s), one or more NPL group(s), or a combination of one or more PL group(s) and one or more NPL group(s); or
R¹ is —Y-A₂-Y—R², and R² is each, independently, hydrogen, a PL group, or an NPL group; or
R¹ is —Y-A′ and R² is —X-A′, wherein each A′ is independently C₃ to C₈ cycloalkyl, aryl, or heteroaryl, and is optionally substituted with one or more PL group(s), one or more NPL group(s), or a combination of one or more PL group(s) and one or more NPL group(s); or
R¹ and R² are, independently, a PL group, or an NPL group; or
R¹ and R² together form a single bond;
each NPL is, independently, —B(OR⁴)₂ or —(NR^{3′})_{q1NPL}—U^{NPL}-LK^{NPL}—(NR^{3″})_{q2NPL}—R^{4′}, wherein:
R³, R^{3′}, and R^{3″} are each, independently, hydrogen, alkyl, or alkoxy;
R⁴ and R^{4′} are each, independently, hydrogen, alkyl, alkenyl, alkynyl, cycloalkyl, aryl, or heteroaryl, wherein each of the alkyl, alkenyl, alkynyl, cycloalkyl, aryl, and heteroaryl is optionally substituted with one or more alkyl or halo groups;
each U^{NPL} is, independently, absent or O, S, S(═O), S(═O)₂, NR³, —C(═O)—, —C(═O)—NR³—, —C(═O)—N═N—NR³—, —C(═O)—NR³—N═N—, —N═N—NR³—, —C(═N—N(R³)₂)—, —C(═NR³)—, —C(═O)O—, —C(═O)S—, —C(═S)—, —O—P(═O)₂O—, —S—C═N—, or —C(═O)—NR³—O—, wherein groups with two chemically nonequivalent termini can adopt both possible orientations;
each LK^{NPL} is, independently, —(CH₂)_{pNPL}— or C₂₋₈ alkenylenyl, wherein each of the —(CH₂)_{pNPL}— and C₂₋₈ alkenylenyl is optionally substituted with one or more substituents, wherein each substituent is, independently, amino, hydroxyl, aminoalkyl, hydroxylalkyl, or alkyl;
each pNPL is, independently, an integer from 0 to 8;
q1NPL and q2NPL are each, independently, 0, 1, or 2;
each PL is, independently, halo, hydroxyethoxymethyl, methoxyethoxymethyl, polyoxyethylene, or —(NR^{5′})_{q1PL}—U^{PL}-LK^{PL}—(NR^{5′})_{q2PL}—V, wherein:
R⁵, R^{5′}, and R^{5″} are each, independently, hydrogen, alkyl, or alkoxy;
each U^{PL} is, independently, absent or O, S, S(═O), S(═O)₂, NR⁵, —C(═O)—, —C(═O)—NR⁵—, —C(═O)—N═N—NR⁵—, —C(═O)—NR⁵—N═N—, —N═N—NR⁵—, —C(═N—N(R⁵)₂)—, —C(═NR⁵)—, —C(═O)O—, —C(═O)S—, —C(═S)—, —O—P(═O)₂O—, —S—C═N—, or —C(═O)—NR⁵—O—, wherein groups with two chemically nonequivalent termini can adopt both possible orientations;
each V is, independently, nitro, cyano, amino, hydroxy, alkoxy, alkylthio, alkylamino, dialkylamino, —NH(CH₂)ₚNH₂ wherein p is 1 to 5, C(═O)NH(CH₂)ₚNH₂ wherein p is 1 to 5, —C(═O)NH(CH₂)ₚNHC(═NH)NH₂ wherein p is 1 to 5, —C(═O)NH(CH₂)ₚNHC(═O)NH₂ wherein p is 1 to 5, —NHC(═O)-alkyl, —N(CH₂CH₂NH₂)₂, diazamino, amidino, guanidino, ureido, carbamoyl, —C(═O)OH, —C(═O)OR^{c}, —C(═O)NH—OH, —O—NH—C(═NH)NH₂, —NH—S(═O)₂OH, S(═O)₂OH, NR^{d}R^{e}, semicarbazone, aryl, cycloalkyl, heterocycloalkyl, or heteroaryl, wherein each of the aryl and cycloalkyl is substituted with one or more substitutents, wherein each of the heterocycloalkyl, and heteroaryl is optionally substituted with one or more substituents, and wherein each of the substituents for the aryl, cycloalkyl, heterocycloalkyl, and heteroaryl is, independently, nitro, cyano, amino, hydroxy, alkoxy, alkylthio, alkylamino, dialkylamino, —NH(CH₂)ₚNH₂ wherein p is 1 to 5, —N(CH₂CH₂NH₂)₂, diazamino, amidino, guanidino, ureido, carbamoyl, —C(═O)OH, —C(═O)OR^{c}, —C(═O)NH—OH, —O—NH—C(═NH)NH₂, —NH—S(═O)₂OH, S(═O)₂OH, NR^{d}R^{e}, semicarbazone, aminosulfonyl, aminoalkoxy, aminoalkylhio, lower acylamino, or benzyloxycarbonyl;
each LK^{PL} is, independently, —(CH₂)_{pPL}— or C₂₋₈ alkenylenyl, wherein each of the —(CH₂)_{pNPL}— and C₂₋₈ alkenylenyl is optionally substituted with one or more substituents, wherein each substituent is, independently, amino, hydroxyl, aminoalkyl, hydroxylalkyl, or alkyl;
each pPL is, independently, an integer from 0 to 8;
q1PL and q2PL are each, independently, 0, 1, or 2; and
m is an integer from 1 to about 20.

7. The method of claim 6 wherein the compound of Formula II, or pharmaceutically acceptable salt thereof, is a compound of Formula IIa:
R¹—X-A₁-X—Y-A₂-Y—X-A₁-X—R² IIa
or pharmaceutically acceptable salt thereof, wherein:
each X is, independently, NR⁸, O, S, or —N(R⁸)N(R⁸)—;
each Y is, independently, C═O, C═S, or O═S═O;
each R⁸ is, independently, hydrogen or alkyl;
A₁ and A₂ are each, independently, optionally substituted arylene or optionally substituted heteroarylene, wherein A₁ and A₂ are each, independently, optionally substituted with one or more PL group(s), one or more NPL group(s), or a combination of one or more PL group(s) and one or more NPL group(s);
R¹ is a PL group or an NPL group;
R² is R¹;
each NPL is —(NR^{3′})_{q1NPL}—U^{NPL}-LK^{NPL}—(NR^{3″})_{q2NPL}—R^{4′}, wherein:
R³, R^{3′}, and R^{3″} are each, independently, hydrogen, alkyl, or alkoxy;
R⁴ and R^{4′} are each, independently, hydrogen, alkyl, alkenyl, alkynyl, cycloalkyl, aryl, or heteroaryl, wherein each of the alkyl, alkenyl, alkynyl, cycloalkyl, aryl, and heteroaryl is optionally substituted with one or more alkyl or halo groups;
U^{NPL} is, independently, absent or O, S, S(═O), S(═O)₂, NR³, —C(═O)—, —C(═O)—NR³—, —C(═O)—N═N—NR³—, —C(═O)—NR³—N═N—, —N═N—NR³—, —C(═N—N(R³)₂)—, —C(═NR³)—, —C(═O)O—, —C(═O)S—, —C(═S)—, —O—P(═O)₂O—, —S—C═N—, or —C(═O)—NR³—O—, wherein groups with two chemically nonequivalent termini can adopt either of the two possible orientations;
each LK^{NPL} is, independently, —(CH₂)_{pNPL}— or C₂₋₈ alkenylenyl, wherein the —(CH₂)_{pNPL}— is optionally substituted with one or more substituents, wherein each substituent is, independently, amino, hydroxyl, or alkyl;
each pNPL is, independently, an integer from 0 to 8;
q1NPL and q2NPL are each, independently, 0, 1, or 2;
each PL is, independently, halo, hydroxyethoxymethyl, methoxyethoxymethyl, polyoxyethylene, or —(NR^{5′})_{q1PL}—U^{PL}-LK^{PL}—(NR^{5′})_{q2PL}—V, wherein:
R⁵, R^{5′}, and R^{5″} are each, independently, hydrogen, alkyl, or alkoxy;
each U^{PL} is, independently, absent or O, S, S(═O), S(═O)₂, NR⁵, —C(═O)—, —C(═O)—NR⁵—, —C(═O)—N═N—NR⁵—, —C(═O)—NR⁵—N═N—, —N═N—NR⁵—, —C(═N—N(R⁵)₂)—, —C(═NR⁵)—, —C(═O)O—, —C(═O)S—, —C(═S)—, —O—P(═O)₂O—, —R⁵⁰—, —R⁵S—, —S—C═N—, or —C(═O)—NR⁵—O—, wherein groups with two chemically nonequivalent termini can adopt both possible orientations;
each V is, independently, nitro, cyano, amino, hydroxy, alkoxy, alkylthio, alkylamino, dialkylamino, —NH(CH₂)ₚNH₂ wherein p is 1 to 5, —C(═O)NH(CH₂)ₚNH₂ wherein p is 1 to 5, —C(═O)NH(CH₂)ₚNHC(═NH)NH₂ wherein p is 1 to 5, —C(═O)NH(CH₂)ₚNHC(═O)NH₂ wherein p is 1 to 5, —NHC(═O)-alkyl, —N(CH₂CH₂NH₂)₂, diazamino, amidino, guanidino, ureido, carbamoyl, —C(═O)OH, —C(═O)OR^{c}, —C(═O)NH—OH, —O—NH—C(═NH)NH₂, —NH—S(═O)₂OH, S(═O)₂OH, NR^{d}R^{e}, semicarbazone, aryl, heterocycloalkyl, or heteroaryl, wherein the aryl is substituted with one or more substitutents, wherein each of the heterocycloalkyl and heteroaryl is optionally substituted with one or more substituents, and wherein each of each of the substituents for the aryl, heterocycloalkyl, and heteroaryl is, independently, nitro, cyano, amino, hydroxy, alkoxy, alkylthio, alkylamino, dialkylamino, —NH(CH₂)ₚNH₂ wherein p is 1 to 5, —N(CH₂CH₂NH₂)₂, diazamino, amidino, guanidino, ureido, carbamoyl, —C(═O)OH, —C(═O)OR^{c}, —C(═O)NH—OH, —O—NH—C(═NH)NH₂, —NH—S(═O)₂OH, S(═O)₂OH, NR^{d}R^{e}, semicarbazone, aminosulfonyl, aminoalkoxy, aminoalkylhio, lower acylamino, or benzyloxycarbonyl;
each LK^{PL} is, independently, —(CH₂)_{pPL}— or C₂₋₈ alkenylenyl, wherein the —(CH₂)_{pNPL}— is optionally substituted with one or more substituents, wherein each substituent is, independently, amino, hydroxyl, or alkyl;
each pPL is, independently, an integer from 0 to 8; and
q1PL and q2PL are each, independently, 0, 1, or 2.

8. The method of claim 6 wherein the low molecular weight heparin is enoxaparin, reviparin, or tinzaparin, and the heparin/low molecular weight heparin derivative is fondaparinux.

9. A compound of Formula III: 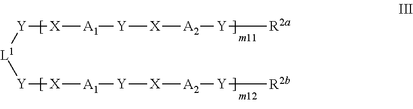
or pharmaceutically acceptable salt thereof, wherein:
each X is, independently, NR⁸;
each Y is C═O;
each R⁸ is, independently, hydrogen or alkyl;
each A₂ is optionally substituted arylene or optionally substituted heteroarylene, and each A₁ is —(CH₂)_{q}—, wherein q is 1 to 7, wherein A₁ and A₂ are each, independently, optionally substituted with one or more PL group(s), one or more NPL group(s), or a combination of one or more PL group(s) and one or more NPL group(s);
R² and R^{2a} are each, independently, hydrogen, a PL group, an NPL group or —X-A₁-Y—R¹¹, wherein R¹¹ is hydrogen, a PL group, or an NPL group;
L¹ is C₁₋₁₀alkylene optionally substituted with one or more substitutents, wherein each substituent is, independently, alkyl, halo, haloalkyl, aminoalkyl, hydroxylalkyl, V, or —(CH₂)_{pPL}—V wherein pPL is an integer from 1 to 5;
each NPL group is, independently, —B(OR⁴)₂ or —(NR^{3′})_{q1NPL}—U^{NPL}-LK^{NPL}—(NR^{3″})_{q2NPL}—R^{4′}, wherein:
R³, R^{3′}, and R^{3″} are each, independently, hydrogen, alkyl, or alkoxy;
R⁴ and R^{4′} are each, independently, hydrogen, alkyl, alkenyl, alkynyl, cycloalkyl, aryl, and heteroaryl, wherein each of the alkyl, alkenyl, alkynyl, cycloalkyl, aryl, or heteroaryl is optionally substituted with one or more substitutents, wherein each substituent is, independently, alkyl, halo, or haloalkyl;
each U^{NPL} is, independently, absent or O, S, S(═O), S(═O)₂, NR³, —C(═O)—, —C(═O)—NR³—, —C(═O)—N═N—NR³—, —C(═O)—NR³—N═N—, —N═N—NR³—, —C(═N—N(R³)₂)—, —C(═NR³)—, —C(═O)O—, —C(═O)S—, —C(═S)—, —O—P(═O)₂O—, —S—C═N—, or —C(═O)—NR³—O—, wherein groups with two chemically nonequivalent termini can adopt both possible orientations;
each LK^{NPL} is, independently, —(CH₂)_{pNPL}— or C₂₋₈ alkenylenyl, wherein each of the —(CH₂)_{pNPL} and C₂₋₈ alkenylenyl is optionally substituted with one or more substituents, wherein each substituent is, independently, amino, hydroxyl, aminoalkyl, hydroxylalkyl, or alkyl;
each pNPL is, independently, an integer from 0 to 8;
q1NPL and q2NPL are each, independently, 0, 1, or 2;
each PL group is, independently, halo, hydroxyethoxymethyl, methoxyethoxymethyl, polyoxyethylene, or —(NR^{5′})_{q1PL}—U^{PL}-LK^{PL}—(NR^{5″})_{q2PL}—V, wherein:
R⁵, R^{5′}, and R^{5″} are each, independently, hydrogen, alkyl, or alkoxy;
each U^{PL} is, independently, absent or O, S, S(═O), S(═O)₂, NR⁵, —C(═O)—, —C(═O)—NR⁵—, —C(═O)—N═N—NR⁵—, —C(═O)—NR⁵—N═N—, —N═N—NR⁵—, —C(═N—N(R⁵)₂)—, —C(═NR⁵)—, —C(═O)O—, —C(═O)S—, —C(═S)—, —O—P(═O)₂O—, —S—C═N—, or —C(═O)—NR⁵—O—, wherein groups with two chemically nonequivalent termini can adopt either of the two possible orientations;
each V is, independently, nitro, cyano, amino, halo, hydroxy, alkoxy, alkylthio, alkylamino, dialkylamino, —NH(CH₂)ₚNH₂ wherein p is 1 to 5, —C(═O)NH(CH₂)ₚNH₂ wherein p is 1 to 5, —C(═O)NH(CH₂)ₚNHC(═NH)NH₂ wherein p is 1 to 5, —C(═O)NH(CH₂)ₚNHC(═O)NH₂ wherein p is 1 to 5, —NHC(═O)-alkyl, —N(CH₂CH₂NH₂)₂, diazamino, amidino, guanidino, ureido, carbamoyl, —C(═O)OH, —C(═O)OR^{c}, —C(═O)NH—OH, —O—NH—C(═NH)NH₂, —NH—S(═O)₂OH, S(═O)₂OH, NR^{d}R^{e}, semicarbazone, aryl, cycloalkyl, heterocycloalkyl, or heteroaryl, wherein each of the aryl and cycloalkyl is substituted with one or more substitutents, wherein each of the heterocycloalkyl and heteroaryl is optionally substituted with one or more substituents, and wherein each of the substituents for the aryl, cycloalkyl, heterocycloalkyl, and heteroaryl is, independently, nitro, cyano, amino, halo, hydroxy, alkoxy, alkylthio, alkylamino, dialkylamino, —NH(CH₂)ₚNH₂ wherein p is 1 to 5, —N(CH₂CH₂NH₂)₂, diazamino, amidino, guanidino, ureido, carbamoyl, —C(═O)OH, —C(═O)OR^{c}, —C(═O)NH—OH, —O—NH—C(═NH)NH₂, —NH—S(═O)₂OH, S(═O)₂OH, NR^{d}R^{e}, semicarbazone, aminosulfonyl, aminoalkoxy, aminoalkylhio, lower acylamino, or benzyloxycarbonyl;
each R^{e} is, independently, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl, or heterocycloalkylalkyl, each optionally substituted by one or more substitutents, wherein each substituent is, independently, OH, amino, halo, C₁₋₆ alkyl, C₁₋₆ haloalkyl, aryl, arylalkyl, heteroaryl, heteroarylalkyl, cycloalkyl, or heterocycloalkyl;
R^{d} and R^{e} are, independently, H, C₁₋₆ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl, or heterocycloalkylalkyl, wherein each of the C₁₋₆ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl and heterocycloalkylalkyl is optionally substituted by OH, amino, halo, C₁₋₆ alkyl, C₁₋₆haloalkyl, C₁₋₆haloalkyl, aryl, arylalkyl, heteroaryl, heteroarylalkyl, cycloalkyl, or heterocycloalkyl;
or R^{d} and R^{e} together with the N atom to which they are attached form a 4-, 5-, 6-, 7-, or 8-membered heterocycloalkyl;
each LK^{PL} is, independently, —(CH₂)_{pPL}— or C₂₋₈ alkenylenyl, wherein each of the —(CH₂)_{pNPL}— and C₂₋₈ alkenylenyl is optionally substituted with one or more substituents, wherein each substituent is, independently, amino, hydroxyl, aminoalkyl, hydroxylalkyl, or alkyl;
each pPL is, independently, an integer from 0 to 8;
q1PL and q2PL are each, independently, 0, 1, or 2;
m11 is an integer from 1 to about 20; and
m12 is an integer from 1 to about 20.

10. The compound of claim 9, or pharmaceutically acceptable salt thereof, wherein:
each moiety of X-A₁-Y—X-A₂-Y is, independently, a moiety of: 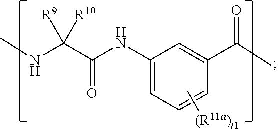
each R⁹ is, independently, H, a PL group, or an NPL group;
each R¹⁰ is, independently, H, a PL group, or an NPL group;
each R^{11a} is, independently, a PL group or an NPL group; and
each t1 is independently 0, 1, or 2.

11. A pharmaceutical composition comprising a compound of claim 9, or pharmaceutically acceptable salt thereof, and a pharmaceutically acceptable carrier.

12. A method for antagonizing unfractionated heparin, low molecular weight heparin, or heparin/low molecular weight heparin derivative comprising administering to a mammal a compound of Formula III: 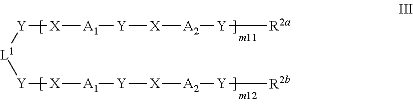
or pharmaceutically acceptable salt thereof, wherein:
each X is, independently, NR⁸;
each Y is C═O;
each R⁸ is, independently, hydrogen or alkyl;
each A₂ is optionally substituted arylene or optionally substituted heteroarylene, and each A₁ is —(CH₂)_{q}—, wherein q is 1 to 7, wherein A₁ and A₂ are each, independently, optionally substituted with one or more PL group(s), one or more NPL group(s), or a combination of one or more PL group(s) and one or more NPL group(s);
R² and R^{2a} are each, independently, hydrogen, a PL group, an NPL group or —X-A₁-Y—R¹¹, wherein R¹¹ is hydrogen, a PL group, or an NPL group;
L¹ is C₁₋₁₀alkylene optionally substituted with one or more substitutents, wherein each substituent is, independently, alkyl, halo, haloalkyl; aminoalkyl, hydroxylalkyl, V, or —(CH₂)_{pPL}—V wherein pPL is an integer from 1 to 5;
each NPL group is, independently, —B(OR⁴)₂ or —(NR^{3′})_{q1NPL}—U^{NPL}-LK^{NPL}—(NR^{3″})_{q2NPL}—R^{4′}, wherein:
R³, R^{3′}, and R^{3″} are each, independently, hydrogen, alkyl, or alkoxy;
R⁴ and R^{4′} are each, independently, hydrogen, alkyl, alkenyl, alkynyl, cycloalkyl, aryl, or heteroaryl, wherein each of the alkyl, alkenyl, alkynyl, cycloalkyl, aryl, and heteroaryl is optionally substituted with one or more substitutents, wherein each substituent is, independently, alkyl, halo, or haloalkyl;
each U^{NPL} is, independently, absent or O, S, S(═O), S(═O)₂, NR³, —C(═O)—, —C(═O)—NR³—, —C(═O)—N═N—NR³—, —C(═O)—NR³—N═N—, —N═N—NR³—, —C(═N—N(R³)₂)—, —C(═NR³)—, —C(═O)O—, —C(═O)S—, —C(═S)—, —O—P(═O)₂O—, —S—C═N—, or —C(═O)—NR³—O—, wherein groups with two chemically nonequivalent termini can adopt both possible orientations;
each LK^{NPL} is, independently, —(CH₂)_{pNPL}— or C₂₋₈ alkenylenyl, wherein each of the —(CH₂)_{pNPL} and C₂₋₈ alkenylenyl is optionally substituted with one or more substituents, wherein each substituent is, independently, amino, hydroxyl, aminoalkyl, hydroxylalkyl, or alkyl;
each pNPL is, independently, an integer from 0 to 8;
q1NPL and q2NPL are each, independently, 0, 1, or 2;
each PL group is, independently, halo, hydroxyethoxymethyl, methoxyethoxymethyl, polyoxyethylene, or —(NR^{5′})_{q1PL}—U^{PL}-LK^{PL}—(NR^{5″})_{q2PL}—V, wherein:
R⁵, R^{5′}, and R^{5″} are each, independently, hydrogen, alkyl, or alkoxy;
each U^{PL} is, independently, absent or O, S, S(═O), S(═O)₂, NR⁵, —C(═O)—, —C(═O)—NR⁵—, —C(═O)—N═N—NR⁵—, —C(═O)—NR⁵—N═N—, —N═N—NR⁵—, —C(═N—N(R⁵)₂)—, —C(═NR⁵)—, —C(═O)O—, —C(═O)S—, —C(═S)—, —O—P(═O)₂O—, —S—C═N—, or —C(═O)—NR⁵—O—, wherein groups with two chemically nonequivalent termini can adopt either of the two possible orientations;
each V is, independently, nitro, cyano, amino, halo, hydroxy, alkoxy, alkylthio, alkylamino, dialkylamino, —NH(CH₂)ₚNH₂ wherein p is 1 to 5, —C(═O)NH(CH₂)ₚNH₂ wherein p is 1 to 5, —C(═O)NH(CH₂)ₚNHC(═NH)NH₂ wherein p is 1 to 5, —C(═O)NH(CH₂)ₚNHC(═O)NH₂ wherein p is 1 to 5, —NHC(═O)-alkyl, —N(CH₂CH₂NH₂)₂, diazamino, amidino, guanidino, ureido, carbamoyl, —C(═O)OH, —C(═O)OR^{c}, —C(═O)NH—OH, —O—NH—C(═NH)NH₂, —NH—S(═O)₂OH, S(═O)₂OH, NR^{d}R^{e}, semicarbazone, aryl, cycloalkyl, heterocycloalkyl, or heteroaryl, wherein each of the aryl and cycloalkyl is substituted with one or more substitutents, wherein each of the heterocycloalkyl and heteroaryl is optionally substituted with one or more substituents, and wherein each of the substituents for the aryl, cycloalkyl, heterocycloalkyl, and heteroaryl is, independently, nitro, cyano, amino, halo, hydroxy, alkoxy, alkylthio, alkylamino, dialkylamino, —NH(CH₂)ₚNH₂ wherein p is 1 to 5, —N(CH₂CH₂NH₂)₂, diazamino, amidino, guanidino, ureido, carbamoyl, —C(═O)OH, —C(═O)OR^{c}, —C(═O)NH—OH, —O—NH—C(═NH)NH₂, —NH—S(═O)₂OH, S(═O)₂OH, NR^{d}R^{e}, semicarbazone, aminosulfonyl, aminoalkoxy, aminoalkylhio, lower acylamino, or benzyloxycarbonyl;
each R^{c} is, independently, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl, or heterocycloalkylalkyl, each optionally substituted by one or more substitutents, wherein each substituent is, independently, OH, amino, halo, C₁₋₆ alkyl, C₁₋₆ haloalkyl, aryl, arylalkyl, heteroaryl, heteroarylalkyl, cycloalkyl, or heterocycloalkyl;
R^{d} and R^{e} are, independently, H, C₁₋₆ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl, or heterocycloalkylalkyl, wherein each of the C₁₋₆ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl and heterocycloalkylalkyl is optionally substituted by OH, amino, halo, C₁₋₆ alkyl, C₁₋₆haloalkyl, C₁₋₆haloalkyl, aryl, arylalkyl, heteroaryl, heteroarylalkyl, cycloalkyl, or heterocycloalkyl;
or R^{d} and R^{e} together with the N atom to which they are attached form a 4-, 5-, 6-, 7-, or 8-membered heterocycloalkyl;
each LK^{PL} is, independently, —(CH₂)_{pPL}— or C₂₋₈ alkenylenyl, wherein each of the —(CH₂)_{pNPL}— and C₂₋₈ alkenylenyl is optionally substituted with one or more substituents, wherein each substituent is, independently, amino, hydroxyl, aminoalkyl, hydroxylalkyl, or alkyl;
each pPL is, independently, an integer from 0 to 8;
q1PL and q2PL are each, independently, 0, 1, or 2;
m11 is an integer from 1 to about 20; and
m12 is an integer from 1 to about 20.

13. The method of claim 12 wherein:
each moiety of X-A₁-Y—X-A₂-Y is, independently, a moiety of: 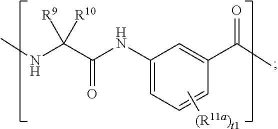
each R⁹ is, independently, H, a PL group, or an NPL group;
each R¹⁰ is, independently, H, a PL group, or an NPL group;
each R^{11a} is, independently, a PL group or an NPL group; and
each t1 is, independently, 0, 1, or 2.

14. The method of claim 12 wherein the low molecular weight heparin is enoxaparin, reviparin, or tinzaparin, and the heparin/low molecular weight heparin derivative is fondaparinux.

15. A compound of Formula IV:
R¹—[—X-A₁-Y—X-A₂-Y—]ₘ₁₃—X-L¹-Y—[—X-A₁-Y—X-A₂-Y—]ₘ₁₄—R² IV
or pharmaceutically acceptable salt thereof, wherein:
each X is, independently, NR⁸;
each Y is C═O;
each R⁸ is, independently, hydrogen or alkyl;
each A₂ is optionally substituted arylene or optionally substituted heteroarylene, and each A₁ is —(CH₂)_{q}—, wherein q is 1 to 7, wherein A₁ and A₂ are each, independently, optionally substituted with one or more PL group(s), one or more NPL group(s), or a combination of one or more PL group(s) and one or more NPL group(s);
R¹ is hydrogen, a PL group, or an NPL group, and R² is —X-A₁-Y—R¹¹, wherein R¹¹ is hydrogen, a PL group, or an NPL group; or
R¹ and R² are each, independently, hydrogen, a PL group, or an NPL group; or
R¹ and R² together are a single bond; or
R¹ is —Y-A₂-X—R¹², wherein R¹² is hydrogen, a PL group, or an NPL group, and R² is hydrogen, a PL group, or an NPL group;
L¹ is C₁₋₁₀alkylene optionally substituted with one or more substitutents, wherein each substituent is, independently, alkyl, halo, haloalkyl; aminoalkyl, hydroxylalkyl, V, or —(CH₂)_{pPL}—V wherein pPL is an integer from 1 to 5;
each V is, independently, hydroxy, amino, alkylamino, dialkylamino, —NH(CH₂)ₚNH₂ wherein p is 1 to 5, —C(═O)NH(CH₂)ₚNH₂ wherein p is 1 to 5, —C(═O)NH(CH₂)ₚNHC(═NH)NH₂ wherein p is 1 to 5, —C(═O)NH(CH₂)ₚNHC(═O)NH₂ wherein p is 1 to 5, —NHC(═O)-alkyl, —N(CH₂CH₂NH₂)₂, guanidino, amidino, ureido, carbamoyl, —C(═O)OH, —C(═O)OR^{c}, —C(═O)NH—OH, —O—NH—C(═NH)NH₂, —NH—S(═O)₂OH, S(═O)₂OH, NR^{d}R^{e}, a substituted aryl group, heterocycloalkyl, or heteroaryl, wherein each of the heterocycloalkyl and heteroaryl is optionally substituted with one more substituents, wherein each substituent is, independently, amino, halo, cyano, nitro, hydroxy, —NH(CH₂)ₚNH₂ wherein p is 1 to 5, —N(CH₂CH₂NH₂)₂, amidino, guanidino, aminosulfonyl, aminoalkoxy, aminoalkylhio, lower acylamino, or benzyloxycarbonyl; and wherein the substituted aryl group is substituted with one more substituents, wherein each substituent is, independently, amino, halo, cyano, nitro, hydroxy, —NH(CH₂)ₚNH₂ wherein p is 1 to 5, —N(CH₂CH₂NH₂)₂, amidino, guanidino, aminosulfonyl, aminoalkoxy, aminoalkylhio, lower acylamino, or benzyloxycarbonyl.
each NPL group is, independently, —B(OR⁴)₂ or —(NR^{3′})_{q1NPL}—U^{NPL}-LK^{NPL}—(NR^{3″})_{q2NPL}—R⁴, wherein:
R³, R^{3′}, and R^{3″} are each, independently, hydrogen, alkyl, or alkoxy;
R⁴ and R^{4′} are each, independently, hydrogen, alkyl, alkenyl, alkynyl, cycloalkyl, aryl, or heteroaryl, wherein each of the alkyl, alkenyl, alkynyl, cycloalkyl, aryl, and heteroaryl is optionally substituted with one or more substitutents, wherein each substituent is, independently, alkyl, halo, or haloalkyl;
each U^{NPL} is, independently, absent or O, S, S(═O), S(═O)₂, NR³, —C(═O)—, —C(═O)—NR³—, —C(═O)—N═N—NR³—, —C(═O)—NR³—N═N—, —N═N—NR³—, —C(═N—N(R³)₂)—, —C(═NR³)—, —C(═O)O—, —C(═O)S—, —C(═S)—, —O—P(═O)₂O—, —S—C═N—, or —C(═O)—NR³—O—, wherein groups with two chemically nonequivalent termini can adopt both possible orientations;
each LK^{NPL} is, independently, —(CH₂)_{pNPL}— or C₂₋₈ alkenylenyl, wherein each of the —(CH₂)_{pNPL} and C₂₋₈ alkenylenyl is optionally substituted with one or more substituents, wherein each substituent is, independently, amino, hydroxyl, aminoalkyl, hydroxylalkyl, or alkyl;
each pNPL is, independently, an integer from 0 to 8;
q1NPL and q2NPL are each, independently, 0, 1, or 2;
each PL group is, independently, halo, hydroxyethoxymethyl, methoxyethoxymethyl, polyoxyethylene, or —(NR^{5′})_{q1PL}—U^{PL}-LK^{PL}—(NR^{5″})_{q2PL}—V, wherein:
R⁵, R^{5′}, and R^{5″} are each, independently, hydrogen, alkyl, or alkoxy;
each U^{PL} is, independently, absent or O, S, S(═O), S(═O)₂, NR⁵, —C(═O)—, —C(═O)—NR⁵—, —C(═O)—N═N—NR⁵—, —C(═O)—NR⁵—N═N—, —N═N—NR⁵—, —C(═N—N(R⁵)₂)—, —C(═NR⁵)—, —C(═O)O—, —C(═O)S—, —C(═S)—, —O—P(═O)₂O—, —S—C═N—, or —C(═O)—NR⁵—O—, wherein groups with two chemically nonequivalent termini can adopt either of the two possible orientations;
each R^{c} is, independently, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl, or heterocycloalkylalkyl, each optionally substituted by one or more substitutents, wherein each substituent is, independently, OH, amino, halo, C₁₋₆ alkyl, C₁₋₆ haloalkyl, aryl, arylalkyl, heteroaryl, heteroarylalkyl, cycloalkyl, or heterocycloalkyl;
R^{d} and R^{e} are, independently, H, C₁₋₆ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl, or heterocycloalkylalkyl, wherein each of the C₁₋₆ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl and heterocycloalkylalkyl is optionally substituted by OH, amino, halo, C₁₋₆ alkyl, C₁₋₆haloalkyl, C₁₋₆haloalkyl, aryl, arylalkyl, heteroaryl, heteroarylalkyl, cycloalkyl, or heterocycloalkyl;
or R^{d} and R^{e} together with the N atom to which they are attached form a 4-, 5-, 6-, 7-, or 8-membered heterocycloalkyl;
each LK^{PL} is, independently, —(CH₂)_{pPL}— or C₂₋₈ alkenylenyl, wherein each of the —(CH₂)_{pNPL}— and C₂₋₈ alkenylenyl is optionally substituted with one or more substituents, wherein each substituent is, independently, amino, hydroxyl, aminoalkyl, hydroxylalkyl, or alkyl;
each pPL is, independently, an integer from 0 to 8;
q1PL and q2PL are each, independently, 0, 1, or 2;
m13 is an integer from 1 to about 10; and
m14 is an integer from 1 to about 10.

16. The compound of claim 15, or pharmaceutically acceptable salt thereof, wherein:
each moiety of X-A₁-Y—X-A₂-Y is, independently, a moiety of: 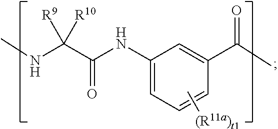
each R⁹ is, independently, H, a PL group, or an NPL group;
each R¹⁰ is, independently, H, a PL group, or an NPL group;
each R^{11a} is, independently, a PL group or an NPL group; and
each t1 is independently 0, 1, or 2.

17. A pharmaceutical composition comprising a compound of claim 15, or pharmaceutically acceptable salt thereof, and a pharmaceutically acceptable carrier.

18. A method for antagonizing unfractionated heparin, low molecular weight heparin, or heparin/low molecular weight heparin derivative comprising administering to a mammal a compound of Formula IV:
R¹—[—X-A₁-Y—X-A₂-Y—]ₘ₁₃—X-L¹-Y—[—X-A₁-Y—X-A₂-Y—]ₘ₁₄—R² IV
or pharmaceutically acceptable salt thereof, wherein:
each X is, independently, NR⁸;
each Y is C═O;
each R⁸ is, independently, hydrogen or alkyl;
each A₂ is optionally substituted arylene or optionally substituted heteroarylene, and each A₁ is —(CH₂)_{q}—, wherein q is 1 to 7, wherein A₁ and A₂ are each, independently, optionally substituted with one or more PL group(s), one or more NPL group(s), or a combination of one or more PL group(s) and one or more NPL group(s);
R¹ is hydrogen, a PL group, or an NPL group, and R² is —X-A₁-Y—R¹¹, wherein R^{H} is hydrogen, a PL group, or an NPL group; or
R¹ and R² are each, independently, hydrogen, a PL group, or an NPL group; or
R¹ and R² together are a single bond; or
R¹ is —Y-A₂-X—R¹², wherein R¹² is hydrogen, a PL group, or an NPL group, and R² is hydrogen, a PL group, or an NPL group;
L¹ is C₁₋₁₀alkylene optionally substituted with one or more substitutents, wherein each substituent is, independently alkyl, halo, haloalkyl; aminoalkyl, hydroxylalkyl, V, or —(CH₂)_{pPL}—V wherein pPL is an integer from 1 to 5;
each V is, independently, hydroxy, amino, alkylamino, dialkylamino, —NH(CH₂)ₚNH₂ wherein p is 1 to 5, —C(═O)NH(CH₂)ₚNH₂ wherein p is 1 to 5, —C(═O)NH(CH₂)ₚNHC(═NH)NH₂ wherein p is 1 to 5, —C(═O)NH(CH₂)ₚNHC(═O)NH₂ wherein p is 1 to 5, —NHC(═O)-alkyl, —N(CH₂CH₂NH₂)₂, guanidino, amidino, ureido, carbamoyl, —C(═O)OH, —C(═O)OR^{c}, —C(═O)NH—OH, —O—NH—C(═NH)NH₂, —NH—S(═O)₂OH, S(═O)₂OH, NR^{d}R^{e}, a substituted aryl group, heterocycloalkyl, or heteroaryl, wherein each of the heterocycloalkyl and heteroaryl is optionally substituted with one more substituents, wherein each substituent is, independently, amino, halo, cyano, nitro, hydroxy, —NH(CH₂)ₚNH₂ wherein p is 1 to 5, —N(CH₂CH₂NH₂)₂, amidino, guanidino, aminosulfonyl, aminoalkoxy, aminoalkylhio, lower acylamino, or benzyloxycarbonyl; and wherein the substituted aryl group is substituted with one more substituents, wherein each substituent is, independently, amino, halo, cyano, nitro, hydroxy, —NH(CH₂)ₚNH₂ wherein p is 1 to 5, —N(CH₂CH₂NH₂)₂, amidino, guanidino, aminosulfonyl, aminoalkoxy, aminoalkylhio, lower acylamino, or benzyloxycarbonyl;
each NPL group is, independently, —B(OR⁴)₂ or —(NR^{3′})_{q1NPL}—U^{NPL}-LK^{NPL}—(NR^{3″})_{q2NPL}—R⁴, wherein:
R³, R^{3′}, and R^{3″} are each, independently, hydrogen, alkyl, or alkoxy;
R⁴ and R^{4′} are each, independently, hydrogen, alkyl, alkenyl, alkynyl, cycloalkyl, aryl, or heteroaryl, wherein each of the alkyl, alkenyl, alkynyl, cycloalkyl, aryl, and heteroaryl is optionally substituted with one or more substitutents, wherein each substituent is, independently, alkyl, halo, or haloalkyl;
each U^{NPL} is, independently, absent or O, S, S(═O), S(═O)₂, NR³, —C(═O)—, —C(═O)—NR³—, —C(═O)—N═N—NR³—, —C(═O)—NR³—N═N—, —N═N—NR³—, —C(═N—N(R³)₂)—, —C(═NR³)—, —C(═O)O—, —C(═O)S—, —C(═S)—, —O—P(═O)₂O—, —S—C═N—, or —C(═O)—NR³—O—, wherein groups with two chemically nonequivalent termini can adopt both possible orientations;
each LK^{NPL} is, independently, —(CH₂)_{pNPL}— or C₂₋₈ alkenylenyl, wherein each of the —(CH₂)_{pNPL} and C₂₋₈ alkenylenyl is optionally substituted with one or more substituents, wherein each substituent is, independently, amino, hydroxyl, aminoalkyl, hydroxylalkyl, or alkyl;
each pNPL is, independently, an integer from 0 to 8;
q1NPL and q2NPL are each, independently, 0, 1, or 2;
each PL group is, independently, halo, hydroxyethoxymethyl, methoxyethoxymethyl, polyoxyethylene, or —(NR^{5′})_{q1PL}—U^{PL}-LK^{PL}—(NR^{5″})_{q2PL}—V, wherein:
R⁵, R^{5′}, and R^{5″} are each, independently, hydrogen, alkyl, or alkoxy;
each U^{PL} is, independently, absent or O, S, S(═O), S(═O)₂, NR⁵, —C(═O)—, —C(═O)—NR⁵—, —C(═O)—N═N—NR⁵—, —C(═O)—NR⁵—N═N—, —N═N—NR⁵—, —C(═N—N(R⁵)₂)—, —C(═NR⁵)—, —C(═O)O—, —C(═O)S—, —C(═S)—, —O—P(═O)₂O—, —S—C═N—, or —C(═O)—NR⁵—O—, wherein groups with two chemically nonequivalent termini can adopt either of the two possible orientations;
each R^{c} is, independently, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl, or heterocycloalkylalkyl, each optionally substituted by one or more substitutents, wherein each substituent is, independently, OH, amino, halo, C₁₋₆ alkyl, C₁₋₆ haloalkyl, aryl, arylalkyl, heteroaryl, heteroarylalkyl, cycloalkyl, or heterocycloalkyl;
R^{d} and R^{e} are, independently, H, C₁₋₆ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl, or heterocycloalkylalkyl, wherein each of the C₁₋₆ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl and heterocycloalkylalkyl is optionally substituted by OH, amino, halo, C₁₋₆ alkyl, C₁₋₆haloalkyl, C₁₋₆haloalkyl, aryl, arylalkyl, heteroaryl, heteroarylalkyl, cycloalkyl, or heterocycloalkyl;
or R^{d} and R^{e} together with the N atom to which they are attached form a 4-, 5-, 6-, 7-, or 8-membered heterocycloalkyl;
each LK^{PL} is, independently, —(CH₂)_{pPL}— or C₂₋₈ alkenylenyl, wherein each of the —(CH₂)_{pNPL}— and C₂₋₈ alkenylenyl is optionally substituted with one or more substituents, wherein each substituent is, independently, amino, hydroxyl, aminoalkyl, hydroxylalkyl, or alkyl;
each pPL is, independently, an integer from 0 to 8;
q1PL and q2PL are each, independently, 0, 1, or 2;
m13 is an integer from 1 to about 10; and
m14 is an integer from 1 to about 10.

19. The method of claim 18 wherein:
each moiety of X-A₁-Y—X-A₂-Y is, independently, a moiety of: 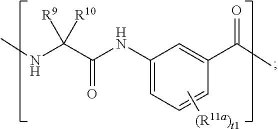
each R⁹ is, independently, H, a PL group, or an NPL group;
each R¹⁰ is, independently, H, a PL group, or an NPL group;
each R^{11a} is, independently, a PL group or an NPL group; and
each t1 is independently 0, 1, or 2.

20. The method of claim 18 wherein the low molecular weight heparin is enoxaparin, reviparin, or tinzaparin, and the heparin/low molecular weight heparin derivative is fondaparinux.

21. A compound of Formula V:
R¹—[—X-A¹-X—Y-A²-Y—]ₘ—R² V
or a pharmaceutically acceptable salt thereof, wherein:
each of the moiety of —X-A¹-X— is, independently, a moiety of Formula XXI-1, XXI-2, XXI-3, XXI-4, XXI-5, XXI-6, XXI-7, or XXI-8: 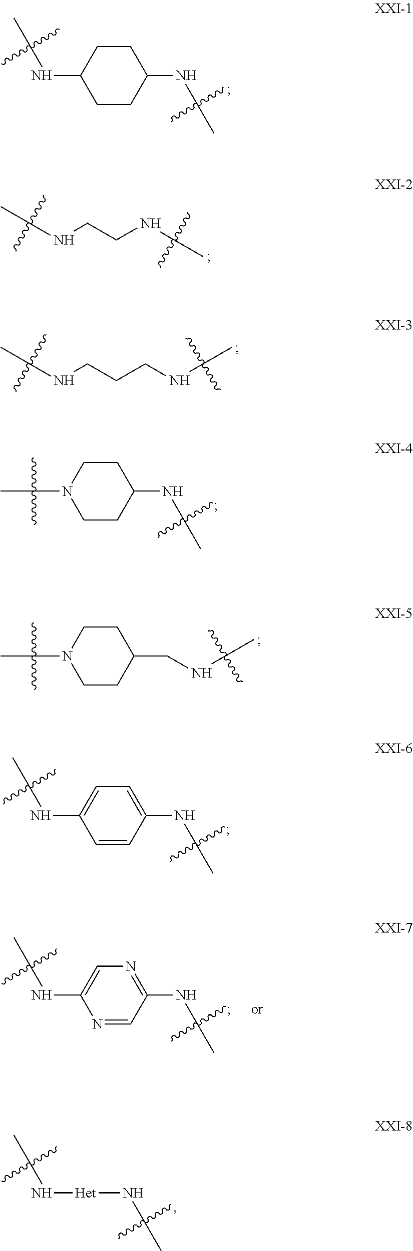
where Het is any 5 or 6-membered ring heterocycle;
each of the moiety of —Y-A²-Y— is, independently, a moiety of Formula XXII-1, XXII-2, XXII-3, XXII-4, or XXII-5: 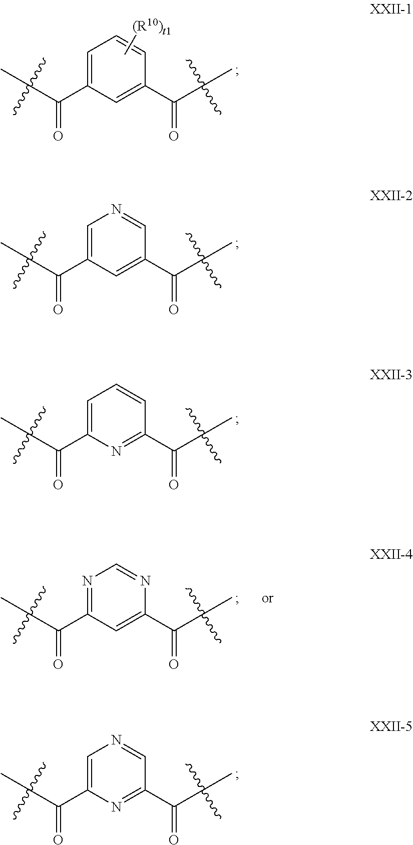
R¹ is hydrogen, —C(═O)R¹¹, or —Y-A²-Y—R¹²;
R² is —OH, —NH₂, —NH(C₁₋₄alkyl), —N(C₁₋₄alkyl)₂, —NH—C(═NH)NH₂, —NH(CH₂)ₚNH₂ wherein p is an integer from 1 to 5, —NH(CH₂)ₚNH(C₁₋₄alkyl) wherein p is an integer from 1 to 5, —NH(CH₂)ₚN(C₁₋₄alkyl)₂ wherein p is an integer from 1 to 5, —NH(CH₂)ₚNHC(═NH)NH₂ wherein p is an integer from 1 to 5, R^{12a}, or —X-A¹-X—R¹³;
each R¹⁰ is, independently, —C(═O)NH₂, —C(═O)NH(CH₂)ₚNH₂ wherein p is an integer from 1 to 5, —C(═O)NH(CH₂)ₚNH(C₁₋₄alkyl) wherein p is an integer from 1 to 5, —C(═O)NH(CH₂)ₚN(C₁₋₄alkyl)₂ wherein p is an integer from 1 to 5, —C(═O)NH(CH₂)ₚNHC(═NH)NH₂ wherein p is an integer from 1 to 5, —OCH₃, or —OR^{10a};
each R^{10a} is, independently, C₁₋₈alkyl substituted with R^{A};
each R^{A} is independently —NH₂, —NH(C₁₋₄alkyl), —N(C₁₋₄alkyl)₂, —NH—C(═NH)NH₂, —C(═O)NH₂, or —C(═O)OH;
each R¹¹ is, independently, C₁₋₈alkyl or aryl, each substituted with 0, 1, 2, or 3 substituents each independently selected from —OCH₃, —OR^{11a}, —C(═O)NH₂, —C(═O)NH(CH₂)ₚNH₂ wherein p is an integer from 1 to 5, —C(═O)NH(CH₂)ₚNH(C₁₋₄alkyl) wherein p is an integer from 1 to 5, —C(═O)NH(CH₂)ₚN(C₁₋₄alkyl)₂ wherein p is an integer from 1 to 5, —C(═O)NH(CH₂)ₚNHC(═NH)NH₂ wherein p is an integer from 1 to 5, —NH₂, —NH(C₁₋₄alkyl), —N(C₁₋₄alkyl)₂, or —NH—C(═NH)NH₂;
each R^{11a} is, independently, C₁₋₈alkyl substituted with R^{B};
each R^{B} is, independently, —NH₂, —NH(C₁₋₄alkyl), —N(C₁₋₄alkyl)₂, —NH—C(═NH)NH₂, or —C(═O)NH₂;
R¹² is —OH, —NH₂, —NH(C₁₋₄alkyl), —N(C₁₋₄alkyl)₂, —NH—C(═NH)NH₂, —NH(CH₂)ₚNH₂ wherein p is an integer from 1 to 5, —NH(CH₂)ₚNH(C₁₋₄alkyl) wherein p is an integer from 1 to 5, —NH(CH₂)ₚN(C₁₋₄alkyl)₂ wherein p is an integer from 1 to 5, —NH(CH₂)ₚNHC(═NH)NH₂ wherein p is an integer from 1 to 5, or R^{12a};
R^{12a} is a moiety of Formula XXXI: 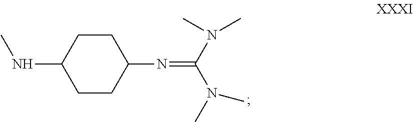
R¹³ is hydrogen or —C(═O)R¹¹;
t1 is 0, 1, or 2; and
m is 1, 2, 3, or 4,
provided that:
(a) the compound of Formula V, or pharmaceutically acceptable salt thereof, comprises at least one moiety of Formula XXI-4;
(b) the compound of Formula V, or pharmaceutically acceptable salt thereof, comprises at least one moiety of Formula XXI-5;
(c) the compound of Formula V, or pharmaceutically acceptable salt thereof, comprises at least two different moieties of Formulas XXI-1, XXI-2, XXI-3, XXI-4, or XXI-5;
(d) the compound of Formula V, or pharmaceutically acceptable salt thereof, comprises at least one moiety of Formula XXI-4 and at least one moiety of Formula XXI-1, XXI-2, XXI-3, or XXI-5;
(e) the compound of Formula V, or pharmaceutically acceptable salt thereof, comprises at least one moiety of Formula XXI-5 and at least one moiety of Formula XXI-1, XXI-2, XXI-3, or XXI-4;
(f) the compound of Formula V, or pharmaceutically acceptable salt thereof, comprises at least one moiety of Formula XXII-2;
(g) the compound of Formula V, or pharmaceutically acceptable salt thereof, comprises at least one moiety of Formula XXII-3;
(h) the compound of Formula V, or pharmaceutically acceptable salt thereof, comprises at least one moiety of Formula XXII-2 and at least one moiety of Formula XXII-1, XXII-3, or XXII-4;
(i) the compound of Formula V, or pharmaceutically acceptable salt thereof, comprises at least one moiety of Formula XXII-3 and at least one moiety of Formula XXII-1, XXII-2, or XXII-4;
(j) the compound of Formula V, or pharmaceutically acceptable salt thereof, comprises at least two different moieties of Formulas XXII-1, XXII-2, XXII-3 and XXII-4;
(k) the compound of Formula V, or pharmaceutically acceptable salt thereof, comprises at least one moiety of Formula XXXI;
(l) the compound of Formula V, or pharmaceutically acceptable salt thereof, comprises at least two different moieties of Formula XXI-6, XXI-7, or XXI-8;
(m) the compound of Formula V, or pharmaceutically acceptable salt thereof, comprises at least one moiety of Formula XXI-6 and at least one moiety of Formula XXI-7 or XXI-8;
(n) the compound of Formula V, or pharmaceutically acceptable salt thereof, comprises at least one moiety of Formula XXI-7 and at least one moiety of Formula XXI-8;
(o) the compound of Formula V, or pharmaceutically acceptable salt thereof, comprises at least one moiety of Formula XXII-5;
(p) the compound of Formula V, or pharmaceutically acceptable salt thereof, comprises at least one moiety of Formula XXII-5 and at least one moiety of Formula XXII-1, XXII-2, XXII-3, or XXII-4;
(q) the compound of Formula V, or pharmaceutically acceptable salt thereof, comprises at least two different moieties of Formulas XXII-1, XXII-2, XXII-3, XXII-4, and XXII-5; or
(r) the compound of Formula V, or pharmaceutically acceptable salt thereof, comprises at least one moiety of Formula XXXI, or a compound selected from Compound 201-427, or a pharmaceutically acceptable salt thereof.

22. The compound of Formula V of claim 21, or pharmaceutically acceptable salt thereof, wherein the moiety of Formula XXII-1 is a moiety of XXII-1-a or XXII-1-b:

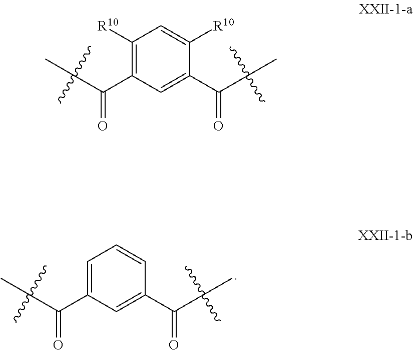

23. A pharmaceutical composition comprising a compound of claim 21, or pharmaceutically acceptable salt thereof, and a pharmaceutically acceptable carrier.

24. A method for antagonizing unfractionated heparin, low molecular weight heparin, or a heparin/low molecular weight heparin derivative comprising administering to a mammal a compound of Formula V:
R¹—[—X-A¹-X—Y-A²-Y—]ₘ—R² V
or a pharmaceutically acceptable salt thereof, wherein:
each of the moiety of —X-A¹-X— is, independently, a moiety of Formula XXI-1, XXI-2, XXI-3, XXI-4, XXI-5, XXI-6, XXI-7, or XXI-8: 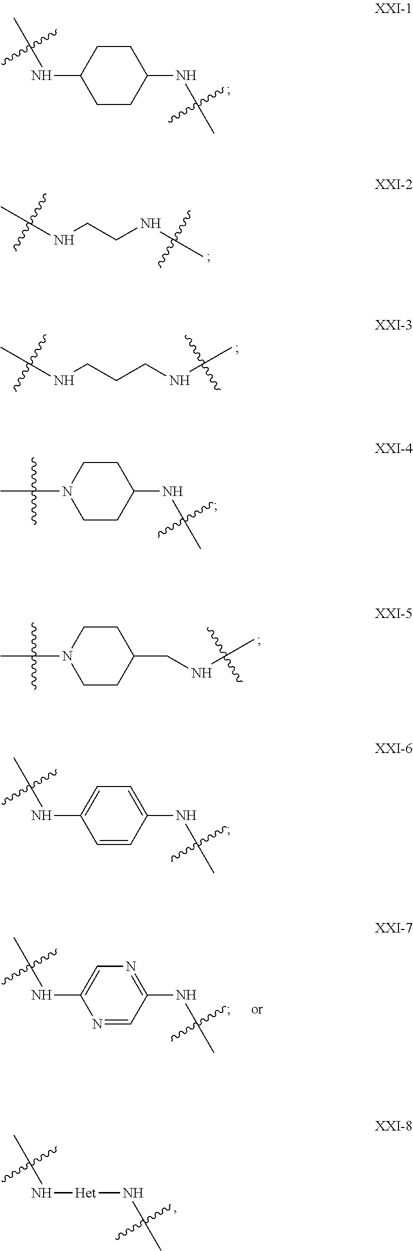
where Het is any 5 or 6-membered ring heterocycle;
each of the moiety of —Y-A²-Y— is, independently, a moiety of Formula XXII-1, XXII-2, XXII-3, XXII-4, or XXII-5: 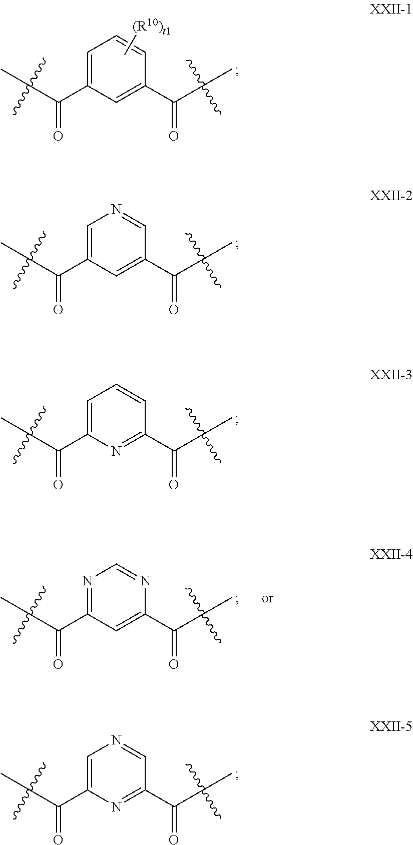
R¹ is hydrogen, —C(═O)R¹¹, or —Y-A²-Y—R¹²;
R² is —OH, —NH₂, —NH(C₁₋₄alkyl), —N(C₁₋₄alkyl)₂, —NH—C(═NH)NH₂, —NH(CH₂)ₚNH₂ wherein p is an integer from 1 to 5, —NH(CH₂)ₚNH(C₁₋₄alkyl) wherein p is an integer from 1 to 5, —NH(CH₂)ₚN(C₁₋₄alkyl)₂ wherein p is an integer from 1 to 5, —NH(CH₂)ₚNHC(═NH)NH₂ wherein p is an integer from 1 to 5, R^{12a}, or —X-A¹-X—R¹³;
each R¹⁰ is, independently, —C(═O)NH₂, C(═O)NH(CH₂)ₚNH₂ wherein p is an integer from 1 to 5, —C(═O)NH(CH₂)ₚNH(C₁₋₄alkyl) wherein p is an integer from 1 to 5, —C(═O)NH(CH₂)ₚN(C₁₋₄alkyl)₂ wherein p is an integer from 1 to 5, —C(═O)NH(CH₂)ₚNHC(═NH)NH₂ wherein p is an integer from 1 to 5, —OCH₃, or —OR^{10a};
each R^{10a} is, independently, C₁₋₈alkyl substituted with R^{A};
each R^{A} is, independently, —NH₂, —NH(C₁₋₄alkyl), —N(C₁₋₄alkyl)₂, —NH—C(═NH)NH₂, —C(═O)NH₂, or —C(═O)OH;
each R¹¹ is, independently, C₁₋₈ alkyl or aryl, each substituted with 0, 1, 2, or 3 substituents each independently selected from —OMe, —OR^{11a}, —C(═O)NH₂, —C(═O)NH(CH₂)ₚNH₂ wherein p is an integer from 1 to 5, —C(═O)NH(CH₂)ₚNH(C₁₋₄alkyl) wherein p is an integer from 1 to 5, —C(═O)NH(CH₂)ₚN(C₁₋₄alkyl)₂ wherein p is an integer from 1 to 5, —C(═O)NH(CH₂)ₚNHC(═NH)NH₂ wherein p is an integer from 1 to 5, —NH₂, —NH(C₁₋₄alkyl), —N(C₁₋₄alkyl)₂, or —NH—C(═NH)NH₂;
each R^{11a} is, independently, C₁₋₈alkyl substituted with R^{B};
each R^{B} is, independently, —NH₂, —NH(C₁₋₄alkyl), —N(C₁₋₄alkyl)₂, —NH—C(═NH)NH₂, or —C(═O)NH₂;
R¹² is —OH, —NH₂, —NH(C₁₋₄alkyl), —N(C₁₋₄alkyl)₂, —NH—C(═NH)NH₂, —NH(CH₂)ₚNH₂ wherein p is an integer from 1 to 5, —NH(CH₂)ₚNH(C₁₋₄alkyl) wherein p is an integer from 1 to 5, —NH(CH₂)ₚN(C₁₋₄alkyl)₂ wherein p is an integer from 1 to 5, —NH(CH₂)ₚNHC(═NH)NH₂ wherein p is an integer from 1 to 5, or R^{12a};
R^{12a} is a moiety of Formula XXXI: 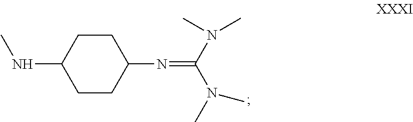
R¹³ is hydrogen or —C(═O)R¹¹;
t1 is 0, 1, or 2; and
m is 1, 2, 3, or 4,
provided that:
(a) the compound of Formula V, or pharmaceutically acceptable salt thereof, comprises at least one moiety of Formula XXI-4;
(b) the compound of Formula V, or pharmaceutically acceptable salt thereof, comprises at least one moiety of Formula XXI-5;
(c) the compound of Formula V, or pharmaceutically acceptable salt thereof, comprises at least two different moieties of Formulas XXI-1, XXI-2, XXI-3, XXI-4, or XXI-5;
(d) the compound of Formula V, or pharmaceutically acceptable salt thereof, comprises at least one moiety of Formula XXI-4 and at least one moiety of Formula XXI-1, XXI-2, XXI-3, or XXI-5;
(e) the compound of Formula V, or pharmaceutically acceptable salt thereof, comprises at least one moiety of Formula XXI-5 and at least one moiety of Formula XXI-1, XXI-2, XXI-3, or XXI-4;
(f) the compound of Formula V, or pharmaceutically acceptable salt thereof, comprises at least one moiety of Formula XXII-2;
(g) the compound of Formula V, or pharmaceutically acceptable salt thereof, comprises at least one moiety of Formula XXII-3;
(h) the compound of Formula V, or pharmaceutically acceptable salt thereof, comprises at least one moiety of Formula XXII-2 and at least one moiety of Formula XXII-1, XXII-3, or XXII-4;
(i) the compound of Formula V, or pharmaceutically acceptable salt thereof, comprises at least one moiety of Formula XXII-3 and at least one moiety of Formula XXII-1, XXII-2, or XXII-4;
(j) the compound of Formula V, or pharmaceutically acceptable salt thereof, comprises at least two different moieties of Formulas XXII-1, XXII-2, XXII-3 and XXII-4;
(k) the compound of Formula V, or pharmaceutically acceptable salt thereof, comprises at least one moiety of Formula XXXI;
(l) the compound of Formula V, or pharmaceutically acceptable salt thereof, comprises at least two different moieties of Formula XXI-6, XXI-7, or XXI-8;
(m) the compound of Formula V, or pharmaceutically acceptable salt thereof, comprises at least one moiety of Formula XXI-6 and at least one moiety of Formula XXI-7 or XXI-8;
(n) the compound of Formula V, or pharmaceutically acceptable salt thereof, comprises at least one moiety of Formula XXI-7 and at least one moiety of Formula XXI-8;
(o) the compound of Formula V, or pharmaceutically acceptable salt thereof, comprises at least one moiety of Formula XXII-5;
(p) the compound of Formula V, or pharmaceutically acceptable salt thereof, comprises at least one moiety of Formula XXII-5 and at least one moiety of Formula XXII-1, XXII-2, XXII-3, or XXII-4;
(q) the compound of Formula V, or pharmaceutically acceptable salt thereof, comprises at least two different moieties of Formulas XXII-1, XXII-2, XXII-3, XXII-4, and XXII-5; or
(r) the compound of Formula V, or pharmaceutically acceptable salt thereof, comprises at least one moiety of Formula XXXI, or a compound selected from Compound 201-427, or a pharmaceutically acceptable salt thereof.

25. The method of claim 24 wherein the low molecular weight heparin is enoxaparin, reviparin, or tinzaparin, and the heparin/low molecular weight heparin derivative is fondaparinux.
